(19) United States
(12) Patent Application Publication
(10) Pub. No.: US 2021/0196828 A1
(43) Pub. Date: Jul. 1, 2021

(54) BACTERIAL DELIVERY VEHICLES FOR IN VIVO DELIVERY OF A DNA PAYLOAD

(71) Applicant: Eligo Bioscience, Paris (FR)

(72) Inventor: Jesus Fernandez Rodriguez, Paris (FR)

(73) Assignee: Eligo Bioscience, Paris (FR)

(21) Appl. No.: 17/138,084

(22) Filed: Dec. 30, 2020

Related U.S. Application Data
(60) Provisional application No. 62/955,278, filed on Dec. 30, 2019.

(51) Int. Cl. A61K 47/26; C12N 15/113; A61K 35/74

(52) U.S. Cl. CPC A61K 47/26; A61K 35/74; C12N 15/113

(57) ABSTRACT

The present disclosure relates generally to bacterial delivery vehicles and their use in efficient transfer of a desired payload into a target bacterial cell of the microbiota of a subject. More specifically, the present disclosure relates to bacterial delivery vehicles with desired host ranges that can be used to efficiently transfer the desired payload in vivo to one or more target bacterial cells of the microbiota of a subject.

### CROSS-REFERENCE TO RELATED APPLICATIONS

This application claims priority to U.S. provisional 62/955,278 filed Dec. 30, 2019, the entire disclosure of which is incorporated by reference herein.

### REFERENCE TO SEQUENCE LISTING SUBMITTED VIA EFS-WEB

This application includes an electronically submitted sequence listing in .txt format. The .txt file contains a sequence listing entitled “01750327.TXT” The sequence listing contained in this .txt file is part of the specification and is hereby incorporated by reference herein in its entirety.

### TECHNICAL FIELD

The present disclosure relates generally to bacterial delivery vehicles and their use in efficient transfer of a desired payload into a target bacterial cell population. More specifically, the present disclosure relates to bacterial delivery vehicles with desired host range that can be used to efficiently transfer in vivo the desired payload to one or more target bacterial cell populations of the microbiome.

### BACKGROUND

Encapsidated DNA in bacterial delivery particles can be used as a method to deliver genetic material into a target bacterial population. Several systems exist that allow the packaging of exogenous DNA into phage particles, for example bacteriophage lambda, in a laboratory setting. Such systems include, for example, a system that directly produces the packaged particles in a bacterial cell and in vitro cell-free systems [1]-[3]. These systems exploit the fact that the addition of a cognate packaging site to an exogenous DNA vector (called phagemid, and more specifically cosmid in the presence of a cos packaging site) allows for the efficient packaging of this payload into a mature viral particle. This approach has been used in many different applications, for example for the generation of cosmid libraries or the transduction of specific genes into bacteria [2], [4]. Most of these transduction assays are performed in laboratory conditions: cells are cultured in a controlled growth media, such as LB, and the transduction protocol is carried out in a buffer with known solute concentrations.

For in vivo applications, such as oral delivery of encapsidated DNA into particles, the need exists for bacterial delivery vehicles that can be given at high enough concentrations to reach all the target cells; hence, a payload that gives high enough titers is essential to optimize the in vivo activity as well as the manufacturing process. The packaged DNA into particles also needs to be able to bind its target cell strongly and long enough for the injection process to occur.

### SUMMARY

Although it was previously shown that the presence of side tail fiber is not necessary for lambda-mediated in vitro transduction experiments in K-12 laboratory strains [5], it is demonstrated herein that side tail fiber is actually necessary to optimize in vivo activity in a treated subject. The present disclosure provides delivery vehicles for in vivo delivery to a target host bacterium of interest, such as those in the gut of a treated subject.

The present disclosure relates to lambdoïd bacterial delivery vehicles and their use in efficient in vivo transfer of a desired payload into a target bacterial cell. The desired payload includes nucleic acid molecules that encode a gene of interest.

A lambdoïd bacterial delivery vehicle for use in in vivo delivery of a DNA payload of interest into a targeted bacterial cell population is provided. In one embodiment, the bacterial delivery vehicle comprises one or more receptor binding protein(s) (RBP). As used herein, a receptor binding protein or RBP is a polypeptide that recognizes, and optionally binds and/or modifies or degrades a substrate located on the bacterial outer envelope, such as, without limitation, bacterial outer membrane, LPS, capsule, protein receptor, channel, structure such as the flagellum, pili, secretion system. The substrate can be, without limitation, any carbohydrate or modified carbohydrate, any lipid or modified lipid, any protein or modified protein, any amino acid sequence, and any combination thereof.

In one embodiment, the bacterial delivery vehicle comprises one or more RBPs selected from the group consisting of a functional lambdoïd side tail fiber protein (herein “STF protein”), a functional lambdoïd gpJ protein and a functional lambdoïd gpH protein. In another embodiment, the bacterial delivery vehicle may comprise two or more proteins selected from the group consisting of a functional lambdoïd side tail fiber protein (herein “STF protein”), a functional lambdoïd gpJ protein and a functional lambdoïd gpH protein. In a particular embodiment, the bacterial delivery vehicle comprises a functional lambdoïd side tail fiber protein (herein “STF protein”) and a functional lambdoïd gpJ protein. In yet another embodiment, the bacterial delivery vehicle comprises a functional lambdoïd side tail fiber protein (herein “STF protein”), a functional lambdoïd gpJ protein and a functional lambdoïd gpH protein. In another aspect, the bacterial delivery vehicle comprises a functional lambdoïd gpJ protein and a functional lambdoïd gpH protein. In another aspect, the bacterial delivery vehicle comprises (i) a functional lambdoïd side tail fiber protein. In addition to a functional STF protein, the bacterial delivery vehicle may further comprise (ii) a functional lambdoïd gpJ protein; and optionally (iii) a functional lambdoïd gpH protein.

In an embodiment, the STF protein, the gpJ protein and/or the gpH protein are a wild type lambda STF, gpJ and/or gpH protein. Alternatively, the STF protein, the gpJ protein and/or the gpH protein are recombinant proteins, preferably non-naturally occurring recombinant proteins. In particular, the recombinant STF protein, gpJ protein and/or gpH protein may be engineered to target the transfer of the DNA payload of interest into the targeted bacterial cell. In a non-limiting example, the recombinant STF protein may be engineered to advantageously possess enzymatic activity such as depolymerase activity and the target bacterial cell may be an encapsulated bacterial cell. Such depolymerase activity is found to increase delivery efficiency, and includes activity associated with an endosialidase such as, for example, a K1F endosialidase or activity associated with a lyase such as, for example, K5 lyase.

Recombinant STF proteins include, for example, engineered chimeric STF proteins and in some instances, the disclosure provides their associated chaperone (also called accessory) proteins. Such chaperone proteins assist in the folding of the chimeric STF protein. The recombinant engineered chimeric STF protein may comprise a fusion between a portion of a STF protein derived from a lambdoïd bacteriophage, preferably a lambda or lambda-like bacteriophage, and a portion of a STF protein derived from a corresponding STF protein derived from a different bacteriophage. Such chimeric STF protein may comprise a fusion between the N-terminal domain of a STF from a lambdoïd bacteriophage, preferably a lambda or lambda-like bacteriophage, and the C-terminal domain of a different STF. In an embodiment, the chimeric STF protein comprises or consists of the amino acid sequence of SEQ ID NO: 14, the amino acid sequence of SEQ ID NO: 16, the amino acid sequence of SEQ ID NO: 17, the amino acid sequence of SEQ ID NO: 19, the amino acid sequence of SEQ ID NO: 21, or the amino acid sequence of SEQ ID NO: 50. In a particular embodiment, the chimeric STF protein is STF-V10 (SEQ ID NO: 44). Other examples of chimeric STF proteins include STF-V10f (SEQ ID NO: 45), STF-V10a (SEQ ID NO: 46) and STF-V10h (SEQ ID NO: 47). The present disclosure also provides synthetic bacterial delivery vehicles that are characterized by the presence of an engineered branched receptor binding multi-subunit protein complex (“branched-RBP”). The engineered branched-RBP comprises two or more associated receptor binding proteins, derived from bacteriophages, which associate with one another based on the presence of interaction domains (IDs). In a particular embodiment, said engineered branched-RBP comprises two or more associated STF, derived from bacteriophages, which associate with one another based on the presence of IDs. The association of one subunit with another can be non-covalent or covalent. Each of the polypeptide subunits contain IDs that function as “anchors” for association of one subunit RBP with another. In specific embodiments the branched-RBP may comprise multiple RBP subunits, including, for example, two, three, four, etc. subunits. The individual RBP subunit may bring different biological functions to the overall engineered branched-RBP. Such functions include but are not limited to host recognition and enzymatic activity. Such enzymatic activity includes depolymerase activity.

Accordingly, bacterial delivery vehicles are provided which enable transfer of a nucleic acid payload, encoding a protein or nucleic acid of interest, into a desired target bacterial host cell wherein said bacterial delivery vehicles are characterized by having a chimeric STF and/or a branched-RBP as disclosed herein.

Bacterial delivery vehicles are also provided that comprise recombinant gpJ proteins. Such gpJ proteins include recombinant gpJ proteins, including chimeric proteins, that permit recognition of a bacterial cell receptor other than the LamB OMP receptor which is the natural lambda phage receptor on the bacterial cell surface (14). The recombinant engineered chimeric gpJ protein may comprise a fusion between a portion of a gpJ protein derived from a lambdoïd bacteriophage, preferably a lambda or lambda-like bacteriophage, and a portion of a gpJ protein derived from a corresponding gpJ protein derived from a different bacteriophage. Such chimeric gpJ protein may comprise a fusion between the N-terminal domain of a gpJ protein from a lambdoïd bacteriophage, preferably a lambda or lambda-like bacteriophage, and the C-terminal domain of a different gpJ protein. In an embodiment, the gpJ protein comprises or consists of the amino acid sequence of SEQ ID NO: 10, 11, 12, 13 or 49.

Bacterial delivery vehicles are also provided that comprise recombinant gpH proteins. Such gpH proteins include recombinant gpH proteins that permit or allow improved entry of bacterial vectors in cells having deficiencies or alterations in permease complexes. The recombinant engineered chimeric gpH protein may comprise a fusion between a portion of a gpH protein derived from a lambdoïd bacteriophage, preferably a lambda or lambda-like bacteriophage, and a portion of a gpH protein derived from a corresponding gpH protein derived from a different bacteriophage. Such chimeric gpH protein may comprise a fusion between the N-terminal domain of a gpH protein from a lambdoïd bacteriophage, preferably a lambda or lambda-like bacteriophage, and the C-terminal domain of a different gpH protein. In an embodiment, the gpH protein comprises or consists of the amino acid sequence of SEQ ID NO: 23 or 24.

In certain aspects, the bacterial delivery vehicles provided herein, are vehicles wherein the recombinant STF protein, gpJ protein and/or gpH protein are engineered to increase the efficiency of transfer of the DNA payload into the targeted bacterial cell. Such bacterial cell may be selected from the group consisting of Yersinia spp., Escherichia spp., Klebsiella spp., Acinetobacter spp., Pseudomonas spp., Helicobacter spp., Vibrio spp, Salmonella spp., Streptococcus spp., Staphylococcus spp., Bacteroides spp., Clostridium spp., Shigella spp., Enterococcus spp., Enterobacter spp., Listeria spp, and mixtures thereof, preferably from the group consisting of E. coli. and other bacterial species of interest such as, for example, Klebsiella, Citrobacter, Agrobacterium, Enterobacter or Pseudomonas, more preferably is E. coli.

The bacterial delivery vehicles disclosed herein provide a means for transfer, including in vivo transfer, of a DNA payload of interest into a targeted host bacterium. In non-limiting aspects, the DNA payload comprises a nucleic acid of interest selected from the group consisting of Cas nuclease gene, a Cas9 nuclease gene, a guide RNA, a CRISPR locus, a toxin gene, a gene encoding an enzyme such as a nuclease or a kinase, a TALEN, a ZFN, a meganuclease, a recombinase, a bacterial receptor, a membrane protein, a structural protein, a secreted protein, a gene encoding resistance to an antibiotic or to a drug in general, a gene encoding a toxic protein or a toxic factor, and a gene encoding a virulence protein or a virulence factor, or any of their combination. In specific embodiments, the nucleic acid of interest encodes a therapeutic protein. Still further, the nucleic acid of interest may encode an antisense nucleic acid molecule.

In one aspect, the bacterial delivery vehicle enables the transfer of a nucleic acid payload that encodes a nuclease that targets cleavage of a host bacterial cell genome or a host bacterial cell plasmid. In some aspects, the cleavage occurs in an antibiotic resistant gene. In another embodiment, the nuclease mediated cleavage of the host bacterial cell genome is designed to stimulate a homologous recombination event for insertion of a nucleic acid of interest into the genome of the bacterial cell.

The present disclosure also provides pharmaceutical or veterinary compositions comprising one or more of the bacterial delivery vehicles disclosed herein and a pharmaceutically acceptable carrier. Also provided is a method for treating a disease or disorder caused by bacteria, preferably a bacterial infection, comprising administering to a subject having a disease or disorder caused by bacteria, preferably a bacterial infection, in need of treatment, the provided pharmaceutical or veterinary composition. The present disclosure also relates to a pharmaceutical or veterinary composition or a bacterial delivery vehicle as disclosed herein for use in the treatment of a disease or disorder caused by bacteria, preferably a bacterial infection. It further relates to the use of a pharmaceutical or veterinary composition or a bacterial delivery vehicle as disclosed herein for the manufacture of a medicament for treating a disease or disorder caused by bacteria, preferably a bacterial infection. The disease or disorder caused by bacteria is preferably selected from a bacterial infection, a metabolic disorder and a pathology involving bacteria of the human microbiome. More preferably, the disease or disorder caused by bacteria is a bacterial infection. A method for reducing the amount of virulent and/or antibiotic resistant bacteria in a bacterial population is provided comprising contacting the bacterial population with the bacterial delivery vehicles disclosed herein. The method may be an in vivo or in vitro method. The present disclosure also relates to a pharmaceutical or veterinary composition or a bacterial delivery vehicle as disclosed herein for use in reducing the amount of virulent and/or antibiotic resistant bacteria in a bacterial population, in particular in a subject having a bacterial infection. It further relates to the use of a pharmaceutical or veterinary composition or a bacterial delivery vehicle as disclosed herein for the manufacture of a medicament for reducing the amount of virulent and/or antibiotic resistant bacteria in a bacterial population, in particular in a subject having a bacterial infection.

In another aspect, the methods and compositions described herein provide long term stable expression of a gene of interest in the microbiome of a host. In such an instance, the delivery vehicle comprises a nucleic acid molecule encoding the gene of interest wherein the nucleic acid is engineered to either integrate into the bacterial chromosome or, alternatively, stably replicate within the targeted microbiome of the host. Once delivered into the bacteria of interest, i.e., the microbiome, the gene of interest will typically be expressed. The methods and compositions described herein encompass in-situ bacterial production of any compound of interest, including therapeutic compounds such as prophylactic and therapeutic vaccines for mammals. The compound of interest can be produced inside the targeted bacteria, secreted from the targeted bacteria or expressed on the surface of the targeted bacteria. In a more particular embodiment, an antigen is expressed on the surface of the targeted bacteria for prophylactic and/or therapeutic vaccination.

### BRIEF DESCRIPTION OF FIGURES

In order to better understand the subject matter that is disclosed herein and to exemplify how it may be carried out in practice, embodiments will now be described, by way of non-limiting example, with reference to the accompanying drawings. With specific reference to the drawings, it is stressed that the particulars shown are by way of example and for purposes of illustrative discussion of embodiments of the disclosure.

FIG. 1. Presence of transductants after oral gavage of lambda-PaPa packaged psgRNAcos cosmids (DNA payload of SEQ ID NO: 1). Black dots, total number of MG-GFP cells. White dots, MG-GFP cells with acquired kanamycin resistance.

FIG. 2. In vivo adaptation of lambda PaPa phages on MG-GFP in the gut of mice (n=3). Each line corresponds to an experiment performed in one mouse. X axis, days.

FIG. 3 Presence of transductants after oral gavage of Ur-lambda packaged pJ23104-GFP cosmids (3 kbp) (DNA payload of SEQ ID NO: 2). Black dots, total number of MG1655-Str cells. White dots, MG-GFP cells with acquired chloramphenicol resistance.

FIG. 4. Presence of transductants after oral gavage of Ur-lambda packaged pJF1 cosmids (7 kb) (DNA payload of SEQ ID NO: 3). Black dots, total number of MG-GFP cells. White dots, MG-GFP cells with acquired chloramphenicol resistance.

FIG. 5. Titration of packaged lambda phagemids with different payload sizes.

FIG. 6A-B. Delivery efficiency after oral gavage of Ur-lambda packaged GG6K and GG8K cosmids (respectively of SEQ ID NO: 6 and SEQ ID NO: 7) with or without stf. FIG. 6A. Packaged phagemids with STF. FIG. 6B Packaged phagemids without STF.

FIG. 7. Alignment of gpJ variants to lambda gpJ. Two insertion points based on protein identity, marked with boxes 1 and 2, were chosen to generate chimeras with the lambda gpJ.

FIG. 8A-D. Apparent titers of different gpJ chimeras. FIG. 8A. 591 chimeras, inserted into lambda gpJ using the second insertion point (box #2 in FIG. 7). Lambda WT refers to the original gpJ variant recognizing LamB. Each lane represents a 10-fold dilution of the produced packaged phagemid, from the most concentrated on the right to the most diluted on the left. FIG. 8B. Apparent titers of gpJ variants lambda WT, Z2145 and 1A2 (respectively of SEQ ID NO: 10, SEQ ID NO: 12 and SEQ ID NO: 13) in three strains: MG-GFP (black bars), MG-delta-LamB (white bars) and H10-waaJ (O157 strain lacking the O157 antigen, grey bars). FIG. 8C. Delivery efficiency (% GFP+ cells) measured in a flow cytometer of H10 wt strain (contains a group 4 capsule) using a lambda packaged phagemid with a gpJ Z2145 variant (SEQ ID NO: 12) or a Z2145 (SEQ ID NO: 12) with WW11.2 stf variant (SEQ ID NO: 16). FIG. 8D. Delivery efficiency (% GFP+ cells) measured in a flow cytometer with MG1655 or MG1656-OmpCO157 transduced with lambda packaged phagemid comprising the gpJ variants A8 (SEQ ID NO: 49) or 1A2 (SEQ ID NO: 13) and a chimeric lambda-P2 STF (SEQ ID NO: 50)

FIG. 9 A-B. Analysis of lambda gpH and generation of engineered variants. FIG. 9A. Protein alignment between lambda gpH and a gpH protein from another lambdoïd prophage found in E. coli. FIG. 9B. Titration of lambda WT gpH variants (left panels—SEQ ID NO: 23) and engineered gpH-IAI (right panels—SEQ ID NO: 24) in MG1655, manZ and manY mutants. Each lane represents a 10-fold dilution of the produced packaged phagemid, from the most concentrated on the right to the most diluted on the left.

FIG. 10. Delivery efficiency of engineered lambda packaged phagemids in other Proteobacteria. Dot titrations of different gpJ and STF combinations on an Enterobacter cloacae strain. 10 μL of packaged phagemids were mixed with 90 μL of bacteria at an OD600 ^{˜}0.7, incubated for 30 min at 37° C. and 10 μL of the reaction plated on LB Agar plus 25 μg/mL chloramphenicol.

FIG. 11. Stability of 1A2-STF118 or 1A2-STF29 packaged phagemids in PBS. Grey bars, PBS only; white bars, PBS plus pancreatin at pH 6.8. Left group of bars, activity in MG1656-OmpCO157; right group of bars, LMR 503 strain. Y axis shows particle titer per

FIG. 12. Overlay of the sedimentation coefficient distribution data of the 3 Eligobiotics® (EB) batches analyzed by svAUC in Example 3. The integration ranges for EB packaged with 3 or 4 copies of the payload are depicted by dotted lines.

FIG. 13. Relative abundance of Eligobiotics® comprising either 3 or 4 copies of their payload. Absorbance signals at 260 and 280 nm for each population defined in svAUC were integrated and used to calculate their relative abundance in each batch of Eligobiotics®.

### DETAILED DESCRIPTION

The present disclosure relates to bacterial delivery vehicles with desired host ranges that can be used to efficiently transfer the desired payload in vivo to one or more target bacterial cells of the microbiota of a subject.

Disclosed herein are methods and compositions for in vivo delivery of a desired payload into the microbiome of a subject. Such delivery vehicles are engineered to contain as part of their payload, nucleic acids encoding RNA molecules or proteins that may be useful for treatment of disorders and diseases of a subject. Such nucleic acids may encode generally, any molecules, compounds and proteins, as non-limiting examples.

The bacterial delivery vehicles provided herein enable transfer of a nucleic acid payload, encoding a protein or nucleic acid of interest, into a desired target bacterial host cell. As used herein, the term “delivery vehicle” refers to any means that allows the transfer of a payload into a bacterium. There are several types of delivery vehicles encompassed by the present disclosure including, without limitation, bacteriophage scaffold, virus scaffold, chemical based delivery vehicle (e.g., cyclodextrin, calcium phosphate, cationic polymers, cationic liposomes), protein-based or peptide-based delivery vehicle, lipid-based delivery vehicle, nanoparticle-based delivery vehicles, non-chemical-based delivery vehicles (e.g., transformation, electroporation, sonoporation, optical transfection), particle-based delivery vehicles (e.g., gene gun, magnetofection, impalefection, particle bombardment, cell-penetrating peptides) or donor bacteria (conjugation). Any combination of delivery vehicles is also encompassed by the present disclosure. The delivery vehicle can refer to a bacteriophage derived scaffold and can be obtained from a natural, evolved or engineered capsid.

In one aspect, bacterial delivery vehicles with desired target host ranges are provided for use in in vivo transfer of a payload to the microbiome of a subject. The bacterial delivery vehicle may comprise one or more proteins selected from the group consisting of a functional lambdoïd side tail fiber protein (herein “STF protein”), a functional lambdoïd gpJ protein and a functional lambdoïd gpH protein. In another embodiment, the bacterial delivery vehicle may comprise two or more proteins selected from the group consisting of a functional lambdoïd side tail fiber protein, a functional lambdoïd gpJ protein and a functional lambdoïd gpH protein. In a particular embodiment, the bacterial delivery vehicle comprises a functional lambdoïd STF protein and a functional lambdoïd gpJ protein. In yet another embodiment, the bacterial delivery vehicle may comprise a functional lambdoïd STF protein, a functional lambdoïd gpJ protein and a functional lambdoïd gpH protein. In another aspect, the bacterial delivery vehicle may comprise a functional lambdoïd gpJ protein and a functional lambdoïd gpH protein. In another aspect, the bacterial delivery vehicle may comprise (i) a functional lambdoïd STF protein. In addition to a functional STF protein, the bacterial delivery vehicle may further comprise (ii) a functional lambdoïd gpJ protein; and optionally (iii) a functional lambdoïd gpH protein.

In an embodiment, the functional STF protein, the functional gpJ protein and/or the functional gpH protein are respectively wild type lambda STF, gpJ and/or gpH proteins. Alternatively, the functional STF protein, the functional gpJ protein and/or the functional gpH protein are recombinant proteins.

As used herein, the term “recombinant protein” refers to non-naturally occurring proteins, in particular engineered proteins obtained by recombination technique. Such recombinant proteins include, for example, engineered chimeric proteins.

As used herein, a functional protein means in general a protein with a biological activity; more specifically a functional wild type, recombinant protein, variant, fusion or fragment herein relates to a wild type, recombinant protein, variant, fusion or fragment contributing to the efficient delivery of a DNA payload into a target strain. The efficiency threshold depends on a number of factors such as the type of protein, type of target strain and type of environment. For instance, STF and gpJ proteins allow for recognition, binding (and in some cases also degradation) of an extracellular epitope such as LPS, capsules and outer membrane proteins; gpH proteins allow for an efficient injection and hence successful passage of the DNA payload through the periplasm.

In the context of the present disclosure, a protein, such as STF, gpJ and gpH proteins, may be determined as being functional by titrating packaged phagemids containing said protein on bacterial cells known to display receptors recognized by said protein and comparing it to the titer obtained with the same packaged phagemids on bacterial cells known to display receptors which are not recognized by said protein.

Such recombinant chimeric STF protein may comprise a fusion between a portion of a STF protein derived from a lambdoïd bacteriophage, preferably a lambda or lambda-like bacteriophage, and a portion of a STF protein derived from a STF protein derived from a different bacteriophage (herein referred to also as a “chimeric receptor binding protein” or “chimeric RBP”), in particular from a different lambdoïd bacteriophage or from a non-lambdoïd bacteriophage. Such chimeric STF protein may comprise a fusion between the N-terminal domain of a STF protein from a lambdoïd bacteriophage, preferably a lambda or lambda-like bacteriophage, and the C-terminal domain of a different STF. As used herein, a receptor binding protein or RBP may be a STF derived polypeptide that recognizes, and optionally binds and/or modifies or degrades a substrate located on the bacterial outer envelope, such as, without limitation, bacterial outer membrane, LPS, capsule, protein receptor, channel, structure such as the flagellum, pili, secretion system. The substrate can be, without limitation, any carbohydrate or modified carbohydrate, any lipid or modified lipid, any protein or modified protein, any amino acid sequence, and any combination thereof.

As used herein, lambdoïd bacteriophages comprise a group of related viruses that infect bacteria. The viruses are termed lambdoïd because one of the first members to be described was lambda (λ). Lambdoid bacteriophages are members of the Caudovirus order (also known as tailed bacteriophages) and include those bacteriophages with similar lifestyles, including, for example, the ability to recombine when intercrossed, possession of identical pairs of cohesive ends, and prophages that are inducible by ultraviolet irradiation. Although members of the order may have genomes that vary at the nucleotide level, they carry regions of sufficient nucleotide sequence identity to guide recombination between themselves typically giving rise to a fully functional phage that has all the necessary genes. (See, for example, Casjens and Hendrix (2015) Virology 479-480:310-330). For purposes of the present disclosure, lambdoïd bacteriophages for use as delivery vehicles, as well as lambdoïd STF, gpH and gpJ proteins for use, would be understood generally by one skilled in the art.

Lambdoid phages can be defined as belonging to the lambda supercluster based on genomic analysis [6]. Within this supercluster, several clusters can be distinguished, each having a prototypical phage. The phage-like clusters and their members (between brackets) are: Lambda-like (lambda (k), HK630, HK629), phi80-like (phi80, HK225, mEp237), N15-like (N15, PY54, phiKO2), HK97-like (HK97, HK022, HK75, HK106, HK140, HK446, HK542, HK544, HK633, mEpX1, mEpX2, mEp234, mEp235, mEp390, ENT39118), ES18-like (ES18, Oslo, SPN3UB), Gifsy-2-like (gifsy-2, gifsy-1, Fels-1, mEp043, mEp213, CP-1639, CTD-Iø, mEp640, FSL SP-016), BP-4795-like (BP-4795, 2851, stx2-1717, YYZ-2008), SfV-like (SfV, SfII, SfIV, SfI, øP27, ST64B), P22-like (P22, L, SPN9CC, ST64T, ST104, ST160, epsilon34, g341, SE1, Emek, φ20, IME10, Sf6, HK620, CUS-3, SPC-P1), APSE-1-like (APSE-1, APSE-2), 933W-like (933W, stx1ø, stx2ø-I, stx2ø-II, stx2-86, min27, ø24B, P13374, TL-2011c, VT2-sakai, VT2ø_272), HK639-like (HK639), øES15-like (øES15), HS2-like (HS2), ENT47970-like (ENT47670), ZF40-like (ZF40), øEt88-like (øEt88).

In the present disclosure a lambdoïd STF protein includes, for example, a protein comprising or consisting of an amino acid sequence having at least 75% identity up to amino acid corresponding to amino acid 130 of lambda STF (Uniprot P03764 SEQ ID NO: 14), in particular up to amino acid 130 of said lambda STF; a lambdoïd gpJ protein includes, for example, a protein comprising or consisting of an amino acid sequence having at least 35% identity up to an amino acid corresponding to amino acid 606 of lambda gpJ (Uniprot P03749 SEQ ID NO: 10), in particular up to amino acid 606 of said lambda gpJ; and a lambdoïd gpH protein includes, for example, a protein comprising or consisting of an amino acid sequence having at least 40% identity over the complete length of lambda gpH (Uniprot P03736 SEQ ID NO: 23) and considering that the stretch of amino acids between positions 189 and 391 may bear little or no identity at all. A lambdoïd bacterial delivery vehicle includes a bacterial delivery vehicle comprising a functional lambdoïd stf protein and/or a functional lambdoïd gpJ protein and/or functional lambdoïd gpH protein, which each may have an altered host range compared to the wild-type lambda phage.

In one aspect, the STF protein includes a protein that comprises or consists of an amino acid sequence with at least 80, 85, 90, 95, 96, 97, 98, or 99% sequence identity with the wild type lambda stf protein amino acid sequence of SEQ ID NO: 14, or with any of the recombinant STF proteins, fusions, variants or fragments disclosed herein. In one aspect, the gpJ protein includes a protein that comprises or consists of an amino acid sequence with at least 80, 85, 90, 95, 96, 97, 98, or 99% sequence identity with the wild type gpJ protein amino acid sequence of SEQ ID NO: 10, or with any of the recombinant gpJ proteins, fusions, variants or fragments disclosed herein. In one aspect, the gpH protein includes a protein that comprises or consists of an amino acid sequence with at least 80, 85, 90, 95, 96, 97, 98, or 99% sequence identity with the wild type gpH protein amino acid sequence of SEQ ID NO: 23, or with any of the recombinant gpH proteins, fusions, variants or fragments disclosed herein. In another aspect, nucleic acids encoding for such wild type, or recombinant, STF, gpH and gpJ proteins are provided herein.

As used herein, the percent homology between two sequences is equivalent to the percent identity between the two sequences. The percent identity is calculated in relation to polymers (e.g., polynucleotide or polypeptide) whose sequences have been aligned. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4: 11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using a BLOSUM62 matrix, a BLOSUM30 matrix or a PAM250 matrix, and a gap weight of 16, 14, 12, 10, 8, 6, or 4 and a length weight of 1, 2, 3, 4, 5, or 6. In a specific embodiment the BLOSUM30 matrix is used with gap open penalty of 12 and gap extension penalty of 4.

A variety of different lambdoïd bacterial delivery vehicles are provided as a means for transfer of a payload into a target bacterial cell population. Such bacterial delivery vehicles include those that comprise one or more wild type lambdoïd STF, gpH and gpJ proteins. Alternatively, the delivery vehicles may comprise one or more wild type STF, gpH, or gpJ proteins combined with one or more recombinant STF, gpH or gpJ proteins, including chimeric proteins, fusions, variants or fragments as disclosed herein. Included are delivery vehicles wherein the three STF, gpH and gpJ proteins are wild-type and those delivery vehicles wherein the three STF, gpH and gpJ proteins are recombinant, fusions, variants or fragments.

The present disclosure provides delivery vehicles, for example, comprising a chimeric receptor binding protein (RBP), wherein the chimeric RBP comprises a fusion between an N-terminal domain of a RBP from a lambdoïd bacteriophage, preferably a lambda or lambda-like bacteriophage, and a C-terminal domain of a different bacteriophage RBP. Such bacteriophage RBPs, from which the chimeric RBP are derived, include, for example, “L-shape fibers”, “side tail fibers (STFs)”, “long tail fibers” or “tailspikes.” Such bacteriophage RBPs, from which the chimeric RBP are derived, may be wild-type RBPs or RBP variants, preferably wild-type RBPs. Such chimeric RBPs include those having an altered host range and/or biological activity such as, for example, depolymerase activity. In an embodiment, the chimeric RBPs have a host range that is directed to specific bacterial cells of the host or subject microbiome. In one specific aspect, the different RBP of the chimeric receptor binding protein (RBP) is derived from any bacteriophage or from any bacteriocin.

Such chimeric RBP may comprise a fusion between the N-terminal domain of a RBP from a lambdoïd bacteriophage, preferably a lambda or lambda-like bacteriophage, and the C-terminal domain of a different RBP. The N-terminal domain is typically fused to the N-terminal end of the C-terminal domain.

By “N-terminal domain” of a RBP, in particular of a STF protein, from a bacteriophage is meant herein an amino acid region of said RBP starting at the N-terminal end of said RBP and ending at positions 80-150, 320-460 or 495-560 of said RBP, said positions being with reference to the lambda bacteriophage STF sequence (SEQ ID NO: 14). By “C-terminal domain” of a RBP, in particular of a STF protein, from a bacteriophage is meant herein an amino acid region of said RBP starting at positions 25-150, 320-460 or 495-560 of said RBP, said positions being with reference to the lambda bacteriophage STF sequence (SEQ ID NO: 14), and ending at the C-terminal end of said RBP.

In an embodiment, the bacterial delivery vehicles contain a chimeric RBP comprising a fusion between an N-terminal domain of a RBP derived from a lambdoïd bacteriophage, preferably a lambda or lambda-like bacteriophage, and a C-terminal domain of a different RBP wherein said N-terminal domain of the chimeric RBP is fused to said C-terminal domain of a different RBP within one of the amino acids regions selected from positions 80-150, 320-460, or 495-560 of the N-terminal domain with reference to the lambda bacteriophage STF sequence (SEQ ID NO: 14). In one aspect, the RBP from the lambdoïd bacteriophage, preferably the lambda or lambda-like bacteriophage, and the different RBP contain homology in one or more of three amino acids regions ranging from positions 80-150, 320-460, and 495-560 of the RBP with reference to the lambda bacteriophage STF sequence (SEQ ID NO: 14). In certain aspects, the homology is around 35% identity for 45 amino acids or more, around 50% identify for 30 amino acids or more, or around 90% identity for 18 amino acids or more within the one or more of three amino acids regions ranging from positions 80-150, 320-460, and 495-560 of the RBP with reference to the lambda bacteriophage STF sequence. In one specific aspect, the different RBP domain of the chimeric RBP is derived from a bacteriophage or a bacteriocin. In one aspect, the chimeric RBP comprises an N-terminal domain of a RBP fused to a C-terminal domain of a different RBP within one of the amino acids regions selected from positions 80-150, 320-460, or 495-560 of the N-terminal RBP domain with reference to the lambda bacteriophage STF sequence (SEQ ID NO: 14). In another non-limiting embodiment, the chimeric RBP comprises an N-terminal domain of a RBP and a C-terminal domain of a different RBP fused within a site of the N-terminal RBP domain having at least 80%, 85%, 90%, 95%, 99% or 100% identity with a site selected from the group consisting of amino acids SAGDAS (SEQ ID NO: 37), ADAKKS (SEQ ID NO: 38), MDETNR (SEQ ID NO: 39), SASAAA (SEQ ID NO: 40), and GAGENS (SEQ ID NO: 41).

In a specific embodiment, the chimeric STF protein comprises a fusion between the N-terminal domain of a lambda bacteriophage STF protein and the C-terminal domain of a STF protein from another bacteriophage, said N-terminal domain being in particular fused to said C-terminal domain within the amino acid region 495-560 of the N-terminal domain with reference to the lambda bacteriophage STF protein sequence (SEQ ID NO: 14). In said embodiment, the chimeric STF variant may be STF-V10 comprising or consisting of the amino acid sequence SEQ ID NO: 44 and typically encoded by the nucleotide sequence SEQ ID NO: 51. Alternatively, in said embodiment, the chimeric STF variant may be WW11.2 comprising or consisting of the amino acid sequence SEQ ID NO: 16 and typically encoded by the nucleotide sequence SEQ ID NO: 30. Still alternatively, in said embodiment, the chimeric STF variant may be STF75 comprising or consisting of the amino acid sequence SEQ ID NO: 17 and typically encoded by the nucleotide sequence SEQ ID NO: 31. In said embodiment, said STF75 may be, in particular be produced, with its associated chaperone protein, which typically comprises or consists of the amino acid sequence SEQ ID NO: 18, and is typically encoded by the nucleic acid sequence SEQ ID NO: 34. Still alternatively, in said embodiment, the chimeric STF variant may be STF23 comprising or consisting of the amino acid sequence SEQ ID NO: 21 and typically encoded by the nucleotide sequence SEQ ID NO: 35. In said embodiment, said STF23 may be, in particular be produced, with its associated chaperone protein, which typically comprises or consists of the amino acid sequence SEQ ID NO: 22 and is typically encoded by the nucleic acid sequence SEQ ID NO: 36. In another embodiment, the chimeric STF protein comprises a fusion between the N-terminal domain of a lambda bacteriophage STF protein and the C-terminal domain of a STF protein from another bacteriophage, said N-terminal domain being in particular fused to said C-terminal domain within the amino acid region 320-460 of the N-terminal domain with reference to the lambda bacteriophage STF protein sequence (SEQ ID NO: 14). In said embodiment, the chimeric STF variant may be STF-EB6 comprising or consisting of the amino acid sequence SEQ ID NO: 19 and typically encoded by the nucleotide sequence SEQ ID NO: 33. In said embodiment, said STF-EB6 may be, in particular be produced, with its associated chaperone protein, which typically comprises or consists of the amino acid sequence SEQ ID NO: 20 and is typically encoded by the nucleic acid sequence SEQ ID NO: 32. Alternatively, in said embodiment, the chimeric STF variant may be STF lambda-P2 comprising or consisting of the amino acid sequence SEQ ID NO: 50, and typically encoded by the nucleotide sequence SEQ ID NO: 56. In said embodiment, said STF lambda-P2 may be, in particular be produced, with its associated chaperone protein, which typically comprises or consists of the amino acid sequence SEQ ID NO: 57 and is typically encoded by the nucleic acid sequence SEQ ID NO: 58. Alternatively, the chimeric STF variant may be STF-V10f comprising or consisting of the amino acid sequence SEQ ID NO: 45, and typically encoded by the nucleic acid sequence SEQ ID NO: 52. Alternatively, the chimeric STF variant may be STF-V10a comprising or consisting of the amino acid sequence SEQ ID NO: 46, and typically encoded by the nucleic acid sequence SEQ ID NO: 53. Alternatively, the chimeric STF variant may be STF-V10h comprising or consisting of the amino acid sequence SEQ ID NO: 48, and typically encoded by the nucleic acid sequence SEQ ID NO: 54.

Recombinant RBP proteins as disclosed herein may need their associated chaperone (also called accessory) proteins for proper folding. Such chaperone proteins assist in the folding of the chimeric RBP protein. For example, the lambda STF protein comprising or consisting of the amino acid sequence SEQ ID NO: 14 needs its associated protein, which typically comprises or consists of the amino acid sequence SEQ ID NO: 15, for proper folding. The need of an associated chaperone protein for proper folding of said chimeric RBP protein typically depends on the RBP from which the C-terminal region of the chimeric RBP is derived. For example, the chimeric STF protein STF75 comprising or consisting of the amino acid sequence SEQ ID NO: 17 needs its associated chaperone protein, which typically comprises or consists of the amino acid sequence SEQ ID NO: 18, and is typically encoded by the nucleic acid sequence SEQ ID NO: 34, for proper folding. For example, the chimeric STF protein STF-EB6 comprising or consisting of the amino acid sequence SEQ ID NO: 19 needs its associated chaperone protein, which typically comprises or consists of the amino acid sequence SEQ ID NO: 20 and is typically encoded by the nucleic acid sequence SEQ ID NO: 32, for proper folding. For example, the chimeric STF protein STF23 comprising or consisting of the amino acid sequence SEQ ID NO: 21 needs its associated chaperone protein, which typically comprises or consists of the amino acid sequence SEQ ID NO: 22 and is typically encoded by the nucleic acid sequence SEQ ID NO: 36, for proper folding. For example, the chimeric STF protein STF lambda-P2 comprising or consisting of the amino acid sequence SEQ ID NO: 50 needs its associated chaperone protein, which typically comprises or consists of the amino acid sequence SEQ ID NO: 57 and is typically encoded by the nucleic acid sequence SEQ ID NO: 58, for proper folding. Said chaperone protein may remain attached to said chimeric STF protein after folding. Accordingly, in some embodiments, the bacterial delivery vehicles disclosed herein may further comprise the chaperone protein associated with the chimeric STF protein that said vehicle comprises. Alternatively, said chaperone protein may not remain attached to said chimeric STF protein after folding, and may for example be proteolysed, in particular auto-proteolysed. Accordingly, in some embodiments, the bacterial delivery vehicles disclosed herein do not comprise the chaperone protein associated with the chimeric STF protein that said vehicle comprises.

The present disclosure also provides synthetic bacterial delivery vehicles that are characterized by the presence of an engineered branched receptor binding multi-subunit protein complex (“branched-RBP”). Such delivery vehicles may be used to transfer a payload of interest into a bacterial cell of the microbiome. The engineered branched-RBP comprises two or more associated receptor binding proteins, derived from bacteriophages, which associate with one another based on the presence of interaction domains (IDs). The association of one subunit with another can be non-covalent or covalent. Each of the polypeptide subunits contain IDs that function as “anchors” for association of one subunit RBP with another. In specific embodiments, the branched-RBP may comprise multiple RBP subunits, including, for example, two, three, four, etc. subunits.

The individual RBP subunit may bring different biological functions to the overall engineered branched-RBP. Such functions include but are not limited to host recognition and enzymatic activity. Such enzymatic activity includes depolymerase activity. The two or more associated receptor binding proteins of the branched-RBP include, but are not limited to, chimeric receptor binding proteins (RBPs) described herein that comprise a fusion between the N-terminal domain of a RBP derived from a lambdoïd bacteriophage, preferably a lambda or lambda-like bacteriophage, and the C-terminal domain of a different RBP wherein said chimeric RBP further comprises an ID domain.

Accordingly, bacterial delivery vehicles are provided which enable transfer of a nucleic acid payload, encoding a protein or nucleic acid of interest, into a desired target bacterial host cell wherein said bacterial delivery vehicles are characterized by having a chimeric-RBP or a branched-RBP as disclosed herein. (For chimeric and branched RBPs see, US provisional application U.S. 62/802,777, U.S. application Ser. No. 16/696,769 and U.S. application Ser. No. 16/726,033, each of which is incorporated by reference in their entirety).

Bacterial delivery vehicles are also provided that comprise recombinant gpJ proteins. Such gpJ proteins include recombinant gpJ proteins, including chimeric proteins, that permit recognition of a bacterial cell receptor other than the LamB OMP receptor. It is known that receptor-recognition activity of gpJ lies in the C-terminal part of the protein, with a fragment as small as 249aa conferring capability of binding to the LamB receptor [5]. In a particular embodiment, such chimeric gpJ protein may comprise a fusion between the N-terminal domain of a gpJ protein from a lambdoïd bacteriophage, preferably a lambda or lambda-like bacteriophage, and the C-terminal domain of a different gpJ protein. The N-terminal domain is typically fused to the N-terminal end of the C-terminal domain.

By “N-terminal domain” of a gpJ protein, from a bacteriophage is meant herein an amino acid region of said gpJ protein starting at the N-terminal end of said gpJ protein and ending at positions 810-825 or 950-970 of said gpJ protein, said positions being with reference to the lambda bacteriophage gpJ protein sequence (SEQ ID NO: 10). By “C-terminal domain” of a gpJ protein from a bacteriophage is meant herein an amino acid region of said gpJ protein starting at positions 810-825 or 950-970 of said gpJ protein, said positions being with reference to the lambda bacteriophage gpJ protein sequence (SEQ ID NO: 10), and ending at the C-terminal end of said gpJ protein.

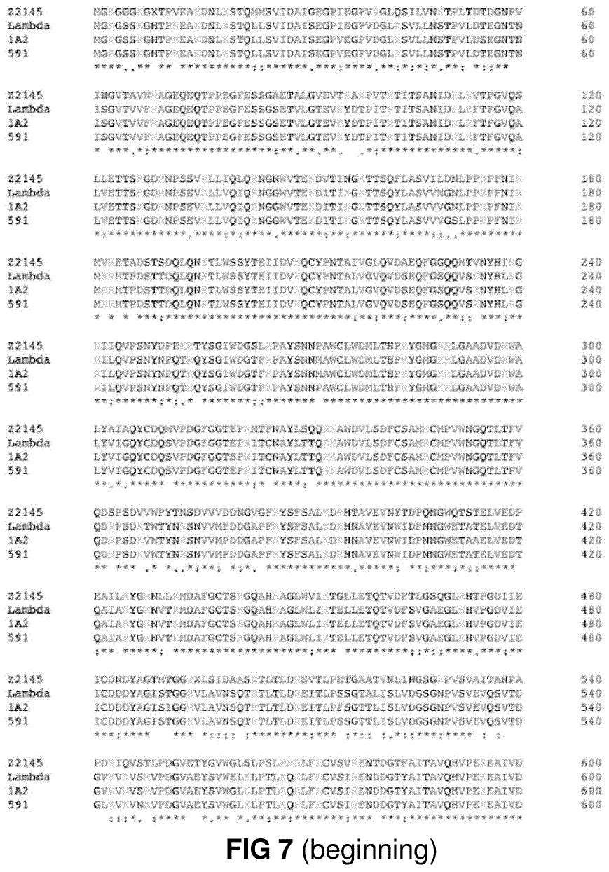

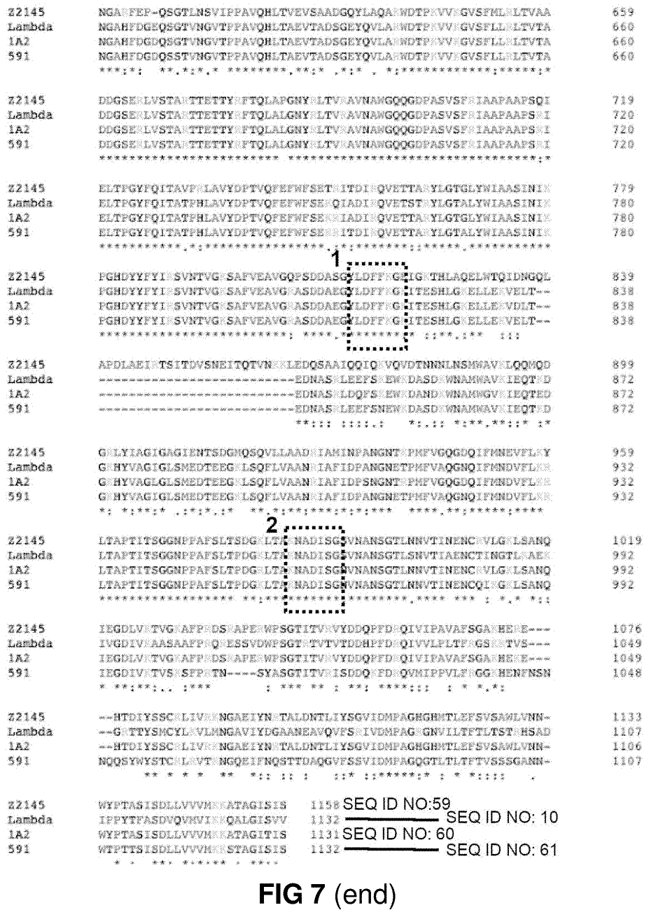

For production of chimeric gpJ proteins, two insertion points (FIG. 7) have been identified. In non-limiting aspects, such insertion sites may be utilized for production of chimeric proteins. Both insertion points yield functional gpJ chimeras with altered receptor binding. In an embodiment, the provided bacterial delivery vehicles contain a chimeric gpJ protein comprising a fusion between an N-terminal domain of a gpJ protein derived from a lambdoïd bacteriophage, preferably a lambda or lambda-like bacteriophage, and a C-terminal domain of a different gpJ protein wherein said N-terminal domain of the chimeric gpJ protein is fused to said C-terminal domain of a different gpJ protein within one of the amino acids regions selected from positions 810-825, or 950-970 of the N-terminal domain with reference to the lambda bacteriophage gpJ protein sequence (SEQ ID NO: 10).

In a specific embodiment, the chimeric gpJ protein comprises a fusion between the N-terminal domain of a lambda bacteriophage gpJ protein and the C-terminal domain of a gpJ protein from a different bacteriophage, which typically recognizes and binds OmpC, said N-terminal domain being in particular fused to said C-terminal domain within the amino acid region 950-970 of the N-terminal domain with reference to the lambda bacteriophage gpJ protein sequence (SEQ ID NO: 10). In said embodiment, the chimeric gpJ variant may be H591 comprising or consisting of the amino acid sequence SEQ ID NO: 11 and typically encoded by the nucleotide sequence SEQ ID NO: 25, said H591 chimeric gpJ variant typically recognizing and binding OmpC. In another embodiment, the chimeric gpJ protein comprises a fusion between the N-terminal domain of a lambda bacteriophage gpJ protein and the C-terminal domain of a gpJ protein from a different bacteriophage, which typically recognizes a receptor present in E. coli O157 strains, said N-terminal domain being in particular fused to said C-terminal domain within the amino acid region 810-825 of the N-terminal domain with reference to the lambda bacteriophage gpJ protein sequence (SEQ ID NO: 10). In said embodiment, the chimeric gpJ variant may be Z2145 comprising or consisting of the amino acid sequence SEQ ID NO: 12 and typically encoded by the nucleotide sequence SEQ ID NO: 26, said Z2145 chimeric gpJ variant typically recognizing a receptor present in O157 strains. In still another embodiment, the chimeric gpJ protein comprises a fusion between the N-terminal domain of a lambda bacteriophage gpJ protein and the C-terminal domain of a gpJ protein from a different bacteriophage, which typically recognizes the OmpC receptor present in O157 strains, said N-terminal domain being in particular fused to said C-terminal domain within the amino acid region 950-970 of the N-terminal domain with reference to the lambda bacteriophage gpJ protein sequence (SEQ ID NO: 10). In said embodiment, the chimeric gpJ variant may be 1A2 comprising or consisting of the amino acid sequence SEQ ID NO: 13 and typically encoded by the nucleotide sequence SEQ ID NO: 27, said 1A2 chimeric gpJ variant typically recognizing the OmpC receptor present in E. coli O157 strains. In still another embodiment, the chimeric gpJ protein comprises a fusion between the N-terminal domain of a lambda bacteriophage gpJ protein and the C-terminal domain of a gpJ protein from a different bacteriophage, which typically recognizes the OmpC receptor present in both O157 and MG1655 strains, said N-terminal domain being in particular fused to said C-terminal domain within the amino acid region 950-970 of the N-terminal domain with reference to the lambda bacteriophage gpJ protein sequence (SEQ ID NO: 10). In said embodiment, the chimeric gpJ variant may be A8 comprising or consisting of the amino acid sequence SEQ ID NO: 49 and typically encoded by the nucleotide sequence SEQ ID NO: 55, said A8 chimeric gpJ variant typically recognizing the OmpC receptor in both E. coli O157 and MG1655 strains.

Bacterial delivery vehicles are also provided that comprise recombinant gpH proteins. Such gpH proteins include recombinant gpH proteins that permit or allow improved entry of bacterial vectors in cells having deficiencies or alterations in permease complexes. The recombinant engineered chimeric gpH protein may comprise a fusion between a portion of a gpH protein derived from a lambdoïd bacteriophage, preferably a lambda or lambda-like bacteriophage, and a portion of a gpH protein derived from a corresponding gpH protein derived from a different bacteriophage. Such chimeric gpH protein may comprise a fusion between the N-terminal domain of a gpH protein from a lambdoïd bacteriophage, preferably a lambda or lambda-like bacteriophage, and the C-terminal domain of a different gpH protein. One such variant is gpH-IAI of amino acid sequence SEQ ID NO: 24 and nucleotide sequence SEQ ID NO: 28.

In a particular embodiment, said bacterial delivery vehicle comprises chimeric STF-V10h variant as disclosed above and chimeric 1A2 variant as disclosed above.

In aspects, the bacterial delivery vehicles provided herein, are vehicles comprising recombinant STF protein(s) (including but not limited to chimeric and branched RBPs), gpJ protein(s) and/or gpH protein(s) that are engineered to increase the efficiency of transfer of the DNA payload into the targeted bacterial cell population. Such bacterial cell populations include for example E. coli. and other bacterial species of interest.

It has also been demonstrated herein that the size of the packaged genome can have effects on the efficiency of packaging.

Nucleic acid molecules encoding the wild type, as well as recombinant STF, gpJ, and gpH proteins disclosed herein are provided. Such nucleic acids may be included in vectors such as bacteriophages, plasmids, phagemids, viruses, and other vehicles which enable transfer and expression of the recombinant STF, gpJ, and gpH encoding nucleic acids.

In a particular embodiment, nucleic acids are included in a single vector. In a more particular embodiment, said vector comprises or consists of the nucleic acid sequence SEQ ID NO: 47.

The bacterial delivery vehicles provided herein enable transfer of a nucleic acid payload, encoding a protein or nucleic acid of interest, into a desired target bacterial host cell. As used herein, the term “delivery vehicle” refers to any means that allows the transfer of a payload into a bacterium. There are several types of delivery vehicles encompassed by the present disclosure including, without limitation, bacteriophage scaffold, virus scaffold, chemical based delivery vehicle (e.g., cyclodextrin, calcium phosphate, cationic polymers, cationic liposomes), protein-based or peptide-based delivery vehicle, lipid-based delivery vehicle, nanoparticle-based delivery vehicles, non-chemical-based delivery vehicles (e.g., transformation, electroporation, sonoporation, optical transfection), particle-based delivery vehicles (e.g., gene gun, magnetofection, impalefection, particle bombardment, cell-penetrating peptides) or donor bacteria (conjugation). Any combination of delivery vehicles is also encompassed by the present disclosure. The delivery vehicle can refer to a bacteriophage derived scaffold and can be obtained from a natural, evolved or engineered capsid.

Delivery vehicles include packaged phagemids, as well as bacteriophage, as disclosed herein. An Eligobiotic® is a packaged phagemid, i.e a payload encapsidated in a phage-derived capsid. The engineering of such delivery vehicles is well known to those skilled in the art. Such engineering techniques may employ production cell lines engineered to express the STF, gpJ and gpH proteins disclosed herein. In one aspect, bacterial delivery vehicles with desired target host ranges are provided for use in transfer of a payload to the microbiome of a host. The bacterial delivery vehicles may be characterized by combinations of wild-type and recombinant STF, gpJ and gpH proteins.

The present disclosure also provides a production cell line producing the bacterial delivery vehicles disclosed herein.

Generation of packaged phagemids and bacteriophage particles are routine techniques well-known to one skilled in the art. In an embodiment, a satellite phage and/or helper phage may be used to promote the packaging of the payload in the delivery vehicles disclosed herein. Helper phages provide functions in trans and are well known to the man skilled in the art. The helper phage comprises all the genes coding for the structural and functional proteins that are indispensable for the payload to be packaged, (i.e. the helper phage provides all the necessary gene products for the assembly of the delivery vehicle). The helper phage may contain a defective origin of replication or packaging signal, or completely lack the latter, and hence it is incapable of self-packaging, thus only bacterial delivery particles carrying the payload or plasmid will be produced. Helper phages may be chosen so that they cannot induce lysis of the host used for the delivery particle production. One skilled in the art would understand that some bacteriophages are defective and need a helper phage for payload packaging. Thus, depending on the bacteriophage chosen to prepare the bacterial delivery particles, the person skilled in the art would know if a helper phage is required. Sequences coding for one or more proteins or regulatory processes necessary for the assembly or production of packaged payloads may be supplied in trans. For example, the STF, gpJ and gpH proteins of the present disclosure may be provided in a plasmid under the control of an inducible promoter or expressed constitutively. In this case, the phage wild-type sequence may or not contain a deletion of the gene or sequence supplied in trans. Additionally, chimeric or modified phage sequences encoding a new function, like an recombinant STF, gpJ or gpH protein, may be directly inserted into the desired position in the genome of the helper phage, hence bypassing the necessity of providing the modified sequence in trans. Methods for both supplying a sequence or protein in trans in the form of a plasmid, as well as methods to generate direct genomic insertions, modifications and mutations are well known to those skilled in the art. In a particular embodiment, said production cell line produces:

a STF protein which comprises or consists of the amino acid sequence of SEQ ID NO: 14 and its associated chaperone comprising or consisting of the amino acid sequence of SEQ ID NO: 15,

a STF protein which comprises or consists of the amino acid sequence of SEQ ID NO: 16,

a STF protein which comprises or consists of the amino acid sequence of SEQ ID NO: 17 and its associated chaperone comprising or consisting of the amino acid sequence of SEQ ID NO: 18,

a STF protein which comprises or consists of the amino acid sequence of SEQ ID NO: 19 and its associated chaperone comprising or consisting of the amino acid sequence of SEQ ID NO: 20,

a STF protein which comprises or consists of the amino acid sequence of SEQ ID NO: 21 and its associated chaperone comprising or consisting of the amino acid sequence of SEQ ID NO: 22,

a STF protein which comprises or consists of the amino acid sequence of SEQ ID NO: 44, or

a STF protein which comprises or consists of the amino acid sequence of SEQ ID NO: 50 and optionally its associated chaperone comprising or consisting of the amino acid sequence of SEQ ID NO: 57.

In a particular embodiment, said helper phage comprises a nucleic acid sequence encoding the chimeric RBP comprising or consisting of the sequence SEQ ID NO: 48, said nucleic acid sequence typically comprising or consisting of the sequence SEQ ID NO: 54, and said helper phase optionally further comprises a nucleic acid sequence encoding the chimeric gpJ variant comprising or consisting of the sequence SEQ ID NO: 13, said nucleic acid sequence typically comprising or consisting of the sequence SEQ ID NO: 27. In a particular embodiment, said helper phage is a lambda phage wherein (i) the nucleic acid encoding a wild-type STF protein has been replaced by a nucleic acid sequence encoding the chimeric RBP comprising or consisting of the sequence SEQ ID NO: 48, said nucleic acid sequence typically comprising or consisting of the sequence SEQ ID NO: 54, (ii) the nucleic acid encoding a wild-type gpJ protein has been replaced by a nucleic acid sequence encoding the chimeric gpJ variant comprising or consisting of the sequence SEQ ID NO: 13, said nucleic acid sequence typically comprising or consisting of the sequence SEQ ID NO: 27, and (iii) the Cos site has been removed, and wherein optionally (iv) the helper prophage contains a mutation which prevents spontaneous cell lysis, such as the Sam7 mutation and (v) the helper prophage contains a thermosensitive version of the master cI repressor, such as the c1857 version.

In an embodiment, the bacterial delivery vehicle disclosed herein comprises a DNA payload of interest. As used herein, the term “payload” refers to any nucleic acid sequence or amino acid sequence, or a combination of both (such as, without limitation, peptide nucleic acid or peptide-oligonucleotide conjugate) transferred into a bacterium with a delivery vehicle. The term “payload” may also refer to a plasmid, a vector or a cargo. The payload can be a phagemid or phasmid obtained from natural, evolved or engineered bacteriophage genome. The payload can also be composed only in part of phagemid or phasmid obtained from natural, evolved or engineered bacteriophage genome.

As shown in Example 1 below, the efficiency of loading of the payload by the bacterial delivery vehicle disclosed herein may depend upon the size of the payload, among others. Accordingly, in a particular embodiment, the payload has a size superior or equal to 4 kb, and preferably inferior or equal to 51 kb.

In said embodiment, the payload may have a size, an integer multiple of which is between 36 kb and 51 kb. In other words, in that embodiment, there is at least an integer n, such as 36 kb≤n×size of the payload≤51 kb.

It has been more particularly demonstrated that it was possible to produce a more uniform population of bacterial delivery vehicles comprising an almost unique number of payload copies when said payload had a size of a specific range.

In a particular embodiment, the payload has a size strictly superior to 10.000 kb and strictly inferior to 12.000 kb. In an alternative embodiment, the payload has a size strictly superior to 12.500 kb and strictly inferior to 16.667 kb, in particular a size strictly superior to 12.500 kb and inferior to 13.000 kb.

In another particular embodiment, the payload has a size superior or equal to 18.000 kb and inferior or equal to 25.000 kb, in particular inferior or equal to 24.000 kb.

The payload may be a nucleic acid plasmid that is able to circularize upon transfer into the target cell and then either replicate or integrate inside the chromosome. Replication of the vector DNA is dependent on the presence of a bacterial origin of replication. Once replicated, inheritance of the plasmid into each of the daughter cells can be mediated by the presence of an active partitioning mechanism and a plasmid addiction system such as toxin/anti-toxin system.

As used herein, the term “nucleic acid” refers to a sequence of at least two nucleotides covalently linked together which can be single-stranded or double-stranded or contains portion of both single-stranded and double-stranded sequence. Nucleic acids can be naturally occurring, recombinant or synthetic. The nucleic acid can be in the form of a circular sequence or a linear sequence or a combination of both forms. The nucleic acid can be DNA, both genomic or cDNA, or RNA or a combination of both. The nucleic acid may contain any combination of deoxyribonucleotides and ribonucleotides, and any combination of bases, including uracil, adenine, thymine, cytosine, guanine, inosine, xanthine, hypoxanthine, isocytosine, 5-hydroxymethylcytosine and isoguanine. Other examples of modified bases that can be used are detailed in Chemical Reviews 2016, 116 (20) 12655-12687. The term “nucleic acid” also encompasses any nucleic acid analogs which may contain other backbones comprising, without limitation, phosphoramide, phosphorothioate, phosphorodithioate, O-methylphosphoroamidite linkage and/or deoxyribonucleotides and ribonucleotides nucleic acids. Any combination of the above features of a nucleic acid is also encompassed by the present disclosure.

Origins of replication known in the art have been identified from species-specific plasmid DNAs (e.g. CoIE1, R1, pT181, pSC101, pMB1, R6K, RK2, p15a and the like), from bacterial virus (e.g. φX174, M13, F1 and P4) and from bacterial chromosomal origins of replication (e.g. oriC). In one embodiment, the phagemid according to the disclosure comprises a bacterial origin of replication that is functional in the targeted bacteria.

Alternatively, the plasmid according to the disclosure does not comprise any functional bacterial origin of replication or contain an origin of replication that is inactive in the targeted bacteria. Thus, the plasmid of the disclosure cannot replicate by itself once it has been introduced into a bacterium by the bacterial virus particle.

In one embodiment, the origin of replication on the plasmid to be packaged is inactive in the targeted bacteria, meaning that this origin of replication is not functional in the bacteria targeted by the bacterial virus particles, thus preventing unwanted plasmid replication.

In one embodiment, the plasmid comprises a bacterial origin of replication that is functional in the bacteria used for the production of the bacterial virus particles.

Plasmid replication depends on host enzymes and on plasmid-controlled cis and trans determinants. For example, some plasmids have determinants that are recognized in almost all gram-negative bacteria and act correctly in each host during replication initiation and regulation. Other plasmids possess this ability only in some bacteria (Kues, U and Stahl, U 1989 Microbiol Rev 53:491-516).

Plasmids are replicated by three general mechanisms, namely theta type, strand displacement, and rolling circle (reviewed by Del Solar et al. 1998 Microhio and Molec Biol. Rev 62:434-464) that start at the origin of replication. These replication origins contain sites that are required for interactions of plasmid and/or host encoded proteins.

Origins of replication used on the plasmid of the disclosure may be of moderate copy number, such as colE1 ori from pBR322 (15-20 copies per cell) or the R6K plasmid (15-20 copies per cell) or may be high copy number, e.g. pUC oris (500-700 copies per cell), pGEM oris (300-400 copies per cell), pTZ oris (>1000 copies per cell) or pBluescript oris (300-500 copies per cell).

In one embodiment, the bacterial origin of replication is selected in the group consisting of ColE1, pMB1 and variants (pBR322, pET, pUC, etc), p15a, ColA, ColE2, pOSAK, pSC101, R6K, IncW (pSa etc), IncFII, pT181, P1, F IncP, IncC, IncJ, IncN, IncP1, IncP4, IncQ, IncH11, RSF1010, CloDF13, NTP16, R1, f5, pPS10, pC194, pE194, BBR1, pBC1, pEP2, pWVO1, pLF1311, pAP1, pWKS1, pLS1, pLS11, pUB6060, pJD4, pIJ101, pSN22, pAMbeta1, pIP501, pIP407, ZM6100(Sa), pCU1, RA3, pMOL98, RK2/RP4/RP1/R68, pB10, R300B, pRO1614, pRO1600, pECB2, pCM1, pFA3, RepFIA, RepFIB, RepFIC, pYVE439-80, R387, phasyl, RA1, TF-FC2, pMV158 and pUB113.

In an embodiment, the bacterial origin of replication is a E. coli origin of replication selected in the group consisting of ColE1, pMB1 and variants (pBR322, pET, pUC, etc), p15a, ColA, ColE2, pOSAK, pSC101, R6K, IncW (pSa etc), IncFII, pT181, P1, F IncP, IncC, IncJ, IncN, IncP1, IncP4, IncQ, IncH11, RSF1010, CloDF13, NTP16, R1, f5 and pPS10.

In an embodiment, the bacterial origin of replication is selected in the group consisting of pC194, pE194, BBR1, pBC1, pEP2, pWVO1, pLF1311, pAP1, pWKS1, pLS1, pLS11, pUB6060, pJD4, pIJ101, pSN22, pAMbeta1, pIP501, pIP407, ZM6100(Sa), pCU1, RA3, pMOL98, RK2/RP4/RP1/R68, pB10, R300B, pRO1614, pRO1600, pECB2, pCM1, pFA3, RepFIA, RepFIB, RepFIC, pYVE439-80, R387, phasyl, RAL TF-FC2, pMV158 and pUB113.

In an embodiment, the bacterial origin of replication is ColE1.

The delivered nucleic acid sequence according to the disclosure may comprise a phage replication origin which can initiate, with complementation of a complete phage genome, the replication of the delivered nucleic acid sequence for later encapsulation into the different capsids.

A phage origin of replication comprised in the delivered nucleic acid sequence of the disclosure can be any origin of replication found in a phage.

In an embodiment, the phage origin of replication can be the wild-type or non-wild type sequence of the M13, f1, φX174, P4, lambda, P2, lambda-like, HK022, mEP237, HK97, HK629, HK630, mEP043, mEP213, mEP234, mEP390, mEP460, mEPx1, mEPx2, phi80, mEP234, T2, T4, T5, T7, RB49, phiX174, R17, PRD1 P1-like, P2-like, P22, P22-like, N15 and N15-like bacteriophages.

In an embodiment, the phage origin of replication is selected in the group consisting of phage origins of replication of M13, f1, φX174, P4, and lambda.

In a particular embodiment, the phage origin of replication is the lambda or P4 origin of replication.

The delivered nucleic acid of interest comprises a nucleic acid sequence under the control of a promoter. In certain embodiments, the nucleic acid of interest is selected from the group consisting of a Cas nuclease gene, a Cas9 nuclease gene, a guide RNA, a CRISPR locus, a toxin gene, a gene encoding an enzyme such as a nuclease or a kinase, a TALEN, a ZFN, a meganuclease, a recombinase, a bacterial receptor, a membrane protein, a structural protein, a secreted protein, a gene encoding resistance to an antibiotic or to a drug in general, a gene encoding a toxic protein or a toxic factor, and a gene encoding a virulence protein or a virulence factor, or any of their combination. In an embodiment, the nucleic acid payload encodes a therapeutic protein. In another embodiment, the nucleic acid payload encodes an antisense nucleic acid molecule.

In one embodiment, the sequence of interest is a programmable nuclease circuit to be delivered to the targeted bacteria. This programmable nuclease circuit is able to mediate in vivo sequence-specific elimination of bacteria that contain a target gene of interest (e.g. a gene that is harmful to humans). Some embodiments of the present disclosure relate to engineered variants of the Type II CRISPR-Cas (Clustered Regularly Interspaced Short Palindromic Repeats-CRISPR-associated) system of Streptococcus pyogenes. Other programmable nucleases that can be used include other CRISPR-Cas systems, engineered TALEN (Transcription Activator-Like Effector Nuclease) variants, engineered zinc finger nuclease (ZFN) variants, natural, evolved or engineered meganuclease or recombinase variants, and any combination or hybrids of programmable nucleases. Thus, the engineered autonomously distributed nuclease circuits provided herein may be used to selectively cleave DNA encoding a gene of interest such as, for example, a toxin gene, a virulence factor gene, an antibiotic resistance gene, a remodeling gene or a modulatory gene (cf. WO2014124226).

Other sequences of interest, such as programmable sequences, can be added to the delivered nucleic acid sequence so as to be delivered to targeted bacteria. In an embodiment, the sequence of interest added to the delivered nucleic acid sequence leads to cell death of the targeted bacteria. For example, the nucleic acid sequence of interest added to the plasmid may encode holins or toxins.

Alternatively, the sequence of interest circuit added to the delivered nucleic acid sequence does not lead to bacteria death. For example, the sequence of interest may encode reporter genes leading to a luminescence or fluorescence signal. Alternatively, the sequence of interest may comprise proteins and enzymes achieving a useful function such as modifying the metabolism of the bacteria or the composition of its environment.

In a particular embodiment, the nucleic sequence of interest is selected in the group consisting of Cas9, a single guide RNA (sgRNA), a CRISPR locus, a gene encoding an enzyme such as a nuclease or a kinase, a TALEN, a ZFN, a meganuclease, a recombinase, a bacterial receptor, a membrane protein, a structural protein, a secreted protein, a gene encoding resistance to an antibiotic or to a drug in general, a gene encoding a toxic protein or a toxic factor and a gene encoding a virulence protein or a virulence factor.

In a particular embodiment, the delivered nucleic acid sequence according to the disclosure comprises a nucleic acid sequence of interest that encodes a bacteriocin, which can be a proteinaceous toxin produced by bacteria to kill or inhibit growth of other bacteria. Bacteriocins are categorized in several ways, including producing strain, common resistance mechanisms, and mechanism of killing. Such bacteriocin had been described from gram negative bacteria (e.g. microcins, colicin-like bacteriocins and tailocins) and from gram positive bacteria (e.g. Class I, Class II, Class III or Class IV bacteriocins).

In one embodiment, the delivered nucleic acid sequence according to the disclosure further comprises a sequence of interest encoding a toxin selected in the group consisting of microcins, colicin-like bacteriocins, tailocins, Class I, Class II, Class III and Class IV bacteriocins.

In a particular embodiment, the corresponding immunity polypeptide (i.e. anti-toxin) may be used to protect bacterial cells (see review by Cotter et al., Nature Reviews Microbiology 11: 95, 2013, which is hereby incorporated by reference in its entirety) for delivered nucleic acid sequence production and encapsidation purpose but is absent in the pharmaceutical composition and in the targeted bacteria in which the delivered nucleic acid sequence of the disclosure is delivered.

In one aspect of the disclosure, the CRISPR system is included in the delivered nucleic acid sequence. The CRISPR system contains two distinct elements, i.e. i) an endonuclease, in this case the CRISPR associated nuclease (Cas or “CRISPR associated protein”) and ii) a guide RNA. The guide RNA is in the form of a chimeric RNA which consists of the combination of a CRISPR (RNAcr) bacterial RNA and a RNAtracr (trans-activating RNA CRISPR) (Jinek et al., Science 2012). The guide RNA combines the targeting specificity of the RNAcr corresponding to the “spacing sequences” that serve as guides to the Cas proteins, and the conformational properties of the RNAtracr in a single transcript. When the guide RNA and the Cas protein are expressed simultaneously in the cell, the target genomic sequence can be permanently modified or interrupted. The modification is advantageously guided by a repair matrix. In general, the CRISPR system includes two main classes depending on the nuclease mechanism of action. Class 1 is made of multi-subunit effector complexes and includes type I, III and IV. Class 2 is made of single-unit effector modules, like Cas9 nuclease, and includes type II (II-A, II-B, II-C, II-C variant), V (V-A, V-B, V-C, V-D, V-E, V-U1, V-U2, V-U3, V-U4, V-U5) and VI (VI-A, VI-B1, VI-B2, VI-C, VI-D)

The sequence of interest according to the present disclosure comprises a nucleic acid sequence encoding Cas protein. A variety of CRISPR enzymes are available for use as a sequence of interest on the plasmid. In some embodiments, the CRISPR enzyme is a Type II CRISPR enzyme. In some embodiments, the CRISPR enzyme catalyzes DNA cleavage. In some other embodiments, the CRISPR enzyme catalyzes RNA cleavage. In one embodiment, the CRISPR enzymes may be coupled to a sgRNA. In certain embodiments, the sgRNA targets a gene selected in the group consisting of an antibiotic resistance gene, virulence protein or factor gene, toxin protein or factor gene, a bacterial receptor gene, a membrane protein gene, a structural protein gene, a secreted protein gene and a gene encoding resistance to a drug in general.

Non-limiting examples of Cas proteins as part of a multi-subunit effector or as a single-unit effector include Cas1, Cas1B, Cas2, Cas3, Cas4, Cas5, Cash, Cas7, Cas8, Cas9 (also known as Csn1 and Csx12), Cas10, Cas11 (SS), Cas12a (Cpf1), Cas12b (C2c1), Cas12c (C2c3), Cas12d (CasY), Cas12e (CasX), C2c4, C2c8, C2c5, C2c10, C2c9, Cas13a (C2c2), Cas13b (C2c6), Cas13c (C2c7), Cas13d, Csa5, Csc1, Csc2, Cse1, Cse2, Csy1, Csy2, Csy3, Csf1, Csf2, Csf3, Csf4, Csm2, Csm3, Csm4, Csm5, Csm6, Cmr1, Cmr3, Cmr4, Cmr5, Cmr6, Csn2, Csb1, Csb2, Csb3, Csx17, Csx14, Csx10, Csx16, CsaX, Csx13, Csx1, Csx15, SdCpf1, CmtCpf1, TsCpf1, CmaCpf1, PcCpf1, ErCpf1, FbCpf1, UbcCpf1, AsCpf1, LbCpf1, homologues thereof, orthologues thereof, variants thereof, or modified versions thereof. In some embodiments, the CRISPR enzyme cleaves both strands of the target nucleic acid at the Protospacer Adjacent Motif (PAM) site.

In a particular embodiment, the CRISPR enzyme is any Cas9 protein, for instance any naturally occurring bacterial Cas9 as well as any variants, homologs or orthologs thereof.

By “Cas9” is meant a protein Cas9 (also called Csn1 or Csx12) or a functional protein, peptide or polypeptide fragment thereof, i.e. capable of interacting with the guide RNA(s) and of exerting the enzymatic activity (nuclease) which allows it to perform the double-strand cleavage of the DNA of the target genome. “Cas9” can thus denote a modified protein, for example truncated to remove domains of the protein that are not essential for the predefined functions of the protein, in particular the domains that are not necessary for interaction with the gRNA (s).

The sequence encoding Cas9 (the entire protein or a fragment thereof) as used in the context of the disclosure can be obtained from any known Cas9 protein (Fonfara et al., Nucleic Acids Res 42 (4), 2014; Koonin et al., Nat Rev Microbiol 15(3), 2017). Examples of Cas9 proteins useful in the present disclosure include, but are not limited to, Cas9 proteins of Streptococcus pyogenes (SpCas9), Streptococcus thermophiles (St1Cas9, St3Cas9), Streptococcus mutans, Staphylococcus aureus (SaCas9), Campylobacter jejuni (CjCas9), Francisella novicida (FnCas9) and Neisseria meningitides (NmCas9).

The sequence encoding Cpf1 (Cas12a) (the entire protein or a fragment thereof) as used in the context of the disclosure can be obtained from any known Cpf1 (Cas12a) protein (Koonin et al., 2017). Examples of Cpf1(Cas12a) proteins useful in the present disclosure include, but are not limited to, Cpf1(Cas12a) proteins of Acidaminococcus sp, Lachnospiraceae bacteriu and Francisella novicida.

The sequence encoding Cas13a (the entire protein or a fragment thereof) can be obtained from any known Cas13a (C2c2) protein (Abudayyeh et al., 2017). Examples of Cas13a (C2c2) proteins useful in the present disclosure include, but are not limited to, Cas13a (C2c2) proteins of Leptotrichia wadei (LwaCas13a).

The sequence encoding Cas13d (the entire protein or a fragment thereof) can be obtained from any known Cas13d protein (Yan et al., 2018). Examples of Cas13d proteins useful in the present disclosure include, but are not limited to, Cas13d proteins of Eubacterium siraeum and Ruminococcus sp.

In a particular embodiment, the nucleic sequence of interest is a CRISPR/Cas9 system for the reduction of gene expression or inactivation a gene selected in the group consisting of an antibiotic resistance gene, virulence factor or protein gene, toxin factor or protein gene, a gene encoding a bacterial receptor, a membrane protein, a structural protein, a secreted protein, and a gene encoding resistance to a drug in general.

In one embodiment, the CRISPR system is used to target and inactivate a virulence factor. A virulence factor can be any substance produced by a pathogen that alters host-pathogen interaction by increasing the degree of damage done to the host. Virulence factors are used by pathogens in many ways, including, for example, in cell adhesion or colonization of a niche in the host, to evade the host's immune response, to facilitate entry to and egress from host cells, to obtain nutrition from the host, or to inhibit other physiological processes in the host. Virulence factors can include enzymes, endotoxins, adhesion factors, motility factors, factors involved in complement evasion, and factors that promote biofilm formation. For example, such targeted virulence factor gene can be E. coli virulence factor gene such as, without limitation, EHEC-HlyA, Stx1 (VT1), Stx2 (VT2), Stx2a (VT2a), Stx2b (VT2b), Stx2c (VT2c), Stx2d (VT2d), Stx2e (VT2e) and Stx2f (VT2f), Stx2h (VT2h), fimA, fimF, fimH, neuC, kpsE, sfa, foc, iroN, aer, iha, papC, papGI, papGII, papGIII, hlyC, cnf1, hra, sat, ireA, usp ompT, ibeA, malX, fyuA, irp2, traT, afaD, ipaH, eltB, estA, bfpA, eaeA, espA, aaiC, aatA, TEM, CTX, SHV, csgA, csgB, csgC, csgD, csgE, csgF, csgG, csgH, T1SS, T2SS, T3SS, T4SS, T5SS, T6SS (secretion systems). For example, such targeted virulence factor gene can be Shigella dysenteriae virulence factor gene such as, without limitation, stx1 and stx2. For example, such targeted virulence factor gene can be Yersinia pestis virulence factor gene such as, without limitation, yscF (plasmid-borne (pCD1) T3SS external needle subunit). For example, such targeted virulence factor gene can be Francisella tularensis virulence factor gene such as, without limitation, fs1A. For example, such targeted virulence factor gene can be Bacillus anthracis virulence factor gene such as, without limitation, pag (Anthrax toxin, cell-binding protective antigen). For example, such targeted virulence factor gene can be Vibrio cholera virulence factor gene such as, without limitation, ctxA and ctxB (cholera toxin), tcpA (toxin co-regulated pilus), and toxT (master virulence regulator). For example, such targeted virulence factor gene can be Pseudomonas aeruginosa virulence factor genes such as, without limitation, pyoverdine (e.g., sigma factor pvdS, biosynthetic genes pvdL, pvdl, pvdJ, pvdH, pvdA, pvdF, pvdQ, pvdN, pvdM, pvdO, pvdP, transporter genes pvdE, pvdR, pvdT, opmQ), siderophore pyochelin (e.g., pchD, pchC, pchB, pchA, pchE, pchF and pchG, and toxins (e.g., exoU, exoS and exoT). For example, such targeted virulence factor gene can be Klebsiella pneumoniae virulence factor genes such as, without limitation, fimA (adherence, type I fimbriae major subunit), and cps (capsular polysaccharide). For example, such targeted virulence factor gene can be Acinetobacter baumannii virulence factor genes such as, without limitation, ptk (capsule polymerization) and epsA (assembly). For example, such targeted virulence factor gene can be Salmonella enterica typhi virulence factor genes such as, without limitation, MIA (invasion, SPI-1 regulator), ssrB (SPI-2 regulator), and those associated with bile tolerance, including efflux pump genes acrA, acrB and tolC. For example, such targeted virulence factor gene can be Fusobacterium nucleatum virulence factor genes such as, without limitation, FadA and TIGIT. For example, such targeted virulence factor gene can be Bacteroides fragilis virulence factor genes such as, without limitation, bft.

In another embodiment, the CRISPR/Cas9 system is used to target and inactivate an antibiotic resistance gene such as, without limitation, GyrB, ParE, ParY, AAC(1), AAC(2′), AAC(3), AAC(6′), ANT(2″), ANT(3″), ANT(4′), ANT(6), ANT(9), APH(2″), APH(3″), APH(3′), APH(4), APH(6), APH(7″), APH(9), ArmA, RmtA, RmtB, RmtC, Sgm, AER, BLA1, CTX-M, KPC, SHV, TEM, BlaB, CcrA, IMP, NDM, VIM, ACT, AmpC, CMY, LAT, PDC, OXA β-lactamase, mecA, Omp36, OmpF, PIB, bla (blaI, blaR1) and mec (mecI, mecR1) operons, Chloramphenicol acetyltransferase (CAT), Chloramphenicol phosphotransferase, Ethambutol-resistant arabinosyltransferase (EmbB), MupA, MupB, Integral membrane protein MprF, Cfr 23 S rRNA methyltransferase, Rifampin ADP-ribosyltransferase (Arr), Rifampin glycosyltransferase, Rifampin monooxygenase, Rifampin phosphotransferase, DnaA, RbpA, Rifampin-resistant beta-subunit of RNA polymerase (RpoB), Erm 23 S rRNA methyltransferases, Lsa, MsrA, Vga, VgaB, Streptogramin Vgb lyase, Vat acetyltransferase, Fluoroquinolone acetyltransferase, Fluoroquinolone-resistant DNA topoisomerases, Fluoroquinolone-resistant GyrA, GyrB, ParC, Quinolone resistance protein (Qnr), FomA, FomB, FosC, FosA, FosB, FosX, VanA, VanB, VanD, VanR, VanS, Lincosamide nucleotidyltransferase (Lin), EreA, EreB, GimA, Mgt, Ole, Macrolide phosphotransferases (MPH), MefA, MefE, Mel, Streptothricin acetyltransferase (sat), Sul1, Sul2, Sul3, sulfonamide-resistant FolP, Tetracycline inactivation enzyme TetX, TetA, TetB, TetC, Tet30, Tet31, TetM, TetO, TetQ, Tet32, Tet36, MacAB-To1C, MsbA, MsrA, VgaB, EmrD, EmrAB-To1C, NorB, GepA, MepA, AdeABC, AcrD, MexAB-OprM, mtrCDE, EmrE, adeR, acrR, baeSR, mexR, phoPQ, mtrR, or any antibiotic resistance gene described in the Comprehensive Antibiotic Resistance Database (CARD https://card.mcmaster.ca/).

In another embodiment, the CRISPR/Cas9 system is used to target and inactivate a bacterial toxin gene. Bacterial toxins can be classified as either exotoxins or endotoxins. Exotoxins are generated and actively secreted; endotoxins remain part of the bacteria. The response to a bacterial toxin can involve severe inflammation and can lead to sepsis. Such toxin can be for example Botulinum neurotoxin, Tetanus toxin, Staphylococus toxins, Diphteria toxin, Anthrax toxin, Alpha toxin, Pertussis toxin, Shiga toxin, Heat-stable enterotoxin (E. coli ST), colibactin, BFT (B. fragilis toxin) or any toxin described in Henkel et al., (Toxins from Bacteria in EXS. 2010; 100: 1-29).

In a particular embodiment, said payload comprises or consists of the nucleic acid sequence SEQ ID NO: 47.

The bacteria targeted by bacterial delivery vehicles disclosed herein can be any bacteria present in a mammal organism. In a certain aspect, the bacteria are targeted through interaction of the chimeric RBPs and/or the branched-RBPs expressed by the delivery vehicles with the bacterial cell. It can be any commensal, symbiotic or pathogenic bacteria of the microbiota or microbiome.

A microbiome may comprise a variety of endogenous bacterial species, any of which may be targeted in accordance with the present disclosure. In some embodiments, the genus and/or species of targeted endogenous bacterial cells may depend on the type of bacteriophages being used for preparing the bacterial delivery vehicles. For example, some bacteriophages exhibit tropism for, or preferentially target, specific host species of bacteria. Other bacteriophages do not exhibit such tropism and may be used to target a number of different genus and/or species of endogenous bacterial cells.

Examples of bacterial cells include, without limitation, cells from bacteria of the genus Yersinia spp., Escherichia spp., Klebsiella spp., Acinetobacter spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Vibrio spp., Bacillus spp., Erysipelothrix spp., Salmonella spp., Streptomyces spp., Streptococcus spp., Staphylococcus spp., Bacteroides spp., Prevotella spp., Clostridium spp., Bifidobacterium spp., Clostridium spp., Brevibacterium spp., Lactococcus spp., Leuconostoc spp., Actinobacillus spp., Selnomonas spp., Shigella spp., Zymonas spp., Mycoplasma spp., Treponema spp., Leuconostoc spp., Corynebacterium spp., Enterococcus spp., Enterobacter spp., Pyrococcus spp., Serratia spp., Morganella spp., Parvimonas spp., Fusobacterium spp., Actinomyces spp., Porphyromonas spp., Micrococcus spp., Bartonella spp., Borrelia spp., Brucelia spp., Campylobacter spp., Chlamydophilia spp., Cutibacterium (formerly Propionibacterium) spp., Ehrlichia spp., Haemophilus spp., Leptospira spp., Listeria spp., Mycoplasma spp., Nocardia spp., Rickettsia spp., Ureaplasma spp., and Lactobacillus spp, and a mixture thereof.

Thus, bacterial delivery vehicles may target (e.g., specifically target) a bacterial cell from any one or more of the foregoing genus of bacteria to specifically deliver the payload of interest according to the disclosure.

In an embodiment, the targeted bacteria can be selected from the group consisting of Yersinia spp., Escherichia spp., Klebsiella spp., Acinetobacter spp., Pseudomonas spp., Helicobacter spp., Vibrio spp, Salmonella spp., Streptococcus spp., Staphylococcus spp., Bacteroides spp., Clostridium spp., Shigella spp., Enterococcus spp., Enterobacter spp., and Listeria spp.

In some embodiments, targeted bacterial cells of the present disclosure are anaerobic bacterial cells (e.g., cells that do not require oxygen for growth). Anaerobic bacterial cells include facultative anaerobic cells such as but not limited to Escherichia coli, Shewanella oneidensis and Listeria. Anaerobic bacterial cells also include obligate anaerobic cells such as, for example, Bacteroides and Clostridium species. In humans, anaerobic bacteria are most commonly found in the gastrointestinal tract. In some particular embodiment, the targeted bacteria are thus bacteria most commonly found in the gastrointestinal tract. Bacteriophages used for preparing the bacterial virus particles, and then the bacterial virus particles, may target (e.g., to specifically target) anaerobic bacterial cells according to their specific spectra known by the person skilled in the art to specifically deliver the plasmid.

In some embodiments, the targeted bacterial cells are, without limitation, Bacteroides thetaiotaomicron, Bacteroides fragilis, Bacteroides distasonis, Bacteroides vulgatus, Clostridium leptum, Clostridium coccoides, Staphylococcus aureus, Bacillus subtilis, Clostridium butyricum, Brevibacterium lactofermentum, Streptococcus agalactiae, Lactococcus lactis, Leuconostoc lactis, Actinobacillus actinobycetemcomitans, cyanobacteria, Escherichia coli, Helicobacter pylori, Selnomonas ruminatium, Shigella sonnei, Zymomonas mobilis, Mycoplasma mycoides, Treponema denticola, Bacillus thuringiensis, Staphilococcus lugdunensis, Leuconostoc oenos, Corynebacterium xerosis, Lactobacillus plantarum, Lactobacillus rhamnosus, Lactobacillus casei, Lactobacillus acidophilus, Enterococcus faecalis, Bacillus coagulans, Bacillus cereus, Bacillus popillae, Synechocystis strain PCC6803, Bacillus liquefaciens, Pyrococcus abyssi, Selenomonas nominantium, Lactobacillus hilgardii, Streptococcus ferus, Lactobacillus pentosus, Bacteroides fragilis, Staphylococcus epidermidis, Streptomyces phaechromogenes, Streptomyces ghanaenis, Klebsiella pneumoniae, Enterobacter cloacae, Enterobacter aerogenes, Serratia marcescens, Morganella morganii, Citrobacter freundii, Propionibacterium freudenreichii, Pseudomonas aeruginosa, Parvimonas micra, Prevotella intermedia, Fusobacterium nucleatum, Prevotella nigrescens, Actinomyces israelii, Porphyromonas endodontalis, Porphyromonas gingivalis Micrococcus luteus, Bacillus megaterium, Aeromonas hydrophila, Aeromonas caviae, Bacillus anthracis, Bartonella henselae, Bartonella Quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Campylobacter coli, Campylobacter fetus, Chlamydia pneumoniae, Chlamydia trachomatis, Chlamydophila psittaci, Clostridium botulinum, Clostridium difficile, Clostridium perfringens, Clostridium tetani, Corynebacterium diphtheria, Cutibacterium acnes (formerly Propionibacterium acnes), Ehrlichia canis, Ehrlichia chaffeensis, Enterococcus faecium, Francisella tularensis, Haemophilus influenza, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumonia, Neisseria gonorrhoeae, Neisseria meningitides, Nocardia asteroids, Rickettsia rickettsia, Salmonella enteritidis, Salmonella typhi, Salmonella paratyphi, Salmonella typhimurium, Shigella flexnerii, Shigella dysenteriae, Staphylococcus saprophyticus, Streptococcus pneumoniae, Streptococcus pyogenes, Gardnerella vaginalis, Streptococcus viridans, Treponema pallidum, Ureaplasma urealyticum, Vibrio cholera, Vibrio parahaemolyticus, Yersinia pestis, Yersinia enterocolitica, Yersinia pseudotuberculosis, Actinobacter baumanii, Pseudomonas aeruginosa, and a mixture thereof. In an embodiment the targeted bacteria of interest are selected from the group consisting of Escherichia coli, Enterococcus faecium, Staphylococcus aureus, Klebsiella pneumoniae, Acinetobacter baumanii, Pseudomonas aeruginosa, Enterobacter cloacae, and Enterobacter aerogenes, and a mixture thereof.

In some embodiments, the targeted bacterial cells are, without limitation, Anaerotruncus, Acetanaerobacterium, Acetitomaculum, Acetivibrio, Anaerococcus, Anaerofilum, Anaerosinus, Anaerostipes, Anaerovorax, Butyrivibrio, Clostridium, Capracoccus, Dehalobacter, Dialister, Dorea, Enterococcus, Ethanoligenens, Faecalibacterium, Fusobacterium, Gracilibacter, Guggenheimella, Hespellia, Lachnobacterium, Lachnospira, Lactobacillus, Leuconostoc, Megamonas, Moryella, Mitsuokella, Oribacterium, Oxobacter, Papillibacter, Proprionispira, Pseudobutyrivibrio, Pseudoramibacter, Roseburia, Ruminococcus, Sarcina, Seinonella, Shuttleworthia, Sporobacter, Sporobacterium, Streptococcus, Subdoligranulum, Syntrophococcus, Thermobacillus, Turibacter, Weisella, Clostridium, Bacteroides, Ruminococcus, Faecalibacterium, Treponema, Phascolarctobacterium, Megasphaera, Faecalibacterium, Bifidobacterium, Lactobacillus, Sutterella, and/or Prevotella.

In other embodiments, the targeted bacteria cells are, without limitation, Achromobacter xylosoxidans, Acidaminococcus fermentans, Acidaminococcus intestinii, Acidaminococcus sp., Acinetobacter baumannii, Acinetobacter junii, Acinetobacter lwoffii, Actinobacillus capsulatus, Actinomyces naeslundii, Actinomyces neuii, Actinomyces odontolyticus, Actinomyces radingae, Adlercreutzia equolifaciens, Aeromicrobium massiliense, Aggregatibacter actinomycetemcomitans, Akkermansia muciniphila, Aliagarivorans marinus, Alistipes finegoldii, Alistipes indistinctus, Alistipes inops, Alistipes onderdonkii, Alistipes putredinis, Alistipes senegalensis, Alistipes shahii, Alistipes timonensis, Alloscardovia omnicolens, Anaerobacter polyendosporus, Anaerobaculum hydrogeniformans, Anaerococcus hydrogenalis, Anaerococcus prevotii, Anaerococcus senegalensis, Anaerofustis stercorihominis, Anaerostipes caccae, Anaerostipes hadrus, Anaerotruncus colihominis, Aneurinibacillus aneurinilyticus, Bacillus licheniformis, Bacillus massilioanorexius, Bacillus massiliosenegalensis, Bacillus simplex, Bacillus smithii, Bacillus subtilis, Bacillus thuringiensis, Bacillus timonensis, Bacteroides xylanisolvens, Bacteroides acidifaciens, Bacteroides caccae, Bacteroides capillosus, Bacteroides cellulosilyticus, Bacteroides clarus, Bacteroides coprocola, Bacteroides coprophilus, Bacteroides dorei, Bacteroides eggerthii, Bacteroides faecis, Bacteroides finegoldii, Bacteroides fluxus, Bacteroides fragilis, Bacteroides gallinarum, Bacteroides intestinalis, Bacteroides nordii, Bacteroides oleiciplenus, Bacteroides ovatus, Bacteroides pectinophilus, Bacteroides plebeius, Bacteroides salanitronis, Bacteroides salyersiae, Bacteroides sp., Bacteroides stercoris, Bacteroides thetaiotaomicron, Bacteroides uniformis, Bacteroides vulgatus, Bacteroides xylanisolvens, Bacteroidespectinophilus ATCC, Barnesiella intestinihominis, Bavariicoccus seileri, Bifidobacterium adolescentis, Bifidobacterium angulatum, Bifidobacterium animalis, Bifidobacterium bifidum, Bifidobacterium breve, Bifidobacterium catenulatum, Bifidobacterium dentium, Bifidobacterium gallicum, Bifidobacterium longum, Bifidobacterium pseudocatenulatum, Bifidobacterium stercoris, Bilophila wadsworthia, Blautia faecis, Blautia hansenii, Blautia hydrogenotrophica, Blautia luti, Blautia obeum, Blautia producta, Blautia wexlerae, Brachymonas chironomi, Brevibacterium senegalense, Bryantella formatexigens, butyrate producing bacterium, Butyricicoccus pullicaecorum, Butyricimonas virosa, Butyrivibrio crossotus, Butyrivibrio fibrisolvens, Caldicoprobacter faecalis, Campylobacter concisus, Campylobacter jejuni, Campylobacter upsaliensis, Catenibacterium mitsuokai, Cedecea davisae, Cellulomonas massiliensis, Cetobacterium somerae, Citrobacter braakii, Citrobacter freundii, Citrobacter pasteurii, Citrobacter sp., Citrobacter youngae, Cloacibacillus evryensis, Clostridiales bacterium, Clostridioides difficile, Clostridium asparagiforme, Clostridium bartlettii, Clostridium boliviensis, Clostridium bolteae, Clostridium hathewayi, Clostridium hiranonis, Clostridium hylemonae, Clostridium leptum, Clostridium methylpentosum, Clostridium nexile, Clostridium orbiscindens, Clostridium ramosum, Clostridium scindens, Clostridium sp, Clostridium sp., Clostridium spiroforme, Clostridium sporogenes, Clostridium symbiosum, Collinsella aerofaciens, Collinsella intestinalis, Collinsella stercoris, Collinsella tanakaei, Coprobacillus cateniformis, Coprobacter fastidiosus, Coprococcus catus, Coprococcus comes, Coprococcus eutactus, Corynebacterium ammoniagenes, Corynebacterium amycolatum, Corynebacterium pseudodiphtheriticum, Cutibacterium acnes, Dermabacter hominis, Desulfitobacterium hafniense, Desulfovibrio fairfieldensis, Desulfovibrio piger, Dialister succinatiphilus, Dielma fastidiosa, Dorea formicigenerans, Dorea longicatena, Dysgonomonas capnocytophagoides, Dysgonomonas gadei, Dysgonomonas mossii, Edwardsiella tarda, Eggerthella lenta, Eisenbergiella tayi, Enorma massiliensis, Enterobacter aerogenes, Enterobacter asburiae, Enterobacter cancerogenus, Enterobacter cloacae, Enterobacter massiliensis, Enterococcus casseliflavus, Enterococcus durans, Enterococcus faecalis, Enterococcus faecium, Enterococcus flavescens, Enterococcus gallinarum, Enterococcus sp., Enterovibrio nigricans, Erysipelatoclostridium ramosum, Escherichia coli, Escherichia sp., Eubacterium biforme, Eubacterium dolichum, Eubacterium hallii, Eubacterium limosum, Eubacterium ramulus, Eubacterium rectale, Eubacterium siraeum, Eubacterium ventriosum, Exiguobacterium marinum, Exiguobacterium undae, Faecalibacterium cf, Faecalibacterium prausnitzii, Faecalitalea cylindroides, Ferrimonas balearica, Finegoldia magna, Flavobacterium daejeonense, Flavonifractor plautii, Fusicatenibacter saccharivorans, Fusobacterium gonidiaformans, Fusobacterium mortiferum, Fusobacterium necrophorum, Fusobacterium nucleatum, Fusobacterium periodonticum, Fusobacterium sp., Fusobacterium ulcerans, Fusobacterium varium, Gallibacterium anatis, Gemmiger formicilis, Gordonibacter pamelaeae, Hafnia alvei, Helicobacter bilis, Helicobacter bills, Helicobacter canadensis, Helicobacter canis, Helicobacter cinaedi, Helicobacter macacae, Helicobacter pametensis, Helicobacter pullorum, Helicobacter pylori, Helicobacter rodentium, Helicobacter winghamensis, Herbaspirillum massiliense, Holdemanella biformis, Holdemania fdiformis, Holdemania filiformis, Holdemania massiliensis, Holdemaniafiliformis, Hungatella hathewayi, Intestinibacter bartlettii, Intestinimonas butyriciproducens, Klebsiella oxytoca, Klebsiella pneumoniae, Kurthia massiliensis, Lachnospira pectinoschiza, Lactobacillus acidophilus, Lactobacillus amylolyticus, Lactobacillus animalis, Lactobacillus antri, Lactobacillus brevis, Lactobacillus buchneri, Lactobacillus casei, Lactobacillus curvatus, Lactobacillus delbrueckii, Lactobacillus fermentum, Lactobacillus gasseri, Lactobacillus helveticus, Lactobacillus hilgardii, Lactobacillus iners, Lactobacillus intestinalis, Lactobacillus johnsonii, Lactobacillus murinus, Lactobacillus paracasei, Lactobacillus plantarum, Lactobacillus reuteri, Lactobacillus rhamnosus, Lactobacillus ruminis, Lactobacillus sakei, Lactobacillus salivarius, Lactobacillus ultunensis, Lactobacillus vaginalis, Lactobacillusplantarum subsp., Leuconostoc mesenteroides, Leuconostoc pseudomesenteroides, Listeria grayi, Listeria innocua, Mannheimia granulomatis, Marvinbryantia formatexigens, Megamonas funiformis, Megamonas hypermegale, Methanobrevibacter smithii, Methanobrevibacter smithiiFl, Micrococcus luteus, Microvirgula aerodenitrificans, Mitsuokella jalaludinii, Mitsuokella multacida, Mollicutes bacterium, Murimonas intestini, Neisseria macacae, Nitriliruptor alkaliphilus, Oceanobacillus massiliensis, Odoribacter laneus, Odoribacter splanchnicus, Ornithobacterium rhinotracheale, Oxalobacter formigenes, Paenibacillus barengoltzii, Paenibacillus chitinolyticus, Paenibacillus lautus, Paenibacillus motobuensis, Paenibacillus senegalensis, Paenisporosarcina quisquiliarum, Parabacteroides distasonis, Parabacteroides goldsteinii, Parabacteroides gordonii, Parabacteroides johnsonii, Parabacteroides merdae, Paraprevotella xylaniphila, Parasutterella excrementihominis, Parvimonas micra, Pediococcus acidilactici, Peptoclostridium difficile, Peptoniphilus harei, Peptoniphilus obesi, Peptoniphilus senegalensis, Peptoniphilus timonensis, Phascolarctobacterium succinatutens, Porphyromonas asaccharolytica, Porphyromonas uenonis, Prevotella baroniae, Prevotella bivia, Prevotella copri, Prevotella dentalis, Prevotella micans, Prevotella multisaccharivorax, Prevotella oralis, Prevotella salivae, Prevotella stercorea, Prevotella veroralis, Propionibacterium acnes, Propionibacterium avidum, Propionibacterium freudenreichii, Propionimicrobium lymphophilum, Proteus mirabilis, Proteuspenneri ATCC, Providencia alcalifaciens, Providencia rettgeri, Providencia rustigianii, Providencia stuartii, Pseudoflavonifractor capillosus, Pseudomonas aeruginosa, Pseudomonas luteola, Ralstonia pickettii, Rheinheimera perlucida, Rheinheimera texasensis, Riemerella columbina, Romboutsia lituseburensis, Roseburia faecis, Roseburia intestinalis, Roseburia inulinivorans, Ruminococcus bicirculans, Ruminococcus bromii, Ruminococcus callidus, Ruminococcus champanellensis, Ruminococcus faecis, Ruminococcus gnavus, Ruminococcus lactaris, Ruminococcus obeum, Ruminococcus sp, Ruminococcus sp., Ruminococcus torques, Sarcina ventriculi, Sellimonas intestinalis, Senegalimassilia anaerobia, Shigella sonnei, Slackia piriformis, Staphylococcus epidermidis, Staphylococcus lentus, Staphylococcus nepalensis, Staphylococcus pseudintermedius, Staphylococcus xylosus, Stenotrophomonas maltophilia, Streptococcus agalactiae, Streptococcus anginosus, Streptococcus australis, Streptococcus caballi, Streptococcus castoreus, Streptococcus didelphis, Streptococcus equinus, Streptococcus gordonii, Streptococcus henryi, Streptococcus hyovaginalis, Streptococcus infantarius, Streptococcus infantis, Streptococcus lutetiensis, Streptococcus merionis, Streptococcus mitis, Streptococcus mutans, Streptococcus oralis, Streptococcus ovis, Streptococcus parasanguinis, Streptococcus plurextorum, Streptococcus porci, Streptococcus pyogenes, Streptococcus salivarius, Streptococcus sobrinus, Streptococcus thermophilus, Streptococcus thoraltensis, Streptomyces albus, Subdoligranulum variabile, Succinatimonas hippei, Sutterella parvirubra, Sutterella wadsworthensis, Terrisporobacter glycolicus, Terrisporobacter mayombei, Thalassobacillus devorans, Timonella senegalensis, Turicibacter sanguinis, unknown sp, unknown sp., Varibaculum cambriense, Veillonella atypica, Veillonella dispar, Veillonella parvula, Vibrio cincinnatiensis, Virgibacillus salexigens and Weissella paramesenteroides.

In other embodiments, the targeted bacteria cells are those commonly found on the skin microbiota and are without limitation Acetobacter farinalis, Acetobacter malorum, Acetobacter orleanensis, Acetobacter sicerae, Achromobacter anxifer, Achromobacter denitrificans, Achromobacter marplatensis, Achromobacter spanius, Achromobacter xylosoxidans subsp. xylosoxidans, Acidovorax konjaci, Acidovorax radicis, Acinetobacter johnsonii, Actinomadura citrea, Actinomadura coerulea, Actinomadura fibrosa, Actinomadura fulvescens, Actinomadura jiaoheensis, Actinomadura luteofluorescens, Actinomadura mexicana, Actinomadura nitritigenes, Actinomadura verrucosospora, Actinomadura yumaensis, Actinomyces odontolyticus, Actinomycetospora atypica, Actinomycetospora corticicola, Actinomycetospora rhizophila, Actinomycetospora rishiriensis, Aeromonas australiensis, Aeromonas bestiarum, Aeromonas bivalvium, Aeromonas encheleia, Aeromonas eucrenophila, Aeromonas hydrophila subsp. hydrophila, Aeromonas piscicola, Aeromonas popoffii, Aeromonas rivuli, Aeromonas salmonicida subsp. pectinolytica, Aeromonas salmonicida subsp. smithia, Amaricoccus kaplicensis, Amaricoccus veronensis, Aminobacter aganoensis, Aminobacter ciceronei, Aminobacter lissarensis, Aminobacter niigataensis, Ancylobacter polymorphus, Anoxybacillus flavithermus subsp. yunnanensis, Aquamicrobium aerolatum, Archangium gephyra, Archangium gephyra, Archangium minus, Archangium violaceum, Arthrobacter viscosus, Bacillus anthracis, Bacillus australimaris, Bacillus drentensis, Bacillus mycoides, Bacillus pseudomycoides, Bacillus pumilus, Bacillus safensis, Bacillus vallismortis, Bosea thiooxidans, Bradyrhizobium huanghuaihaiense, Bradyrhizobium japonicum, Brevundimonas aurantiaca, Brevundimonas intermedia, Burkholderia aspalathi, Burkholderia choica, Burkholderia cordobensis, Burkholderia diffusa, Burkholderia insulsa, Burkholderia rhynchosiae, Burkholderia terrestris, Burkholderia udeis, Buttiauxella gaviniae, Caenimonas terrae, Capnocytophaga gingivalis, Chitinophaga dinghuensis, Chryseobacterium gleum, Chryseobacterium greenlandense, Chryseobacterium jejuense, Chryseobacterium piscium, Chryseobacterium sediminis, Chryseobacterium tructae, Chryseobacterium ureilyticum, Chryseobacterium vietnamense, Corynebacterium accolens, Corynebacterium afermentans subsp. lipophilum, Corynebacterium minutissimum, Corynebacterium sundsvallense, Cupriavidus metallidurans, Cupriavidus nantongensis, Cupriavidus necator, Cupriavidus pampae, Cupriavidus yeoncheonensis, Curtobacterium flaccumfaciens, Devosia epidermidihirudinis, Devosia riboflavina, Devosia riboflavina, Diaphorobacter oryzae, Dietzia psychralcaliphila, Ensifer adhaerens, Ensifer americanus, Enterococcus malodoratus, Enterococcus pseudoavium, Enterococcus viikkiensis, Enterococcus xiangfangensis, Erwinia rhapontici, Falsirhodobacter halotolerans, Flavobacterium araucananum, Flavobacterium Gluconobacter frateurii, Gluconobacter thailandicus, Gordonia alkanivorans, Halomonas aquamarina, Halomonas axialensis, Halomonas meridiana, Halomonas olivaria, Halomonas songnenensis, Halomonas variabilis, Herbaspirillum chlorophenolicum, Herbaspirillum frisingense, Herbaspirillum hiltneri, Herbaspirillum huttiense subsp. putei, Herbaspirillum lusitanum, Herminiimonas fonticola, Hydrogenophaga intermedia, Hydrogenophaga pseudoflava, Klebsiella oxytoca, Kosakonia sacchari, Lactobacillus delbrueckii subsp. bulgaricus, Lactobacillus modestisalitolerans, Lactobacillus plantarum subsp. argentoratensis, Lactobacillus xiangfangensis, Lechevalieria roselyniae, Lentzea albida, Lentzea californiensis, Leuconostoc carnosum, Leuconostoc citreum, Leuconostoc gelidum subsp. gasicomitatum, Leuconostoc mesenteroides subsp. suionicum, Luteimonas aestuarii, Lysobacter antibioticus, Lysobacter koreensis, Lysobacter oryzae, Magnetospirillum moscoviense, Marinomonas alcarazii, Marinomonas primoryensis, Massilia aurea, Massilia jejuensis, Massilia kyonggiensis, Massilia timonae, Mesorhizobium acaciae, Mesorhizobium qingshengii, Mesorhizobium shonense, Methylobacterium haplocladii, Methylobacterium platani, Methylobacterium pseudosasicola, Methylobacterium zatmanii, Microbacterium oxydan, Micromonospora chaiyaphumensis, Micromonospora chalcea, Micromonospora citrea, Micromonospora coxensis, Micromonospora echinofusca, Micromonospora halophytica, Micromonospora kangleipakensis, Micromonospora maritima, Micromonospora nigra, Micromonospora purpureochromogene, Micromonospora rhizosphaerae, Micromonospora saelicesensis, Microvirga subterranea, Microvirga zambiensis, Mycobacterium alvei, Mycobacterium avium subsp. silvaticum, Mycobacterium colombiense, Mycobacterium conceptionense, Mycobacterium conceptionense, Mycobacterium farcinogenes, Mycobacterium fortuitum subsp. fortuitum, Mycobacterium goodii, Mycobacterium insubricum, Mycobacterium llatzerense, Mycobacterium neoaurum, Mycobacterium neworleansense, Mycobacterium obuense, Mycobacterium peregrinum, Mycobacterium saopaulense, Mycobacterium septicum, Mycobacterium setense, Mycobacterium smegmatis, Neisseria subflava, Nocardia lijiangensis, Nocardia thailandica, Novosphingobium barchaimii, Novosphingobium lindaniclasticum, Novosphingobium lindaniclasticum, Novosphingobium mathurense, Ochrobactrum pseudogrignonense, Oxalicibacterium solurbis, Paraburkholderia glathei, Paraburkholderia humi, Paraburkholderia phenazinium, Paraburkholderia phytofirmans, Paraburkholderia sordidicola, Paraburkholderia terricola, Paraburkholderia xenovorans, Paracoccus laeviglucosivorans, Patulibacter ginsengiterrae, Polymorphospora rubra, Porphyrobacter colymbi, Prevotella jejuni, Prevotella melaninogenica, Propionibacterium acnes subsp. elongatum, Proteus vulgaris, Providencia rustigianii, Pseudoalteromonas agarivorans, Pseudoalteromonas atlantica, Pseudoalteromonas paragorgicola, Pseudomonas asplenii, Pseudomonas asuensis, Pseudomonas benzenivorans, Pseudomonas cannabina, Pseudomonas cissicola, Pseudomonas congelans, Pseudomonas costantinii, Pseudomonas ficuserectae, Pseudomonas frederiksbergensis, Pseudomonas graminis, Pseudomonas jessenii, Pseudomonas koreensis, Pseudomonas koreensis, Pseudomonas kunmingensis, Pseudomonas marginalis, Pseudomonas mucidolens, Pseudomonas panacis, Pseudomonas plecoglossicida, Pseudomonas poae, Pseudomonas pseudoalcaligenes, Pseudomonas putida, Pseudomonas reinekei, Pseudomonas rhizosphaerae, Pseudomonas seleniipraecipitans, Pseudomonas umsongensis, Pseudomonas zhaodongensis, Pseudonocardia alaniniphila, Pseudonocardia ammonioxydans, Pseudonocardia autotrophica, Pseudonocardia kongjuensis, Pseudonocardia yunnanensis, Pseudorhodoferax soli, Pseudoxanthomonas daejeonensis, Pseudoxanthomonas indica, Pseudoxanthomonas kaohsiungensis, Psychrobacter aquaticus, Psychrobacter arcticus, Psychrobacter celer, Psychrobacter marincola, Psychrobacter nivimaris, Psychrobacter okhotskensis, Psychrobacter okhotskensis, Psychrobacter piscatorii, Psychrobacter pulmonis, Ramlibacter ginsenosidimutans, Rheinheimera japonica, Rheinheimera muenzenbergensis, Rheinheimera soli, Rheinheimera tangshanensis, Rheinheimera texasensis, Rheinheimera tilapiae, Rhizobium alamii, Rhizobium azibense, Rhizobium binae, Rhizobium daejeonense, Rhizobium endophyticum, Rhizobium etli, Rhizobium fabae, Rhizobium freirei, Rhizobium gallicum, Rhizobium loessense, Rhizobium sophoriradicis, Rhizobium taibaishanense, Rhizobium vallis, Rhizobium vignae, Rhizobium vignae, Rhizobium yanglingense, Rhodococcus baikonurensis, Rhodococcus enclensis, Rhodoferax saidenbachensis, Rickettsia canadensis, Rickettsia heilongjiangensis, Rickettsia honei, Rickettsia raoultii, Roseateles aquatilis, Roseateles aquatilis, Salmonella enterica subsp. salamae, Serratia ficaria, Serratia myotis, Serratia vespertilionis, Shewanella aestuarii, Shewanella decolorationis, Sphingobium amiense, Sphingobium baderi, Sphingobium barthaii, Sphingobium chlorophenolicum, Sphingobium cupriresistens, Sphingobium czechense, Sphingobium fuliginis, Sphingobium indicum, Sphingobium indicum, Sphingobium japonicum, Sphingobium lactosutens, Sphingomonas dokdonensis, Sphingomonas pseudosanguinis, Sphingopyxis chilensis, Sphingopyxis fribergensis, Sphingopyxis granuli, Sphingopyxis indica, Sphingopyxis witflariensis, Staphylococcus agnetis, Staphylococcus aureus subsp. aureus, Staphylococcus epidermidis, Staphylococcus hominis subsp. novobiosepticus, Staphylococcus nepalensis, Staphylococcus saprophyticus subsp. bovis, Staphylococcus sciuri subsp. carnaticus, Streptomyces caeruleatus, Streptomyces canarius, Streptomyces capoamus, Streptomyces ciscaucasicus, Streptomyces griseorubiginosus, Streptomyces olivaceoviridis, Streptomyces panaciradicis, Streptomyces phaeopurpureus, Streptomyces pseudovenezuelae, Streptomyces resistomycificus, Tianweitania sediminis, Tsukamurella paurometabola, Variovorax guangxiensis, Vogesella alkaliphila, Xanthomonas arboricola, Xanthomonas axonopodis, Xanthomonas cassavae, Xanthomonas cucurbitae, Xanthomonas cynarae, Xanthomonas euvesicatoria, Xanthomonas fragariae, Xanthomonas gardneri, Xanthomonas perforans, Xanthomonas pisi, Xanthomonas populi, Xanthomonas vasicola, Xenophilus aerolatus, Yersinia nurmii, Abiotrophia defectiva, Acidocella aminolytica, Acinetobacter guangdongensis, Acinetobacter parvus, Acinetobacter radioresistens, Acinetobacter soli, Acinetobacter variabilis, Actinomyces cardiffensis, Actinomyces dentalis, Actinomyces europaeus, Actinomyces gerencseriae, Actinomyces graevenitzii, Actinomyces haliotis, Actinomyces johnsonii, Actinomyces massiliensis, Actinomyces meyeri, Actinomyces meyeri, Actinomyces naeslundii, Actinomyces neuii subsp. anitratus, Actinomyces odontolyticus, Actinomyces oris, Actinomyces turicensis, Actinomycetospora corticicola, Actinotignum schaalii, Aerococcus christensenii, Aerococcus urinae, Aeromicrobium flavum, Aeromicrobium massiliense, Aeromicrobium tamlense, Aeromonas sharmana, Aggregatibacter aphrophilus, Aggregatibacter segnis, Agrococcus baldri, Albibacter methylovorans, Alcaligenes faecalis subsp. faecalis, Algoriphagus ratkowskyi, Alkalibacterium olivapovliticus, Alkalibacterium pelagium, Alkalibacterium pelagium, Alloprevotella cava, Alsobacter metallidurans, Amaricoccus kaplicensis, Amaricoccus veronensis, Anaerococcus hydrogenalis, Anaerococcus lactolyticus, Anaerococcus murdochii, Anaerococcus octavius, Anaerococcus prevotii, Anaerococcus vaginalis, Aquabacterium citratiphilum, Aquabacterium olei, Aquabacterium olei, Aquabacterium parvum, Aquincola tertiaricarbonis, Arcobacter venerupis, Arsenicicoccus bolidensis, Arthrobacter russicus, Asticcacaulis excentricus, Atopobium deltae, Atopobium parvulum, Atopobium rimae, Atopobium vaginae, Aureimonas altamirensis, Aureimonas rubiginis, Azospira oryzae, Azospirillum oryzae, Bacillus circulans, Bacillus drentensis, Bacillus fastidiosus, Bacillus lehensis, Bacillus oceanisediminis, Bacillus rhizosphaerae, Bacteriovorax stolpii, Bacteroides coagulans, Bacteroides dorei, Bacteroides Bacteroides ovatus, Bacteroides stercoris, Bacteroides uniformis, Bacteroides vulgatus, Bdellovibrio bacteriovorus, Bdellovibrio exovorus, Belnapia moabensis, Belnapia soli, Blautia hansenii, Blautia obeum, Blautia wexlerae, Bosea lathyri, Brachybacterium fresconis, Brachybacterium muris, Brevibacterium ammoniilyticum, Brevibacterium casei, Brevibacterium epidermidis, Brevibacterium iodinum, Brevibacterium luteolum, Brevibacterium paucivorans, Brevibacterium pityocampae, Brevibacterium sanguinis, Brevundimonas albigilva, Brevundimonas diminuta, Brevundimonas vancanneytii, Caenimonas terrae, Calidifontibacter indicus, Campylobacter concisus, Campylobacter gracilis, Campylobacter hominis, Campylobacter rectus, Campylobacter showae, Campylobacter ureolyticus, Capnocytophaga gingivalis, Capnocytophaga leadbetteri, Capnocytophaga ochracea, Capnocytophaga sputigena, Cardiobacterium hominis, Cardiobacterium valvarum, Carnobacterium divergens, Catonella morbi, Caulobacter henricii, Cavicella subterranea, Cellulomonas xylanilytica, Cellvibrio vulgaris, Chitinimonas taiwanensis, Chryseobacterium arachidis, Chryseobacterium daecheongense, Chryseobacterium formosense, Chryseobacterium formosense, Chryseobacterium greenlandense, Chryseobacterium indologenes, Chryseobacterium piscium, Chryseobacterium rigui, Chryseobacterium solani, Chryseobacterium taklimakanense, Chryseobacterium ureilyticum, Chryseobacterium ureilyticum, Chryseobacterium zeae, Chryseomicrobium aureum, Cloacibacterium haliotis, Cloacibacterium normanense, Cloacibacterium normanense, Collinsella aerofaciens, Comamonas denitrificans, Comamonas terrigena, Corynebacterium accolens, Corynebacterium afermentans subsp. lipophilum, Corynebacterium ammoniagenes, Corynebacterium amycolatum, Corynebacterium aurimucosum, Corynebacterium aurimucosum, Corynebacterium coyleae, Corynebacterium durum, Corynebacterium freiburgense, Corynebacterium glaucum, Corynebacterium glyciniphilum, Corynebacterium imitans, Corynebacterium jeikeium, Corynebacterium jeikeium, Corynebacterium kroppenstedtii, Corynebacterium lipophiloflavum, Corynebacterium massiliense, Corynebacterium mastitidis, Corynebacterium matruchotii, Corynebacterium minutissimum, Corynebacterium mucifaciens, Corynebacterium mustelae, Corynebacterium mycetoides, Corynebacterium pyruviciproducens, Corynebacterium simulans, Corynebacterium singulars, Corynebacterium sputi, Corynebacterium suicordis, Corynebacterium tuberculostearicum, Corynebacterium tuberculostearicum, Corynebacterium ureicelerivorans, Corynebacterium variabile, Couchioplanes caeruleus subsp. caeruleus, Cupriavidus metallidurans, Curtobacterium herbarum, Dechloromonas agitata, Deinococcus actinosclerus, Deinococcus antarcticus, Deinococcus caeni, Deinococcus ficus, Deinococcus geothermalis, Deinococcus radiodurans, Deinococcus wulumuqiensis, Deinococcus xinjiangensis, Dermabacter hominis, Dermabacter vaginalis, Dermacoccus nishinomiyaensis, Desemzia incerta, Desertibacter roseus, Dialister invisus, Dialister micraerophilus, Dialister propionicifaciens, Dietzia aurantiaca, Dietzia cercidiphylli, Dietzia timorensis, Dietzia timorensis, Dokdonella koreensis, Dokdonella koreensis, Dolosigranulum pigrum, Eikenella corrodens, Elizabethkingia miricola, Elstera litoralis, Empedobacter brevis, Enhydrobacter aerosaccus, Enterobacter xiangfangensis, Enterococcus aquimarinus, Enterococcus faecalis, Enterococcus olivae, Erwinia rhapontici, Eubacterium eligens, Eubacterium infirmum, Eubacterium rectale, Eubacterium saphenum, Eubacterium sulci, Exiguobacterium mexicanum, Facklamia tabacinasalis, Falsirhodobacter halotolerans, Finegoldia magna, Flavobacterium cutihirudinis, Flavobacterium lindanitolerans, Flavobacterium resistens, Friedmanniella capsulata, Fusobacterium nucleatum subsp. polymorphum, Gemella haemolysans, Gemella morbillorum, Gemella palaticanis, Gemella sanguinis, Gemmobacter aquaticus, Gemmobacter caeni, Gordonia jinhuaensis, Gordonia kroppenstedtii, Gordonia polyisoprenivorans, Gordonia polyisoprenivorans, Granulicatella adiacens, Granulicatella elegans, Haemophilus parainfluenzae, Haemophilus sputorum, Halomonas sulfidaeris, Herpetosiphon aurantiacus, Hydrocarboniphaga effusa, Idiomarina marls, Janibacter anophelis, Janibacter hoylei, Janibacter indicus, Janibacter limosus, Janibacter melonis, Jeotgalicoccus halophilus, Jonquetella anthropi, Kaistia geumhonensis, Kingella denitrificans, Kingella orails, Klebsiella oxytoca, Knoellia aerolata, Knoellia locipacati, Kocuria atrinae, Kocuria carniphila, Kocuria kristinae, Kocuria palustris, Kocuria turfanensis, Lachnoanaerobaculum saburreum, Lachnoanaerobaculum saburreum, Lactobacillus crispatus, Lactobacillus finers, Lactococcus lactis subsp. lactis, Lactococcus lactis subsp. lactis, Lactococcus piscium, Lapillicoccus jejuensis, Lautropia mirabilis, Legionella beliardensis, Leptotrichia buccalis, Leptotrichia goodfellowii, Leptotrichia hofstadii, Leptotrichia hongkongensis, Leptotrichia shahii, Leptotrichia trevisanii, Leptotrichia wadei, Luteimonas terricola, Lysinibacillus fusiformis, Lysobacter spongiicola, Lysobacter xinjiangensis, Macrococcus caseolyticus, Marmoricola pocheonensis, Marmoricola scoriae, Massilia alkalitolerans, Massilia alkalitolerans, Massilia aurea, Massilia plicata, Massilia timonae, Megamonas rupellensis, Meiothermus silvanus, Methylobacterium dankookense, Methylobacterium goesingense, Methylobacterium goesingense, Methylobacterium isbiliense, Methylobacterium jeotgali, Methylobacterium oxalidis, Methylobacterium platani, Methylobacterium pseudosasicola, Methyloversatilis universalis, Microbacterium foliorum, Microbacterium hydrothermale, Microbacterium hydrothermale, Microbacterium lacticum, Microbacterium lacticum, Microbacterium laevaniformans, Microbacterium paludicola, Microbacterium petrolearium, Microbacterium phyllosphaerae, Microbacterium resistens, Micrococcus antarcticus, Micrococcus cohnii, Micrococcus flavus, Micrococcus lylae, Micrococcus terreus, Microlunatus aurantiacus, Micropruina glycogenica, Microvirga aerilata, Microvirga aerilata, Microvirga subterranea, Microvirga vignae, Microvirga zambiensis, Microvirgula aerodenitrificans, Mogibacterium timidum, Moraxella atlantae, Moraxella catarrhalis, Morganella morganii subsp. morganii, Morganella psychrotolerans, Murdochiella asaccharolytica, Mycobacterium asiaticum, Mycobacterium chubuense, Mycobacterium crocinum, Mycobacterium gadium, Mycobacterium holsaticum, Mycobacterium iranicum, Mycobacterium longobardum, Mycobacterium neoaurum, Mycobacterium neoaurum, Mycobacterium obuense, Negativicoccus succinicivorans, Neisseria bacilliformis, Neisseria oralis, Neisseria sicca, Neisseria subflava, Nesterenkonia lacusekhoensis, Nesterenkonia rhizosphaerae, Nevskia persephonica, Nevskia ramosa, Niabella yanshanensis, Niveibacterium umoris, Nocardia niwae, Nocardia thailandica, Nocardioides agariphilus, Nocardioides dilutus, Nocardioides ganghwensis, Nocardioides hwasunensis, Nocardioides nanhaiensis, Nocardioides sediminis, Nosocomiicoccus ampullae, Noviherbaspirillum malthae, Novosphingobium lindaniclasticum, Novosphingobium rosa, Ochrobactrum rhizosphaerae, Olsenella uli, Ornithinimicrobium murale, Ornithinimicrobium tianjinense, Oryzobacter terrae, Ottowia beijingensis, Paenalcaligenes suwonensis, Paenibacillus agaridevorans, Paenibacillus phoenicis, Paenibacillus xylanexedens, Paludibacterium yongneupense, Pantoea cypripedii, Parabacteroides distasonis, Paraburkholderia andropogonis, Paracoccus alcaliphilus, Paracoccus angustae, Paracoccus kocurii, Paracoccus laeviglucosivorans, Paracoccus sediminis, Paracoccus sphaerophysae, Paracoccus yeei, Parvimonas micra, Parviterribacter multiflagellatus, Patulibacter ginsengiterrae, Pedobacter aquatilis, Pedobacter ginsengisoli, Pedobacter xixiisoli, Peptococcus niger, Peptoniphilus coxii, Peptoniphilus gorbachii, Peptoniphilus harei, Peptoniphilus koenoeneniae, Peptoniphilus lacrimalis, Peptostreptococcus anaerobius, Peptostreptococcus stomatis, Phascolarctobacterium faecium, Phenylobacterium haematophilum, Phenylobacterium kunshanense, Pluralibacter gergoviae, Polymorphobacter multimanifer, Porphyromonas bennonis, Porphyromonas endodontalis, Porphyromonas gingivalis, Porphyromonas gingivicanis, Porphyromonas pasteri, Porphyromonas pogonae, Porphyromonas somerae, Povalibacter uvarum, Prevotella aurantiaca, Prevotella baroniae, Prevotella bivia, Prevotella buccae, Prevotella buccalis, Prevotella copri, Prevotella corporis, Prevotella denticola, Prevotella enoeca, Prevotella histicola, Prevotella intermedia, Prevotella jejuni, Prevotella jejuni, Prevotella maculosa, Prevotella melaninogenica, Prevotella melaninogenica, Prevotella micans, Prevotella multiformis, Prevotella nanceiensis, Prevotella nigrescens, Prevotella oris, Prevotella oulorum, Prevotella pallens, Prevotella pleuritidis, Prevotella saccharolytica, Prevotella salivae, Prevotella shahii, Prevotella timonensis, Prevotella veroralis, Propionibacterium acidifaciens, Propionibacterium acnes subsp. acnes, Propionibacterium acnes subsp. acnes, Propionibacterium acnes subsp. elongatum, Propionibacterium granulosum, Propionimicrobium lymphophilum, Propionispira arcuata, Pseudokineococcus lusitanus, Pseudomonas aeruginosa, Pseudomonas chengduensis, Pseudonocardia benzenivorans, Pseudorhodoplanes sinuspersici, Psychrobacter sanguinis, Ramlibacter ginsenosidimutans, Rheinheimera aquimaris, Rhizobium alvei, Rhizobium daejeonense, Rhizobium larrymoorei, Rhizobium rhizoryzae, Rhizobium soli, Rhizobium taibaishanense, Rhizobium vignae, Rhodanobacter glycinis, Rhodobacter veldkampii, Rhodococcus enclensis, Rhodococcus fascians, Rhodococcus fascians, Rhodovarius lipocyclicus, Rivicola pingtungensis, Roseburia inulinivorans, Rosenbergiella nectarea, Roseomonas aerilata, Roseomonas aquatica, Roseomonas mucosa, Roseomonas rosea, Roseomonas vinacea, Rothia aeria, Rothia amarae, Rothia dentocariosa, Rothia endophytica, Rothia mucilaginosa, Rothia nasimurium, Rubellimicrobium mesophilum, Rubellimicrobium roseum, Rubrobacter bracarensis, Rudaea cellulosilytica, Ruminococcus gnavus, Runella zeae, Saccharopolyspora rectivirgula, Salinicoccus qingdaonensis, Scardovia wiggsiae, Sediminibacterium ginsengisoli, Selenomonas artemidis, Selenomonas infelix, Selenomonas noxia, Selenomonas sputigena, Shewanella aestuarii, Shuttleworthia satelles, Simonsiella muelleri, Skermanella aerolata, Skermanella stibiiresistens, Slackia exigua, Smaragdicoccus niigatensis, Sneathia sanguinegens, Solirubrobacter soli, Sphingobacterium caeni, Sphingobacterium daejeonense, Sphingobacterium hotanense, Sphingobacterium kyonggiense, Sphingobacterium multivorum, Sphingobacterium nematocida, Sphingobacterium spiritivorum, Sphingobium amiense, Sphingobium indicum, Sphingobium lactosutens, Sphingobium subterraneum, Sphingomonas abaci, Sphingomonas aestuarii, Sphingomonas canadensis, Sphingomonas daechungensis, Sphingomonas dokdonensis, Sphingomonas echinoides, Sphingomonas fonticola, Sphingomonas fonticola, Sphingomonas formosensis, Sphingomonas gei, Sphingomonas hankookensis, Sphingomonas hankookensis, Sphingomonas koreensis, Sphingomonas kyeonggiensis, Sphingomonas laterariae, Sphingomonas mucosissima, Sphingomonas oligophenolica, Sphingomonas pseudosanguinis, Sphingomonas sediminicola, Sphingomonas yantingensis, Sphingomonas yunnanensis, Sphingopyxis indica, Spirosoma rigui, Sporacetigenium mesophilum, Sporocytophaga myxococcoides, Staphylococcus auricularis, Staphylococcus epidermidis, Staphylococcus epidermidis, Staphylococcus hominis subsp. novobiosepticus, Staphylococcus lugdunensis, Staphylococcus pettenkoferi, Stenotrophomonas koreensis, Stenotrophomonas rhizophila, Stenotrophomonas rhizophila, Streptococcus agalactiae, Streptococcus canis, Streptococcus cristatus, Streptococcus gordonii, Streptococcus infantis, Streptococcus intermedius, Streptococcus mutans, Streptococcus oligofermentans, Streptococcus oralis, Streptococcus sanguinis, Streptomyces iconiensis, Streptomyces yanglinensis, Tabrizicola aquatica, Tahibacter caeni, Tannerella forsythia, Tepidicella xavieri, Tepidimonas fonticaldi, Terracoccus luteus, Tessaracoccus flavescens, Thermus thermophilus, Tianweitania sediminis, Tianweitania sediminis, Treponema amylovorum, Treponema denticola, Treponema lecithinolyticum, Treponema medium, Turicella otitidis, Turicibacter sanguinis, Undibacterium oligocarboniphilum, Undibacterium squillarum, Vagococcus salmoninarum, Varibaculum cambriense, Vibrio metschnikovii, Xanthobacter tagetidis, Xenophilus aerolatus, Xenophilus arseniciresistens, Yimella lutea, Zimmermannella alba, Zimmermannella bifida and Zoogloea caeni.

In other embodiments, the targeted bacteria cells are those commonly found in the vaginal microbiota and are, without limitation, Acinetobacter antiviralis, Acinetobacter baumannii, Acinetobacter calcoaceticus, Acinetobacter johnsonii, Actinobaculum massiliense, Actinobaculum schaalii, Actinomyces europaeus, Actinomyces graevenitzii, Actinomyces israelii, Actinomyces meyeri, Actinomyces naeslundii, Actinomyces neuii, Actinomyces odontolyticus, Actinomyces turicensis, Actinomyces urogenitalis, Actinomyces viscosus, Aerococcus christensenii, Aerococcus urinae, Aerococcus viridans, Aeromonas encheleia, Aeromonas salmonicida, Afipia massiliensis, Agrobacterium tumefaciens, Algoriphagus aquatilis, Aliivibrio wodanis, Alistipes finegoldii, Alloiococcus otitis, Alloprevotella tannerae, Alloscardovia omnicolens, Altererythrobacter epoxidivorans, Ammoniphilus oxalaticus, Amnibacterium kyonggiense, Anaerococcus hydrogenalis, Anaerococcus lactolyticus, Anaerococcus murdochii, Anaerococcus obesiensis, Anaerococcus prevotii, Anaerococcus tetradius, Anaerococcus vaginalis, Anaeroglobus geminatus, Anoxybacillus pushchinoensis, Aquabacterium parvum, Arcanobacterium phocae, Arthrobacter aurescens, Asticcacaulis excentricus, Atopobium minutum, Atopobium parvulum, Atopobium rimae, Atopobium vaginae, Avibacterium gallinarum, Bacillus acidicola, Bacillus atrophaeus, Bacillus cereus, Bacillus cibi, Bacillus coahuilensis, Bacillus gaemokensis, Bacillus methanolicus, Bacillus oleronius, Bacillus pumilus, Bacillus shackletonii, Bacillus sporothermodurans, Bacillus subtilis, Bacillus wakoensis, Bacillus weihenstephanensis, Bacteroides barnesiae, Bacteroides coagulans, Bacteroides dorei, Bacteroides faecis, Bacteroides forsythus, Bacteroides fragilis, Bacteroides nordii, Bacteroides ovatus, Bacteroides salyersiae, Bacteroides stercoris, Bacteroides uniformis, Bacteroides vulgatus, Bacteroides xylanisolvens, Bacteroides zoogleoformans, Barnesiella viscericola, Bhargavaea cecembensis, Bifidobacterium adolescentis, Bifidobacterium bifidum, Bifidobacterium breve, Bifidobacterium dentium, Bifidobacterium logum subsp. infantis, Bifidobacterium longum, Bifidobacterium pseudocatenulatum, Bifidobacterium scardovii, Bilophila wadsworthia, Blautia hydrogenotrophica, Blautia obeum, Blautia producta, Brachybacterium faecium, Bradyrhizobium japonicum, Brevibacterium mcbrellneri, Brevibacterium otitidis, Brevibacterium paucivorans, Bulleidia extructa, Burkholderia fungorum, Burkholderia phenoliruptix, Caldicellulosiruptor saccharolyticus, Caldimonas taiwanensis, Campylobacter gracilis, Campylobacter hominis, Campylobacter sputorum, Campylobacter ureolyticus, Capnocytophaga ochracea, Cardiobacterium hominis, Catonella morbi, Chlamydia trachomatis, Chlamydophila abortus, Chondromyces robustus, Chryseobacterium aquaticum, Citrobacter youngae, Cloacibacterium normanense, Clostridium cavendishii, Clostridium colicanis, Clostridium jejuense, Clostridium perfringens, Clostridium ramosum, Clostridium sordellii, Clostridium viride, Comamonas terrigena, Corynebacterium accolens, Corynebacterium appendicis, Corynebacterium coyleae, Corynebacterium glucuronolyticum, Corynebacterium glutamicum, Corynebacterium jeikeium, Corynebacterium kroppenstedtii, Corynebacterium lipophiloflavum, Corynebacterium minutissimum, Corynebacterium mucifaciens, Corynebacterium nuruki, Corynebacterium pseudogenitalium, Corynebacterium pyruviciproducens, Corynebacterium singulare, Corynebacterium striatum, Corynebacterium tuberculostearicum, Corynebacterium xerosis, Cryobacterium psychrophilum, Curtobacterium flaccumfaciens, Cutibacterium acnes, Cutibacterium avidum, Cytophaga xylanolytica, Deinococcus radiophilus, Delftia tsuruhatensis, Desulfovibrio desulfuricans, Dialister invisus, Dialister micraerophilus, Dialister pneumosintes, Dialister propionicifaciens, Dickeya chrysanthemi, Dorea longicatena, Eggerthella lenta, Eggerthia catenaformis, Eikenella corrodens, Enhydrobacter aerosaccus, Enterobacter asburiae, Enterobacter cloacae, Enterococcus avium, Enterococcus durans, Enterococcus faecalis, Enterococcus faecium, Enterococcus hirae, Erwinia persicina, Erwinia rhapontici, Erwinia toletana, Escherichia coli, Escherichia fergusonii, Eubacterium brachy, Eubacterium eligens, Eubacterium nodatum, Eubacterium rectale, Eubacterium saphenum, Eubacterium siraeum, Eubacterium sulci, Eubacterium yurii, Exiguobacterium acetylicum, Facklamia ignava, Faecalibacterium prausnitzii, Filifactor alocis, Finegoldia magna, Fusobacterium gonidiaformans, Fusobacterium nucleatum, Fusobacterium periodonticum, Gardnerella vaginalis, Gemella asaccharolytica, Gemella bergeri, Gemella haemolysans, Gemella sanguinis, Geobacillus stearothermophilus, Geobacillus thermocatenulatus, Geobacillus thermoglucosidasius, Geobacter grbiciae, Granulicatella elegans, Haemophilus ducreyi, Haemophilus haemolyticus, Haemophilus parahaemolyticus, Haemophilus parainfluenzae, Hafnia alvei, Halomonas meridiana, Halomonas phoceae, Halomonas venusta, Herbaspirillum seropedicae, Janthinobacterium lividum, Jonquetella anthropi, Klebsiella granulomatis, Klebsiella oxytoca, Klebsiella pneumoniae, Lactobacillus acidophilus, Lactobacillus amylovorus, Lactobacillus brevis, Lactobacillus coleohominis, Lactobacillus crispatus, Lactobacillus curvatus, Lactobacillus delbrueckii, Lactobacillus fermentum, Lactobacillus gasseri, Lactobacillus helveticus, Lactobacillus iners, Lactobacillus jensenii, Lactobacillus johnsonii, Lactobacillus kalixensis, Lactobacillus kefiranofaciens, Lactobacillus kimchicus, Lactobacillus kitasatonis, Lactobacillus mucosae, Lactobacillus panis, Lactobacillus paracasei, Lactobacillus plantarum, Lactobacillus pontis, Lactobacillus reuteri, Lactobacillus rhamnosus, Lactobacillus salivarius, Lactobacillus ultunensis, Lactobacillus vaginalis, Lactococcus lactis, Leptotrichia buccalis, Leuconostoc carnosum, Leuconostoc citreum, Leuconostoc garlicum, Leuconostoc lactis, Leuconostoc mesenteroides, Lysinimonas kribbensis, Mageeibacillus indolicus, Maribacter orientalis, Marinomonas protea, Marinospirillum insulare, Massilia timonae, Megasphaera elsdenii, Megasphaera micronuciformis, Mesorhizobium amorphae, Methylobacterium radiotolerans, Methylotenera versatilis, Microbacterium halophilum, Micrococcus luteus, Microterricola viridarii, Mobiluncus curtisii, Mobiluncus mulieris, Mogibacterium timidum, Moorella glycerini, Moraxella osloensis, Morganella morganii, Moryella indoligenes, Murdochiella asaccharolytica, Mycoplasma alvi, Mycoplasma genitalium, Mycoplasma hominis, Mycoplasma muris, Mycoplasma salivarium, Negativicoccus succinicivorans, Neisseria flava, Neisseria gonorrhoeae, Neisseria mucosa, Neisseria subflava, Nevskia ramosa, Nevskia soli, Nitriliruptor alkaliphilus, Odoribacter splanchnicus, Oligella urethralis, Olsenella uli, Paenibacillus amylolyticus, Paenibacillus humicus, Paenibacillus pabuli, Paenibacillus pasadenensis, Paenibacillus pini, Paenibacillus validus, Pantoea agglomerans, Parabacteroides merdae, Paraburkholderia caryophylli, Paracoccus yeei, Parastreptomyces abscessus, Parvimonas micra, Pectobacterium betavasculorum, Pectobacterium carotovorum, Pediococcus acidilactici, Pediococcus ethanolidurans, Pedobacter alluvionis, Pedobacter wanjuense, Pelomonas aquatica, Peptococcus niger, Peptoniphilus asaccharolyticus, Peptoniphilus gorbachii, Peptoniphilus harei, Peptoniphilus indolicus, Peptomphilus lacrimalis, Peptomphilus massiliensis, Peptostreptococcus anaerobius, Peptostreptococcus massiliae, Peptostreptococcus stomatis, Photobacterium angustum, Photobacterium frigidiphilum, Photobacterium phosphoreum, Porphyromonas asaccharolytica, Porphyromonas bennonis, Porphyromonas catoniae, Porphyromonas endodontalis, Porphyromonas gingivalis, Porphyromonas somerae, Porphyromonas uenonis, Prevotella amnii, Prevotella baroniae, Prevotella bergensis, Prevotella bivia, Prevotella buccae, Prevotella buccalis, Prevotella colorans, Prevotella copri, Prevotella corporis, Prevotella dentalis, Prevotella denticola, Prevotella disiens, Prevotella intermedia, Prevotella loescheii, Prevotella marshii, Prevotella melaninogenica, Prevotella micans, Prevotella nigrescens, Prevotella oris, Prevotella pleuritidis, Prevotella ruminicola, Prevotella shahii, Prevotella stercorea, Prevotella timonensis, Prevotella veroralis, Propionimicrobium lymphophilum, Proteus mirabilis, Pseudomonas abietamphila, Pseudomonas aeruginosa, Pseudomonas amygdali, Pseudomonas azotoformans, Pseudomonas chlororaphis, Pseudomonas cuatrocienegasensis, Pseudomonas fluorescens, Pseudomonas fulva, Pseudomonas lutea, Pseudomonas mucidolens, Pseudomonas oleovorans, Pseudomonas orientalis, Pseudomonas pseudoalcaligenes, Pseudomonas psychrophila, Pseudomonas putida, Pseudomonas synxantha, Pseudomonas syringae, Pseudomonas tolaasii, Pseudopropionibacterium propionicum, Rahnella aquatilis, Ralstonia pickettii, Ralstonia solanacearum, Raoultella planticola, Rhizobacter dauci, Rhizobium etli, Rhodococcus fascians, Rhodopseudomonas palustris, Roseburia intestinalis, Roseburia inulinivorans, Rothia mucilaginosa, Ruminococcus bromii, Ruminococcus gnavus, Ruminococcus torques, Sanguibacter keddieii, Sediminibacterium salmoneum, Selenomonas bovis, Serratia fonticola, Serratia liquefaciens, Serratia marcescens, Shewanella algae, Shewanella amazonensis, Shigella boydii, Shigella sonnei, Slackia exigua, Sneathia amnii, Sneathia sanguinegens, Solobacterium moorei, Sorangium cellulosum, Sphingobium amiense, Sphingobium japonicum, Sphingobium yanoikuyae, Sphingomonas wittichii, Sporosarcina aquimarina, Staphylococcus aureus, Staphylococcus auricularis, Staphylococcus capitis, Staphylococcus epidermidis, Staphylococcus haemolyticus, Staphylococcus hominis, Staphylococcus lugdunensis, Staphylococcus saprophyticus, Staphylococcus schleiferi, Staphylococcus simiae, Staphylococcus simulans, Staphylococcus warneri, Stenotrophomonas maltophilia, Stenoxybacter acetivorans, Streptococcus agalactiae, Streptococcus anginosus, Streptococcus australis, Streptococcus equinus, Streptococcus gallolyticus, Streptococcus infantis, Streptococcus intermedius, Streptococcus lutetiensis, Streptococcus marimammalium, Streptococcus mitis, Streptococcus mutans, Streptococcus oxalis, Streptococcus parasanguinis, Streptococcus phocae, Streptococcus pseudopneumoniae, Streptococcus salivarius, Streptococcus sanguinis, Streptococcus thermophilus, Sutterella wadsworthensis, Tannerella forsythia, Terrahaemophilus aromaticivorans, Treponema denticola, Treponema maltophilum, Treponema parvum, Treponema vincentii, Trueperella bernardiae, Turicella otitidis, Ureaplasma parvum, Ureaplasma urealyticum, Varibaculum cambriense, Variovorax paradoxus, Veillonella atypica, Veillonella dispar, Veillonella montpellierensis, Veillonella parvula, Virgibacillus proomii, Viridibacillus arenosi, Viridibacillus arvi, Weissella cibaria, Weissella soli, Xanthomonas campestris, Xanthomonas vesicatoria, Zobellia laminariae and Zoogloea ramigera.

In one embodiment, the targeted bacteria are Escherichia coli. In a particular embodiment, said targeted bacteria are Shiga-Toxin producing E. coli (STEC).

The targeted bacterial cell population may comprise one or several bacteria of interest as defined above. In particular, the targeted bacterial cell population may comprise Escherichia coli and one or several other bacteria of interest as defined above.

Thus, bacteriophages used for preparing the bacterial delivery vehicles, and then the bacterial delivery vehicles, may target (e.g., specifically target) a bacterial cell from any one or more of the foregoing genus and/or species of bacteria to specifically deliver the payload of interest.

In one embodiment, the targeted bacteria are pathogenic bacteria. The targeted bacteria can be virulent bacteria.

The targeted bacteria can be antibacterial resistance bacteria, including those selected from the group consisting of extended-spectrum beta-lactamase-producing (ESBL) Escherichia coli, ESBL Klebsiella pneumoniae, vancomycin-resistant Enterococcus (VRE), methicillin-resistant Staphylococcus aureus (MRSA), multidrug-resistant (MDR) Acinetobacter baumannii, MDR Enterobacter spp., and a combination thereof. The targeted bacteria can be selected from the group consisting of extended-spectrum beta-lactamase-producing (ESBL) Escherichia coli strains.

Alternatively, the targeted bacterium can be a bacterium of the microbiome of a given species, including a bacterium of the human microbiota.

The present disclosure is directed to a bacterial delivery vehicle containing the payload as described herein. The bacterial delivery vehicles are prepared from bacterial virus. The bacterial delivery vehicles are chosen in order to be able to introduce the payload into the targeted bacteria.

Bacterial viruses, from which the bacterial delivery vehicles disclosed herein may be derived, include bacteriophages. Optionally, the bacteriophage is selected from the Order Caudovirales consisting of, based on the taxonomy of Krupovic et al, Arch Virol, 2015, the family Myoviridae, the family Podoviridae, the family Siphoviridae, and the family Ackermannviridae.

Bacteriophages may be selected from the family Myoviridae (such as, without limitation, genus Cp220virus, Cp1virus, Ea214virus, Felixo1virus, Mooglevirus, Suspvirus, Hp1virus, P2virus, Kayvirus, P100virus, Silviavirus, Spolvirus, Tsarbombavirus, Twortvirus, Cc31virus, Jd18virus, Js98virus, Kpl5virus, Moonvirus, Rb49virus, Rb69virus, S16virus, Schizot4virus, Sp18virus, T4virus, Cr3virus, Selvirus, V5virus, Abouovirus, Agatevirus, Agrican357virus, Ap22virus, Arvlvirus, B4virus, Bastillevirus, Bc431virus, Bcep78virus, Bcepmuvirus, Biquartavirus, Bxzlvirus, Cd119virus, Cp5lvirus, Cvm10virus, Eah2virus, Elvirus, Hapunavirus, Jimmervirus, Kpp10virus, M12virus, Machinavirus, Marthavirus, Msw3virus, Muvirus, Myohalovirus, Nitivirus, Plvirus, Pakpunavirus, Pbunavirus, Phikzvirus, Rheph4virus, Rsl2virus, Rslunavirus, Secunda5virus, Sep1virus, Spn3virus, Svunavirus, Tglvirus, Vhmlvirus and Wphvirus).

Bacteriophages may be selected from the family Podoviridae (such as, without limitation, genus Fri1virus, Kp32virus, Kp34virus, Phikmvvirus, Pradovirus, Sp6virus, T7virus, Cp1virus, P68virus, Phi29virus, Nona33virus, Pocjvirus, Tl2011virus, Bcep22virus, Bpp1virus, Cba41virus, Dfl12virus, Ea92virus, Epsilon15virus, F116virus, G7cvirus, Jwalphavirus, Kf1virus, Kpp25virus, Lit1virus, Luz24virus, Luz7virus, N4virus, Nonanavirus, P22virus, Pagevirus, Phieco32virus, Prtbvirus, Sp58virus, Una961virus and Vp5virus).

Bacteriophages may be selected from the family Siphoviridae (such as, without limitation, genus Camvirus, Likavirus, R4virus, Acadianvirus, Coopervirus, Pg1virus, Pipefishvirus, Rosebushvirus, Brujitavirus, Che9cvirus, Hawkeyevirus, Plotvirus, Jerseyvirus, Klgvirus, Sp31virus, Lmdlvirus, Una4virus, Bongovirus, Reyvirus, Buttersvirus, Charlievirus, Redivirus, Baxtervirus, Nymphadoravirus, Bignuzvirus, Fishburnevirus, Phayoncevirus, Kp36virus, Roguelvirus, Rtpvirus, T1virus, T1svirus, Ab18virus, Amigovirus, Anatolevirus, Andromedavirus, Attisvirus, Barnyardvirus, Bernal13virus, Biseptimavirus, Bronvirus, C2virus, C5virus, Cba181virus, Cbastvirus, Cecivirus, Che8virus, Chivirus, Cjwlvirus, Corndogvirus, Cronusvirus, D3112virus, D3virus, Decurrovirus, Demosthenesvirus, Doucettevirus, E125virus, Eiauvirus, Ff47virus, Gaiavirus, Gilesvirus, Gordonvirus, Gordtnkvirus, Harrisonvirus, Hk578virus, Hk97virus, Jenstvirus, Jwxvirus, Kelleziovirus, Korravirus, L5virus, lambdavirus, Laroyevirus, Liefievirus, Marvinvirus, Mudcatvirus, N15virus, Nonagvirus, Nplvirus, Omegavirus, P12002virus, P12024virus, P23virus, P70virus, Pa6virus, Pamx74virus, Patiencevirus, Pbi1virus, Pepy6virus, Pfr1virus, Phic31virus, Phicbkvirus, Phietavirus, Phifelvirus, Phijl1virus, Pis4avirus, Psavirus, Psimunavirus, Rdjlvirus, Rer2virus, Sap6virus, Send513virus, Septima3virus, Seuratvirus, Sextaecvirus, Sfi11virus, Sfi21dtivirus, Sitaravirus, Sk1virus, Slashvirus, Smoothievirus, Soupsvirus, Spbetavirus, Ssp2virus, T5virus, Tankvirus, Tin2virus, Titanvirus, Tm4virus, Tp2lvirus, Tp84virus, Triavirus, Trigintaduovirus, Vegasvirus, Vendettavirus, Wbetavirus, Wildcatvirus, Wizardvirus, Woesvirus, Xp10virus, Ydn12virus and Yuavirus).

Bacteriophages may be selected from the family Ackermannviridae (such as, without limitation, genus Ag3virus, Limestonevirus, Cba120virus and Vilvirus).

Optionally, the bacteriophage is not part of the order Caudovirales but from families with unassigned order such as, without limitation, family Tectiviridae (such as genus Alphatectivirus, Betatectivirus), family Corticoviridae (such as genus Corticovirus), family Inoviridae (such as genus Fibrovirus, Habenivirus, Inovirus, Lineavirus, Plectrovirus, Saetivirus, Vespertiliovirus), family Cystoviridae (such as genus Cystovirus), family Leviviridae (such as genus Allolevivirus, Levivirus), family Microviridae (such as genus Alpha3microvirus, G4microvirus, Phix174microvirus, Bdellomicrovirus, Chlamydiamicrovirus, Spiromicrovirus) and family Plasmaviridae (such as genus Plasmavirus).

Optionally, the bacteriophage is targeting Archea not part of the Order Caudovirales but from families with unassigned order such as, without limitation, Ampullaviridae, FuselloViridae, Globuloviridae, Guttaviridae, Lipothrixviridae, Pleolipoviridae, Rudiviridae, Salterprovirus and Bicaudaviridae.

A non-exhaustive listing of bacterial genera and their known host-specific bacteria viruses is presented in the following paragraphs. The chimeric RBPs and/or the branched RBPs and/or the recombinant gpJ proteins and/or the recombinant gpH proteins, and the bacterial delivery vehicles disclosed herein may be engineered, as non-limiting examples, from the following phages. Synonyms and spelling variants are indicated in parentheses. Homonyms are repeated as often as they occur (e.g., D, D, d). Unnamed phages are indicated by “NN” beside their genus and their numbers are given in parentheses.

Bacteria of the genus Actinomyces can be infected by the following phages: Av-I, Av-2, Av-3, BF307, CT1, CT2, CT3, CT4, CT6, CT7, CT8 and 1281.

Bacteria of the genus Aeromonas can be infected by the following phages: AA-I, Aeh2, N, PM1, TP446, 3, 4, 11, 13, 29, 31, 32, 37, 43, 43-10T, 51, 54, 55R.1, 56, 56RR2, 57, 58, 59.1, 60, 63, Aeh1, F, PM2, 1, 25, 31, 40RR2.8t, (syn=44R), (syn=44RR2.8t), 65, PM3, PM4, PM5 and PM6.

Bacteria of the genus Bacillus can be infected by the following phages: A, aiz1, A1-K-I, B, BCJA1, BC1, BC2, BLL1, BL1, BP142, BSL1, BSL2, BS1, BS3, BS8, BS15, BS18, BS22, BS26, BS28, BS31, BS104, BS105, BS106, BTB, B1715V1, C, CK-I, Coll, Corl, CP-53, CS-I, CSi, D, D, D, D5, entl, FPB, FP9, FSi, FS2, FS3, FS5, FS8, FS9, G, GH8, GT8, GV-I, GV-2, GT-4, g3, g12, g13, g14, g16, g17, g21, g23, g24, g29, H2, ken1, KK-88, Kum1, Kyu1, J7W-1, LP52, (syn=LP-52), L7, Mex1, MJ-I, mor2, MP-7, MP10, MP12, MP14, MP15, Neo1, N^{o}2, N5, N6P, PBC1, PBLA, PBP1, P2, S-a, SF2, SF6, Shal, Sill, 5P02, (syn=ΦSPP1), SPβ, STI, STi, SU-Il, t, TbI, Tb2, Tb5, TbIO, Tb26, Tb51, Tb53, Tb55, Tb77, Tb97, Tb99, Tb560, Tb595, Td8, Td6, Td15, TgI, Tg4, Tg6, Tg7, Tg9, TgIO, TgIl, Tg13, Tg15, Tg21, Tin1, Tin7, Tin8, Tin13, Tm3, Toc1, Tog1, tol1, TP-I, TP-10vir, TP-15c, TP-16c, TP-17c, TP-19, TP35, TP51, TP-84, Tt4, Tt6, type A, type B, type C, type D, type E, VA-9, W, wx23, wx26, Yun1, α, γ, p1 1, φmed-2, φT, φ3T, φ75, φlO5, (syn=φlO5), IA, IB, 1-97A, 1-97B, 2, 2, 3, 3, 3, 5, 12, 14, 20, 30, 35, 36, 37, 38, 41C, 51, 63, 64, 138D, I, II, IV, NN-Bacillus (13), ale1, AR1, AR2, AR3, AR7, AR9, Bace-11, (syn=11), Bastille, BL1, BL2, BL3, BL4, BLS, BL6, BL8, BL9, BP124, BS28, BS80, Ch, CP-51, CP-54, D-5, darl, den1, DP-7, entl, FoSi, FoS2, FS4, FS6, FS7, G, gall, gamma, GE1, GF-2, GSi, GT-I, GT-2, GT-3, GT-4, GT-5, GT-6, GT-7, GV-6, g15, 19, 110, ISi, K, MP9, MP13, MP21, MP23, MP24, MP28, MP29, MP30, MP32, MP34, MP36, MP37, MP39, MP40, MP41, MP43, MP44, MP45, MP47, MP50, NLP-I, No. 1, N17, N19, PBS1, PK1, PMB1, PMB12, PMJ1, S, SPO1, SP3, SP5, SP6, SP7, SP8, SP9, SPlO, SP-15, SP50, (syn=SP-50), SP82, SST, subl, SW, Tg8, Tg12, Tg13, Tg14, thul, thuΛ, thuS, Tin4, Tin23, TP-13, TP33, TP50, TSP-I, type V, type VI, V, Vx, β22, φe, φNR2, φ25, φ63, 1, 1, 2, 2C, 3NT, 4, 5, 6, 7, 8, 9, 10, 12, 12, 17, 18, 19, 21, 138, III, 4 (B. megateriwn), 4 (B. sphaericus), AR13, BPP-IO, BS32, BS107, B1, B2, GA-I, GP-IO, GV-3, GV-5, g8, MP20, MP27, MP49, Nf, PPS, PP6, SFS, Tg18, TP-I, Versailles, φ15, φ29, 1-97, 837/IV, m\ddot{ι}-Bacillus (1), Bat10, BSL10, BSLI1, BS6, BSI1, BS16, BS23, BSlOl, BS102, g18, morl, PBL1, SN45, thu2, thu3, TmI, Tm2, TP-20, TP21, TP52, type F, type G, type IV, HN-BacMus (3), BLE, (syn=0c), BS2, BS4, BS5, BS7, B10, B12, BS20, BS21, F, MJ-4, PBA12, AP50, AP50-04, AP50-11, AP50-23, AP50-26, AP50-27 and Bam35. The following Bacillus-specific phages are defective: DLP10716, DLP-11946, DPB5, DPB12, DPB21, DPB22, DPB23, GA-2, M, No. IM, PBLB, PBSH, PBSV, PBSW, PBSX, PBSY, PBSZ, phi, SPa, type 1 and μ.

Bacteria of the genus Bacteroides can be infected by the following phages: ad 12, Baf-44, Baf-48B, Baf-64, Bf-I, Bf-52, B40-8, F1, β1, φA1, φBrO1, φBrO2, 11, 67.1, 67.3, 68.1, mt-Bacteroides (3), Bf42, Bf71, HN-Bdellovibrio (1) and BF-41.

Bacteria of the genus Bordetella can be infected by the following phages: 134 and NN-Bordetella (3).

Bacteria of the genus Borrellia can be infected by the following phages: NN-Borrelia (1) and NN-Borrelia (2).

Bacteria of the genus Brucella can be infected by the following phages: A422, Bk, (syn=Berkeley), BM29, FOi, (syn=F01), (syn=FQ1), D, FP2, (syn=FP2), (syn=FD2), Fz, (syn=Fz75/13), (syn=Firenze 75/13), (syn=Fi), Fi, (syn=Fl), Fim, (syn=FIm), (syn=Fim), FiU, (syn=F1U), (syn=FiU), F2, (syn=F2), F3, (syn=F3), F4, (syn=F4), F5, (syn=F5), F6, F7, (syn=F7), F25, (syn=F25), (syn=£25), F25U, (syn=F25u), (syn=F25U), (syn=F25V), F44, (syn-F44), F45, (syn=F45), F48, (syn=F48), I, Im, M, MC/75, M51, (syn=M85), P, (syn=D), S708, R, Tb, (syn=TB), (syn=Tbilisi), W, (syn=Wb), (syn=Weybridge), X, 3, 6, 7, 10/1, (syn=10), (syn=F8), (syn=F8), 12m, 24/11, (syn=24), (syn=F9), (syn=F9), 45/111, (syn=45), 75, 84, 212/XV, (syn=212), (syn=Fi0), (syn=FlO), 371/XXIX, (syn=371), (syn=Fn), (syn=F11) and 513.

Bacteria of the genus Burkholderia can be infected by the following phages: CP75, NN-Burkholderia (1) and 42.

Bacteria of the genus Campylobacter can be infected by the following phages: C type, NTCC12669, NTCC12670, NTCC12671, NTCC12672, NTCC12673, NTCC12674, NTCC12675, NTCC12676, NTCC12677, NTCC12678, NTCC12679, NTCC12680, NTCC12681, NTCC12682, NTCC12683, NTCC12684, 32f, 111c, 191, NN-Campylobacter (2), Vfi-6, (syn=V19), VfV-3, V2, V3, V8, V16, (syn=Vfi-1), V19, V20(V45), V45, (syn=V-45) and NN-Campylobacter (1).

Bacteria of the genus Chlamydia can be infected by the following phages: Chpl.

Bacteria of the genus Clostridium can be infected by the following phages: CAK1, CAS, Cal, CEβ, (syn=1C), CEγ, Cldl, c-n71, c-203 Tox−, DEβ, (syn=ID), (syn=1Dt0X+), HM3, KM1, KT, Ms, NA1, (syn=Naltox+), PA135Oe, Pfó, PL73, PL78, PL81, P1, P50, P5771, P19402, 1Ct0X+, 2Ct0X\ 2D3 (syn=2Dt0X+), 3C, (syn=3Ctox+), 4C, (syn=4Ct0X+), 56, III-1, NN-Clostridium (61), NB1t0X+, α1, CA1, HMT, HM2, PF15 P-23, P-46, Q-05, Q-oe, Q-16, Q-21, Q-26, Q-40, Q-46, 5111, SA02, WA01, WA03, Wm, W523, 80, C, CA2, CA3, CPT1, CPT4, c1, c4, c5, HM7, H11/A1, H18/Ax, FWS23, Hi58ZA1, K2ZA1, K21ZS23, ML, NA2t0X; Pf2, Pf3, Pf4, S9ZS3, S41ZA1, S44ZS23, α2, 41, 112ZS23, 214/S23, 233/Ai, 234/S23, 235/S23, II-1, 11-2, 11-3, NN-Clostridium (12), CAI, F1, K, S2, 1, 5 and NN-Clostridium (8).

Bacteria of the genus Corynebacterium can be infected by the following phages: CGK1 (defective), A, A2, A3, AlO1, A128, A133, A137, A139, A155, A182, B, BF, B17, B18, B51, B271, B275, B276, B277, B279, B282, C, capi, CC1, CG1, CG2, CG33, CL31, Cog, (syn=CGS), D, E, F, H, H-I, hqi, hq2, 11ZH33, Ii/31, J, K, K, (syn=Ktox″), L, L, (syn=Ltox+), M, MC-I, MC-2, MC-3, MC-4, MLMa, N, O, ovi, ov2, ov3, P, P, R, RP6, RS29, S, T, U, UB1, ub2, UH1, UH3, uh3, uh5, uh6, β, (syn=βtox+), βhv64, βvir, γ, (syn=γtoχ−), γ19, δ, (syn=δ′ox+), p, (syn=ptoχ−), Φ9, Φ984, ω, IA, 1/1180, 2, 2/1180, 5/1180, 5ad/9717, 7/4465, 8/4465, 8ad/10269, 10/9253, 13Z9253, 15/3148, 21/9253, 28, 29, 55, 2747, 2893, 4498 and 5848.

Bacteria of the genus Enterococcus can be infected by the following phages: DF78, F1, F2, 1, 2, 4, 14, 41, 867, D1, SB24, 2BV, 182, 225, C2, C2F, E3, E62, DS96, H24, M35, P3, P9, SB1O1, S2, 2BII, 5, 182a, 705, 873, 881, 940, 1051, 1057, 21096C, NN-Enterococcus (1), PE1, F1, F3, F4, VD13, 1, 200, 235 and 341.

Bacteria of the genus Erysipelothrix can be infected by the following phage: NN-Eiysipelothrix (1).

Bacteria of the genus Escherichia can be infected by the following phages: BW73, B278, D6, D108, E, E1, E24, E41, FI-2, FI-4, FI-5, HI8A, Ffl8B, i, MINI, Mu, (syn=mu), (syn=MuI), (syn=Mu-I), (syn=MU-I), (syn=MuI), (syn=μ), 025, PhI-5, Pk, PSP3, P1, P1D, P2, P4 (defective), S1, Wφ, φK13, φR73 (defective), φ1, φ2 , φ7, φ92, ψ (defective), 7 A, 8φ, 9φ, 15 (defective), 18, 28-1, 186, 299, HH-Escherichia (2), AB48, CM, C4, C16, DD-VI, (syn=Dd-Vi), (syn=DDVI), (syn=DDVi), E4, E7, E28, FI1, FI3, H, H1, H3, H8, K3, M, N, ND-2, ND-3, ND4, ND-5, ND6, ND-7, Ox-I (syn=OX1), (syn=HF), Ox-2 (syn=0x2), (syn=0X2), Ox-3, Ox-4, Ox-5, (syn=0X5), Ox-6, (syn=66F), (syn=φ66t), (syn=φ66t−)5 0111, PhI-I, RB42, RB43, RB49, RB69, S, Sal-I, Sal-2, Sal-3, Sal-4, Sal-5, Sal-6, TC23, TC45, TuII*-6, (syn=TuII*), TuIP-24, TuII*46, TuIP-60, T2, (syn=ganuTia), (syn=γ), (syn=PC), (syn=P.C.), (syn=T-2), (syn=T2), (syn=P4), T4, (syn=T-4), (syn=T4), T6, T35, α1, 1, IA, 3, (syn=Ac3), 3A, 3T+, (syn=3), (syn=M1), (syn=φ5), 9266Q, CFO103, HK620, J, K, K1F, m59, no. A, no. E, no. 3, no. 9, N4, sd, (syn=Sd), (syn=SD), (syn=Sa)3 (syn=sd), (syn=SD), (syn=CD), T3, (syn=T-3), (syn=T3), T7, (syn=T-7), (syn=T7), WPK, W31, ΔH, φC3888, φK3, φK7, φK12, φV-1, Φ04-CF, Φ05, Φ06, Φ07, φ1, φ1.2, φ20, φ95, φ263, φlO92, φl, φll, (syn=φW), Ω8, 1, 3, 7, 8, 26, 27, 28-2, 29, 30, 31, 32, 38, 39, 42, 933W, NN-Escherichia (1), Esc-7-11, AC30, CVX-5, C1, DDUP, EC1, EC2, E21, E29, F1, F26S, F27S, Hi, HK022, HK97, (syn=ΦHK97), HK139, HK253, HK256, K7, ND-I, no.D, PA-2, q, S2, T1, (syn=α), (syn=P28), (syn=T-I), (syn=Tx), T3C, T5, (syn=T-5), (syn=T5), UC-I, w, β4, γ2, λ (syn=lambda), (syn=Φλ), ΦD326, γ, Φ06, Φ7, Φ10, φ80, χ, (syn=χi), (syn=φχ), (syn=φχi), 2, 4, 4A, 6, 8A, 102, 150, 168, 174, 3000, AC6, AC7, AC28, AC43, AC50, AC57, AC81, AC95, HK243, KlO, ZG/3A, 5, 5A, 21EL, H19-J and 933H.

Bacteria of the genus Fusobacterium can be infected by the following phages: NN-Fusobacterium (2), fv83-554/3, fv88-531/2, 227, fv2377, fv2527 and fv8501.

Bacteria of the genus Haemophilus can be infected by the following phages: HP1, S2 and N3.

Bacteria of the genus Helicobacter can be infected by the following phages: HP1 and ^{∨∨}Helicobacter (1).

Bacteria of the genus Klebsiella can be infected by the following phages: AIO-2, KI4B, K16B, K19, (syn=K19), K114, K115, K121, K128, K129, KI32, K133, K135, K1106B, K1171B, K1181B, K1832B, AIO-I, AO-I, AO-2, AO-3, FC3-10, K, K11, (syn=KI1), K12, (syn=K12), K13, (syn=K13), (syn=K1 70/11), K14, (syn=K14), K15, (syn=K15), K16, (syn=K16), K17, (syn=K17), K18, (syn=K18), K119, (syn=K19), K127, (syn=K127), K131, (syn=K131), K135, K1171B, II, VI, IX, CI-I, K14B, K18, K111, K112, K113, K116, K117, K118, K120, K122, K123, K124, K126, K130, K134, K1106B, KIi65B, K1328B, KLXI, K328, P5046, 11, 380, III, IV, VII, VIII, FC3-11, K12B, (syn=K12B), K125, (syn=K125), K142B, (syn=K142), (syn=K142B), K1181B, (syn=KI1 81), (syn=K1181B), K1765/!, (syn=K1765/1), K1842B, (syn=K1832B), K1937B, (syn=K1937B), L1, φ28, 7, 231, 483, 490, 632 and 864/100.

Bacteria of the genus Lepitospira can be infected by the following phages: LE1, LE3, LE4 and ˜NN-Leptospira (1).

Bacteria of the genus Listeria can be infected by the following phages: A511, 01761, 4211, 4286, (syn=BO54), A005, A006, A020, A500, A502, A511, A1 18, A620, A640, B012, B021, B024, B025, B035, B051, B053, B054, B055, B056, BlO1, BI 1O, B545, B604, B653, C707, D441, HSO47, HlOG, H8/73, H19, H21, H43, H46, H107, H108, HI lO, H163/84, H312, H340, H387, H391/73, H684/74, H924A, PSA, U153, φMLUP5, (syn=P35), 00241, 00611, 02971A, 02971C, 5/476, 5/911, 5/939, 5/11302, 5/11605, 5/11704, 184, 575, 633, 699/694, 744, 900, 1090, 1317, 1444, 1652, 1806, 1807, 1921/959, 1921/11367, 1921/11500, 1921/11566, 1921/12460, 1921/12582, 1967, 2389, 2425, 2671, 2685, 3274, 3550, 3551, 3552, 4276, 4277, 4292, 4477, 5337, 5348/11363, 5348/11646, 5348/12430, 5348/12434, 10072, 11355C, 11711A, 12029, 12981, 13441, 90666, 90816, 93253, 907515, 910716 and NN-Lisferia (15).

Bacteria of the genus Morganella can be infected by the following phage: 47.

Bacteria of the genus Mycobacterium can be infected by the following phages: 13, AG1, ALi, ATCC 11759, A2, B.C3, BG2, BK1, BKS, butyricum, B-I, B5, B7, B30, B35, Clark, C1, C2, DNAIII, DSP1, D4, D29, GS4E, (syn=GS4E), GS7, (syn=GS-7), (syn=GS7), IPa, lacticola, Legendre, Leo, L5, (syn=ΦL-5), MC-I, MC-3, MC-4, minetti, MTPHI 1, Mx4, MyF3P/59a, phlei, (syn=phlei 1), phlei 4, Polonus II, rabinovitschi, smegmatis, TM4, TM9, TMlO, TM20, Y7, Y10, φ630, IB, IF, IH, 1/1, 67, 106, 1430, B1, (syn=Bol), B24, D, D29, F—K, F—S, HP, Polonus I, Roy, R1, (syn=R1-Myb), (syn=Ri), 11, 31, 40, 50, 103a, 103b, 128, 3111-D, 3215-D and NN-Mycobacterium (1).

Bacteria of the genus Neisseria can be infected by the following phages: Group I, group II and NP1.

Bacteria of the genus Nocardia can be infected by the following phages: MNP8, NJ-L, NS-8, N5 and TtiN-Nocardia.

Bacteria of the genus Proteus can be infected by the following phages: Pm5, 13vir, 2/44, 4/545, 6/1004, 13/807, 20/826, 57, 67b, 78, 107/69, 121, 9/0, 22/608, 30/680, PmI, Pm3, Pm4, Pm6, Pm7, Pm9, PmIO, PmI1, Pv2, π1, φm, 7/549, 9B/2, 10A/31, 12/55, 14, 15, 16/789, 17/971, 19A/653, 23/532, 25/909, 26/219, 27/953, 32A/909, 33/971, 34/13, 65, 5006M, 7480b, VI, 13/3a, Clichy 12, n2600, yx7, 1/1004, 5/742, 9, 12, 14, 22, 24/860, 2600/D52, Pm8 and 24/2514.

Bacteria of the genus Providencia can be infected by the following phages: PL25, PL26, PL37, 9211/9295, 9213/921 Ib, 9248, 7/R49, 7476/322, 7478/325, 7479, 7480, 9000/9402 and 9213/921 Ia.

Bacteria of the genus Pseudomonas can be infected by the following phages: PfI, (syn=Pf-I), Pf2, Pf3, PP7, PRR1, 7s, im-Pseudomonas (1), AI-I, AI-2, B 17, B89, CB3, Col 2, Col 11, Col 18, Col 21, C154, C163, C167, C2121, E79, F8, ga, gb, H22, K1, M4, N2, Nu, PB-I, (syn=PB!), pfl6, PMN17, PP1, PP8, Psal, PsPl, PsP2, PsP3, PsP4, PsP5, PS3, PS17, PTB80, PX4, PX7, PYO1, PYO2, PYO5, PYO6, PYO9, PYO1O, PYO13, PYO14, PYO16, PYO18, PYO19, PYO20, PYO29, PYO32, PYO33, PYO35, PYO36, PYO37, PYO38, PYO39, PYO41, PYO42, PYO45, PYO47, PYO48, PYO64, PYO69, PYO103, P1K, SLP1, SL2, S2, UNL-I, wy, Yai, Ya4, Yan, φBE, φCTX, φC17, φKZ, (syn=ΦKZ), φ-LT, Φmu78, φNZ, φPLS-1, φST-1, φW-14, φ-2, 1/72, 2/79, 3, 3/DO, 4/237, 5/406, 6C, 6/6660, 7, 7v, 7/184, 8/280, 9/95, 10/502, 11/DE, 12/100, 12S, 16, 21, 24, 25F, 27, 31, 44, 68, 71, 95, 109, 188, 337, 352, 1214, HN-Pseudomonas (23), A856, B26, CI—I, CI-2, C5, D, gh-1, F1 16, HF, H90, K5, K6, K1 04, K109, K166, K267, N4, N5, O6N-25P, PE69, Pf, PPN25, PPN35, PPN89, PPN91, PP2, PP3, PP4, PP6, PP7, PP8, PP56, PP87, PP1 14, PP206, PP207, PP306, PP651, Psp231a, Pssy401, Pssy9220, psi, PTB2, PTB20, PTB42, PX1, PX3, PX1O, PX12, PX14, PYO70, PYO71, R, SH6, SH133, tf, Ya5, Ya7, ΦBS, ΦKf77, φ-MC, ΦmnF82, φPLS27, φPLS743, φS-1, 1, 2, 2, 3, 4, 5, 6, 7, 7, 8, 9, 10, 11, 12, 12B, 13, 14, 15, 14, 15, 16, 17, 18, 19, 20, 20, 21, 21, 22, 23, 23, 24, 25, 31, 53, 73, 119x, 145, 147, 170, 267, 284, 308, 525, NN-Pseudomonas (5), af, A7, B3, B33, B39, BI-I, C22, D3, D37, D40, D62, D3112, F7, FlO, g, gd, ge, gξ Hw12, Jb 19, KF1, L^{o}, OXN-32P, O6N-52P, PCH-I, PC13-1, PC35-1, PH2, PH51, PH93, PH132, PMW, PM13, PM57, PM61, PM62, PM63, PM69, PM105, PM1 13, PM681, PM682, PO4, PP1, PP4, PPS, PP64, PP65, PP66, PP71, PP86, PP88, PP92, PP401, PP711, PP891, Pssy41, Pssy42, Pssy403, Pssy404, Pssy420, Pssy923, PS4, PS-IO, Pz, SD1, SL1, SL3, SL5, SM, φC5, φC11, φC11-1, φC13, φC15, φMO, φX, φO4, φ1 1, φ240, 2, 2F, 5, 7m, 11, 13, 13/441, 14, 20, 24, 40, 45, 49, 61, 73, 148, 160, 198, 218, 222, 236, 242, 246, 249, 258, 269, 295, 297, 309, 318, 342, 350, 351, 357-1, 400-1, HN-Pseudomonas (6), GlO1, M6, M6a, L1, PB2, Pssy15, Pssy4210, Pssy4220, PYO12, PYO34, PYO49, PYO50, PYO51, PYO52, PYO53, PYO57, PYO59, PYO200, PX2, PX5, SL4, φO3, φO6 and 1214.

Bacteria of the genus Rickettsia can be infected by the following phage: NN-Rickettsia.

Bacteria of the genus Salmonella can be infected by the following phages: b, Beccles, CT, d, Dundee, f, FeIs 2, GI, GUI, GVI, GVIII, k, K, i, j, L, 01, (syn=0-1), (syn=O1), (syn=O-I), (syn=7), 02, 03, P3, P9a, PlO, Sab3, Sab5, San1S, San17, SI, Taunton, ViI, (syn=ViI), 9, imSalmonella (1), N-I, N-5, N-IO, N-17, N-22, 11, 12, 16-19, 20.2, 36, 449C/C178, 966A/C259, a, B.A.O.R., e, G4, GUI, L, LP7, M, MG40, N-18, PSA68, P4, P9c, P22, (syn=P22), (syn=PLT22), (syn=PLT22), P22a1, P22-4, P22-7, P22-11, SNT-I, SNT-2, SP6, Villi, ViIV, ViV, ViVI, ViVII, Worksop, Sj5, ε34, 1,37, 1(40), (syn=φ1[40]), 1,422, 2, 2.5, 3b, 4, 5, 6,14(18), 8, 14(6,7), 10, 27, 28B, 30, 31, 32, 33, 34, 36, 37, 39, 1412, SNT-3, 7-11, 40.3, c, C236, C557, C625, C966N, g, GV, G5, Gl 73, h, IRA, Jersey, MB78, P22-1, P22-3, P22-12, Sab1, Sab2, Sab2, Sab4, San1, San2, San3, San4, San6, San7, San8, San9, San13, San14, San16, San18, San19, San20, San21, San22, San23, San24, San25, San26, SasL1, SasL2, SasL3, SasL4, SasL5, S1BL, SII, ViII, φ1, 1, 2, 3a, 3a1, 1010, Ym-Salmonella (1), N-4, SasL6 and 27.

Bacteria of the genus Serratia can be infected by the following phages: A2P, PS20, SMB3, SMP, SMPS, SM2, V40, V56, ic, ΦCP-3, ΦCP-6, 3M, 10/1a, 20A, 34CC, 34H, 38T, 345G, 345P, 501B, SMB2, SMP2, BC, BT, CW2, CW3, CW4, CWS, Lt232, L2232, L34, L.228, SLP, SMPA, V.43, σ, φCW1, ΦCP6-1, ΦCP6-2, ΦCP6-5, 3T, 5, 8, 9F, 10/1, 20E, 32/6, 34B, 34CT, 34P, 37, 41, 56, 56D, 56P, 60P, 61/6, 74/6, 76/4, 101/8900, 226, 227, 228, 229F, 286, 289, 290F, 512, 764a, 2847/10, 2847/1Oa, L.359 and SMB1.

Bacteria of the genus Shigella can be infected by the following phages: Fsa, (syn=a), FSD2d, (syn=D2d), (syn=W2d), FSD2E, (syn=W2e), fv, F6, f7.8, H-Sh, PES, P90, SfII, Sh, SHm, SHrv, (syn=HIV), SHvi, (syn=HVI), SHVvm, (syn=HVIII), SKy66, (syn=gamma 66), (syn=yββ), (syn=γ66b), SKm, (syn=SIIIb)5 (syn=UI), SKw, (syn=Siva), (syn=IV), SIC™, (syn=SIVA.), (syn=IVA), SKvi, (syn=KVI), (syn=Svi), (syn=VI), SKvm, (syn=Svm), (syn=VIII), SKVHIA, (syn=SvmA), (syn=VIIIA), STvi, STK, STx1, STxn, S66, W2, (syn=D2c), (syn=D20), φ1, φIVb 3-SO-R, 8368-SO-R, F7, (syn=FS7), (syn=K29), F10, (syn=F S10), (syn=K31), I1, (syn=alfa), (syn=FSa), (syn=K1 8), (syn=α), I2, (syn=α), (syn=K19), SG33, (syn=G35), (syn=SO-35/G), SG35, (syn=SO-55/G), SG3201, (syn=SO-3201/G), SHn, (syn=HII), SHv, (syn=SHV), SHx, SHX, SKn, (syn=K2), (syn=KII), (syn=Sn), (syn=SsII), (syn=II), SKrv, (syn=Sm), (syn=SsIV), (syn=IV), SK1Va, (syn=Swab), (syn=SsIVa), (syn=IVa), SKV, (syn=K4), (syn=KV), (syn=SV), (syn=SsV), (syn=V), SKx, (syn=K9), (syn=KX), (syn=SX), (syn=SsX), (syn=X), STV, (syn=T35), (syn=35-50-R), STvm, (syn=T8345), (syn=8345-SO—S-R), W1, (syn=D8), (syn=FSD8), W2a, (syn=D2A), (syn=FS2a), DD-2, Sf6, FSi, (syn=F1), SF6, (syn=F6), SG42, (syn=SO-42/G), SG3203, (syn=SO-3203/G), SKF12, (syn=SsF12), (syn=F12), (syn=F12), STn, (syn=1881-SO-R), γ66, (syn=gamma 66a), (syn=Ssγ66), φ2, BI1, DDVII, (syn=DD7), FSD2b, (syn=W2B), FS2, (syn=F2), (syn=F2), FS4, (syn=F4), (syn=F4), FS5, (syn=F5), (syn=F5), FS9, (syn=F9), (syn=F9), FI 1, P2-S0-S, SG36, (syn=SO-36/G), (syn=G36), SG3204, (syn=SO-3204/G), SG3244, (syn=SO-3244/G), SHi, (syn=HI), SHvπ, (syn=HVII), SHK, (syn=HIX), SHx 1, SHxπ, (syn=HXn), SKI, KI, (syn=S1), (syn=SsI), SKVII, (syn=KVII), (syn=Svπ), (syn=SsVII), SKIX, (syn=KIX), (syn=S1x), (syn=SsIX), SKXII, (syn=KXII), (syn=Sxn), (syn=SsXII), STi, STffl, STrv, STVi, STvπ, S70, S206, U2-S0-S, 3210-SO-S, 3859-SO-S, 4020-SO-S, φ3, φ5, φ7, φ8, φ9, γ1O, φ11, φ13, φ14, φ18, SHm, (syn=Hai), SHχi, (syn=HXt) and SKxI, (syn=KXI), (syn=Sχi), (syn=SsXI), (syn=XI).

Bacteria of the genus Staphylococcus can be infected by the following phages: A, EW, K, Ph5, Ph9, PhIO, Ph13, P1, P2, P3, P4, P8, P9, P10, RG, SB-i, (syn=Sb-I), S3K, Twort, ΦSK311, φ812, 06, 40, 58, 119, 130, 131, 200, 1623, STC1, (syn=stc1), STC2, (syn=stc2), 44AHJD, 68, AC1, AC2, A6″C″, A9″C″, b581, CA-I, CA-2, CA-3, CA-4, CA-5, DI1, L39x35, L54a, M42, N1, N2, N3, N4, N5, N7, N8, NlO, N11, N12, N13, N14, N16, Ph6, Ph12, Ph14, UC-18, U4, U15, S1, S2, S3, S4, S5, X2, Z1, φB5-2, φD, ω, 11, (syn=φ11), (syn=P11-M15), 15, 28, 28A, 29, 31, 31B, 37, 42D, (syn=P42D), 44A, 48, 51, 52, 52A, (syn=P52A), 52B, 53, 55, 69, 71, (syn=P71), 71A, 72, 75, 76, 77, 79, 80, 80a, 82, 82A, 83 A, 84, 85, 86, 88, 88A, 89, 90, 92, 95, 96, 102, 107, 108, 111, 129-26, 130, 130A, 155, 157, 157A, 165, 187, 275, 275A, 275B, 356, 456, 459, 471, 471A, 489, 581, 676, 898, 1139, 1154A, 1259, 1314, 1380, 1405, 1563, 2148, 2638A, 2638B, 2638C, 2731, 2792A, 2792B, 2818, 2835, 2848A, 3619, 5841, 12100, AC3, A8, AlO, A13, b594n, D, HK2, N9, N15, P52, P87, S1, S6, Z4, φRE, 3A, 3B, 3C, 6, 7, 16, 21, 42B, 42C, 42E, 44, 47, 47A5 47C, 51, 54, 54x1, 70, 73, 75, 78, 81, 82, 88, 93, 94, 101, 105, 110, 115, 129/16, 174, 594n, 1363/14, 2460 and mS-Staphylococcus (1).

Bacteria of the genus Streptococcus can be infected by the following phages: EJ-I, NN-Streptococais (1), a, C1, FL0Ths, H39, Cp-I, Cp-5, Cp-7, Cp-9, Cp-I0, AT298, A5, alO/J2, alO/J5, alO/J9, A25, BTI1, b6, CA1, c20-1, c20-2, DP-I, Dp-4, DTI, ET42, elO, FA101, FEThs, Fκ, FKKIOI, FKLIO, FKP74, FKH, FLOThs, FyI01, fl, F10, F20140/76, g, GT-234, HB3, (syn=HB-3), HB-623, HB-746, M102, O1205, φO1205, PST, PO, P1, P2, P3, P5, P6, P8, P9, P9, P12, P13, P14, P49, P50, P51, P52, P53, P54, P55, P56, P57, P58, P59, P64, P67, P69, P71, P73, P75, P76, P77, P82, P83, P88, sc, sch, sf, SfI1 1, (syn=SFiI1), (syn=(syn=φSfil 1), (syn=φSfil 1), sfil9, (syn=SFil9), (syn=φSFil9), (syn=φSfil9), Sfi21, (syn=SFi21), (syn=φSFi21), (syn=φSfi21), ST0, STX, st2, ST2, ST4, S3, (syn=φS3), s265, Φ17, φ42, Φ57, φ80, φ81, φ82, φ83, φ84, φ85, φ86, φ87, φ88, φ89, φ90, φ91, φ92, φ93, φ94, φ95, φ96, φ97, φ98, φ99, φ100, φ101, φ102, φ227, Φ7201, ω1, ω2, ω3, ω4, ω5, ω6, ω8, ω1O, 1, 6, 9, 1OF, 12/12, 14, 17SR, 19S, 24, 50/33, 50/34, 55/14, 55/15, 70/35, 70/36, 71/ST15, 71/45, 71/46, 74F, 79/37, 79/38, 80/J4, 80/J9, 80/ST16, 80/15, 80/47, 80/48, 101, 103/39, 103/40, 121/41, 121/42, 123/43, 123/44, 124/44, 337/ST17 and mStreptococcus (34).

Bacteria of the genus Treponema can be infected by the following phage: NN-Treponema (1).

Bacteria of the genus Vibrio can be infected by the following phages: CTXΦ, fs, (syn=si), fs2, Ivpf5, Vfl2, Vf33, VPIΦ, VSK, v6, 493, CP-T1, ET25, kappa, K139, Labol,) XN-69P, OXN-86, 06N-21P, PB-I, P147, rp-1, SE3, VA-I, (syn=VcA-I), VcA-2, VP1, VP2, VP4, VP7, VP8, VP9, VPlO, VP17, VP18, VP19, X29, (syn=29 d′Herelle), t, ΦHAWI-1, ΦHAWI-2, ΦHAWI-3, ΦHAWI-4, ΦHAWI-5, ΦHAWI-6, ΦHAWI-7, XHAWI-8, ΦHAWI-9, ΦHAWI-10, ΦHC1-1, ΦHC1-2, ΦHC1-3, ΦHC1-4, ΦHC2-1, >HC2-2, ΦHC2-3, ΦHC2-4, ΦHC3-1, ΦHC3-2, ΦHC3-3, ΦHD1S-1, ΦHID1S-2, ΦHID2S-1, ΦHD2S-2, ΦHD2S-3, ΦHD2S-4, ΦHD2S-5, ΦHDO-1, ΦHDO-2, ΦHDO-3, ΦHDO-4, ΦHDO-5, ΦHDO-6, ΦKL-33, ΦKL-34, ΦKL-35, ΦKL-36, ΦKWH-2, ΦKWH-3, ΦKWH-4, ΦMARQ-1, ΦMARQ-2, ΦMARQ-3, ΦMOAT-1, ΦO139, ΦPEL1A-1, ΦPEL1A-2, ΦPEL8A-1, ΦPEL8A-2, ΦPEL8A-3, ΦPEL8C-1, ΦPEL8C-2, ΦPEL13A-1, ΦPEL13B-1, ΦPEL13B-2, ΦPEL13B-3, ΦPEL13B-4, ΦPEL13B-5, ΦPEL13B-6, ΦPEL13B-7, ΦPEL13B-8, ΦPEL13B-9, ΦPEL13B-10, φVP143, φVP253, Φ16, φ138, 1-II, 5, 13, 14, 16, 24, 32, 493, 6214, 7050, 7227, II, (syn=group II), (syn==φ2), V, VIII, ˜-m-Vibrio (13), KVP20, KVP40, nt-1, O6N-22P, P68, e1, e2, e3, e4, e5, FK, G, I, K, nt-6, N1, N2, N3, N4, N5, O6N-34P, OXN-72P, OXN-85P, OXN-100P, P, Ph-I, PL163/10, Q, S, T, φ92, 1-9, 37, 51, 57, 70A-8, 72A-4, 72A-10, 110A-4, 333, 4996, I (syn=group I), III (syn=group III), VI, (syn=A-Saratov), VII, IX, X, HN-Vibrio (6), pA1, 7, 7-8, 70A-2, 71A-6, 72A-5, 72A-8, 108A-10, 109A-6, 109A-8,110A-1, 110A-5, 110A-7, by-1, OXN-52P, P13, P38, P53, P65, P108, Pill, TP13 VP3, VP6, VP12, VP13, 70A-3, 70A-4, 70A-10, 72A-1, 108A-3, 109-B1, 110A-2, 149, (syn=y149), IV, (syn=group IV), NN-Vibrio (22), VPS, VPI1, VP15, VP16, α1, α2, α3a, α3b, 353B and HN-Vibrio (7).

Bacteria of the genus Yersinia can be infected by the following phages: H, H—I, H-2, H-3, H-4, Lucas 110, Lucas 303, Lucas 404, YerA3, YerA7, YerA20, YerA41, 3/M64-76, 5/G394-76, 6/C753-76, 8/C239-76, 9/F18167, 1701, 1710, PST, 1/F2852-76, D'Herelle, EV, H, Kotljarova, PTB, R, Y, YerA41, φYerO3-12, 3, 4/C1324-76, 7/F783-76, 903, 1/M6176 and Yer2AT.

In an embodiment, the bacteriophage is selected in the group consisting of Salmonella virus SKML39, Shigella virus AG3, Dickeya virus Limestone, Dickeya virus RC2014, Escherichia virus CBA120, Escherichia virus PhaxI, Salmonella virus 38, Salmonella virus Det7, Salmonella virus GG32, Salmonella virus PM10, Salmonella virus SFP10, Salmonella virus SH19, Salmonella virus SJ3, Escherichia virus ECML4, Salmonella virus Marshall, Salmonella virus Maynard, Salmonella virus SJ2, Salmonella virus STML131, Salmonella virus ViI, Erwinia virus Ea2809, Klebsiella virus 0507KN21, Serratia virus IME250, Serratia virus MAM1, Campylobacter virus CP21, Campylobacter virus CP220, Campylobacter virus CPt10, Campylobacter virus IBB35, Campylobacter virus CP81, Campylobacter virus CP30A, Campylobacter virus CPX, Campylobacter virus NCTC12673, Erwinia virus Ea214, Erwinia virus M7, Escherichia virus AYO145A, Escherichia virus EC6, Escherichia virus HY02, Escherichia virus JH2, Escherichia virus TP1, Escherichia virus VpaE1, Escherichia virus wV8, Salmonella virus FelixO1, Salmonella virus HB2014, Salmonella virus Mushroom, Salmonella virus UAB87, Citrobacter virus Moogle, Citrobacter virus Mordin, Escherichia virus SUSP1, Escherichia virus SUSP2, Aeromonas virus phiO18P, Haemophilus virus HP1, Haemophilus virus HP2, Pasteurella virus F108, Vibrio virus K139, Vibrio virus Kappa, Burkholderia virus phi52237, Burkholderia virus phiE122, Burkholderia virus phiE202, Escherichia virus 186, Escherichia virus P4, Escherichia virus P2, Escherichia virus Wphi, Mannheimia virus PHL101, Pseudomonas virus phiCTX, Ralstonia virus RSA1, Salmonella virus Fels2, Salmonella virus PsP3, Salmonella virus SopEphi, Yersinia virus L413C, Staphylococcus virus G1, Staphylococcus virus G15, Staphylococcus virus JD7, Staphylococcus virus K, Staphylococcus virus MCE2014, Staphylococcus virus P108, Staphylococcus virus Rodi, Staphylococcus virus 5253, Staphylococcus virus S25-4, Staphylococcus virus SA12, Listeria virus A511, Listeria virus P100, Staphylococcus virus Remus, Staphylococcus virus SAll, Staphylococcus virus Stau2, Bacillus virus Camphawk, Bacillus virus SPO1, Bacillus virus BCP78, Bacillus virus TsarBomba, Staphylococcus virus Twort, Enterococcus virus phiEC24C, Lactobacillus virus Lb338-1, Lactobacillus virus LP65, Enterobacter virus PG7, Escherichia virus CC31, Klebsiella virus JD18, Klebsiella virus PKO111, Escherichia virus Bp7, Escherichia virus IME08, Escherichia virus JS10, Escherichia virus J598, Escherichia virus QL01, Escherichia virus VR5, Enterobacter virus Eap3, Klebsiella virus KP15, Klebsiella virus KP27, Klebsiella virus Matisse, Klebsiella virus Miro, Citrobacter virus Merlin, Citrobacter virus Moon, Escherichia virus JSE, Escherichia virus phi1, Escherichia virus RB49, Escherichia virus HX01, Escherichia virus JS09, Escherichia virus RB69, Shigella virus UTAM, Salmonella virus S16, Salmonella virus STML198, Vibrio virus KVP40, Vibrio virus nt1, Vibrio virus ValKK3, Escherichia virus VR7, Escherichia virus VR20, Escherichia virus VR25, Escherichia virus VR26, Shigella virus SP18, Escherichia virus AR1, Escherichia virus C40, Escherichia virus E112, Escherichia virus ECML134, Escherichia virus HYO1, Escherichia virus Ime09, Escherichia virus RB3, Escherichia virus RB14, Escherichia virus T4, Shigella virus Pss1, Shigella virus Shf12, Yersinia virus D1, Yersinia virus PST, Acinetobacter virus 133, Aeromonas virus 65, Aeromonas virus Aeh1, Escherichia virus RB16, Escherichia virus RB32, Escherichia virus RB43, Pseudomonas virus 42, Cronobacter virus CR3, Cronobacter virus CR8, Cronobacter virus CR9, Cronobacter virus PBES02, Pectobacterium virus phiTE, Cronobacter virus GAP31, Escherichia virus 4MG, Salmonella virus SE1, Salmonella virus SSE121, Escherichia virus FFH2, Escherichia virus FV3, Escherichia virus JES2013, Escherichia virus V5, Brevibacillus virus Abouo, Brevibacillus virus Davies, Bacillus virus Agate, Bacillus virus Bobb, Bacillus virus Bp8pC, Erwinia virus Deimos, Erwinia virus Ea35-70, Erwinia virus RAY, Erwinia virus Simmy50, Erwinia virus SpecialG, Acinetobacter virus AB1, Acinetobacter virus AB2, Acinetobacter virus AbC62, Acinetobacter virus AP22, Arthrobacter virus ArV1, Arthrobacter virus Trina, Bacillus virus AvesoBmore, Bacillus virus B4, Bacillus virus Bigbertha, Bacillus virus Riley, Bacillus virus Spock, Bacillus virus Troll, Bacillus virus Bastille, Bacillus virus CAM003, Bacillus virus Bc431, Bacillus virus Bcp1, Bacillus virus BCP82, Bacillus virus BM15, Bacillus virus Deepblue, Bacillus virus JBP901, Burkholderia virus Bcep1, Burkholderia virus Bcep43, Burkholderia virus Bcep781, Burkholderia virus BcepNY3, Xanthomonas virus OP2, Burkholderia virus BcepMu, Burkholderia virus phiE255, Aeromonas virus 44RR2, Mycobacterium virus Alice, Mycobacterium virus Bxzl, Mycobacterium virus Dandelion, Mycobacterium virus HyRo, Mycobacterium virus 13, Mycobacterium virus Nappy, Mycobacterium virus Sebata, Clostridium virus phiC2, Clostridium virus phiCD27, Clostridium virus phiCD119, Bacillus virus CP51, Bacillus virus JL, Bacillus virus Shanette, Escherichia virus CVM10, Escherichia virus ep3, Erwinia virus Asesino, Erwinia virus EaH2, Pseudomonas virus EL, Halomonas virus HAP1, Vibrio virus VP882, Brevibacillus virus Jimmer, Brevibacillus virus Osiris, Pseudomonas virus Ab03, Pseudomonas virus KPP10, Pseudomonas virus PAKP3, Sinorhizobium virus M7, Sinorhizobium virus M12, Sinorhizobium virus N3, Erwinia virus Machina, Arthrobacter virus Brent, Arthrobacter virus Jawnski, Arthrobacter virus Martha, Arthrobacter virus Sonny, Edwardsiella virus MSW3, Edwardsiella virus PEi21, Escherichia virus Mu, Shigella virus SfMu, Halobacterium virus phiH, Bacillus virus Grass, Bacillus virus NIT1, Bacillus virus SPG24, Aeromonas virus 43, Escherichia virus P1, Pseudomonas virus CAb1, Pseudomonas virus CAb02, Pseudomonas virus JG004, Pseudomonas virus PAKP1, Pseudomonas virus PAKP4, Pseudomonas virus PaP1, Burkholderia virus BcepF1, Pseudomonas virus 141, Pseudomonas virus Ab28, Pseudomonas virus DL60, Pseudomonas virus DL68, Pseudomonas virus F8, Pseudomonas virus JG024, Pseudomonas virus KPP12, Pseudomonas virus LBL3, Pseudomonas virus LMA2, Pseudomonas virus PB1, Pseudomonas virus SN, Pseudomonas virus PA7, Pseudomonas virus phiKZ, Rhizobium virus RHEph4, Ralstonia virus RSF1, Ralstonia virus RSL2, Ralstonia virus RSL1, Aeromonas virus 25, Aeromonas virus 31, Aeromonas virus Aes12, Aeromonas virus Aes508, Aeromonas virus AS4, Stenotrophomonas virus IME13, Staphylococcus virus IPLAC1C, Staphylococcus virus SEP1, Salmonella virus SPN3US, Bacillus virus 1, Geobacillus virus GBSV1, Yersinia virus R1RT, Yersinia virus TG1, Bacillus virus G, Bacillus virus PBS1, Microcystis virus Ma-LMM01, Vibrio virus MAR, Vibrio virus VHML, Vibrio virus VP585, Bacillus virus BPS13, Bacillus virus Hakuna, Bacillus virus Megatron, Bacillus virus WPh, Acinetobacter virus AB3, Acinetobacter virus Abp1, Acinetobacter virus Fri1, Acinetobacter virus IME200, Acinetobacter virus PD6A3, Acinetobacter virus PDAB9, Acinetobacter virus phiAB1, Escherichia virus K30, Klebsiella virus K5, Klebsiella virus K11, Klebsiella virus Kp1, Klebsiella virus KP32, Klebsiella virus KpV289, Klebsiella virus F19, Klebsiella virus K244, Klebsiella virus Kp2, Klebsiella virus KP34, Klebsiella virus KpV41, Klebsiella virus KpV71, Klebsiella virus KpV475, Klebsiella virus SU503, Klebsiella virus SU552A, Pantoea virus Limelight, Pantoea virus Limezero, Pseudomonas virus LKA1, Pseudomonas virus phiKMV, Xanthomonas virus f20, Xanthomonas virus f30, Xylella virus Prado, Erwinia virus Era103, Escherichia virus K5, Escherichia virus K1-5, Escherichia virus K1E, Salmonella virus SP6, Escherichia virus T7, Kluyvera virus Kvp1, Pseudomonas virus gh1, Prochlorococcus virus PSSP7, Synechococcus virus P60, Synechococcus virus Syn5, Streptococcus virus Cp1, Streptococcus virus Cp7, Staphylococcus virus 44AHJD, Streptococcus virus C1, Bacillus virus B103, Bacillus virus GA1, Bacillus virus phi29, Kurthia virus 6, Actinomyces virus Avl, Mycoplasma virus P1, Escherichia virus 24B, Escherichia virus 933W, Escherichia virus Min27, Escherichia virus PA28, Escherichia virus Stx2 II, Shigella virus 7502Stx, Shigella virus POCJ13, Escherichia virus 191, Escherichia virus PA2, Escherichia virus TL2011, Shigella virus VASD, Burkholderia virus Bcep22, Burkholderia virus Bcepi102, Burkholderia virus Bcepmig1, Burkholderia virus DC1, Bordetella virus BPP1, Burkholderia virus BcepC6B, Cellulophaga virus Cba41, Cellulophaga virus Cba172, Dinoroseobacter virus DFL12, Erwinia virus Ea9-2, Erwinia virus Frozen, Escherichia virus phiV10, Salmonella virus Epsilon15, Salmonella virus SPN1S, Pseudomonas virus F116, Pseudomonas virus H66, Escherichia virus APEC5, Escherichia virus APEC7, Escherichia virus Bp4, Escherichia virus EC1UPM, Escherichia virus ECBP1, Escherichia virus G7C, Escherichia virus IME11, Shigella virus Sb1, Achromobacter virus Axp3, Achromobacter virus JWAlpha, Edwardsiella virus KF1, Pseudomonas virus KPP25, Pseudomonas virus R18, Pseudomonas virus Ab09, Pseudomonas virus LIT1, Pseudomonas virus PA26, Pseudomonas virus Ab22, Pseudomonas virus CHU, Pseudomonas virus LUZ24, Pseudomonas virus PAA2, Pseudomonas virus PaP3, Pseudomonas virus PaP4, Pseudomonas virus TL, Pseudomonas virus KPP21, Pseudomonas virus LUZ7, Escherichia virus N4, Salmonella virus 9NA, Salmonella virus SP069, Salmonella virus BTP1, Salmonella virus HK620, Salmonella virus P22, Salmonella virus ST64T, Shigella virus Sf6, Bacillus virus Page, Bacillus virus Palmer, Bacillus virus Pascal, Bacillus virus Pony, Bacillus virus Pookie, Escherichia virus 172-1, Escherichia virus ECB2, Escherichia virus NJ01, Escherichia virus phiEco32, Escherichia virus Septima11, Escherichia virus SU10, Brucella virus Pr, Brucella virus Tb, Escherichia virus Pollock, Salmonella virus FSL SP-058, Salmonella virus FSL SP-076, Helicobacter virus 1961P, Helicobacter virus KHP30, Helicobacter virus KHP40, Hamiltonella virus APSE1, Lactococcus virus KSY1, Phormidium virus WMP3, Phormidium virus WMP4, Pseudomonas virus 119X, Roseobacter virus SIO1, Vibrio virus VpV262, Vibrio virus VC8, Vibrio virus VP2, Vibrio virus VPS, Streptomyces virus Amela, Streptomyces virus phiCAM, Streptomyces virus Aaronocolus, Streptomyces virus Caliburn, Streptomyces virus Danzina, Streptomyces virus Hydra, Streptomyces virus Izzy, Streptomyces virus Lannister, Streptomyces virus Lika, Streptomyces virus Sujidade, Streptomyces virus Zemlya, Streptomyces virus ELB20, Streptomyces virus R4, Streptomyces virus phiHau3, Mycobacterium virus Acadian, Mycobacterium virus Baee, Mycobacterium virus Reprobate, Mycobacterium virus Adawi, Mycobacterium virus Bane1, Mycobacterium virus BrownCNA, Mycobacterium virus Chrisnmich, Mycobacterium virus Cooper, Mycobacterium virus JAMaL, Mycobacterium virus Nigel, Mycobacterium virus Stinger, Mycobacterium virus Vincenzo, Mycobacterium virus Zemanar, Mycobacterium virus Apizium, Mycobacterium virus Manad, Mycobacterium virus Oline, Mycobacterium virus Osmaximus, Mycobacterium virus Pg1, Mycobacterium virus Soto, Mycobacterium virus Suffolk, Mycobacterium virus Athena, Mycobacterium virus Bernardo, Mycobacterium virus Gadjet, Mycobacterium virus Pipefish, Mycobacterium virus Godines, Mycobacterium virus Rosebush, Mycobacterium virus Babsiella, Mycobacterium virus Brujita, Mycobacterium virus Che9c, Mycobacterium virus Sbash, Mycobacterium virus Hawkeye, Mycobacterium virus Plot, Salmonella virus AG11, Salmonella virus Entl, Salmonella virus f18SE, Salmonella virus Jersey, Salmonella virus L13, Salmonella virus LSPA1, Salmonella virus SE2, Salmonella virus SETP3, Salmonella virus SETP7, Salmonella virus SETP13, Salmonella virus SP101, Salmonella virus SS3e, Salmonella virus wks13, Escherichia virus K1G, Escherichia virus K1H, Escherichia virus Klind1, Escherichia virus Klind2, Salmonella virus SP31, Leuconostoc virus Lmd1, Leuconostoc virus LN03, Leuconostoc virus LN04, Leuconostoc virus LN12, Leuconostoc virus LN6B, Leuconostoc virus P793, Leuconostoc virus 1A4, Leuconostoc virus Ln8, Leuconostoc virus Ln9, Leuconostoc virus LN25, Leuconostoc virus LN34, Leuconostoc virus LNTR3, Mycobacterium virus Bongo, Mycobacterium virus Rey, Mycobacterium virus Butters, Mycobacterium virus Michelle, Mycobacterium virus Charlie, Mycobacterium virus Pipsqueaks, Mycobacterium virus Xeno, Mycobacterium virus Panchino, Mycobacterium virus Phrann, Mycobacterium virus Redi, Mycobacterium virus Skinnyp, Gordonia virus BaxterFox, Gordonia virus Yeezy, Gordonia virus Kita, Gordonia virus Zirinka, Gorrdonia virus Nymphadora, Mycobacterium virus Bignuz, Mycobacterium virus Brusacoram, Mycobacterium virus Donovan, Mycobacterium virus Fishburne, Mycobacterium virus Jebeks, Mycobacterium virus Malithi, Mycobacterium virus Phayonce, Enterobacter virus F20, Klebsiella virus 1513, Klebsiella virus KLPN1, Klebsiella virus KP36, Klebsiella virus PKP126, Klebsiella virus Sushi, Escherichia virus AHP42, Escherichia virus AHS24, Escherichia virus AKS96, Escherichia virus C119, Escherichia virus E41c, Escherichia virus Eb49, Escherichia virus Jk06, Escherichia virus KP26, Escherichia virus Rogue1, Escherichia virus ACGM12, Escherichia virus Rtp, Escherichia virus ADB2, Escherichia virus JMPW1, Escherichia virus JMPW2, Escherichia virus T1, Shigella virus PSf2, Shigella virus Shfl1, Citrobacter virus Stevie, Escherichia virus TLS, Salmonella virus SP126, Cronobacter virus Esp2949-1, Pseudomonas virus Ab18, Pseudomonas virus Ab19, Pseudomonas virus PaMx11, Arthrobacter virus Amigo, Propionibacterium virus Anatole, Propionibacterium virus B3, Bacillus virus Andromeda, Bacillus virus Blastoid, Bacillus virus Curly, Bacillus virus Eoghan, Bacillus virus Finn, Bacillus virus Glittering, Bacillus virus Riggi, Bacillus virus Taylor, Gordonia virus Attis, Mycobacterium virus Barnyard, Mycobacterium virus Konstantine, Mycobacterium virus Predator, Mycobacterium virus Bernal13, Staphylococcus virus 13, Staphylococcus virus 77, Staphylococcus virus 108PVL, Mycobacterium virus Bron, Mycobacterium virus Faithl, Mycobacterium virus Joedirt, Mycobacterium virus Rumpelstiltskin, Lactococcus virus bIL67, Lactococcus virus c2, Lactobacillus virus c5, Lactobacillus virus Ld3, Lactobacillus virus Ld17, Lactobacillus virus Ld25A, Lactobacillus virus LLKu, Lactobacillus virus phiLdb, Cellulophaga virus Cba121, Cellulophaga virus Cba171, Cellulophaga virus Cba181, Cellulophaga virus ST, Bacillus virus 250, Bacillus virus IEBH, Mycobacterium virus Ardmore, Mycobacterium virus Avani, Mycobacterium virus Boomer, Mycobacterium virus Che8, Mycobacterium virus Che9d, Mycobacterium virus Deadp, Mycobacterium virus Dlane, Mycobacterium virus Dorothy, Mycobacterium virus Dotproduct, Mycobacterium virus Drago, Mycobacterium virus Fruitloop, Mycobacterium virus Gumbie, Mycobacterium virus Ibhubesi, Mycobacterium virus Llij, Mycobacterium virus Mozy, Mycobacterium virus Mutaforma13, Mycobacterium virus Pacc40, Mycobacterium virus PMC, Mycobacterium virus Ramsey, Mycobacterium virus Rockyhorror, Mycobacterium virus SG4, Mycobacterium virus Shauna1, Mycobacterium virus Shilan, Mycobacterium virus Spartacus, Mycobacterium virus Taj, Mycobacterium virus Tweety, Mycobacterium virus Wee, Mycobacterium virus Yoshi, Salmonella virus Chi, Salmonella virus FSLSP030, Salmonella virus FSLSP088, Salmonella virus iEPS5, Salmonella virus SPN19, Mycobacterium virus 244, Mycobacterium virus Bask21, Mycobacterium virus CJW1, Mycobacterium virus Eureka, Mycobacterium virus Kostya, Mycobacterium virus Porky, Mycobacterium virus Pumpkin, Mycobacterium virus Sirduracell, Mycobacterium virus Toto, Mycobacterium virus Corndog, Mycobacterium virus Firecracker, Rhodobacter virus RcCronus, Pseudomonas virus D3112, Pseudomonas virus DMS3, Pseudomonas virus FHA0480, Pseudomonas virus LPB1, Pseudomonas virus MP22, Pseudomonas virus MP29, Pseudomonas virus MP38, Pseudomonas virus PA1KOR, Pseudomonas virus D3, Pseudomonas virus PMG1, Arthrobacter virus Decurro, Gordonia virus Demosthenes, Gordonia virus Katyusha, Gordonia virus Kvothe, Propionibacterium virus B22, Propionibacterium virus Doucette, Propionibacterium virus E6, Propionibacterium virus G4, Burkholderia virus phi6442, Burkholderia virus phi1026b, Burkholderia virus phiE125, Edwardsiella virus eiAU, Mycobacterium virus Ff47, Mycobacterium virus Muddy, Mycobacterium virus Gaia, Mycobacterium virus Giles, Arthrobacter virus Captnmurica, Arthrobacter virus Gordon, Gordonia virus GordTnk2, Paenibacillus virus Harrison, Escherichia virus EK99P1, Escherichia virus HK578, Escherichia virus JL1, Escherichia virus SSL2009a, Escherichia virus YD2008s, Shigella virus EP23, Sodalis virus S01, Escherichia virus HK022, Escherichia virus HK75, Escherichia virus HK97, Escherichia virus HK106, Escherichia virus HK446, Escherichia virus HK542, Escherichia virus HK544, Escherichia virus HK633, Escherichia virus mEp234, Escherichia virus mEp235, Escherichia virus mEpX1, Escherichia virus mEpX2, Escherichia virus mEp043, Escherichia virus mEp213, Escherichia virus mEp237, Escherichia virus mEp390, Escherichia virus mEp460, Escherichia virus mEp505, Escherichia virus mEp506, Brevibacillus virus Jenst, Achromobacter virus 83-24, Achromobacter virus JWX, Arthrobacter virus Kellezzio, Arthrobacter virus Kitkat, Arthrobacter virus Bennie, Arthrobacter virus DrRobert, Arthrobacter virus Glenn, Arthrobacter virus HunterDalle, Arthrobacter virus Joann, Arthrobacter virus Korra, Arthrobacter virus Preamble, Arthrobacter virus Pumancara, Arthrobacter virus Wayne, Mycobacterium virus Alma, Mycobacterium virus Arturo, Mycobacterium virus Astro, Mycobacterium virus Backyardigan, Mycobacterium virus BBPiebs31, Mycobacterium virus Benedict, Mycobacterium virus Bethlehem, Mycobacterium virus Billknuckles, Mycobacterium virus Bruns, Mycobacterium virus Bxb1, Mycobacterium virus Bxz2, Mycobacterium virus Che12, Mycobacterium virus Cuco, Mycobacterium virus D29, Mycobacterium virus Doom, Mycobacterium virus Ericb, Mycobacterium virus Euphoria, Mycobacterium virus George, Mycobacterium virus Gladiator, Mycobacterium virus Goose, Mycobacterium virus Hammer, Mycobacterium virus Heldan, Mycobacterium virus Jasper, Mycobacterium virus JC27, Mycobacterium virus Jeffabunny, Mycobacterium virus JHC117, Mycobacterium virus KBG, Mycobacterium virus Kssjeb, Mycobacterium virus Kugel, Mycobacterium virus L5, Mycobacterium virus Lesedi, Mycobacterium virus LHTSCC, Mycobacterium virus lockley, Mycobacterium virus Marcell, Mycobacterium virus Microwolf, Mycobacterium virus Mrgordo, Mycobacterium virus Museum, Mycobacterium virus Nepal, Mycobacterium virus Packman, Mycobacterium virus Peaches, Mycobacterium virus Perseus, Mycobacterium virus Pukovnik, Mycobacterium virus Rebeuca, Mycobacterium virus Redrock, Mycobacterium virus Ridgecb, Mycobacterium virus Rockstar, Mycobacterium virus Saintus, Mycobacterium virus Skipole, Mycobacterium virus Solon, Mycobacterium virus Switzer, Mycobacterium virus SWU1, Mycobacterium virus Ta17a, Mycobacterium virus Tiger, Mycobacterium virus Timshel, Mycobacterium virus Trixie, Mycobacterium virus Turbido, Mycobacterium virus Twister, Mycobacterium virus U2, Mycobacterium virus Violet, Mycobacterium virus Wonder, Escherichia virus DE3, Escherichia virus HK629, Escherichia virus HK630, Escherichia virus lambda, Arthrobacter virus Laroye, Mycobacterium virus Halo, Mycobacterium virus Liefie, Mycobacterium virus Marvin, Mycobacterium virus Mosmoris, Arthrobacter virus Circum, Arthrobacter virus Mudcat, Escherichia virus N15, Escherichia virus 9g, Escherichia virus JenKl, Escherichia virus JenPl, Escherichia virus JenP2, Pseudomonas virus NP1, Pseudomonas virus PaMx25, Mycobacterium virus Baka, Mycobacterium virus Courthouse, Mycobacterium virus Littlee, Mycobacterium virus Omega, Mycobacterium virus Optimus, Mycobacterium virus Thibault, Polaribacter virus P12002L, Polaribacter virus P12002S, Nonlabens virus P12024L, Nonlabens virus P12024S, Thermus virus P23-45, Thermus virus P74-26, Listeria virus LP26, Listeria virus LP37, Listeria virus LP110, Listeria virus LP114, Listeria virus P70, Propionibacterium virus ATCC29399BC, Propionibacterium virus ATCC29399BT, Propionibacterium virus Attacne, Propionibacterium virus Keiki, Propionibacterium virus Kubed, Propionibacterium virus Lauchelly, Propionibacterium virus MrAK, Propionibacterium virus Ouroboros, Propionibacterium virus P91, Propionibacterium virus P105, Propionibacterium virus P144, Propionibacterium virus P1001, Propionibacterium virus P1.1, Propionibacterium virus P100A, Propionibacterium virus P100D, Propionibacterium virus P101A, Propionibacterium virus P104A, Propionibacterium virus PA6, Propionibacterium virus Pacnes201215, Propionibacterium virus PAD20, Propionibacterium virus PAS50, Propionibacterium virus PHL009M11, Propionibacterium virus PHL025M00, Propionibacterium virus PHL037M02, Propionibacterium virus PHL041M10, Propionibacterium virus PHL060L00, Propionibacterium virus PHL067M01, Propionibacterium virus PHL070N00, Propionibacterium virus PHL071N05, Propionibacterium virus PHL082M03, Propionibacterium virus PHL092M00, Propionibacterium virus PHL095N00, Propionibacterium virus PHL111M01, Propionibacterium virus PHL112N00, Propionibacterium virus PHL113M01, Propionibacterium virus PHL114L00, Propionibacterium virus PHL116M00, Propionibacterium virus PHL117M00, Propionibacterium virus PHL117M01, Propionibacterium virus PHL132N00, Propionibacterium virus PHL141N00, Propionibacterium virus PHL151M00, Propionibacterium virus PHL151N00, Propionibacterium virus PHL152M00, Propionibacterium virus PHL163M00, Propionibacterium virus PHL171M01, Propionibacterium virus PHL179M00, Propionibacterium virus PHL194M00, Propionibacterium virus PHL199M00, Propionibacterium virus PHL301M00, Propionibacterium virus PHL308M00, Propionibacterium virus Pirate, Propionibacterium virus Procrassl, Propionibacterium virus SKKY, Propionibacterium virus Solid, Propionibacterium virus Stormborn, Propionibacterium virus Wizzo, Pseudomonas virus PaMx28, Pseudomonas virus PaMx74, Mycobacterium virus Patience, Mycobacterium virus PBI1, Rhodococcus virus Pepy6, Rhodococcus virus Poco6, Propionibacterium virus PFR1, Streptomyces virus phiBT1, Streptomyces virus phiC31, Streptomyces virus TG1, Caulobacter virus Karma, Caulobacter virus Magneto, Caulobacter virus phiCbK, Caulobacter virus Rogue, Caulobacter virus Swift, Staphylococcus virus 11, Staphylococcus virus 29, Staphylococcus virus 37, Staphylococcus virus 53, Staphylococcus virus 55, Staphylococcus virus 69, Staphylococcus virus 71, Staphylococcus virus 80, Staphylococcus virus 85, Staphylococcus virus 88, Staphylococcus virus 92, Staphylococcus virus 96, Staphylococcus virus 187, Staphylococcus virus 52a, Staphylococcus virus 80alpha, Staphylococcus virus CNPH82, Staphylococcus virus EW, Staphylococcus virus IPLA5, Staphylococcus virus IPLA7, Staphylococcus virus IPLA88, Staphylococcus virus PH15, Staphylococcus virus phiETA, Staphylococcus virus phiETA2, Staphylococcus virus phiETA3, Staphylococcus virus phiMR11, Staphylococcus virus phiMR25, Staphylococcus virus phiNM1, Staphylococcus virus phiNM2, Staphylococcus virus phiNM4, Staphylococcus virus SAP26, Staphylococcus virus X2, Enterococcus virus FL1, Enterococcus virus FL2, Enterococcus virus FL3, Lactobacillus virus ATCC8014, Lactobacillus virus phiJL1, Pediococcus virus cIP1, Aeromonas virus pIS4A, Listeria virus LP302, Listeria virus PSA, Methanobacterium virus psiM1, Roseobacter virus RDJL1, Roseobacter virus RDJL2, Rhodococcus virus RER2, Enterococcus virus BC611, Enterococcus virus IMEEF1, Enterococcus virus SAP6, Enterococcus virus VD13, Streptococcus virus SPQS1, Mycobacterium virus Papyrus, Mycobacterium virus Send513, Burkholderia virus KL1, Pseudomonas virus 73, Pseudomonas virus Ab26, Pseudomonas virus Kakheti25, Escherichia virus Cajan, Escherichia virus Seurat, Staphylococcus virus SEP9, Staphylococcus virus Sextaec, Streptococcus virus 858, Streptococcus virus 2972, Streptococcus virus ALQ132, Streptococcus virus 01205, Streptococcus virus Sfil1, Streptococcus virus 7201, Streptococcus virus DT1, Streptococcus virus phiAbc2, Streptococcus virus Sfil9, Streptococcus virus Sfi21, Paenibacillus virus Diva, Paenibacillus virus HblOc2, Paenibacillus virus Rani, Paenibacillus virus Shelly, Paenibacillus virus Sitara, Paenibacillus virus Willow, Lactococcus virus 712, Lactococcus virus ASCC191, Lactococcus virus ASCC273, Lactococcus virus ASCC281, Lactococcus virus ASCC465, Lactococcus virus ASCC532, Lactococcus virus Bibb29, Lactococcus virus bIL170, Lactococcus virus CB13, Lactococcus virus CB14, Lactococcus virus CB19, Lactococcus virus CB20, Lactococcus virus jj50, Lactococcus virus P2, Lactococcus virus P008, Lactococcus virus skl, Lactococcus virus S14, Bacillus virus Slash, Bacillus virus Stahl, Bacillus virus Staley, Bacillus virus Stills, Gordonia virus Bachita, Gordonia virus ClubL, Gordonia virus OneUp, Gordonia virus Smoothie, Gordonia virus Soups, Bacillus virus SPbeta, Vibrio virus MARIO, Vibrio virus SSP002, Escherichia virus AKFV33, Escherichia virus BF23, Escherichia virus DT57C, Escherichia virus EPS7, Escherichia virus FFH1, Escherichia virus H8, Escherichia virus s1ur09, Escherichia virus T5, Salmonella virus 118970sa12, Salmonella virus Shivani, Salmonella virus SPC35, Salmonella virus Stitch, Arthrobacter virus Tank, Tsukamurella virus TIN2, Tsukamurella virus TIN3, Tsukamurella virus TIN4, Rhodobacter virus RcSpartan, Rhodobacter virus RcTitan, Mycobacterium virus Anaya, Mycobacterium virus Angelica, Mycobacterium virus Crimd, Mycobacterium virus Fionnbarth, Mycobacterium virus Jaws, Mycobacterium virus Larva, Mycobacterium virus Macncheese, Mycobacterium virus Pixie, Mycobacterium virus TM4, Bacillus virus BMBtp2, Bacillus virus TP21, Geobacillus virus Tp84, Staphylococcus virus 47, Staphylococcus virus 3a, Staphylococcus virus 42e, Staphylococcus virus IPLA35, Staphylococcus virus phi12, Staphylococcus virus phiSLT, Mycobacterium virus 32HC, Rhodococcus virus RGL3, Paenibacillus virus Vegas, Gordonia virus Vendetta, Bacillus virus Wbeta, Mycobacterium virus Wildcat, Gordonia virus Twister6, Gordonia virus Wizard, Gordonia virus Hotorobo, Gordonia virus Monty, Gordonia virus Woes, Xanthomonas virus CP1, Xanthomonas virus OP1, Xanthomonas virus phi17, Xanthomonas virus Xop411, Xanthomonas virus Xp10, Streptomyces virus TP1604, Streptomyces virus YDN12, Alphaproteobacteria virus phiJ1001, Pseudomonas virus LKO4, Pseudomonas virus M6, Pseudomonas virus MP1412, Pseudomonas virus PAE1, Pseudomonas virus Yua, Pseudoalteromonas virus PM2, Pseudomonas virus phi6, Pseudomonas virus phi8, Pseudomonas virus phil2, Pseudomonas virus phil3, Pseudomonas virus phi2954, Pseudomonas virus phiNN, Pseudomonas virus phiYY, Vibrio virus fsl, Vibrio virus VGJ, Ralstonia virus RS603, Ralstonia virus RSM1, Ralstonia virus RSM3, Escherichia virus M13, Escherichia virus 122, Salmonella virus IKe, Acholeplasma virus L51, Vibrio virus fs2, Vibrio virus VFJ, Escherichia virus If1, Propionibacterium virus B5, Pseudomonas virus Pf1, Pseudomonas virus Pf3, Ralstonia virus PE226, Ralstonia virus RSS1, Spiroplasma virus SVTS2, Stenotrophomonas virus PSH1, Stenotrophomonas virus SMA6, Stenotrophomonas virus SMA7, Stenotrophomonas virus SMAS, Vibrio virus CTXphi, Vibrio virus KSF1, Vibrio virus VCY, Vibrio virus Vf33, Vibrio virus VfO3K6, Xanthomonas virus Cflc, Spiroplasma virus C74, Spiroplasma virus R8A2B, Spiroplasma virus SkV1CR23x, Escherichia virus FI, Escherichia virus Qbeta, Escherichia virus BZ13, Escherichia virus MS2, Escherichia virus alpha3, Escherichia virus ID21, Escherichia virus ID32, Escherichia virus ID62, Escherichia virus NC28, Escherichia virus NC29, Escherichia virus NC35, Escherichia virus phiK, Escherichia virus St1, Escherichia virus WA45, Escherichia virus G4, Escherichia virus ID52, Escherichia virus Talmos, Escherichia virus phiX174, Bdellovibrio virus MAC1, Bdellovibrio virus MH2K, Chlamydia virus Chpl, Chlamydia virus Chp2, Chlamydia virus CPAR39, Chlamydia virus CPG1, Spiroplasma virus SpV4, Acholeplasma virus L2, Pseudomonas virus PR4, Pseudomonas virus PRD1, Bacillus virus AP50, Bacillus virus Bam35, Bacillus virus GIL16, Bacillus virus Wip1, Escherichia virus phi80, Escherichia virus RB42, Escherichia virus T2, Escherichia virus T3, Escherichia virus T6, Escherichia virus VT2-Sa, Escherichia virus VT1-Sakai, Escherichia virus VT2-Sakai, Escherichia virus CP-933V, Escherichia virus P27, Escherichia virus Stx2phi-I, Escherichia virus Stx1phi, Escherichia virus Stx2phi-II, Escherichia virus CP-1639, based on the Escherichia virus BP-4795, Escherichia virus 86, Escherichia virus Min27, Escherichia virus 2851, Escherichia virus 1717, Escherichia virus YYZ-2008, Escherichia virus ECO26 P06, Escherichia virus ECO103_P15, Escherichia virus ECO103_P12, Escherichia virus ECO111_P16, Escherichia virus ECO111_P11, Escherichia virus VT2phi_272, Escherichia virus TL-2011c, Escherichia virus P13374, Escherichia virus Sp5.

In one embodiment, the bacterial virus particles typically target E. coli and include the capsid of a bacteriophage selected in the group consisting of BW73, B278, D6, D108, E, E1, E24, E41, FI-2, FI-4, FI-5, HI8A, Ff18B, i, MM, Mu, 025, PhI-5, Pk, PSP3, P1, P1D, P2, P4, S1, Wφ, φK13, φ1, φ2, φ7, φ92, 7 A, 8φ, 9φ, 18, 28-1, 186, 299, HH-Escherichia (2), AB48, CM, C4, C16, DD-VI, E4, E7, E28, FI1, FI3, H, H1, H3, H8, K3, M, N, ND-2, ND-3, ND4, ND-5, ND6, ND-7, Ox-I, Ox-2, Ox-3, Ox-4, Ox-5, Ox-6, PhI-I, RB42, RB43, RB49, RB69, S, SaI-I, Sal-2, Sal-3, Sal-4, Sal-5, Sal-6, TC23, TC45, TuII*-6, TuIP-24, TuII*46, TuIP-60, T2, T4, T6, T35, α1, 1, IA, 3, 3A, 3T+, 5φ, 9266Q, CFO103, HK620, J, K, K1F, m59, no. A, no. E, no. 3, no. 9, N4, sd, T3, T7, WPK, W31, ΔH, φC3888, φK3, φK7, φK12, φV-1, Φ04-CF, Φ05, Φ06, Φ07, φ1, φ1.2, φ20, φ95, φ263, φlO92, Ω8, 1, 3, 7, 8, 26, 27, 28-2, 29, 30, 31, 32, 38, 39, 42, 933W, NN-Escherichia (1), Esc-7-11, AC30, CVX-5, C1, DDUP, EC1, EC2, E21, E29, F1, F26S, F27S, Hi, HK022, HK97, HK139, HK253, HK256, K7, ND-I, PA-2, q, S2, T1,), T3C, T5, UC-I, w, β4, γ2, λ, ΦD326, φγ, Φ06, Φ7, Φ10, φ80, χ, 2, 4, 4A, 6, 8A, 102, 150, 168, 174, 3000, AC6, AC7, AC28, AC43, AC50, AC57, AC81, AC95, HK243, K10, ZG/3A, 5, 5A, 21EL, H19-J and 933H.

The present disclosure provides pharmaceutical or veterinary compositions comprising one or more of the bacterial delivery vehicles disclosed herein and a pharmaceutically acceptable carrier. Generally, for pharmaceutical use, the bacterial delivery vehicles may be formulated as a pharmaceutical preparation or compositions comprising at least one bacterial delivery vehicle and at least one pharmaceutically acceptable carrier, diluent or excipient, and optionally one or more further pharmaceutically active compounds. Such a formulation may be in a form suitable for oral administration, for parenteral administration (such as by intravenous, intramuscular or subcutaneous injection or intravenous infusion), for topical administration, for administration by inhalation, by a skin patch, by an implant, by a suppository, etc. Such administration forms may be solid, semi-solid or liquid, depending on the manner and route of administration. For example, formulations for oral administration may be provided with an enteric coating that will allow the synthetic bacterial delivery vehicles in the formulation to resist the gastric environment and pass into the intestines. More generally, synthetic bacterial delivery vehicle formulations for oral administration may be suitably formulated for delivery into any desired part of the gastrointestinal tract. In addition, suitable suppositories may be used for delivery into the gastrointestinal tract. Various pharmaceutically acceptable carriers, diluents and excipients useful in bacterial delivery vehicle compositions are known to the skilled person.

Also provided are methods for treating a disease or disorder caused by bacteria such as bacterial infection using the bacterial delivery vehicles or compositions disclosed herein. The methods include administering the bacterial delivery vehicles or compositions disclosed herein to a subject having a bacterial infection in need of treatment. In some embodiments, the subject is a mammal. In some embodiments, the subject is a human.

The pharmaceutical or veterinary composition according to the disclosure may further comprise a pharmaceutically acceptable vehicle. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. Suitable solid vehicles include, for example calcium phosphate, magnesium stearate, talc, sugars, lactose, dextrin, starch, gelatin, cellulose, polyvinylpyrrolidone, low melting waxes and ion exchange resins.

The pharmaceutical or veterinary composition may be prepared as a sterile solid composition that may be suspended at the time of administration using sterile water, saline, or other appropriate sterile injectable medium. The pharmaceutical or veterinary compositions of the disclosure may be administered orally in the form of a sterile solution or suspension containing other solutes or suspending agents (for example, enough saline or glucose to make the solution isotonic), bile salts, acacia, gelatin, sorbitan monoleate, polysorbate 8o (oleate esters of sorbitol and its anhydrides copolymerized with ethylene oxide) and the like. The particles according to the disclosure can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for enteral administration include sterile solutions, emulsions, and suspensions.

The bacterial delivery vehicles according to the disclosure may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilizers, emulsifiers, buffers, preservatives, sweeteners, flavouring agents, suspending agents, thickening agents, colours, viscosity regulators, stabilizers or osmo-regulators. Suitable examples of liquid vehicles for oral and enteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for enteral administration. The liquid vehicle for pressurized compositions can be a halogenated hydrocarbon or other pharmaceutically acceptable propellant.

For transdermal administration, the pharmaceutical or veterinary composition can be formulated into ointment, cream or gel form and appropriate penetrants or detergents could be used to facilitate permeation, such as dimethyl sulfoxide, dimethyl acetamide and dimethylformamide.

For transmucosal administration, nasal sprays, rectal or vaginal suppositories can be used. The active compounds can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

The diseases or disorders caused by bacteria may be selected from the group consisting of abdominal cramps, acne vulgaris, acute epiglottitis, arthritis, bacteraemia, bloody diarrhea, botulism, Brucellosis, brain abscess, chancroid venereal disease, Chlamydia, Crohn's disease, conjunctivitis, cholecystitis, colorectal cancer, polyposis, dysbiosis, Lyme disease, diarrhea, diphtheria, duodenal ulcers, endocarditis, erysipelothricosis, enteric fever, fever, glomerulonephritis, gastroenteritis, gastric ulcers, Guillain-Barre syndrome tetanus, gonorrhoea, gingivitis, inflammatory bowel diseases, irritable bowel syndrome, leptospirosis, leprosy, listeriosis, tuberculosis, Lady Widermere syndrome, Legionaire's disease, meningitis, mucopurulent conjunctivitis, multi-drug resistant bacterial infections, multi-drug resistant bacterial carriage, myonecrosis-gas gangrene, Mycobacterium avium complex, neonatal necrotizing enterocolitis, nocardiosis, nosocomial infection, otitis, periodontitis, phalyngitis, pneumonia, peritonitis, purpuric fever, Rocky Mountain spotted fever, shigellosis, syphilis, sinusitis, sigmoiditis, septicaemia, subcutaneous abscesses, tularaemia, tracheobronchitis, tonsillitis, typhoid fever, ulcerative colitis, urinary infection, whooping cough.

The disease or disorder caused by bacteria may be a bacterial infection selected from the group consisting of skin infections such as acne, intestinal infections such as esophagitis, gastritis, enteritis, colitis, sigmoiditis, rectitis, and peritonitis, urinary tract infections, vaginal infections, female upper genital tract infections such as salpingitis, endometritis, oophoritis, myometritis, parametritis and infection in the pelvic peritoneum, respiratory tract infections such as pneumonia, intra-amniotic infections, odontogenic infections, endodontic infections, fibrosis, meningitis, bloodstream infections, nosocomial infection such as catheter-related infections, hospital acquired pneumonia, post-partum infection, hospital acquired gastroenteritis, hospital acquired urinary tract infections, and a combination thereof. In an embodiment, the infection according to the disclosure is caused by a bacterium presenting an antibiotic resistance. In a particular embodiment, the infection is caused by a bacterium as listed above in the targeted bacteria. In another embodiment, the infection according to the disclosure is caused by a bacterium expressing toxin, such as shiga-toxin. In a particular embodiment, the infection is caused by a Shiga-Toxin producing E. coli (STEC).

The disease or disorder caused by bacteria may also be a metabolic disorder, for example, obesity and/or diabetes. The disclosure thus also concerns a pharmaceutical or veterinary composition as disclosed herein for use in the treatment of a metabolic disorder including, for example, obesity and/or diabetes. It further concerns a method for treating a metabolic disorder comprising administering a therapeutically efficient amount of the pharmaceutical or veterinary composition as disclosed herein, and the use of a pharmaceutical or veterinary composition as disclosed herein for the manufacture of a medicament for treating a metabolic disorder.

The disease or disorder caused by bacteria may also be a pathology involving bacteria of the human microbiome. Thus, in a particular embodiment, the disclosure concerns a pharmaceutical or veterinary composition as disclosed herein for use in the treatment of pathologies involving bacteria of the human microbiome, such as inflammatory and auto-immune diseases, cancers, infections or brain disorders. It further concerns a method for treating a pathology involving bacteria of the human microbiome comprising administering a therapeutically efficient amount of the pharmaceutical or veterinary composition as disclosed herein, and the use of a pharmaceutical or veterinary composition as disclosed herein for the manufacture of a medicament for treating a pathology involving bacteria of the human microbiome. Indeed, some bacteria of the microbiome, without triggering any infection, can secrete molecules that will induce and/or enhance inflammatory or auto-immune diseases or cancer development. More specifically, the present disclosure relates also to modulating microbiome composition to improve the efficacy of immunotherapies based, for example, on CAR-T (Chimeric Antigen Receptor T) cells, TIL (Tumor Infiltrating Lymphocytes) and Tregs (Regulatory T cells) also known as suppressor T cells. Modulation of the microbiome composition to improve the efficacy of immunotherapies may also include the use of immune checkpoint inhibitors well known in the art such as, without limitation, PD-1 (programmed cell death protein 1) inhibitor, PD-L1 (programmed death ligand 1) inhibitor and CTLA-4 (cytotoxic T lymphocyte associated protein 4).

Some bacteria of the microbiome can also secrete molecules that will affect the brain, such as serotonin and melatonin for use in the treatment of depression, dementia or sleep disorder.

Therefore, a further object of the disclosure is a method for controlling the microbiome of a subject, comprising administering an effective amount of the pharmaceutical or veterinary composition as disclosed herein in said subject.

In a particular embodiment, the disclosure also relates to a method for personalized treatment for an individual in need of treatment for a disease or disorder such as bacterial infection comprising: i) obtaining a biological sample from the individual and determining a group of bacterial DNA sequences from the sample; ii) based on the determining of the sequences, identifying one or more pathogenic bacterial strains or species that were in the sample; and iii) administering to the individual a pharmaceutical or veterinary composition according to the disclosure capable of recognizing each pathogenic bacterial strain or species identified in the sample and to deliver the packaged plasmid. The disclosure also relates to a pharmaceutical or veterinary composition according to the disclosure for use in the treatment of a disease or disorder such as bacterial infection wherein the pharmaceutical or veterinary composition is obtained by the method comprising: i) obtaining a biological sample from the individual to be treated and determining a group of bacterial DNA sequences from the sample; ii) based on the determining of the sequences, identifying one or more pathogenic bacterial strains or species that were in the sample, and iii) preparing the pharmaceutical or veterinary composition capable of recognizing each pathogenic bacterial strain or species identified in the sample and to deliver the packaged plasmid.

In an embodiment, the biological sample comprises pathological and non-pathological bacterial species, and subsequent to administering the pharmaceutical or veterinary composition according to the disclosure to the individual, the amount of pathogenic bacteria on or in the individual are reduced, but the amount of non-pathogenic bacteria is not reduced.

In another particular embodiment, the disclosure concerns a pharmaceutical or veterinary composition according to the disclosure for use to improve the effectiveness of drugs. Indeed, some bacteria of the microbiome, without being pathogenic by themselves, are known to be able to metabolize drugs and to modify them in ineffective or harmful molecules.

In another particular embodiment, the disclosure concerns a composition that may further comprise at least one additional active ingredient, for instance a prebiotic and/or a probiotic and/or an antibiotic, and/or another antibacterial or antibiofilm agent, and/or any agent enhancing the targeting of the bacterial delivery vehicle to a bacteria and/or the delivery of the payload into a bacteria.

As used herein, a “prebiotic” refers to an ingredient that allows specific changes, both in the composition and/or activity in the gastrointestinal microbiota that may confer benefits upon the host. A prebiotic can be a comestible food or beverage or ingredient thereof. A prebiotic may be a selectively fermented ingredient. Prebiotics may include complex carbohydrates, amino acids, peptides, minerals, or other essential nutritional components for the survival of the bacterial composition. Prebiotics include, but are not limited to, amino acids, biotin, fructo-oligosaccharide, galacto-oligosaccharides, hemicelluloses (e.g., arabinoxylan, xylan, xyloglucan, and glucomannan), inulin, chitin, lactulose, mannan oligosaccharides, oligofructose-enriched inulin, gums (e.g., guar gum, gum arabic and carrageenan), oligofructose, oligodextrose, tagatose, resistant maltodextrins (e.g., resistant starch), trans-galactooligosaccharide, pectins (e.g., xylogalactouronan, citrus pectin, apple pectin, and rhamnogalacturonan-I), dietary fibers (e.g., soy fiber, sugarbeet fiber, pea fiber, corn bran, and oat fiber) and xylooligosaccharides.

As used herein, a “probiotic” refers to a dietary supplement based on living microbes which, when taken in adequate quantities, has a beneficial effect on the host organism by strengthening the intestinal ecosystem. Probiotic can comprise a non-pathogenic bacterial or fungal population, e.g., an immunomodulatory bacterial population, such as an anti-inflammatory bacterial population, with or without one or more prebiotics. They contain a sufficiently high number of living and active probiotic microorganisms that can exert a balancing action on gut flora by direct colonisation. It must be noted that, for the purposes of the present description, the term “probiotic” is taken to mean any biologically active form of probiotic, preferably including but not limited to lactobacilli, bifidobacteria, streptococci, enterococci, propionibacteria or saccharomycetes but even other microorganisms making up the normal gut flora, or also fragments of the bacterial wall or of the DNA of these microorganisms. These compositions are advantageous in being suitable for safe administration to humans and other mammalian subjects and are efficacious for the treatment, prevention, of a disease or disorder caused by bacteria such as bacterial infection. Probiotics include, but are not limited to lactobacilli, bifidobacteria, streptococci, enterococci, propionibacteria, saccharomycetes, lactobacilli, bifidobacteria, or proteobacteria.

The antibiotic can be selected from the group consisting of penicillins such as penicillin G, penicillin K, penicillin N, penicillin O, penicillin V, methicillin, benzylpenicillin, nafcillin, oxacillin, cloxacillin, dicloxacillin, ampicillin, amoxicillin, pivampicillin, hetacillin, bacampicillin, metampicillin, talampicillin, epicillin, carbenicillin, ticarcillin, temocillin, mezlocillin, and piperacillin; cephalosporins such as cefacetrile, cefadroxil, cephalexin, cefaloglycin, cefalonium, cefaloridine, cefalotin, cefapirin, cefatrizine, cefazaflur, cefazedone, cefazolin, cefradine, cefroxadine, ceftezole, cefaclor, cefonicid, cefprozil, cefuroxime, cefuzonam, cefmetazole, cefotetan, cefoxitin, loracarbef, cefbuperazone, cefminox, cefotetan, cefoxitin, cefotiam, cefcapene, cefdaloxime, cefdinir, cefditoren, cefetamet, cefixime, cefmenoxime, cefodizime, cefotaxime, cefovecin, cefpimizole, cefpodoxime, cefteram, ceftamere, ceftibuten, ceftiofur, ceftiolene, ceftizoxime, ceftriaxone, cefoperazone, ceftazidime, latamoxef, cefclidine, cefepime, cefluprenam, cefoselis, cefozopran, cefpirome, cefquinome, flomoxef, ceftobiprole, ceftaroline, ceftolozane, cefaloram, cefaparole, cefcanel, cefedrolor, cefempidone, cefetrizole, cefivitril, cefmatilen, cefmepidium, cefoxazole, cefrotil, cefsumide, ceftioxide, cefuracetime, and nitrocefin; polymyxins such as polysporin, neosporin, polymyxin B, and polymyxin E, rifampicins such as rifampicin, rifapentine, and rifaximin; Fidaxomicin; quinolones such as cinoxacin, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, grepafloxacin, levofloxacin, pazufloxacin, temafloxacin, tosufloxacin, clinafloxacin, gatifloxacin, gemifloxacin, moxifloxacin, sitafloxacin, trovafloxacin, prulifloxacin, delafloxacin, nemonoxacin, and zabofloxacin; sulfonamides such as sulfafurazole, sulfacetamide, sulfadiazine, sulfadimidine, sulfafurazole, sulfisomidine, sulfadoxine, sulfamethoxazole, sulfamoxole, sulfanitran, sulfadimethoxine, sulfametho-xypyridazine, sulfametoxydiazine, sulfadoxine, sulfametopyrazine, and terephtyl; macrolides such as azithromycin, clarithromycin, erythromycin, fidaxomicin, telithromycin, carbomycin A, josamycin, kitasamycin, midecamycin, oleandomycin, solithromycin, spiramycin, troleandomycin, tylosin, and roxithromycin; ketolides such as telithromycin, and cethromycin; fluoroketolides such as solithromycin; lincosamides such as lincomycin, clindamycin, and pirlimycin; tetracyclines such as demeclocycline, doxycycline, minocycline, oxytetracycline, and tetracycline; aminoglycosides such as amikacin, dibekacin, gentamicin, kanamycin, neomycin, netilmicin, sisomicin, tobramycin, paromomycin, and streptomycin; ansamycins such as geldanamycin, herbimycin, and rifaximin; carbacephems such as loracarbef; carbapenems such as ertapenem, doripenem, imipenem (or cilastatin), and meropenem; glycopeptides such as teicoplanin, vancomycin, telavancin, dalbavancin, and oritavancin; lincosamides such as clindamycin and lincomycin; lipopeptides such as daptomycin; monobactams such as aztreonam; nitrofurans such as furazolidone, and nitrofurantoin; oxazolidinones such as linezolid, posizolid, radezolid, and torezolid; teixobactin, clofazimine, dapsone, capreomycin, cycloserine, ethambutol, ethionamide, isoniazid, pyrazinamide, rifabutin, arsphenamine,chloramphenicol, fosfomycin, fusidic acid, metronidazole, mupirocin, platensimycin, quinupristin (or dalfopristin), thiamphenicol, tigecycline, tinidazole, trimethoprim, alatrofloxacin, fidaxomicin, nalidixic acid, rifampin, derivatives and combination thereof.

In another particular embodiment, the disclosure concerns the in-situ bacterial production of any compound of interest, including therapeutic compound such as prophylactic and therapeutic vaccine for mammals. The compound of interest can be produced inside the targeted bacteria, secreted from the targeted bacteria or expressed on the surface of the targeted bacteria. In a more particular embodiment, an antigen is expressed on the surface of the targeted bacteria for prophylactic and/or therapeutic vaccination.

The present disclosure also relates to a non-therapeutic use of the bacterial delivery particles. For instance, the non-therapeutic use can be a cosmetic use or a use for improving the well-being of a subject, in particular a subject who does not suffer from a disease. Accordingly, the present disclosure also relates to a cosmetic composition or a non-therapeutic composition comprising the bacterial delivery particles of the disclosure.

The present disclosure will be further illustrated by the examples below.

### EXAMPLES

### Example 1

Lambda-packaged cosmids are derived from lambda PaPa, a variant of the wild-type Ur-lambda phage with a frameshift mutation in the stf gene [7] leading to a truncated protein which is an inactive STF protein, i.e a protein with no biological activity. Lambda Papa has been used as the de facto wild-type lambda phage in the majority of laboratory studies, because as opposed to wild-type Ur-lambda, it makes larger plaques that are easier to handle. The stf gene codes for the side tail fiber protein, which in the case of phage lambda recognizes the secondary receptor on the cell surface, OmpC.

This secondary receptor allows for transient binding of the phage particle on the cell surface in order to scan the surface and position the injection machinery in contact with the primary receptor (LamB in the case of lambda, interaction mediated by the lambda protein gpJ). Since the STF binding is reversible, it allows the phage to “walk” on the cell surface until a primary receptor is found and the infection process starts. These protein complexes are sometimes referred to as “L-shape fibers”, such as in T5, “side tail fibers” such as in lambda, “long tail fibers” as in T4, or tailspikes such as in phage P22 [7]-[10]. For some phages, the presence of this second set of proteins is necessary for the infection process to occur, such as T4 [8]. In some other phages, like lambda, this second set of proteins is not necessary for the infection process to happen, but it may allow for a more efficient attachment to the target cell [7].

The wild-type length of the lambda phage genome is 48.5 kbp. It is well known that lambda can only package DNA from 37.7 kbp to 51 kbp [11], or about 78% to 105% of the wild-type length. Smaller DNA payloads do not build up enough pressure inside the capsid for packaging termination to occur and larger ones make the capsid too unstable. Additionally, smaller genomes have been shown to be ejected with much lower efficiency in the presence of higher external osmotic pressures [12]: as the length of the encapsidated DNA decreases, the ejection force decreases in an exponential fashion between the two size extremes 37.7 to 51 kbp. It is also known that the smaller the payload is, the lower the efficiency at which packaged particles will form [11]. Combining these two observations, it can be concluded that a smaller DNA payload will be detrimental if high enough titers for in vivo experiments are needed.

Most of the phagemids packaged using the lambda system (and many others) are much smaller than the wild-type length of the phage. Packaging is possible because the cosmid forms concatemers via the sigma replication pathway: when the concatemers fall between 74% and 105% of the wild-type lambda genome length, packaging is terminated and a mature packaged cosmid is formed [11]. This means that these particles, in contrast to a wild-type lambda genome, will not have packaged DNA of a homogeneous length: the whole range of 74% to 105% genome lengths will be present. For example, in [11] a cosmid of 12.8 kb was shown to be packaged as trimers and tetramers, which correspond to 38.4 kb and 51.2 kb (in the range of allowed packaged sizes); the 38.4 kb variant was found in about 15% of the particles while the 51.2 kb was found in about 40%. Similarly, a 4.6 kb plasmid was shown to be packaged as a 9-mer, 10-mer and 11-mer (41.4 kb, 46 kb and 50.6 kb). In this case, the 41 kb variant was the most common with about 20% of the particles having this size, followed by the 50.6 kb variant with about 12%; the 46 kb variant was only present in about 5% of the particles.

For in vivo applications, such as oral delivery of encapsidated DNA particles, packaged phagemids will need to be given at high enough concentrations to reach all the target cells; hence, a payload that gives high enough titers is essential to optimize the in vivo activity as well as the manufacturing process.

Finally, besides a payload with a proper size, the packaged particle needs to be able to bind its target cell strongly and long enough for the injection process to occur. It was previously shown that the presence of STF is not necessary for lambda-mediated in vitro transduction experiments in K-12 laboratory strains [7].

Here, it has been unexpectedly shown that both a suitable payload that is packaged as concatemers of the correct length as well as a functional side tail fiber greatly increase efficiency when performing in vivo delivery assays of lambda-based packaged phagemids. It has also unexpectedly demonstrated herein that for strains other than model K12 strains, STF and/or gpJ from other phages that can specifically recognize surface antigens/receptors of the target bacteria can be used to generate engineered lambda viral particles that mediate efficient delivery both in vitro and in vivo.

A mouse model, using E. coli strain MG1655, or derivatives, with engineered streptomycin resistance was developed using an established colonization protocol [13]. Mice (Balb/c ByJ, 7 weeks) were treated with a short course of streptomycin, which is known to reduce the natural coliform intestinal population [14]. This treatment allows for exogenous E. coli to be administered and colonize empty ecological niches while maintaining other species that are present in the natural microbiota. In detail, animals were treated with a 5-day course of 5 g/L streptomycin in drinking water. The treatment was stopped 5 days before gavaging of the packaged phagemids to avoid any bias and selection of resistance due to antibiotic evolutionary pressure.

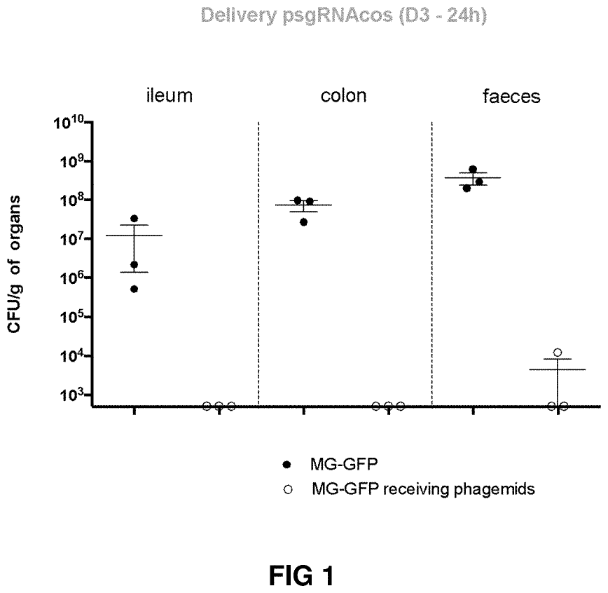

To test the in vivo delivery efficiency of packaged phagemids, psgRNAcos, the 2.5 kb cosmid of SEQ ID NO: 1 carrying a kanamycin resistance gene was packaged into lambda PaPa particles, encoding a non-functional stf gene. To achieve this, the cosmid was transformed in an E. coli strain carrying the lambda prophage lacking a cos site but otherwise possessing all the machinery for the induction of the lambda phage lytic cycle as well as the DNA packaging system. The cI repressor of the lambda prophage carries mutations making it thermosensitive and enabling the induction of the lytic cycle through temperature change. The cells containing the lambda cosmid were grown at 30° C. in liquid LB media. At an OD600 of 0.6, the culture was shifted to 42° C. for 25 minutes to induce the entry into lytic cycle. After that, cells were shifted back to 37° C. for 3 hours to allow for virion assembly containing the lambda cosmid. Cells were then centrifuged and washed in lambda buffer (10 mM Tris pH 7.5, 100 mM NaCl, 10 mM MgSO₄). Chloroform was added and the sample was spun down at 17,000 g for 5 minutes. Finally, the aqueous phase was collected and filtered through a 0.2 μm pore-size filter. The titer of the packaged phagemids was measured by performing a transduction assay in vitro using strain MG-GFP as a recipient. MG-GFP is a derivative of strain MG1655 with a gfp fluorescent reporter gene and an ampicillin resistance gene inserted in the chromosome. The titer was determined to be about 10⁶ particles/μl (not shown). This packaged phagemid stock was used to transduce strain MG-GFP in the gut of mice colonized by MG-GFP for 5 days. Feces were collected at different time points, homogenized and plated on LB agar plates with kanamycin to monitor the number of transduced MG-GFP cells. Kanamycin-resistant colonies were counted in different parts of the mice intestinal tract (psgRNAcos encodes a kanamycin marker). As depicted in FIG. 1. almost no detectable transductants were observed anywhere.

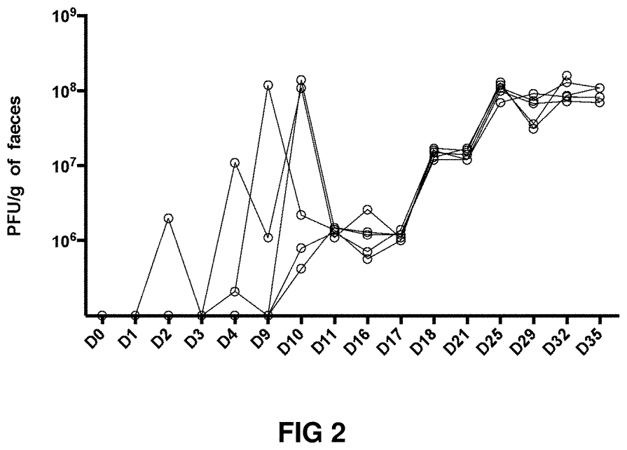

The low delivery efficiencies observed are consistent with the fact that the lambda PaPa phage replicates very poorly in the gut of mice colonized by MG-GFP, as measured by counting plaque forming units over time in the feces of mice colonized by MG-GFP and infected with lambda PaPa. To identify the reason for this poor efficiency of lambda PaPa in the mouse gut, an in vivo evolution assay was performed to select improved variants of this phage. Mice colonized by MG-GFP were gavaged with an initial total dose of 10⁷ lambda PaPa. Every day, faeces were collected and tested for their ability to form plaques on lawns of MG1655. They were also homogenized in 1×PBS at 40 mg/mL, filtered through a 0.22 μm pore membrane and re-fed to the mice. As can be seen in FIG. 2, initially there were virtually no PFUs detected, as lambda Papa was not able to bind its host in the mouse gut. Some peaks were observed in the amount of PFU-forming particles whose amount oscillated from high to undetectable levels up to day 17, where a steady increase in the number of PFU was detected.

Strikingly, when the genomes of the phage particles obtained on day 35 were sequenced, it was observed that the ORF of the stf gene had been restored: the lambda PaPa particles had reverted the mutation to give a full-length stf gene (although with mutations in K338E, T391M and A395S with respect to the canonical STF sequence of SEQ ID NO: 14). Additionally, it was observed that the gpJ gene, responsible for binding to the LamB receptor, had also mutated in three positions. These mutations were tested to determine if they altered the receptor being used by this phage on wild-type MG1655 and MG1655-delta-LamB. No difference was observed, which confirmed that the new Ur-lambda particles still use LamB as their primary receptor.

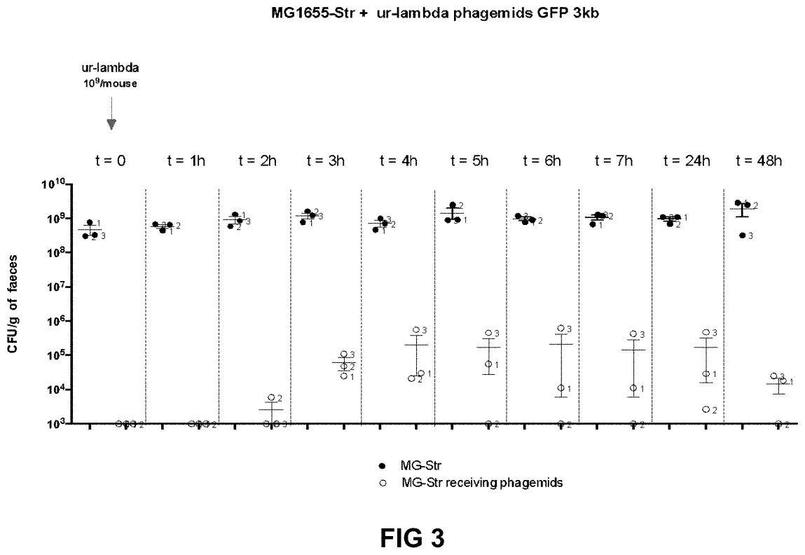

With the ORF of the stf gene restored in the original lambda packaged phagemid production strain, but keeping the original STF sequence of SEQ ID NO: 14 and not the mutated version obtained in the experiment above, the same experiment was performed with a packaged phagemid containing a DNA payload of 3 kb in size (pJ23104-GFP of SEQ ID NO: 2) but this time encoding a chloramphenicol marker. The sequence of the gpJ gene was not changed at this stage. The packaged phagemids were gavaged to mice colonized with MG1655-Strp (with streptomycin resistance). Feces were homogenized at 40 mg/mL in 1×PBS and serially diluted and plated in Drigalski plates supplemented or not with 25 μg/mL chloramphenicol. The amount of chloramphenicol resistant cells was hence counted in the faeces up to 48 h after transduction. As can be seen in FIG. 3, the amount of transduced cells increased as compared to lambda PaPa packaged phagemids, but the delivery efficiency was very low (about 1 in 1000 cells). When the same experiment was attempted with packaged Ur-lambda particles with a modified gpJ gene to mimic the particles obtained in the in vivo evolution assay, the same results were observed. It was concluded that there must be another factor that prevents the packaged phagemids from working optimally in vivo, as no significant differences were seen in in vitro transduction assays.

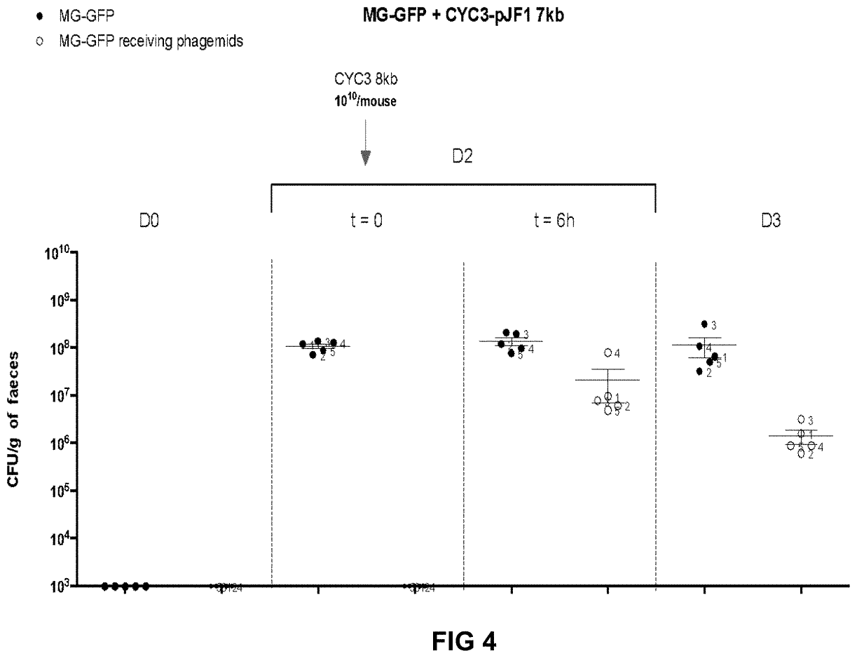

An experiment was then done in which a packaged phagemid was produced with a larger DNA payload, pJF1 of SEQ ID NO: 3 (around 7 kb) in Ur-lambda capsids and administered to the mice. It is important to note that the gpJ gene was not changed, so these particles encode the wild-type gpJ lambda of SEQ ID NO: 10 and still use LamB as their primary receptor. The same protocol was followed for in vivo delivery as for the smaller DNA payload of SEQ ID NO: 2. Surprisingly, as can be seen in FIG. 4, the addition of both a functional stf gene and a DNA payload that is packaged at higher titers due to its length drastically increases the delivery efficiency in vivo.

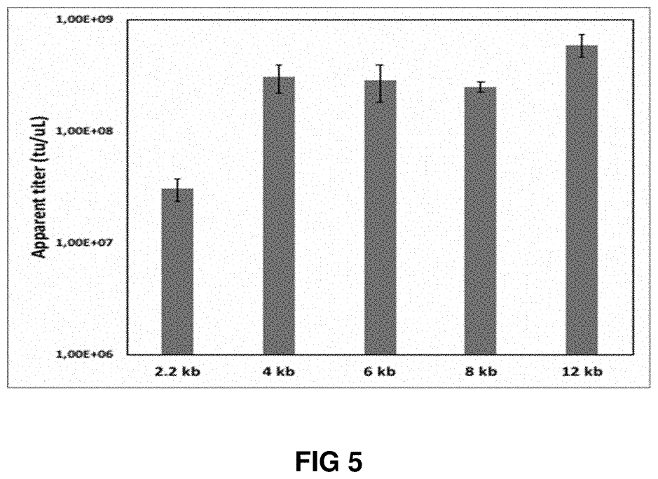

Since strong differences were observed depending on the size of the DNA payload used, a set of experiments were performed where DNA payload of different sizes of SEQ ID NO: 4 to 8 were packaged into lambda capsids delivery vehicles and their titers assessed before the in vivo assays. The production was carried out in the same conditions for all payloads. After production and lysis following the protocol mentioned above, a step of TFF filtration with a 100 kDa pore size was carried out on the cleared lysates, and buffer exchanged for 1×PBS. The TFF step allows for the concentration of the particles about 1 log compared to the cleared lysates. As can be seen in FIG. 5 smaller DNA payloads (2.2 kb) are produced at titers that are at least one log lower than their larger counterparts in our production system.

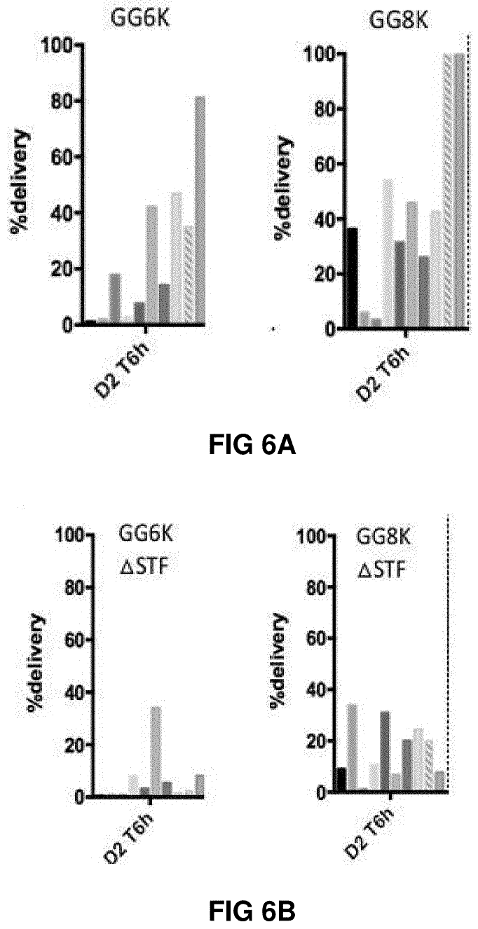

Finally, to verify the roles of DNA payload size, titers and STF presence, the 6 kb and 8 kb variants (GG6K of SEQ ID NO: 6 and GG8K of SEQ ID NO: 7, respectively) shown in FIG. 5 were packaged in two lambda phagemid versions: lambda particles containing an intact full STF with its chaperone protein and lambda particles produced in a strain where the stf and tfa chaperone genes were seamlessly deleted from the prophage genome. Both versions encode the wild type lambda gpJ gene of SEQ ID NO: 10. After clearing, the lysates were passed through a TFF device with a 100 kDa filter as described for the phagemids in FIG. 5 and their titers measured (about 10⁸/μL). 10¹⁰ total packaged phagemids of each DNA payload were gavaged to mice and the delivery efficiency calculated as described above. Strikingly, as can be seen in FIG. 6A, the addition of a functional STF greatly increases the delivery efficiency in vivo, which in some cases is 100%.

Significantly, these results highlight the importance of a functional STF in in vivo conditions, which does not reflect the results obtained in vitro, where no obvious difference can be observed using lambda Papa or Ur-lambda packaged phagemids by plating.

This set of experiments brings three conclusions that have a major impact for the development of a successful approach to use packaged phagemids in vivo, especially for decolonization purposes. First, a DNA payload with a proper size to obtain high enough titers is necessary. If the DNA payload is too small, an intensive downstream processing focused on concentration of the particles to suitable titers must be put in place, which may slow down the production process and increase its cost. Second, the presence of a functional STF is essential to obtain the high delivery efficiencies needed for in vivo experiments, and it has been previously shown that STF can be engineered for each target strain (see for instance US provisional application U.S. 62/802,777, U.S. application Ser. No. 16/696,769 and U.S. application Ser. No. 16/726,033 each of which is incorporated by reference in their entirety). Third, the primary receptor, LamB in this case, may not be optimal for the pursued application. As described below, it is shown that one can engineer phagemid particles to include gpJ variants (in the case of lambda) that bind other receptors than LamB.

### Engineering the Lambda gpJ Protein

The lambda phage uses the bacterial LamB OMP as its main entry receptor [15], which is recognized by the gpJ protein situated at the tip of the phage particle. This event triggers DNA ejection into the bacterial cytoplasm and is usually viewed as an “irreversible” binding process [15]. However, a bacterial strain can become resistant to lambda phage entry if the LamB receptor is mutated, masked or downregulated; in particular, downregulation of the LamB gene has been observed for MG1655 strains in some mouse models, and this downregulation is caused by genotypic change: a mutation in the malT gene, regulating LamB expression levels, causes a drastic decrease in the number of these receptors in the membrane [16], [17]. In turn, bacteriophages have evolved different strategies to bypass these defense mechanisms. For instance, mutating the gpJ protein allows them to use a different receptor [18], [19]. It is also known that the receptor-recognition activity of gpJ lies in its C-terminal part, with a fragment as small as 249 aa conferring the capability of binding to LamB receptors [5].

Therefore, having different combinations of STF and gpJ that allow entry in specific conditions or different bacterial strains is crucial for the successful utilization of lambda-derived packaged phagemids as therapeutic agents, especially for in vivo applications. For this reason, several gpJ chimeras have been engineered that recognize receptors other than LamB, as well as identified variants that allow entry in some E. coli strains but not others.

To achieve this, gpJ homologs from different E. coli prophage genomes were searched and a protein alignment to find the areas in the C-terminal part that may be involved in recognizing the bacterial OMP receptor was performed (FIG. 7).

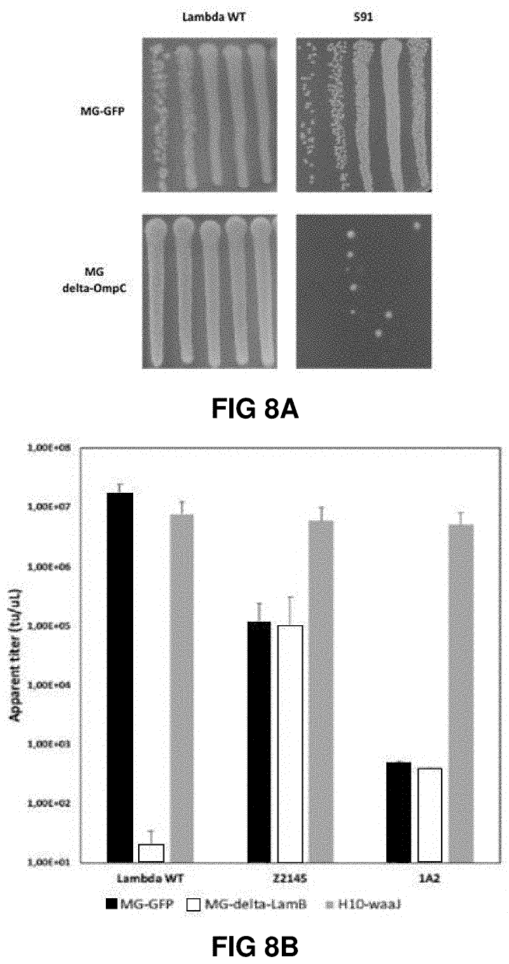

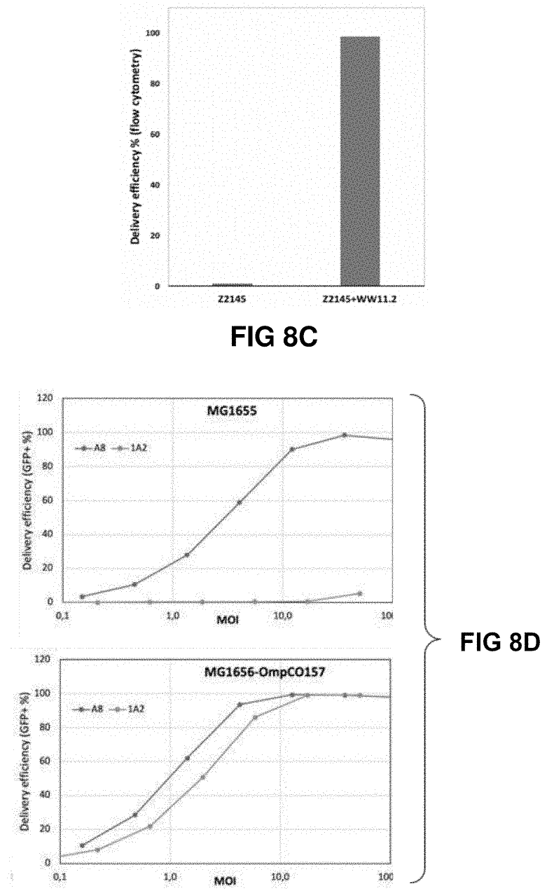

As shown in FIG. 7 all gpJ variants share high sequence identity in the majority of their length (up to about amino acid 820) except for their C-termini. To verify that the receptor specificity is contained within this area of the gpJ protein, different fusions were tested using two insertion points (SEQ ID NO: 42 and SEQ ID NO: 43) as shown in FIG. 7. These chimeras share the N-terminus of the lambda gpJ and differ in their C-termini, which comes from other E. coli prophages. The gpJ variants were seamlessly inserted into the lambda production strain and packaged phagemids containing DNA payload p7.3 kb (encoding a GFP and a chloramphenicol resistance gene) of SEQ ID NO: 9 produced as described above. Four strains were used to titrate these packaged phagemids: MG-GFP (a variant of MG1655 that encodes a GFP in the genome), MG-delta-LamB (KEIO variant lacking the LamB receptor), H10-waaJ, a O157 strain lacking the waaJ gene, which prevents the expression of the O157 capsular antigen [20]) and MG1656-OmpCO157, a modified MG1655 in which the original OmpC has been exchanged for the OmpC variant found in O157 strains. The apparent titers shown in FIG. 8 are used as a measure of the efficiency of the packaged phagemids, since the OD600 of all strains was kept constant (0.8). As can be seen in FIG. 8., both insertion points yield functional gpJ chimeras, but surprisingly the recognized receptor has changed from the one recognized originally by lambda gpJ; for instance, as shown in FIG. 8A, the variant H591 of SEQ ID NO: 11 now uses OmpC. Interestingly, two of the gpJ variants (Z2145 of amino acid sequence SEQ ID NO: 12 and 1A2 of amino acid sequence SEQ ID NO: 13), show reduced or virtually no entry in MG1655, respectively, while they are able to recognize a receptor present in O157 strains (FIG. 8). Neither variant uses the LamB receptor, as the titers in the MG-GFP and MG-delta-LamB strains are the same (FIG. 8B). Finally, another gpJ variant was constructed and named A8 (SEQ ID NO: 49) and its delivery efficiency was tested on both MG1655 (containing its endogenous OmpC variant) and MG1656-OmpCO157. To do this, serial 1:3 dilutions of phagemids containing the A8 or 1A2 gpJ variants and a P2-stf chimera (protein of sequence SEQ ID NO: 50, typically encoded by the nucleic acid sequence SEQ ID NO: 56) were incubated with a fixed amount of MG1655 or MG1656-OmpCO157 cells at an OD600=0.025; by doing this, different MOIs were obtained. After incubation at 37° C. for 45 min, the GFP levels of each MOI were measured using a flow cytometer and plotted against the MOI. As was observed previously, the 1A2 gpJ variant was only able to recognize the receptor in MG1656-OmpCO157; but surprisingly, the A8 variant was able to recognize both the OmpC receptor in MG1655 and MG1656-OmpCO157.

In the light of these results, it seems that the approach of generating gpJ chimeras can produce not only variants that recognize different OMPs in the same strain, but also variants that show different strain specificity.

However, changing the primary receptor is a futile effort if the strain is encapsulated: since the packaged phagemid will not have access to the receptor due to a physical masking of the cell surface, the delivery efficiency will be drastically reduced. It is for this reason that for the activity tests of the gpJ fusions shown above, the naked H10-waaJ O157 strain was used, since it is known that O157 strains produce a group IV capsular antigen that masks the cellular surface [21]. In this case, a combination of a functional STF and gpJ is necessary to obtain high delivery efficiencies.

To do this, stf and the tfa (chaperone) genes from the lambda prophage in the production strain were seamlessly deleted. Chimeric stfs were then complemented in trans with a plasmid carrying DAPG-inducible versions of these chimeric stfs. Indeed, when using a STF variant referred to as WW11.2 and represented by SEQ ID NO: 16 that shows specificity for the O157 antigen, in combination with the gpJ variant Z2145 of amino acid sequence SEQ ID NO: 12 shown above, very efficient entry in encapsulated O157 strain were obtained, as can be seen in FIG. 8C.

### Engineering the Lambda Tape Measure Protein, gpH

Additionally, it has been shown that another possible mechanism for bacteria to protect from phage injection is the mutation of periplasmic proteins that are believed to assist in the formation of a channel across the periplasm while the DNA is being injected [22]-[24]. Lambda phage can regain its activity in these mutants by modifying the gpH gene, the tape measure protein, although the exact position of these mutations is not known. In the lambda phage, the protein involved in the interaction with the mannose permease complex is gpH. Recently, it has been described that some E. coli strains become resistant to infection by HK97, a lambdoïd phage, by mutations in the glucose transporter protein PtsG; the gpH protein of HK97 is inhibited and injection into these mutants cannot occur. The authors describe that by changing a region of the gpH protein in the HK97 phage they could bypass the PtsG mutation and the engineered HK97 becomes infectious again.

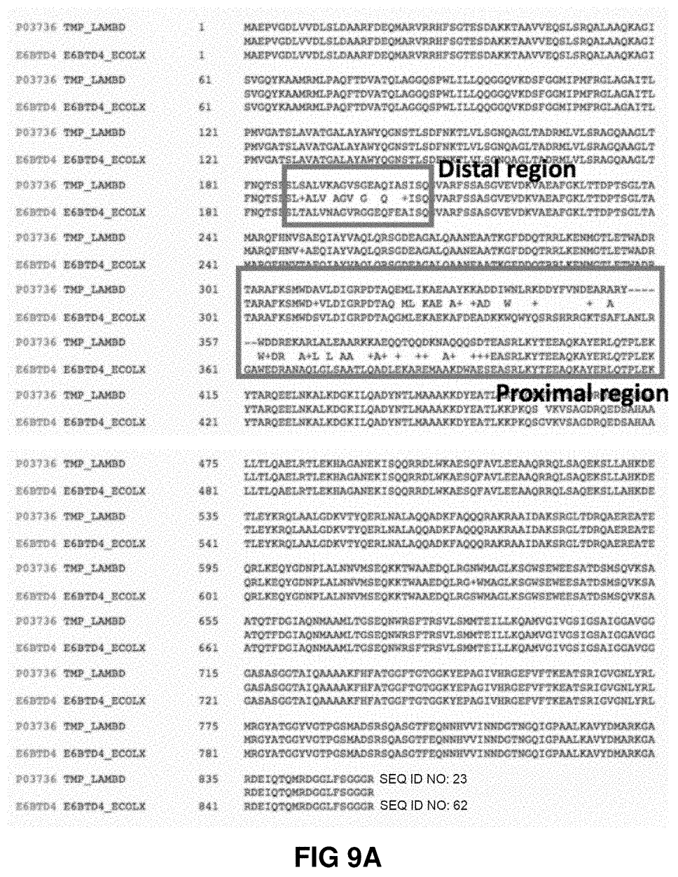

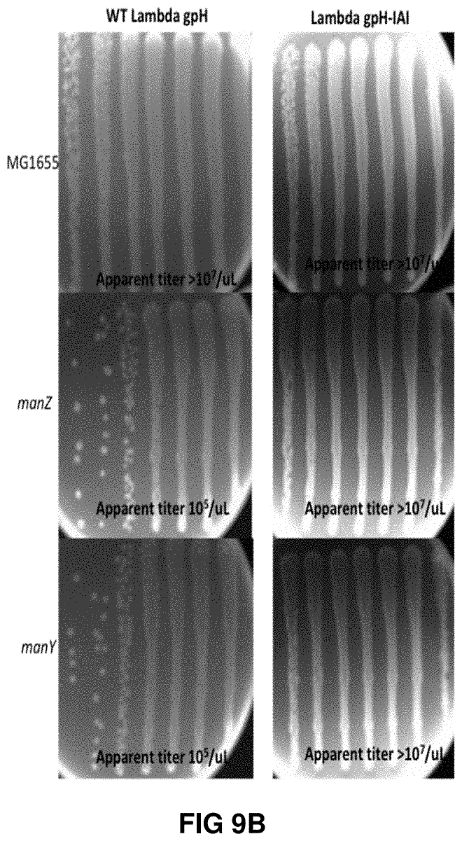

To test if this hypothesis is generalizable to the lambda phage, alignments of the lambda gpH protein to other lambdoïd prophages present in E. coli was performed. As shown in FIG. 9A, other variants of gpH can be found where there is an extremely high percentage of identity across the length of gpH except for two regions (labeled distal and proximal) which show heterogeneity in their sequence.

The gpH gene was modified in the lambda packaged phagemid production strain, as was done for the gpJ variants, to include both variable regions and packaged phagemids were produced as described above. This gpH variant of SEQ ID NO: 24 was termed gpH-IAI. In this case, three strains were used for titration: MG1655, KEIO manY and KEIO manZ, which contain deletions of two components of the mannose permease complex. As can be seen in FIG. 9B, the deletion of manZ and manY causes a 2-log decrease in the apparent titer as compared to MG1655; however, using the modified gpH lambda phage variant of SEQ ID NO: 24 fully restores its activity, even in the manZ and manY strains. These results show that modifying the gpH gene in lambda phage can be used to allow or improve entry in strains that show deficiencies or changes in the permease complexes.

With this battery of experiments, it is shown that one is able to engineer the three main mechanisms involved in lambdoïd phage recognition and injection into its host: capsule/secondary receptor (through STF); primary receptor recognition (through gpJ); and permease/periplasmic channel formation (through gpH). These modifications can be used to engineer lambdoïd phages and packaged phagemids with modified tropism and injection efficiency in a wide number of E. coli strains.

### Using Lambda Phagemids to Efficiently Deliver to Different, Unmodified Proteobacteria

Finally, it has been shown that lambdoïd-derived packaged phagemids can be used to deliver in other Proteobacteria different from E. coli, such as Klebsiella, Agrobacterium, or Pseudomonas. [25]-[27]. However, this approach requires the transformation of the receiver strains with a plasmid encoding the E. coli LamB receptor, since other species do not possess it. While this is a valid approach for cells grown in laboratory conditions, it becomes infeasible if bacteria present in natural conditions are to be targeted with lambda-derived packaged phagemids (for instance, the gut), since the plasmid encoding the receptor cannot be easily transferred.

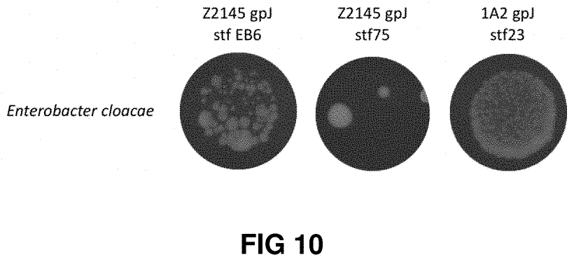

It was tested if the lambda-derived packaged phagemids could be used to deliver the p7.3 kb payload of SEQ ID NO: 9 to other Proteobacteria, such as Enterobacter. To achieve this, different lambda packaged phagemids were engineered to contain several combinations of gpJ, Z2145 of SEQ ID NO: 12 and 1A2 of SEQ ID NO: 13 and, STF variants STF-EB6 of SEQ ID NO: 19 (with its chaperone (or accessory) protein of SEQ ID NO: 20), STF75 of SEQ ID NO: 17 (with its accessory protein of SEQ ID NO: 18) and STF23 of SEQ ID NO: 21 (with its accessory protein of SEQ ID NO: 22). FIG. 10 shows that the same principles hold as observed for E. coli strains: the delivery efficiency depends strongly on the choice of gpJ and STF used; for some combinations, entry in these bacteria is inefficient (although transductants can be readily seen) but changing the STF and gpJ allows for a much higher delivery efficiency.

These results show that injection elements of the lambda capsid (gpJ, stf, gpH) can be engineered to achieve high delivery efficiency in other type of bacteria than E. coli. Accordingly, the present example shows that it is advantageous to use one or more of these approaches (DNA payload size optimization and selection of a proper gpJ/STF/gpH combination) to generate optimized lambdoïd-based delivery vehicles to be used in in vivo experiments involving transfer of DNA.

### Example 2

### Using Lambda Phagemids to Efficiently Deliver in Bacteria In Vivo

Tests were conducted to determine if a payload inside packaged phagemids with lambda STF fusions based on homology points could be delivered to bacteria in the gastrointestinal tract of a host without such STF being affected by proteolytic activity present in the gastrointestinal tract, in particular by pancreatin (i.e. low to no delivery of the payload to bacteria).

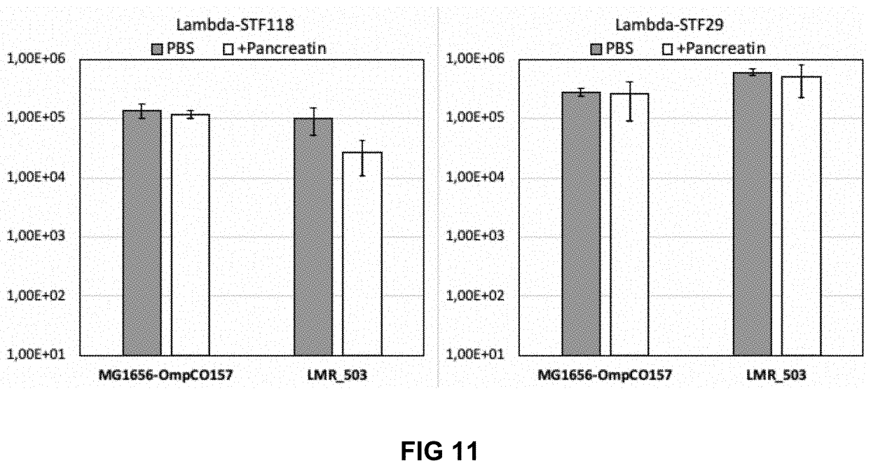

In principle, the structure of homology-based STF chimeras should be reminiscent of the original proteins, as the amino acid sequence of the fusion points has not been majorly modified. This means that if the original STFs had evolved to be pancreatin resistant, it is quite probable that the chimera will also be resistant. To prove this, two different STF chimeras with lambda STF functional when targeting LMR_503 strain (lambda-STF29, SEQ ID NO: 63 with its accessory protein of sequence SEQ ID NO: 65; and lambda-STF118, SEQ ID NO: 64 with its accessory protein of sequence SEQ ID NO: 66) were engineered. The insertion point of STF29 is ADAKKS (SEQ ID NO: 38) and the insertion point of STF118 is MDETNR (SEQ ID NO: 39). Eligobiotics® harboring the 1A2 gpJ and each of the STF chimeras were produced and titrated on MG1656-OmpCO157 or LMR 503 after treatment with or without pancreatin at pH 6.8. Briefly, the readout strain for chimeric STF activity is LMR 503 and the readout for gpJ activity is MG1656-OmpCO157. As can be seen in FIG. 11, these STF chimeras based on homology points show virtually no degradation in the presence of pancreatin, as predicted based on STF chimeras homology-designed fusion points.

### Example 3

### Effect of DNA Payload Size on Eligobiotics® Packaging

To evaluate the effect of DNA payload size on the number of payloads packaged in Eligobiotics® (EB), 3 different payloads were used to produce Eligobiotics® as summarized in Table 1.

TABLE 1
Batches of Eligobiotics ® produced

| Eligobiotic ® code/batch number | Payload name | Size (kb) |
|---|---|---|
| eb512/EB003-DS-008 | p1085 | 12.125 |
| eb393/EB003-DS-009 | p779 | 12.428 |
| eb827/EB003-DS-011 | p1392 | 11.615 |

After fermentation, lysis (3 h incubation at 37° C. with 0.1% Triton X-100, 2000 U/L Benzonase) and clarification on a Zeta Plus Capsule (3M), Eligobiotics® were purified by anion exchange chromatography on a Sartobind Q capsule (Sartorius). This initial purification was followed by a buffer exchange and concentration step by tangential flow filtration on a Pellicon 2 minicassette Biomax 300 kDa (Millipore). A final polishing step of size exclusion chromatography on Sepharose 6FF resin (GE Healthcare) was performed to yield the purified Eligobiotics.

Analysis of Eligobiotics® DNA content was performed by analytical ultracentrifugation in a Beckman Coulter Optima AUC using an AN50Ti rotor at 6 krpm. The sedimentation coefficients of different EBs present in solution for each EB batch were extracted from sedimentation velocity data (acquired at 260 and 280 nm).

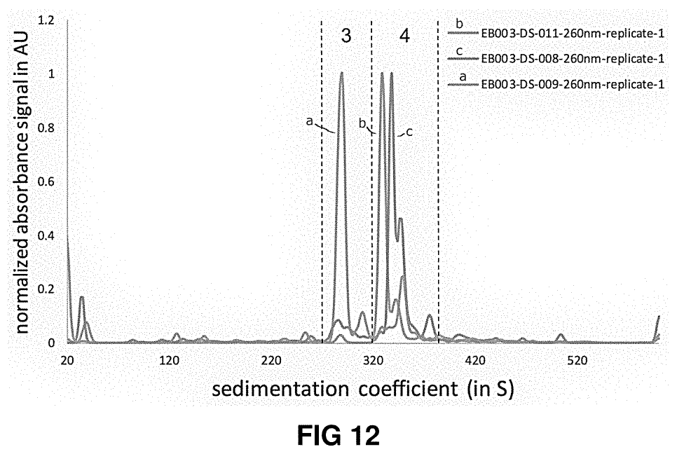

Based on the molecular weight calculated from their sedimentation coefficient and their 260/280 nm ratios, the different populations of EBs detected could be separated as Eligobiotics® containing either 3 copies (centered on 290 S) or 4 copies (centered on 330-340 S) of the payload (FIG. 12).

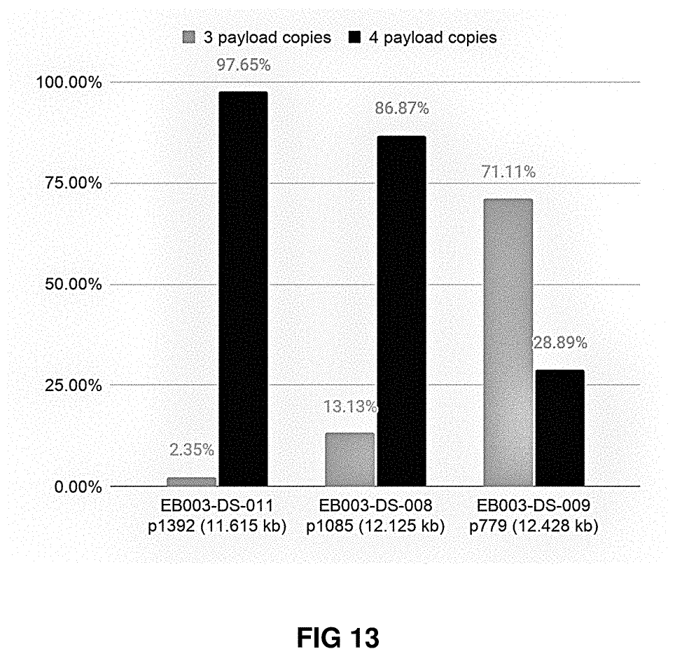

Important differences were observed between Eligobiotics® depending on the size of the packaged payload. Although Eligobiotics® packaging the smaller p1392 (11.615 kb) yielded almost exclusively particles containing 4 copies of the payload, small increases (up to 800 bp) in the size of the payload correlate with a shift towards packaging 3 copies. As such, Eligobiotics® produced with p779 (12.428 kb) packaged preferentially 3 copies of the payload while approximately a third of the EBs contained 4 copies of the payload (FIG. 13).

Thus, it appears that p1392 is close to an ideal size to package exclusively 4 copies of payload in phage-derived capsids, yielding an homogenous population. Increasing the size of the payload compared to p1392 generates more heterogeneous Eligobiotics® populations, with increasing proportions of EBs containing 3 copies of payload. From this dataset, it appears that there is a lower limit for concatemer packaging close to 36 kb, as described in the literature [28]. p1085, with a size of 12.125 kb, could package 3 copies per head (36.375 kb) or 4 copies per head (48.5 kb), although the 4 copies species is preferred as seen in FIG. 13. Increasing the size to 12.428 kb would allow packaging of 3 copies per head (37.284 kb) and 4 copies per head (49.712 kb); in this case, 4 copies are preferred. From these two data points, it was inferred that the lower limit for packaging is indeed around 36 kb but with a lower efficiency. Increasing the size just by 909 bp completely shifts the packaged species to 4 copies: the limit for optimal efficiency of packaging, probably driven by a pressure signal in the capsid, lies within these two sizes. Finally, the 11.615 kb payload packages virtually only 4 copies per head (46.46 kb), as the 3-copy species is slightly below the packaging limit, even at low efficiency (34.845 kb).

From these data, it can also be predicted which sizes would give packaging of single and multimeric species, as shown below in Tables 2 and 3. Smaller sizes yielding single packaged species are generally preferred for several reasons, including ease of manipulation and lower probability of introducing unwanted restriction sites. Finally, sizes that allow for very efficient packaged species that are not too small (26-39 kb) or too large (50-51 kb) are also preferred in some cases as it has been shown that the amount of DNA present in the capsid may alter the packaging and stability of the particles due to intracapsid pressure [29]-[30]. Finally, sizes that are large enough to allow for production of packaged phagemids at high titer are also more particularly preferred.

SEQUENCES

| Name | SEQ ID NO: | Type |
|---|---|---|
| psgRNAcos (p184) | 1 | DNA |
| pJ23104-GFP (p211) | 2 | DNA |
| pJF1 (p344) | 3 | DNA |
| GG2K (p502) | 4 | DNA |
| GG4K (p710) | 5 | DNA |
| GG6K (p504) | 6 | DNA |
| GG8K (p711) | 7 | DNA |
| GG12K (p499) | 8 | DNA |
| p7.3kb (p513) | 9 | DNA |
| Lambda gpJ (Uniprot P03749) | 10 | Amino acid |
| gpJ 591 | 11 | Amino acid |
| gpJ Z2145 | 12 | Amino acid |
| 1A2 | 13 | Amino acid |
| Lambda STF (Uniprot P03764) | 14 | Amino acid |
| Lambda tfa (Uniprot P03740) | 15 | Amino acid |
| STF lambda-WW11.2 | 16 | Amino acid |
| STF lambda-75 | 17 | Amino acid |
| Accessory protein lambda-STF75 | 18 | Amino acid |
| STF lambda-EB6 | 19 | Amino acid |
| Accessory protein lambda-EB6 | 20 | Amino acid |
| STF lambda-23 | 21 | Amino acid |
| Accessory protein lambda-STF23 | 22 | Amino acid |
| Lambda gpH (Uniprot P03736) | 23 | Amino acid |
| gpH-IAI | 24 | Amino acid |
| H591 | 25 | DNA |
| Z2145 | 26 | DNA |
| 1A2 | 27 | DNA |
| Lambda stf | 28 | DNA |
| Lambda tfa | 29 | DNA |
| Stf lambda-WW11.2 | 30 | DNA |
| Stf lambda-75 | 31 | DNA |
| Accessory protein lambda-stf75 | 32 | DNA |
| Stflambda-EB6 | 33 | DNA |
| Accessory protein kambda-EB6 | 34 | DNA |
| Stf lambda-23 | 35 | DNA |
| Accessory protein lambda-stf23 | 36 | DNA |
| Insertion site SAGDAS | 37 | Amino acid |
| Insertion site ADAKKS | 38 | Amino acid |
| Insertion site MDETNR | 39 | Amino acid |
| Insertion site SASAAA | 40 | Amino acid |
| Insertion site GAGENS | 41 | Amino acid |
| Insertion point 1 FIG. 7 | 42 | Amino acid |
| Insertion point 2 FIG. 7 | 43 | Amino acid |
| STF-V10 | 44 | Amino acid |
| STF-V10f | 45 | Amino acid |
| STF-V10a | 46 | Amino acid |
| Example of payload sequence | 47 | DNA |
| STF-V10h | 48 | Amino acid |
| A8 | 49 | Amino acid |
| STF lambda-P2 | 50 | Amino acid |
| stf-V10 | 51 | DNA |
| stf-V10f | 52 | DNA |
| stf-V10a | 53 | DNA |
| stf-V10h | 54 | DNA |
| A8 | 55 | DNA |
| Stf lambda-P2 | 56 | DNA |
| P2 accessory protein 1 | 57 | Protein |
| P2 accessory protein 1 | 58 | DNA |
| Sequence of Z2145 gpJ of FIG. 7 | 59 | Protein |
| Sequence of 1A2 gpJ of FIG. 7 | 60 | Protein |
| Sequence of 591 gpJ of FIG. 7 | 61 | Protein |
| Sequence of E6BTD4-ECOLX gpH of | 62 | Protein |
| FIG. 9A | | |
| Lambda-STF29 | 63 | Protein |
| Lambda-STF118 | 64 | Protein |
| Lambda-STF29 accessory protein | 65 | Protein |
| Lambda-STF118 accessory protein | 66 | Protein |

### REFERENCES

[1] J. Collins and B. Hohn, “Cosmids: a type of plasmid gene-cloning vector that is packageable in vitro in bacteriophage lambda heads.,” Proc. Natl. Acad. Sci. U.S.A., vol. 75, no. 9, pp. 4242-4246, September 1978.
[2] J. E. Cronan, “Cosmid-Based System for Transient Expression and Absolute Off-to-On Transcriptional Control of Escherichia coli Genes,”J. Bacteriol., vol. 185, no. 22, pp. 6522-6529, November 2003, doi: 10.1128/JB.185.22.6522-6529.2003.
[3] J. E. Cronan, “Improved Plasmid-Based System for Fully Regulated Off-To-On Gene Expression in Escherichia coli: Application to Production of Toxic Proteins,” Plasmid, vol. 69, no. 1, pp. 81-89, January 2013, doi: 10.1016/j.plasmid.2012.09.003.
[4] J. D. Haley, “Cosmid library construction,” Methods Mol. Biol. Clifton N.J., vol. 4, pp. 257-283, 1988, doi: 10.1385/0-89603-127-6:257.
[5] J. Wang, M. Hofnung, and A. Charbit, “The C-terminal portion of the tail fiber protein of bacteriophage lambda is responsible for binding to LamB, its receptor at the surface of Escherichia coli K-12,”J. Bacteriol., vol. 182, no. 2, pp. 508-512, January 2000, doi: 10.1128/jb.182.2.508-512.2000.
[6] J. H. Grose and S. R. Casjens, “Understanding the enormous diversity of bacteriophages: the tailed phages that infect the bacterial family Enterobacteriaceae,” Virology, vol. 0, pp. 421-443, November 2014, doi: 10.1016/j.virol.2014.08.024.
[7] R. W. Hendrix and R. L. Duda, “Bacteriophage lambda PaPa: not the mother of all lambda phages,” Science, vol. 258, no. 5085, pp. 1145-1148, November 1992.
[8] M. G. Rossmann, V. V. Mesyanzhinov, F. Arisaka, and P. G. Leiman, “The bacteriophage T4 DNA injection machine,” Curr. Opin. Struct. Biol., vol. 14, no. 2, pp. 171-180, April 2004, doi: 10.1016/j.sbi.2004.02.001.
[9] Y. Zivanovic et al., “Insights into Bacteriophage T5 Structure from Analysis of Its Morphogenesis Genes and Protein Components,”J. Virol., vol. 88, no. 2, pp. 1162-1174, January 2014, doi: 10.1128/JVI.02262-13.
[10] M. A. Speed, T. Morshead, D. I. Wang, and J. King, “Conformation of P22 tailspike folding and aggregation intermediates probed by monoclonal antibodies,” Protein Sci. Publ. Protein Soc., vol. 6, no. 1, pp. 99-108, January 1997, doi: 10.1002/pro.5560060111.
[11] T. Miwa and K. Matsubara, “Formation of oligomeric structures from plasmid DNA carrying cos lambda that is packaged into bacteriophage lambda heads.,” J. Bacteriol., vol. 153, no. 1, pp. 100-108, January 1983.
[12] P. Grayson et al., “The effect of genome length on ejection forces in bacteriophage lambda,” Virology, vol. 348, no. 2, pp. 430-436, May 2006, doi: 10.1016/j.virol.2006.01.003.
[13] S. Vimont et aL, “The CTX-M-15-producing Escherichia coli clone 025b: H4-ST131 has high intestine colonization and urinary tract infection abilities,” PloS One, vol. 7, no. 9, p. e46547, 2012, doi: 10.1371/journal.pone.0046547.
[14] M. L. Myhal, D. C. Laux, and P. S. Cohen, “Relative colonizing abilities of human fecal and K 12 strains of Escherichia coli in the large intestines of streptomycin-treated mice,” Eur. J. Clin. Microbiol., vol. 1, no. 3, pp. 186-192, June 1982.
[15] S. Chatterjee and E. Rothenberg, “Interaction of Bacteriophage with Its E. coli Receptor, LamB,” Viruses, vol. 4, no. 11, pp. 3162-3178, November 2012, doi: 10.3390/v4113162.
[16] A. Charbit, K. Gehring, H. Nikaido, T. Ferenci, and M. Hofnung, “Maltose transport and starch binding in phage-resistant point mutants of maltoporin. Functional and topological implications,” J. Mol. Biol., vol. 201, no. 3, pp. 487-496, June 1988.
[17] M. D. Paepe, V. Gaboriau-Routhiau, D. Rainteau, S. Rakotobe, F. Taddei, and N. Cerf-Bensussan, “Trade-Off between Bile Resistance and Nutritional Competence Drives Escherichia coli Diversification in the Mouse Gut,” PLOS Genet., vol. 7, no. 6, p. e1002107, June 2011, doi: 10.1371/journal.pgen.1002107.
[18] J. R. Meyer, D. T. Dobias, J. S. Weitz, J. E. Barrick, R. T. Quick, and R. E. Lenski, “Repeatability and contingency in the evolution of a key innovation in phage lambda,” Science, vol. 335, no. 6067, pp. 428-432, January 2012, doi: 10.1126/science.1214449.
[19] C. Werts, V. Michel, M. Hofnung, and A. Charbit, “Adsorption of bacteriophage lambda on the LamB protein of Escherichia coli K-12: point mutations in gene J of lambda responsible for extended host range,”J. Bacteriol., vol. 176, no. 4, pp. 941-947, February 1994, doi: 10.1128/jb.176.4.941-947.1994.
[20] K. S. Choi et aL, “Protection from Hemolytic Uremic Syndrome by Eyedrop Vaccination with Modified Enterohemorrhagic E. coli Outer Membrane Vesicles,” PLoS ONE, vol. 9, no. 7, July 2014, doi: 10.1371/journal.pone.0100229.
[21] Y. Shifrin et al., “Transient Shielding of Intimin and the Type III Secretion System of Enterohemorrhagic and Enteropathogenic Escherichia coli by a Group 4 Capsule,”J. Bacteriol., vol. 190, no. 14, pp. 5063-5074, July 2008, doi: 10.1128/JB.00440-08.
[22] C. A. Roessner and G. M. Ihler, “Proteinase sensitivity of bacteriophage lambda tail proteins gpJ and pH in complexes with the lambda receptor.,”J. Bacteriol., vol. 157, no. 1, pp. 165-170, January 1984.
[23] D. Scandella and W. Arber, “Phage lambda DNA injection into Escherichia coli pel-mutants is restored by mutations in phage genes V or H,” Virology, vol. 69, no. 1, pp. 206-215, January 1976.
[24] I. Martin-Verstraete, V. Michel, and A. Charbit, “The levanase operon of Bacillus subtilis expressed in Escherichia coli can substitute for the mannose permease in mannose uptake and bacteriophage lambda infection,” J. Bacteriol., vol. 178, no. 24, pp. 7112-7119, December 1996, doi: 10.1128/jb.178.24.7112-7119.1996.
[25] G. E. de Vries, C. K. Raymond, and R. A. Ludwig, “Extension of bacteriophage lambda host range: selection, cloning, and characterization of a constitutive lambda receptor gene,” Proc. Natl. Acad. Sci. U.S.A, vol. 81, no. 19, pp. 6080-6084, October 1984.
[26] M. S. Francis, A. F. Parker, R. Morona, and C. J. Thomas, “Bacteriophage Lambda as a Delivery Vector for Tn10-Derived Transposons in Xenorhabdus bovienii,” Appl. Environ. Microbiol., vol. 59, no. 9, pp. 3050-3055, September 1993.
[27] R. A. Ludwig, “Gene tandem-mediated selection of coliphage A-receptive Agrobacterium, Pseudomonas, and Rhizobium strains,” Proc. Natl. Acad. Sci. U.S.A, vol. 84, no. 10, pp. 3334-3338, May 1987.
[28] T. Miwa and K. Matsubara, “Formation of oligomeric structures from plasmid DNA carrying cos lambda that is packaged into bacteriophage lambda heads.” J Bacteriol. vol. 153, no. 1, pp. 100-108, January 1983.
[29] I. Ivanovska, G. Wuite, B. Jonsson, and A. Evilevitch, “Internal DNA pressure modifies stability of WT phage” Proc. Natl. Acad. Sci. U.S.A., vol. 104, no. 23, pp. 9603-9608, June 2007.
[30] E. Nurmemmedov, M. Castelnovo, E. Medina, C. Enrique Catalano and A. Evilevitch, “Challenging packaging limits and infectivity of phage λ.” J. Mol. Biol. vol. 415, no. 2, pp. 263-73, January 2012

1. A lambdoïd bacterial delivery vehicle for use in in vivo delivery of a DNA payload of interest into a targeted bacterial cell.

2. The bacterial delivery vehicle of claim 1 wherein the bacterial delivery vehicle is a bacteriophage.

3. The bacterial delivery vehicle of claim 1, wherein the bacterial delivery vehicle is a bacteriophage comprising a wild type side tail fiber (STF) protein, a wild type gpH protein and a wild type gpJ protein.

4. The bacterial delivery vehicle of claim 2, wherein the bacterial delivery vehicle is a wild-type bacteriophage.

5. The bacterial delivery vehicle of claim 1 wherein the bacterial delivery vehicle is a packaged phagemid.

6. The bacterial delivery vehicle of claim 1, comprising one or more proteins selected from the group consisting of a functional lambdoïd bacteriophage STF protein, a functional lambdoïd bacteriophage gpJ protein and a functional lambdoïd bacteriophage gpH protein.

7. The bacterial delivery vehicle of claim 1, comprising an STF protein, a gpJ protein and/or a gpH protein, wherein the STF protein, the gpJ protein and/or the gpH protein are wild type lambda STF, gpJ and/or gpH proteins.

8. The bacterial delivery vehicle of claim 6, wherein the STF protein, the gpJ protein and/or the gpH protein are non-naturally occurring recombinant proteins.

9. The bacterial delivery vehicle of claim 8, wherein:
(i) the recombinant STF protein is a chimeric protein comprising a fusion between a portion of a STF protein derived from a lambdoïd bacteriophage and a portion of a STF protein derived from a corresponding STF protein from a different bacteriophage;
(ii) the recombinant gpJ protein is a chimeric protein comprising a fusion between a portion of a gpJ protein derived from a lambdoïd bacteriophage and a portion of a gpJ protein derived from a corresponding gpJ protein from a different bacteriophage; and/or.
(iii) the recombinant gpH protein is a chimeric protein comprising a fusion between a portion of a gpH protein derived from a lambdoïd bacteriophage and a portion of a gpH protein derived from a corresponding gpH protein from a different bacteriophage.

10. The bacterial delivery vehicle of claim 8, wherein:
(i) the STF protein comprises or consists of the amino acid sequence of SEQ ID NO: 14, the amino acid sequence of SEQ ID NO: 16, the amino acid sequence of SEQ ID NO: 17, the amino acid sequence of SEQ ID NO: 19, the amino acid sequence of SEQ ID NO: 21, the amino acid sequence of SEQ ID NO: 44, or the amino acid sequence of SEQ ID NO: 50;
(ii) the gpJ protein comprises or consists of the amino acid sequence of SEQ ID NO: 10, 11, 12, 13 or 49; and/or
(iii) the gpH protein comprises or consists of the amino acid sequence of SEQ ID NO: 23 or 24.

11. The bacterial delivery vehicle of claim 8, wherein the recombinant STF protein, gpJ protein and/or gpH protein are engineered to allow transfer of the DNA payload of interest into the targeted bacterial cell.

12. The bacterial delivery vehicle of claim 8, wherein the recombinant STF protein has enzyme activity such as depolymerase activity and the targeted bacterial cell is an encapsulated bacterial cell.

13. The bacterial delivery vehicle of claim 8, wherein the recombinant STF protein, gpJ protein and/or gpH protein are engineered to increase the efficiency of transfer of the DNA payload into the targeted bacterial cell.

14. The bacterial delivery vehicle of claim 1, wherein said bacterial delivery vehicle comprises said DNA payload of interest.

15. The bacterial delivery vehicle of claim 1, wherein the DNA payload comprises a nucleic acid of interest selected from the group consisting of Cas nuclease gene, a Cas9 nuclease gene, a guide RNA, a CRISPR locus, a toxin gene, a gene encoding an enzyme such as a nuclease or a kinase, a TALEN, a ZFN, a meganuclease, a recombinase, a bacterial receptor, a membrane protein, a structural protein, a secreted protein, a gene encoding resistance to an antibiotic or to a drug in general, a gene encoding a toxic protein or a toxic factor, and a gene encoding a virulence protein or a virulence factor, and any of their combination.

16. The bacterial delivery vehicle of claim 15, wherein the nucleic acid of interest is a gene encoding a nuclease.

17. The bacterial delivery vehicle of claim 16, wherein the nuclease is selected from the group consisting of a Cas nuclease, a Cas9 nuclease, a TALEN, a ZFN and a meganuclease.

18. The bacterial delivery vehicle of claim 16, wherein the nuclease targets cleavage of a host bacterial cell chromosome or a host bacterial cell plasmid.

19. The bacterial delivery vehicle of claim 18, wherein the cleavage occurs in an antibiotic resistant gene.

20. The bacterial delivery vehicle of claim 1, wherein the DNA payload has a size greater than 10.000 kb and less than 12.000 kb, or has a size greater than 12.500 kb and less than 16.667 kb, or has a size greater than or equal to 18.000 kb and less than or equal to 25.000 kb.

21. The bacterial delivery vehicle of claim 1, wherein the DNA payload comprises or consists of the sequence SEQ ID NO: 47.

22. The bacterial delivery vehicle of claim 1, comprising a recombinant STF protein comprising or consisting of the sequence SEQ ID NO: 48 and a recombinant gpJ protein comprising or consisting of the sequence SEQ ID NO: 13, wherein the DNA payload comprises or consists of the sequence SEQ ID NO: 47, and wherein said targeted bacterial cell is a Shiga-Toxin producing E. coli (STEC).

23. The bacterial delivery vehicle of claim 1, wherein the DNA payload comprises a nucleic acid of interest encoding a therapeutic protein.

24. The bacterial delivery vehicle of claim 1, wherein the DNA payload comprises a nucleic acid of interest encoding an antisense nucleic acid molecule.

25. A pharmaceutical or veterinary composition comprising the bacterial delivery vehicle of claim 1 and a pharmaceutically acceptable carrier.

26. A method for in vivo delivery of a DNA payload of interest into a subject comprising, administering to said subject the pharmaceutical or veterinary composition of claim 25.

27. A method for treating a disease or disorder caused by bacteria comprising administering to a subject having said disease or disorder caused by bacteria in need of treatment the pharmaceutical or veterinary composition of claim 25.

28. The method of claim 27, wherein said disease or disorder is a bacterial infection, a metabolic disorder or a pathology involving bacteria of the human microbiome.

29. The method of claim 28 wherein said bacterial infection is a STEC infection.

30. A method for reducing the amount of virulent and/or antibiotic resistant bacteria in a bacterial population comprising contacting the bacterial population with the bacterial delivery vehicle of claim 1.


SEQUENCE LISTING (66 sequences)

SEQ ID NO: 1 - DNA, 2528 bp, Artificial Sequence
  psgRNAcos (p184)
tgcagcgcgatcgtaatcaggatcccatggtacgcgtgctagaggcatcaaataaaacgaaaggctcagtcgaaagactgggcctttcgttttatctgttgtttgtcggtgaacgctctcctgagtaggacaaatccgccgccctagaccctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgaggctttttggggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccgacgtctaagaaaccattattatcatgacattaacctataaaaataggcgtatcacgaggccctttcgtcttcgggcccattaagttctgtgctaggaggtgactgaagtatattttaggaattctaaagatctttgacagctagctcagtcctaggtataatactagttgagaccagtctaggtctcggttttagagctagaaatagcaagttaaaataaggctagtccgttatcaacttgaaaaagtggcaccgagtcggtgctttttttggtag

SEQ ID NO: 2 - DNA, 3274 bp, Artificial Sequence
  pJ23104-GFP (p211)
aaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtactagtggatttgacagctagctcagtcctaggtattgtgctagcgaattcattaaagaggagaaaggtacccatgagtaaaggtgaagaactgttcaccggtgttgttccgatcctggttgaactggatggtgatgttaacggccacaaattctctgttcgtggtgaaggtgaaggtgatgcaaccaacggtaaactgaccctgaaattcatctgcactaccggtaaactgccggttccatggccgactctggtgactaccctgacctatggtgttcagtgtttttctcgttacccggatcacatgaagcagcatgatttcttcaaatctgcaatgccggaaggttatgtacaggagcgcaccatttctttcaaagacgatggcacctacaaaacccgtgcagaggttaaatttgaaggtgatactctggtgaaccgtattgaactgaaaggcattgatttcaaagaggacggcaacatcctgggccacaaactggaatataacttcaactcccataacgtttacatcaccgcagacaaacagaagaacggtatcaaagctaacttcaaaattcgccataacgttgaagacggtagcgtacagctggcggaccactaccagcagaacactccgatcggtgatggtccggttctgctgccggataaccactacctgtccacccagtctaaactgtccaaagacccgaacgaaaagcgcgaccacatggtgctgctggagttcgttactgcagcaggtatcacgcacggcatggatgaactctacaaataaaacgcatgagaaagcccccggaagatcaccttccgggggcttttttattgcgctcgggtagcaccagagctcgccgcagccgaacgccccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggagggactagtggattcgggtagcaccagaagtctatagcacgtgagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctg

SEQ ID NO: 3 - DNA, 7240 bp, Artificial Sequence
  pJF1 (p344)
gattttacggctagctcagtcctaggtacaatgctagcgaatcattaaagaggagaaaggtactatggcacgtaccccgtcacgtagtagcattggtagcctgcgtagtccgcatacccataaagcaattctgaccagtaccatcgagatcctgaaagaatgtggttatagcggactgagcattgaaagcgttgcacgtcgtgccggagcaagcaaaccgaccatttatcgttggtggacgaataaagcagcactgattgccgaagtgtatgaaaatgaaagcgaacaggtgcgtaaatttccggatctgggtagctttaaagcagatctggattttttactgcgtaatttatggaaagtttggcgtgaaactatttgcggtgaagcatttcgttgtgttattgcagaagctcagctggatcctgcaaccctgacccagttaaaggatcaatttatggaacgtcgtcgtgagatgccgaaaaaactggttgaaaatgccattagcaatggtgaactgccgaaagataccaatcgtgaacttcttctggatatgatttttggtttttgttggtatcgcctgttaaccgaacagctgaccgttgaacaggatattgaagaatttaccttccttctgattaatggtgtttgtccgggtactcagcgttaactaggccataatcgctaccaaattccagaaaacagacgctttcgagcgtcttttttcgttttggtcacgacgtactgaatctgattcgttaccaattgacatgatacgaaacgtaccgtatcgttaaggttactggagcttaaaaaggagaaagtttctatggataagaaatacagcataggcttagacatcggcacaaatagcgtcggatgggcggtgatcactgatgaatataaagttccgtctaaaaagttcaaggtactgggtaatacagatcgccatagtatcaaaaagaacttaatcggtgcgcttctgttcgattccggcgaaaccgcagaagcaacacgtctgaaacgcaccgctcgtcgccgttacacccgtcgtaaaaaccgcatctgctacctgcaagaaatcttctctaacgaaatggctaaagtagatgacagcttttttcaccgtctggaagaatcatttctggtggaagaagataaaaagcacgaacgtcatccaatcttcggcaacattgtggacgaagtagcgtatcacgaaaaatacccgactatctatcacctgcgcaaaaagctggtcgattcgacggataaggccgatctgcgtctgatctatctggccttagcgcatatgattaagttccgtggtcatttcctgatcgaaggcgacctgaatccagacaacagcgatgtagacaaactgttcatccagctggtgcaaacctataaccagctgtttgaagaaaacccaattaatgctagcggtgttgacgcgaaagcgatcttgtccgcacgcctgtccaaatcccgtcgtctggaaaacttaattgcgcaactgccgggtgagaagaaaaacggactgttcggcaatctgatcgctcttagcttgggactgaccccgaacttcaaaagcaacttcgatctggcagaggacgcaaaacttcaacttagcaaagatacgtatgacgatgacttggataacttactggcccagatcggagatcagtacgctgatctgtttctggcggcaaagaacttatcagacgctattctcctgtctgatattcttcgtgtgaataccgaaatcaccaaagcaccgctttctgcatccatgattaaacgctatgacgaacatcaccaagatctgactcttctgaaagcgctggtacggcaacaactgccggagaagtacaaggagatcttctttgaccaatccaaaaacggctacgcgggttatattgacgggggtgcaagccaagaggagttctacaaattcatcaagccaatcttagaaaaaatggatggcacggaagaattacttgttaaactgaatcgtgaggatctgcttcgtaaacagcgtaccttcgacaacggtagcattccgcaccagatccacttaggtgaactgcacgctatcctgcgtcgccaagaggatttttacccgttcctgaaagataatcgtgaaaaaatcgaaaaaatcctgacctttcgtatcccgtattatgtcggcccgctggcgcgtggcaactcccgtttcgcgtggatgactcgcaaatccgaagaaactattaccccgtggaacttcgaggaagtggttgacaaaggcgcaagcgcccaatccttcatcgagcgcatgactaactttgataaaaacctgccgaacgaaaaggtactgccgaaacactcccttctgtacgaatacttcaccgtgtacaacgagctgactaaagtaaagtatgtgactgagggcatgcgtaaacctgcattcctgagcggtgaacagaaaaaagcaattgttgatttactgtttaaaaccaaccgtaaagtaaccgttaaacagctgaaagaggactacttcaagaaaatcgaatgcttcgactccgtcgagattagtggagttgaagatcgttttaatgcaagtttaggcacgtatcacgatttattaaagatcattaaagacaaagatttcttggacaacgaagaaaatgaggacatcttagaggacatcgtcctgaccctgactctgttcgaagatcgtgaaatgattgaagaacgcttgaagacgtatgctcacctgtttgacgataaagtaatgaaacaactgaaacgtcgccgttatactggctggggccgtctgagccgtaaactgattaacggtatccgtgacaaacagtccggtaaaactattctggacttcctgaaatctgacggcttcgcaaaccgtaacttcatgcaactgattcacgacgattccctgaccttcaaagaggacatccagaaagctcaggtttctggtcaaggtgattctctgcacgagcatatcgccaatttagcaggtagtccggcgatcaaaaaaggtatcctgcaaaccgtgaaagtggtggatgagcttgtgaaagttatgggtcgtcacaaaccggaaaacattgttatcgagatggctcgtgaaaaccaaacgacccagaagggacagaaaaactcccgcgaacgcatgaaacgtatcgaggagggtattaaagaacttggctctcagattctgaaagaacaccctgttgaaaatacccaactgcaaaatgaaaaactgtacctgtactacctgcaaaatggtcgtgacatgtatgtagatcaggagctggacatcaaccgcctctccgattacgacgttgaccacattgttccgcagtcttttctgaaagatgattccattgataacaaagtactcacccgtagcgataaaaaccgtgggaagagtgacaacgttccatcggaagaagtagttaagaaaatgaagaactattggcgtcaactgcttaacgcgaaactgattactcaacgtaaatttgataacctgaccaaagctgaacgtggcggtttgtctgagctggataaggcgggttttattaaacgtcaactggtagaaactcgccagattacaaaacatgttgctcagattctggactctcgtatgaacactaaatacgatgaaaatgacaaactgatccgcgaagttaaggttattaccctgaaatctaagctggtttccgacttccgtaaagatttccaattctataaagtgcgcgagattaacaactatcaccacgcgcacgacgcatatctgaatgcagttgttggcacggcactgatcaaaaaatatccgaaactggaaagcgaatttgtgtacggcgattataaagtttacgacgtgcgcaaaatgatcgccaaatctgaacaggaaattggcaaagcaaccgctaaatactttttctactcaaacattatgaatttcttcaaaaccgaaatcaccttagcgaatggcgaaattcgtaaacgccctctgatcgaaaccaacggcgaaacgggtgagatcgtgtgggacaaaggtcgtgatttcgctactgtccgcaaagttctgtccatgcctcaagtaaacatcgttaaaaagactgaggtacagactggcggtttcagcaaggaatccattctgccgaaacgcaactccgacaaactgatcgcgcgtaagaaagactgggatccgaagaaatacggtggcttcgattctccaaccgtggcatacagcgttctggtagtcgccaaagtcgaaaagggtaaatcaaaaaaactgaaatcagtgaaagaacttttaggcatcaccattatggaacgtagctctttcgaaaaaaacccgattgacttcctcgaagcgaaggggtacaaggaagtaaagaaagatctgattatcaaactgccgaagtattccctgttcgaactggaaaatggtcgtaaacgtatgttagcgtctgcgggtgaactgcaaaaagggaacgaattggcccttccgtccaagtacgtgaacttcctgtatctggcctcgcactacgagaaactgaaaggtagtccggaagataatgagcagaaacagctgttcgtggaacagcacaaacactatctggacgagattattgaacagatttctgagtttagcaaacgcgtaattctggcggacgcgaatctggataaagtcctgagcgcctacaataaacaccgtgataaaccgatccgtgaacaggcagaaaacatcattcacctgttcacgctgactaatcttggtgctccggcagccttcaaatacttcgacaccacgatcgatcgtaaacgttacacctccactaaagaagtcttagatgcaactcttattcaccagagcatcactggcctgtatgaaactcgtattgatctgagtcagttgggcggtgactaataacgttaaagtcagtttcacctgttttacgttaaaacccgcttcggcgggtttttacttttgggtttagccgaacgccccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtaaagggactagtggatgtttgatctcaaaaaaagcaccgactcggtgccactttttcaagttgataacggactagccttattttaacttgctatttctagctctaaaacctgagaagacatctcctgcgcgtgctagcattatacctaggactgagctagctgtcagtcgggtagcaccagaagtctatagcatgtgcatacctttggtcgaaaaaaaaagcccgcactgtcaggtgcgggcttttttcagtgtttccttgccggattacgccccgccctgccactcatcgcagtattgttgtaattcattaagcattctgccgacatggaagccatcacaaacggcatgatgaacttggatcgccagtggcattaacaccttgtcgccttgcgtataatattttcccatagtgaaaacgggggcgaagaagttgtccatatttgctacgtttaaatcaaaactggtgaaactcacccacggattggcactgacgaaaaacatattttcgataaaccctttagggaaatatgctaagttttcaccgtaacacgccacatcttgactatatatgtgtagaaactgccggaaatcgtcgtggtattctgaccagagcgatgaaaacgtttcagtttgctcatggaaaacggtgtaacaagggtgaacactatcccatatcaccagctcaccgtctttcattgccatacgaaactccggatgtgcattcatcaggcgggcaagaatgtgaataaaggccggataaaacttgtgcttatttttctttacggtttttaaaaaggccgtaatatccagctgaacggtttggttataggtgcactgagcaactgactggaatgcctcaaaatgttctttacgatgccattgacttatatcaactgtagtatatccagtgatttttttctccattttagcttccttagcttgcgaaatctcgataactcaaaaaatagtagtgatcttatttcattatggtgaaagttgtcttacgtgcaacattttcgcaaaaagttggcgctttatcaacactgtccctcctgttcagctactgacggtactgcggaactgactaaagtagtgcgtaacggcaaaagcaccgccggacatcagcgctagcggagtgtatactggcttactatgttggcactgatgagggtgtaagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacctgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggataaggctaaactgaaaggacaagttttggcgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcac

SEQ ID NO: 4 - DNA, 2165 bp, Artificial Sequence
  GG2K (p502)
ctgatgagggtgtaagtgaagtgcttcatgtggcaggagaaaaaaggctgcatcggtgcgtcagcagaatatgtgatacaggatatattccgcttcctcgctcactgactcgctacgctcggtcgttcgactgtggcgagcggaaatggcttacgaacggggcggagatttcctggaagatgccaggaagatacttaacagggaagtgagagggtcgcggcaaagccgtttttccataggctccgcccccctgacaagcatcacgaaatctgacgctcaaatcagtggtggcgaaacctgacaggactataaagataccaggcgtttccccctggcggctccctcgtgcgctctcctgttcctgcctttcggtttgccggtgtcattcctctgttacggccgagtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggataaggctaaactgaaaggacaagttttggcgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcaccggcgcgccgtgaccaattattgaaggccgctaacgcggcctttttttgtttctggtatcccgaatggagcgacttctccccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtaaagggtaagtatgaaggtcgtgtactccgtaacttcgacccacattccttaaagagtgcatgtgcatattttgttatcaataaaaaaggccgcgatttgcggccttattgttcgtcttgccggattacgccccgccctgccactcatcgcagtattgttgtaattcattaagcattctgccgacatggaagccatcacaaacggcatgatgaacttggatcgccagtggcattaacaccttgtcgccttgcgtataatattttcccatagtgaaaacgggggcgaagaagttgtccatatttgctacgtttaaatcaaaactggtgaaactcacccacggattggcactgacgaaaaacatattttcgataaaccctttagggaaatatgctaagttttcaccgtaacacgccacatcttgactatatatgtgtagaaactgccggaaatcgtcgtggtattctgaccagagcgatgaaaacgtttcagtttgctcatggaaaacggtgtaacaagggtgaacactatcccatatcaccagctcaccgtctttcattgccatacgaaactccggatgtgcattcatcaggcgggcaagaatgtgaataaaggccggataaaacttgtgcttatttttctttacggtttttaaaaaggccgtaatatccagctgaacggtttggttataggtgcactgagcaactgactggaatgcctcaaaatgttctttacgatgccattgacttatatcaactgtagtatatccagtgatttttttctccattttagcttccttagcttgcgaaatctcgataactcaaaaaatagtagtgatcttatttcattatggtgaaagttgtcttacgtgcaacattttcgcaaaaagttggcgctttatcaacactgtcggaatgacaaatggttccaattattgaacacccttcggggtgtttttttgtttctggtttcccgaggccggcctgcgctagcggagtgtatactggcttactatgttggca

SEQ ID NO: 5 - DNA, 4073 bp, Artificial Sequence
  GG4K (p710)
ttcgcaaaaagttggcgctttatcaacactgtcggaatgacaaatggttccaattattgaacacccttcggggtgtttttttgtttctggtttcccgaggccggcctgcgctagcggagtgtatactggcttactatgttggcactgatgagggtgtaagtgaagtgcttcatgtggcaggagaaaaaaggctgcatcggtgcgtcagcagaatatgtgatacaggatatattccgcttcctcgctcactgactcgctacgctcggtcgttcgactgtggcgagcggaaatggcttacgaacggggcggagatttcctggaagatgccaggaagatacttaacagggaagtgagagggtcgcggcaaagccgtttttccataggctccgcccccctgacaagcatcacgaaatctgacgctcaaatcagtggtggcgaaacctgacaggactataaagataccaggcgtttccccctggcggctccctcgtgcgctctcctgttcctgcctttcggtttgccggtgtcattcctctgttacggccgagtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggataaggctaaactgaaaggacaagttttggcgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcaccggcgcgccgtgaccaattattgaaggccgctaacgcggcctttttttgtttctggtatcccgaatggagcgacttctccccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtaaagggtaagtatgaaggtcgtgtactccatcgctaccaaattccagaaaacagacgctttcgagcgtcttttttcgttttggtcacgacgtactgaaatccatgtcttctcagtcagatctataataaagactttagcgcgtacagcaaaggccgtccgaatctgcacaccctttactggaaagcattatttgacgaacgtaacctgcaagatgtggtgtataaactgaacggtgaggcggaacttttctaccgtaaacagagtatcccgaagaaaatcacgcatccggcaaaagaagctattgccaacaaaaacaaagacaacccgaagaaagaatcagtattcgaatatgacctgatcaaagataaacgtttcaccgaagataagttctttttccactgtccgattaccatcaacttcaaatctagcggtgcgaacaagttcaacgatgaaattaacttattactgaaagagaaagctaatgacgtacacatcttatctattgatcgcggtgaacgtcatttagcatactatacactggtagatggtaaaggtaatattattaaacaggatactttcaatattatcggtaatgaccgtatgaaaaccaactatcacgataagctggcggcgatcgaaaaagatcgtgattctgcgcgtaaagattggaagaaaattaacaatatcaaagaaatgaaagaaggctatctgagccaagtggtgcacgagatcgcaaaactggtgattgaatataacgctatcgtggttttcgaagatctgaactttggttttaaacgtggtcgcttcaaagtagaaaaacaggtgtaccaaaaactggaaaaaatgctgattgaaaaactgaactatctggtttttaaagacaacgaatttgacaaaacgggtggcgtactccgtgcctatcagctgaccgctccgttcgaaacgttcaagaaaatgggtaaacaaacggggattatctattatgtgccagctggtttcacctccaagatttgtccagttacgggcttcgttaaccagctgtacccgaaatacgagagcgttagcaaatctcaagaatttttcagcaaattcgacaagatctgctataatctggataaaggctatttcgagttcagcttcgattacaaaaacttcggcgataaagcggctaaaggtaagtggactattgctagctttggtagccgtctgattaactttcgcaactccgacaaaaaccataattgggacacgcgtgaagtgtatccgaccaaagaactggaaaaattactgaaagactattccatcgaatatggtcatggggagtgcattaaagcggcgatttgcggtgaatccgataagaaatttttcgccaaactgaccagcgtgcttaacaccattctgcaaatgcgtaattctaaaacgggtacggagctggactacctgatttctccggtagccgacgttaacggcaacttcttcgattctcgtcaagcaccgaaaaatatgccacaagacgcggatgccaacggtgcataccatatcggcttaaaaggcttaatgttattaggccgtatcaagaataatcaggagggcaagaaattaaatctggttatcaaaaacgaagaatacttcgagttcgttcagaatcgtaacaattaatgtatgcttaagcagctcggtaccaaagacgaacaataagacgctgaaaagcgtcttttttcgttttggtcctgttgcggcgcgatagtgtgaacatgctatagacttctggtgctacccgactgacagctagctcagtcctaggtataatgctagcaatttctactgttgtagatagaagagcttcatcgctcttcatcgagacgaacaataaggcctccctaacggggggccttttttattgataacaaaagtaacttcgagcttgtctacctccgtaaattgcacccacattccttaaagagtgcatgtgcatattttgttatcaataaaaaaggccgcgatttgcggccttattgttcgtcttgccggattacgccccgccctgccactcatcgcagtattgttgtaattcattaagcattctgccgacatggaagccatcacaaacggcatgatgaacttggatcgccagtggcattaacaccttgtcgccttgcgtataatattttcccatagtgaaaacgggggcgaagaagttgtccatatttgctacgtttaaatcaaaactggtgaaactcacccacggattggcactgacgaaaaacatattttcgataaaccctttagggaaatatgctaagttttcaccgtaacacgccacatcttgactatatatgtgtagaaactgccggaaatcgtcgtggtattctgaccagagcgatgaaaacgtttcagtttgctcatggaaaacggtgtaacaagggtgaacactatcccatatcaccagctcaccgtctttcattgccatacgaaactccggatgtgcattcatcaggcgggcaagaatgtgaataaaggccggataaaacttgtgcttatttttctttacggtttttaaaaaggccgtaatatccagctgaacggtttggttataggtgcactgagcaactgactggaatgcctcaaaatgttctttacgatgccattgacttatatcaactgtagtatatccagtgatttttttctccattttagcttccttagcttgcgaaatctcgataactcaaaaaatagtagtgatcttatttcattatggtgaaagttgtcttacgtgcaacatt

SEQ ID NO: 6 - DNA, 6491 bp, Artificial Sequence
  GG6K (p504)
gcccgcagaaagggtatgaaaaattcgaatttaatatagaggactgccgtaagttcatcgacttctataaacagagcatttccaaacatccggaatggaaagacttcggcttccgtttctctgacactcagcgctataatagcatcgacgagttctaccgcgaagtggagaatcagggctataaactgaccttcgagaacattagtgagtcgtacatcgactccgttgtgaatcagggtaaactgtacctgtttcagatctataataaagactttagcgcgtacagcaaaggccgtccgaatctgcacaccctttactggaaagcattatttgacgaacgtaacctgcaagatgtggtgtataaactgaacggtgaggcggaacttttctaccgtaaacagagtatcccgaagaaaatcacgcatccggcaaaagaagctattgccaacaaaaacaaagacaacccgaagaaagaatcagtattcgaatatgacctgatcaaagataaacgtttcaccgaagataagttctttttccactgtccgattaccatcaacttcaaatctagcggtgcgaacaagttcaacgatgaaattaacttattactgaaagagaaagctaatgacgtacacatcttatctattgatcgcggtgaacgtcatttagcatactatacactggtagatggtaaaggtaatattattaaacaggatactttcaatattatcggtaatgaccgtatgaaaaccaactatcacgataagctggcggcgatcgaaaaagatcgtgattctgcgcgtaaagattggaagaaaattaacaatatcaaagaaatgaaagaaggctatctgagccaagtggtgcacgagatcgcaaaactggtgattgaatataacgctatcgtggttttcgaagatctgaactttggttttaaacgtggtcgcttcaaagtagaaaaacaggtgtaccaaaaactggaaaaaatgctgattgaaaaactgaactatctggtttttaaagacaacgaatttgacaaaacgggtggcgtactccgtgcctatcagctgaccgctccgttcgaaacgttcaagaaaatgggtaaacaaacggggattatctattatgtgccagctggtttcacctccaagatttgtccagttacgggcttcgttaaccagctgtacccgaaatacgagagcgttagcaaatctcaagaatttttcagcaaattcgacaagatctgctataatctggataaaggctatttcgagttcagcttcgattacaaaaacttcggcgataaagcggctaaaggtaagtggactattgctagctttggtagccgtctgattaactttcgcaactccgacaaaaaccataattgggacacgcgtgaagtgtatccgaccaaagaactggaaaaattactgaaagactattccatcgaatatggtcatggggagtgcattaaagcggcgatttgcggtgaatccgataagaaatttttcgccaaactgaccagcgtgcttaacaccattctgcaaatgcgtaattctaaaacgggtacggagctggactacctgatttctccggtagccgacgttaacggcaacttcttcgattctcgtcaagcaccgaaaaatatgccacaagacgcggatgccaacggtgcataccatatcggcttaaaaggcttaatgttattaggccgtatcaagaataatcaggagggcaagaaattaaatctggttatcaaaaacgaagaatacttcgagttcgttcagaatcgtaacaattaatgtatgcttaagcagctcggtaccaaagacgaacaataagacgctgaaaagcgtcttttttcgttttggtcctgttgcggcgcgatagtgtgaacatgctatagacttctggtgctacccgactgacagctagctcagtcctaggtataatgctagcaatttctactgttgtagatagaagagcttcatcgctcttcatcgagacgaacaataaggcctccctaacggggggccttttttattgataacaaaagtaacttcgagcttgtctacctccgtaaattgcacccacattccttaaagagtgcatgtgcatattttgttatcaataaaaaaggccgcgatttgcggccttattgttcgtcttgccggattacgccccgccctgccactcatcgcagtattgttgtaattcattaagcattctgccgacatggaagccatcacaaacggcatgatgaacttggatcgccagtggcattaacaccttgtcgccttgcgtataatattttcccatagtgaaaacgggggcgaagaagttgtccatatttgctacgtttaaatcaaaactggtgaaactcacccacggattggcactgacgaaaaacatattttcgataaaccctttagggaaatatgctaagttttcaccgtaacacgccacatcttgactatatatgtgtagaaactgccggaaatcgtcgtggtattctgaccagagcgatgaaaacgtttcagtttgctcatggaaaacggtgtaacaagggtgaacactatcccatatcaccagctcaccgtctttcattgccatacgaaactccggatgtgcattcatcaggcgggcaagaatgtgaataaaggccggataaaacttgtgcttatttttctttacggtttttaaaaaggccgtaatatccagctgaacggtttggttataggtgcactgagcaactgactggaatgcctcaaaatgttctttacgatgccattgacttatatcaactgtagtatatccagtgatttttttctccattttagcttccttagcttgcgaaatctcgataactcaaaaaatagtagtgatcttatttcattatggtgaaagttgtcttacgtgcaacattttcgcaaaaagttggcgctttatcaacactgtcggaatgacaaatggttccaattattgaacacccttcggggtgtttttttgtttctggtttcccgaggccggcctgcgctagcggagtgtatactggcttactatgttggcactgatgagggtgtaagtgaagtgcttcatgtggcaggagaaaaaaggctgcatcggtgcgtcagcagaatatgtgatacaggatatattccgcttcctcgctcactgactcgctacgctcggtcgttcgactgtggcgagcggaaatggcttacgaacggggcggagatttcctggaagatgccaggaagatacttaacagggaagtgagagggtcgcggcaaagccgtttttccataggctccgcccccctgacaagcatcacgaaatctgacgctcaaatcagtggtggcgaaacctgacaggactataaagataccaggcgtttccccctggcggctccctcgtgcgctctcctgttcctgcctttcggtttgccggtgtcattcctctgttacggccgagtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggataaggctaaactgaaaggacaagttttggcgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcaccggcgcgccgtgaccaattattgaaggccgctaacgcggcctttttttgtttctggtatcccgaatggagcgacttctccccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtaaagggtaagtatgaaggtcgtgtactccatcgctaccaaattccagaaaacagacgctttcgagcgtcttttttcgttttggtcacgacgtactgaatctgattcgttaccaattgacatgatacgaaacgtaccgtatcgttaaggttactagaagagcgattgggctcttcaatgtccatctatcaggagtttgttaacaagtattccctgtctaaaaccctgcgttttgaactgatcccgcagggcaaaactttggaaaacattaaagcgcgtggcctgattctggatgacgaaaaacgtgcaaaggattacaagaaagctaaacagatcatcgacaaatatcaccagttctttatcgaagaaattctgtcgtcggtgtgcatcagtgaggatctgttacagaattattctgatgtatactttaaacttaaaaagtccgatgacgataatctgcaaaaagatttcaagtcagccaaagataccatcaagaaacagatctcagaatatattaaagatagcgaaaagttcaaaaacctgtttaaccaaaacctcattgatgctaagaaaggccaagaatctgacctgatcttatggctgaaacagagcaaagataacggcattgaactgttcaaagctaatagcgacatcaccgatattgatgaagcgctcgaaatcatcaagtctttcaaaggctggacgacgtatttcaaaggttttcatgaaaaccgtaagaatgtatattcgagcaacgatattccgacctctattatttatcgtatcgtggacgacaacctgccgaagtttctggaaaacaaagcgaaatatgaatctctgaaagacaaagcaccggaagctattaactatgaacagatcaagaaagatctggcggaagaactgaccttcgacatcgactataaaacctccgaagttaaccagcgtgttttctcactggacgaggttttcgaaatcgctaatttcaacaattacctgaatcaatctggcatcaccaaattcaacaccattattggtggcaaatttgttaacggcgaaaacaccaagcgtaagggcatcaacgaatacattaacctgtatagccaacaaatcaacgacaaaaccctgaaaaagtataaaatgtccgttctgtttaaacagattttatcggacaccgaatctaaatccttcgtaattgataaactggaagatgatagcgacgttgtcaccacgatgcagagcttttatgagcagattgcggcgttcaaaaccgtggaagagaaatctattaaagaaactctgtccctgctctttgacgacctcaaagcgcagaaactagatctgtctaagatttactttaaaaacgacaaatctctgaccgatctcagtcaacaagttttcgatgactatagcgtgatcggcacggcagttttggaatacatcacccaacaaatcgcgccgaaaaatctggacaacccgtccaagaaggaacaggaactgattgcaaagaaaacagaaaaagctaaatacctgagcttagaaactatcaaactggcacttgaggaatttaataaacatcgtgatattgataaacagtgtcgttttgaggaaattctggcgaactttgcggcaatcccgatgatcttcgacgaaattgctcaaaacaaagacaatctggcgcagatctctatcaagtaccagaatcagggtaagaaagatctgcttcaagcatctgcggaggacgatgtgaaagcaattaaagacttattagatcagacgaataacttattacacaagctcaaaatcttccacatcagccagagcgaggacaaggcgaacattctggataaagatgaacacttctatctggtgttcgaagaatgttacttcgaactggcaaacatcgtccctctctacaataaaatccgcaactacatcacgcagaagccttactctgacgagaaattcaaactgaacttcgaaaacagcacgctggcgaacggctgggataagaacaaagagccggacaacaccgcaatcctgttcatcaaagacgacaaatactatctgggcgtaatgaacaagaagaacaacaagatcttcgacgataaagcgatcaaagaaaacaagggtgaaggctataagaaaatcgtgtacaagctcctgccgggtgcgaataaaatgttaccgaaagtgttcttttccgcgaaaagcatcaaattctacaacccgtctgaggatattctgcgcatccgcaatcatagcacgcacactaaaaacggta

SEQ ID NO: 7 - DNA, 7980 bp, Artificial Sequence
  GG8K (p711)
actgtcggaatgacaaatggttccaattattgaacacccttcggggtgtttttttgtttctggtttcccgaggccggcctgcgctagcggagtgtatactggcttactatgttggcactgatgagggtgtaagtgaagtgcttcatgtggcaggagaaaaaaggctgcatcggtgcgtcagcagaatatgtgatacaggatatattccgcttcctcgctcactgactcgctacgctcggtcgttcgactgtggcgagcggaaatggcttacgaacggggcggagatttcctggaagatgccaggaagatacttaacagggaagtgagagggtcgcggcaaagccgtttttccataggctccgcccccctgacaagcatcacgaaatctgacgctcaaatcagtggtggcgaaacctgacaggactataaagataccaggcgtttccccctggcggctccctcgtgcgctctcctgttcctgcctttcggtttgccggtgtcattcctctgttacggccgagtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggataaggctaaactgaaaggacaagttttggcgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcaccggcgcgccgtgaccaattattgaaggccgctaacgcggcctttttttgtttctggtatcccgaatggagcgacttctccccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtaaagggtaagtatgaaggtcgtgtactccatcgctaccaaattccagaaaacagacgctttcgagcgtcttttttcgttttggtcacgacgtactgaatctgattcgttaccaattgacatgatacgaaacgtaccgtatcgttaaggttactagaagagcgattgggctcttcaatgtccatctatcaggagtttgttaacaagtattccctgtctaaaaccctgcgttttgaactgatcccgcagggcaaaactttggaaaacattaaagcgcgtggcctgattctggatgacgaaaaacgtgcaaaggattacaagaaagctaaacagatcatcgacaaatatcaccagttctttatcgaagaaattctgtcgtcggtgtgcatcagtgaggatctgttacagaattattctgatgtatactttaaacttaaaaagtccgatgacgataatctgcaaaaagatttcaagtcagccaaagataccatcaagaaacagatctcagaatatattaaagatagcgaaaagttcaaaaacctgtttaaccaaaacctcattgatgctaagaaaggccaagaatctgacctgatcttatggctgaaacagagcaaagataacggcattgaactgttcaaagctaatagcgacatcaccgatattgatgaagcgctcgaaatcatcaagtctttcaaaggctggacgacgtatttcaaaggttttcatgaaaaccgtaagaatgtatattcgagcaacgatattccgacctctattatttatcgtatcgtggacgacaacctgccgaagtttctggaaaacaaagcgaaatatgaatctctgaaagacaaagcaccggaagctattaactatgaacagatcaagaaagatctggcggaagaactgaccttcgacatcgactataaaacctccgaagttaaccagcgtgttttctcactggacgaggttttcgaaatcgctaatttcaacaattacctgaatcaatctggcatcaccaaattcaacaccattattggtggcaaatttgttaacggcgaaaacaccaagcgtaagggcatcaacgaatacattaacctgtatagccaacaaatcaacgacaaaaccctgaaaaagtataaaatgtccgttctgtttaaacagattttatcggacaccgaatctaaatccttcgtaattgataaactggaagatgatagcgacgttgtcaccacgatgcagagcttttatgagcagattgcggcgttcaaaaccgtggaagagaaatctattaaagaaactctgtccctgctctttgacgacctcaaagcgcagaaactagatctgtctaagatttactttaaaaacgacaaatctctgaccgatctcagtcaacaagttttcgatgactatagcgtgatcggcacggcagttttggaatacatcacccaacaaatcgcgccgaaaaatctggacaacccgtccaagaaggaacaggaactgattgcaaagaaaacagaaaaagctaaatacctgagcttagaaactatcaaactggcacttgaggaatttaataaacatcgtgatattgataaacagtgtcgttttgaggaaattctggcgaactttgcggcaatcccgatgatcttcgacgaaattgctcaaaacaaagacaatctggcgcagatctctatcaagtaccagaatcagggtaagaaagatctgcttcaagcatctgcggaggacgatgtgaaagcaattaaagacttattagatcagacgaataacttattacacaagctcaaaatcttccacatcagccagagcgaggacaaggcgaacattctggataaagatgaacacttctatctggtgttcgaagaatgttacttcgaactggcaaacatcgtccctctctacaataaaatccgcaactacatcacgcagaagccttactctgacgagaaattcaaactgaacttcgaaaacagcacgctggcgaacggctgggataagaacaaagagccggacaacaccgcaatcctgttcatcaaagacgacaaatactatctgggcgtaatgaacaagaagaacaacaagatcttcgacgataaagcgatcaaagaaaacaagggtgaaggctataagaaaatcgtgtacaagctcctgccgggtgcgaataaaatgttaccgaaagtgttcttttccgcgaaaagcatcaaattctacaacccgtctgaggatattctgcgcatccgcaatcatagcacgcacactaaaaacggtagcccgcagaaagggtatgaaaaattcgaatttaatatagaggactgccgtaagttcatcgacttctataaacagagcatttccaaacatccggaatggaaagacttcggcttccgtttctctgacactcagcgctataatagcatcgacgagttctaccgcgaagtggagaatcagggctataaactgaccttcgagaacattagtgagtcgtacatcgactccgttgtgaatcagggtaaactgtacctgtttcagatctataataaagactttagcgcgtacagcaaaggccgtccgaatctgcacaccctttactggaaagcattatttgacgaacgtaacctgcaagatgtggtgtataaactgaacggtgaggcggaacttttctaccgtaaacagagtatcccgaagaaaatcacgcatccggcaaaagaagctattgccaacaaaaacaaagacaacccgaagaaagaatcagtattcgaatatgacctgatcaaagataaacgtttcaccgaagataagttctttttccactgtccgattaccatcaacttcaaatctagcggtgcgaacaagttcaacgatgaaattaacttattactgaaagagaaagctaatgacgtacacatcttatctattgatcgcggtgaacgtcatttagcatactatacactggtagatggtaaaggtaatattattaaacaggatactttcaatattatcggtaatgaccgtatgaaaaccaactatcacgataagctggcggcgatcgaaaaagatcgtgattctgcgcgtaaagattggaagaaaattaacaatatcaaagaaatgaaagaaggctatctgagccaagtggtgcacgagatcgcaaaactggtgattgaatataacgctatcgtggttttcgaagatctgaactttggttttaaacgtggtcgcttcaaagtagaaaaacaggtgtaccaaaaactggaaaaaatgctgattgaaaaactgaactatctggtttttaaagacaacgaatttgacaaaacgggtggcgtactccgtgcctatcagctgaccgctccgttcgaaacgttcaagaaaatgggtaaacaaacggggattatctattatgtgccagctggtttcacctccaagatttgtccagttacgggcttcgttaaccagctgtacccgaaatacgagagcgttagcaaatctcaagaatttttcagcaaattcgacaagatctgctataatctggataaaggctatttcgagttcagcttcgattacaaaaacttcggcgataaagcggctaaaggtaagtggactattgctagctttggtagccgtctgattaactttcgcaactccgacaaaaaccataattgggacacgcgtgaagtgtatccgaccaaagaactggaaaaattactgaaagactattccatcgaatatggtcatggggagtgcattaaagcggcgatttgcggtgaatccgataagaaatttttcgccaaactgaccagcgtgcttaacaccattctgcaaatgcgtaattctaaaacgggtacggagctggactacctgatttctccggtagccgacgttaacggcaacttcttcgattctcgtcaagcaccgaaaaatatgccacaagacgcggatgccaacggtgcataccatatcggcttaaaaggcttaatgttattaggccgtatcaagaataatcaggagggcaagaaattaaatctggttatcaaaaacgaagaatacttcgagttcgttcagaatcgtaacaattaatgtatgcttaagcagctcggtaccaaagacgaacaataagacgctgaaaagcgtcttttttcgttttggtcctgttgcggcgcgatagtgtgaacatgctatagacttctggtgctacccgactgacagctagctcagtcctaggtataatgctagcaatttctactgttgtagatagaagagcttcatcgctcttcatcgagacgaacaataaggcctccctaacggggggccttttttattgataacaaaagtaacttcgagcttgtctacctcctagcactttatagctagctcagcccttggtacaatgctagcgttttcattaaagaggagaaaggaagccatgagtaaaggtgaggaattatttactggtgttgttccgatcttagttgaactggacggcgatgttaacggtcataaattcagtgttcgtggtgaaggtgaaggtgatgcaaccaacggtaagctgaccctgaaattcatctgcactactggaaaattaccagtaccgtggcctactctggtgactaccctgacctatggtgttcagtgtttttctcgttaccctgaccacatgaagcaacatgatttcttcaaatctgcaatgccggaaggttatgtacaggagcgcaccatttctttcaaagacgatggcacgtataaaacccgtgcagaggttaaatttgaaggtgacactctggtgaatcgtattgaactgaaaggcattgatttcaaagaggacggcaatattttaggccacaaactggaatataacttcaactcccataacgtttacatcaccgcagacaaacagaagaacggtatcaaagctaacttcaaaattcgccataacgttgaagatggtagcgtacagctggcggatcattaccaacagaacactccgattggagatgctcctgttttactgccggataaccactacctgtccacccagtctaaactgtcgaaggatccgaacgaaaagcgcgaccacatggtgttattagagttcgttaccgctagtggtatcacgcacggtatggatgaactctacaaataagacgaacaataaggggagcgggaaaccgctccccttttttattgataacaaaagtaaattgcacgctgatagtctcccaattgcgaaggaccaaaacgaaaaaacaccctttcgggtgtcttttctggaatttggtaccgagtactaggtatcgtgtaagtagcgaaggcccgtacgcgagataaactgctaggcaaccgcgactctacgactggtgctcgatttaatttcgctgacgtaaagaaattatcggcagtgcgtcaactgccgtatctttatcttaattaggtagttggacaagcccttgaaagaaatagcaagagcctgcctctctattgaagtcacggcgaaagtcgggtagaaatcaaagaaagcagaaattaaatcggagtaacactaaggtgggataactccgtaactgactacgcctttctctagactttacttgaccagatacactgtctttgacacgttgaaggattagagcaatcaaatccaagactggctaagcacgaagcaactcttgagtgttaaaaagttatctcctgtattcgggaagcgggtactagaagattgcagggactccgacgttaagtaaattacaaagtaataagtatcgttcaggatcacgttaccgcaataagaagcgagaataatataatttccgaagtgcttaccccagtagtgactattcctataacccttctgagtgtccggcattccttaaagagtgcatgtgcatattttgttatcaataaaaaaggccgcgatttgcggccttattgttcgtcttgccggattacgccccgccctgccactcatcgcagtattgttgtaattcattaagcattctgccgacatggaagccatcacaaacggcatgatgaacttggatcgccagtggcattaacaccttgtcgccttgcgtataatattttcccatagtgaaaacgggggcgaagaagttgtccatatttgctacgtttaaatcaaaactggtgaaactcacccacggattggcactgacgaaaaacatattttcgataaaccctttagggaaatatgctaagttttcaccgtaacacgccacatcttgactatatatgtgtagaaactgccggaaatcgtcgtggtattctgaccagagcgatgaaaacgtttcagtttgctcatggaaaacggtgtaacaagggtgaacactatcccatatcaccagctcaccgtctttcattgccatacgaaactccggatgtgcattcatcaggcgggcaagaatgtgaataaaggccggataaaacttgtgcttatttttctttacggtttttaaaaaggccgtaatatccagctgaacggtttggttataggtgcactgagcaactgactggaatgcctcaaaatgttctttacgatgccattgacttatatcaactgtagtatatccagtgatttttttctccattttagcttccttagcttgcgaaatctcgataactcaaaaaatagtagtgatcttatttcattatggtgaaagttgtcttacgtgcaacattttcgcaaaaagttggcgctttatcaac

SEQ ID NO: 8 - DNA, 12262 bp, Artificial Sequence
  GG12K (p499)
actgtcggaatgacaaatggttccaattattgaacacccttcggggtgtttttttgtttctggtttcccgaggccggcctgcgctagcggagtgtatactggcttactatgttggcactgatgagggtgtaagtgaagtgcttcatgtggcaggagaaaaaaggctgcatcggtgcgtcagcagaatatgtgatacaggatatattccgcttcctcgctcactgactcgctacgctcggtcgttcgactgtggcgagcggaaatggcttacgaacggggcggagatttcctggaagatgccaggaagatacttaacagggaagtgagagggtcgcggcaaagccgtttttccataggctccgcccccctgacaagcatcacgaaatctgacgctcaaatcagtggtggcgaaacctgacaggactataaagataccaggcgtttccccctggcggctccctcgtgcgctctcctgttcctgcctttcggtttgccggtgtcattcctctgttacggccgagtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggataaggctaaactgaaaggacaagttttggcgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcaccggcgcgccgtgaccaattattgaaggccgctaacgcggcctttttttgtttctggtatcccgaatggagcgacttctccccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtaaagggtaagtatgaaggtcgtgtactccatcgctaccaaattccagaaaacagacgctttcgagcgtcttttttcgttttggtcacgacgtactgaatctgattcgttaccaattgacatgatacgaaacgtaccgtatcgttaaggttactagaagagcgattgggctcttcaatgtccatctatcaggagtttgttaacaagtattccctgtctaaaaccctgcgttttgaactgatcccgcagggcaaaactttggaaaacattaaagcgcgtggcctgattctggatgacgaaaaacgtgcaaaggattacaagaaagctaaacagatcatcgacaaatatcaccagttctttatcgaagaaattctgtcgtcggtgtgcatcagtgaggatctgttacagaattattctgatgtatactttaaacttaaaaagtccgatgacgataatctgcaaaaagatttcaagtcagccaaagataccatcaagaaacagatctcagaatatattaaagatagcgaaaagttcaaaaacctgtttaaccaaaacctcattgatgctaagaaaggccaagaatctgacctgatcttatggctgaaacagagcaaagataacggcattgaactgttcaaagctaatagcgacatcaccgatattgatgaagcgctcgaaatcatcaagtctttcaaaggctggacgacgtatttcaaaggttttcatgaaaaccgtaagaatgtatattcgagcaacgatattccgacctctattatttatcgtatcgtggacgacaacctgccgaagtttctggaaaacaaagcgaaatatgaatctctgaaagacaaagcaccggaagctattaactatgaacagatcaagaaagatctggcggaagaactgaccttcgacatcgactataaaacctccgaagttaaccagcgtgttttctcactggacgaggttttcgaaatcgctaatttcaacaattacctgaatcaatctggcatcaccaaattcaacaccattattggtggcaaatttgttaacggcgaaaacaccaagcgtaagggcatcaacgaatacattaacctgtatagccaacaaatcaacgacaaaaccctgaaaaagtataaaatgtccgttctgtttaaacagattttatcggacaccgaatctaaatccttcgtaattgataaactggaagatgatagcgacgttgtcaccacgatgcagagcttttatgagcagattgcggcgttcaaaaccgtggaagagaaatctattaaagaaactctgtccctgctctttgacgacctcaaagcgcagaaactagatctgtctaagatttactttaaaaacgacaaatctctgaccgatctcagtcaacaagttttcgatgactatagcgtgatcggcacggcagttttggaatacatcacccaacaaatcgcgccgaaaaatctggacaacccgtccaagaaggaacaggaactgattgcaaagaaaacagaaaaagctaaatacctgagcttagaaactatcaaactggcacttgaggaatttaataaacatcgtgatattgataaacagtgtcgttttgaggaaattctggcgaactttgcggcaatcccgatgatcttcgacgaaattgctcaaaacaaagacaatctggcgcagatctctatcaagtaccagaatcagggtaagaaagatctgcttcaagcatctgcggaggacgatgtgaaagcaattaaagacttattagatcagacgaataacttattacacaagctcaaaatcttccacatcagccagagcgaggacaaggcgaacattctggataaagatgaacacttctatctggtgttcgaagaatgttacttcgaactggcaaacatcgtccctctctacaataaaatccgcaactacatcacgcagaagccttactctgacgagaaattcaaactgaacttcgaaaacagcacgctggcgaacggctgggataagaacaaagagccggacaacaccgcaatcctgttcatcaaagacgacaaatactatctgggcgtaatgaacaagaagaacaacaagatcttcgacgataaagcgatcaaagaaaacaagggtgaaggctataagaaaatcgtgtacaagctcctgccgggtgcgaataaaatgttaccgaaagtgttcttttccgcgaaaagcatcaaattctacaacccgtctgaggatattctgcgcatccgcaatcatagcacgcacactaaaaacggtagcccgcagaaagggtatgaaaaattcgaatttaatatagaggactgccgtaagttcatcgacttctataaacagagcatttccaaacatccggaatggaaagacttcggcttccgtttctctgacactcagcgctataatagcatcgacgagttctaccgcgaagtggagaatcagggctataaactgaccttcgagaacattagtgagtcgtacatcgactccgttgtgaatcagggtaaactgtacctgtttcagatctataataaagactttagcgcgtacagcaaaggccgtccgaatctgcacaccctttactggaaagcattatttgacgaacgtaacctgcaagatgtggtgtataaactgaacggtgaggcggaacttttctaccgtaaacagagtatcccgaagaaaatcacgcatccggcaaaagaagctattgccaacaaaaacaaagacaacccgaagaaagaatcagtattcgaatatgacctgatcaaagataaacgtttcaccgaagataagttctttttccactgtccgattaccatcaacttcaaatctagcggtgcgaacaagttcaacgatgaaattaacttattactgaaagagaaagctaatgacgtacacatcttatctattgatcgcggtgaacgtcatttagcatactatacactggtagatggtaaaggtaatattattaaacaggatactttcaatattatcggtaatgaccgtatgaaaaccaactatcacgataagctggcggcgatcgaaaaagatcgtgattctgcgcgtaaagattggaagaaaattaacaatatcaaagaaatgaaagaaggctatctgagccaagtggtgcacgagatcgcaaaactggtgattgaatataacgctatcgtggttttcgaagatctgaactttggttttaaacgtggtcgcttcaaagtagaaaaacaggtgtaccaaaaactggaaaaaatgctgattgaaaaactgaactatctggtttttaaagacaacgaatttgacaaaacgggtggcgtactccgtgcctatcagctgaccgctccgttcgaaacgttcaagaaaatgggtaaacaaacggggattatctattatgtgccagctggtttcacctccaagatttgtccagttacgggcttcgttaaccagctgtacccgaaatacgagagcgttagcaaatctcaagaatttttcagcaaattcgacaagatctgctataatctggataaaggctatttcgagttcagcttcgattacaaaaacttcggcgataaagcggctaaaggtaagtggactattgctagctttggtagccgtctgattaactttcgcaactccgacaaaaaccataattgggacacgcgtgaagtgtatccgaccaaagaactggaaaaattactgaaagactattccatcgaatatggtcatggggagtgcattaaagcggcgatttgcggtgaatccgataagaaatttttcgccaaactgaccagcgtgcttaacaccattctgcaaatgcgtaattctaaaacgggtacggagctggactacctgatttctccggtagccgacgttaacggcaacttcttcgattctcgtcaagcaccgaaaaatatgccacaagacgcggatgccaacggtgcataccatatcggcttaaaaggcttaatgttattaggccgtatcaagaataatcaggagggcaagaaattaaatctggttatcaaaaacgaagaatacttcgagttcgttcagaatcgtaacaattaatgtatgcttaagcagctcggtaccaaagacgaacaataagacgctgaaaagcgtcttttttcgttttggtcctgttgcggcgcgatagtgtgaacatgctatagacttctggtgctacccgactgacagctagctcagtcctaggtataatgctagcaatttctactgttgtagatagaagagcttcatcgctcttcatcgagacgaacaataaggcctccctaacggggggccttttttattgataacaaaagtaacttcgagcttgtctacctcctagcactttatagctagctcagcccttggtacaatgctagcgttttcattaaagaggagaaaggaagccatgagtaaaggtgaggaattatttactggtgttgttccgatcttagttgaactggacggcgatgttaacggtcataaattcagtgttcgtggtgaaggtgaaggtgatgcaaccaacggtaagctgaccctgaaattcatctgcactactggaaaattaccagtaccgtggcctactctggtgactaccctgacctatggtgttcagtgtttttctcgttaccctgaccacatgaagcaacatgatttcttcaaatctgcaatgccggaaggttatgtacaggagcgcaccatttctttcaaagacgatggcacgtataaaacccgtgcagaggttaaatttgaaggtgacactctggtgaatcgtattgaactgaaaggcattgatttcaaagaggacggcaatattttaggccacaaactggaatataacttcaactcccataacgtttacatcaccgcagacaaacagaagaacggtatcaaagctaacttcaaaattcgccataacgttgaagatggtagcgtacagctggcggatcattaccaacagaacactccgattggagatgctcctgttttactgccggataaccactacctgtccacccagtctaaactgtcgaaggatccgaacgaaaagcgcgaccacatggtgttattagagttcgttaccgctagtggtatcacgcacggtatggatgaactctacaaataagacgaacaataaggggagcgggaaaccgctccccttttttattgataacaaaagtaaattgcacgctgatagtctcccaattgcgaaggaccaaaacgaaaaaacaccctttcgggtgtcttttctggaatttggtaccgagtactaggtatcgtgtaagtagcgaaggcccgtacgcgagataaactgctaggcaaccgcgactctacgactggtgctcgatttaatttcgctgacgtaaagaaattatcggcagtgcgtcaactgccgtatctttatcttaattaggtagttggacaagcccttgaaagaaatagcaagagcctgcctctctattgaagtcacggcgaaagtcgggtagaaatcaaagaaagcagaaattaaatcggagtaacactaaggtgggataactccgtaactgactacgcctttctctagactttacttgaccagatacactgtctttgacacgttgaaggattagagcaatcaaatccaagactggctaagcacgaagcaactcttgagtgttaaaaagttatctcctgtattcgggaagcgggtactagaagattgcagggactccgacgttaagtaaattacaaagtaataagtatcgttcaggatcacgttaccgcaataagaagcgagaataatataatttccgaagtgcttaccccagtagtgactattcctataacccttctgagtgtccggaggcggaaatttgccacgaaagagaaagtatttccccgacaataataaaggggcgctcctcagcttttccacttggttgggtaagctaggcaactctgaaaggagtttcggcgaattgaagccgacagctttgaattgttttaggggcgttattcgagggcaatcggagctaacttcaagactacttctttgttgaatactaaatagtgcaaaggtcgtgtttcctcaaggatactccgctaacaatataggattccaatcagattcagcactggcggtacgggtgttgcggtgaggcgttcgggtttacggctcgaagctagcacggtaggaagcctgacaatcaccaagcaaaagggccgtcgaaggcccacaagatacgaaagctctcgaagccttatccttgaccgatccacctatttaggcagttacgcacaaaagctacccaataatccgtgacaggcacaatatcacggaacaaaaccgaaaactctcgtacacggttaggttttcgctaggaagaataaacctctatcttgattataagaaggctccccaagcacccccaaaaccgaaatagcggtttgcaataagggacaagttacgagtgtagacacgcagaattatccagcctttagtctttaggaaggcaaagctattgtacgcggtagccgtcgtagcaatttaccaactgtagaattattggacacacgtaggaagggcttacagttgaagtttaataaggtcacacgcaaaaccgctaaggaataatcgcaccgttagcgaaagaatatttcagagcggttagtaaaggttgagtaaagtgagattccaaagtgagcctttataaaaagtaaagagctataataaaaccgtcgagcagaaaacaatcgcctgaaatctcaagcacgttgccctttctaacgtcgctaaggtttcgtaaacccgtttgattaggaagaagaataagtaacccgattaggtttgagatcgcgggttatcggtttggattaaaagtggataccagcggagtcaacgccgacgcaaacgtacagtgatccaatcctgttgcacggtcaagcacaatcagctcgcaagatcttggaatagtgtgcccaacagtttagttgagggccacgttccgactacaagttgcttcaagaggggaatttggatttggcaatagccccccgtttctacctcaagaggcgacgagtattaaccgcgccagctgtcggcacaagggccaaagaagattccaatttcttattcccgaataacctccgaatccctgcgggaaaatcaccgaccgaatagcctagaagcaagggggaacagataggtataattagcttaagagagtaccagccgtgacaacagcgtagtaaccacaaacttacgctggggcttctttggcggatttttacagatactaacaaggtgatttgaagtaccttagttgaggatttaaacgcgctatccggtaatctccaaattgggaaataccgttcaaagagggctagaattacttaaaagccttcacaccgcctgcgctatacgcgcccactctcccgtttatccgtccaagcggaagcagggcgatcctccgctaagatattcttacgtgtaacgtagctaagtatcccaaatagctggcgtacgcgttgaacaccgcctagaggatcgtgactcgccggacgagcgtgttattggggacttacgccagcgtagactacaacgcgcccagattaaccctgcacgtattgccttgaataacgtactaatctctccggctctcgacaatctatcgagcgactcgattatcaacgggtgtcttgcagttctaatctcttgcccccgcccgtaatagcctccaagagattgaagatagtaaagggcaagagctgattcggcgttgaaggatagcggactttcggtcaaccacaattccccactcgacaaaaccagccgtgcgaataactctgaaagtacaagcaacccaagagggctgagcctaaactcagctaattcctaagtgagctaaagactcgaagtgacagctcttaataaatagagcgggaacgtcgaacggtcgtgaaagtaatagtacaacgggtattaacttactgaggatattgcttgaagctgtaccgttttattgggtgaacgaataagatccagcaattcagccaaagaagctaccaatttttagtttaagagtgtcacgtctgacctcgcgggtagattgccgaacgtagagcttacgagccagcggaaacagtagccgcaggataagtaaggggagtaagtgatcgaacgaatcagaagtgacaatatacttaggctggatctcgtcccgtgaatcccaaccctcaccaactacgagataagaggtaagccaaaaatcgacttggtggcgaccaacgactgttccccccctgtaactaatcgttccgtcaaaacctgacttacttcaaggccaattccaagcgcaaacaataccgtcctagttcttcggttaagtttccgaagtaggagtgagcctacctccgtttgcgtcttgttaccactgacccagctatttactttgtattgcctgcaatcgaatttctgaactctcagatagtggggataacgggaaagttcctatatttgcgaactaacttagccgtccacctcgaagctacctactcacacccaccccgcgcggggtaaataaggcactaatcccagctgagagctggcgtagcacttagccacaagttaattaacagttgtctggtagtttggcggtattaggaagatcctagaagcaaggcagagttagttctaacctaaagccacaaataagacaggttgccaaagcccgccggaaattaaatcttgctcagttcggtaacggagtttccctcccgcgtacttaattcccaataagaaacgcgcccaagtcctatcaggcaaaattcagccccttcccgtgttagaacgagggtaaaaatacaagccgattgaacaagggttgggggcttcaaatcgtcgtttaccccactttacaacggagattaagtagttcaccctatagtacgaagcagaactatttcgaggggcgtgcaataatcgaatcttctgcggttgacttaacacgctagggacgtgccctcgattcaatcgaaggtactcctactcagactgcctcacacccagctagtcactgagcgataaaattgacccgccctctagggaagcgagtacgtcccaaagggctccggacagggctatataggagagtttgatctcgccccgacaactgcaaccctcaactcccttagataatattgttagccgaagttgcacgacccgccgtccacggactgctcttagggtgtggctccttaatctgacaacgtgcaacccctatcgaagtcgattgtttctgcgaaaggtgttgtcctaatagtcccgaaatttggcccttgtaggtgtgaaaccacttagcttcgcgccgtagtcctaaaggcccacctattgactttgtttcgggtagcactaggaatcttaacaatttgaatttggacgtggaacgcgtacaccttaatctccgaataattctagggatttggaagtcctctacgttgacacacctacactgctcgaagtaaatatacgaataacgcgggcctcgcggagccgttccgaatcgtcacgtgttcgtttactgttaattggtggcaaataagcaatatcgtagtccgtcaggcccagccctgttatccacggcgttatttgtcaaattgcgtagaactggattgactgcctgacaatacctaattatcggtacgaagtccccgaatctgtcgggctatttcactaatactttccaaacgccccgtatccaagaagaacgaatttatccacgctcccgtctttgggacgaataccgctacaagtggacagaggatcggtacgggcctctaataaatccaacactctacgccctcttcaagagctagaagaacagggtgcagttggaaagggaattatttcgtaaggcgagccaataccgtaattaattcggaagagttaacacgattggaagtaggaatagtttctaaccacggttactaatcctaataacggaacgctgtctgatagattagtgtcagcgctcggtaccaaagaaaaataaaaagacgctgaaaagcgtctttttatttttcggtccagtgtaactcaggcaaaagcacgtaatattcgtactttcttcctccgtaagcgtcacccacattccttaaagagtgcatgtgcatattttgttatcaataaaaaaggccgcgatttgcggccttattgttcgtcttgccggattacgccccgccctgccactcatcgcagtattgttgtaattcattaagcattctgccgacatggaagccatcacaaacggcatgatgaacttggatcgccagtggcattaacaccttgtcgccttgcgtataatattttcccatagtgaaaacgggggcgaagaagttgtccatatttgctacgtttaaatcaaaactggtgaaactcacccacggattggcactgacgaaaaacatattttcgataaaccctttagggaaatatgctaagttttcaccgtaacacgccacatcttgactatatatgtgtagaaactgccggaaatcgtcgtggtattctgaccagagcgatgaaaacgtttcagtttgctcatggaaaacggtgtaacaagggtgaacactatcccatatcaccagctcaccgtctttcattgccatacgaaactccggatgtgcattcatcaggcgggcaagaatgtgaataaaggccggataaaacttgtgcttatttttctttacggtttttaaaaaggccgtaatatccagctgaacggtttggttataggtgcactgagcaactgactggaatgcctcaaaatgttctttacgatgccattgacttatatcaactgtagtatatccagtgatttttttctccattttagcttccttagcttgcgaaatctcgataactcaaaaaatagtagtgatcttatttcattatggtgaaagttgtcttacgtgcaacattttcgcaaaaagttggcgctttatcaac

SEQ ID NO: 9 - DNA, 7300 bp, Artificial Sequence
  p7.3kb (p513)
cctttagggaaatatgctaagttttcaccgtaacacgccacatcttgactatatatgtgtagaaactgccggaaatcgtcgtggtattctgaccagagcgatgaaaacgtttcagtttgctcatggaaaacggtgtaacaagggtgaacactatcccatatcaccagctcaccgtctttcattgccatacgaaactccggatgtgcattcatcaggcgggcaagaatgtgaataaaggccggataaaacttgtgcttatttttctttacggtttttaaaaaggccgtaatatccagctgaacggtttggttataggtgcactgagcaactgactggaatgcctcaaaatgttctttacgatgccattgacttatatcaactgtagtatatccagtgatttttttctccattttagcttccttagcttgcgaaatctcgataactcaaaaaatagtagtgatcttatttcattatggtgaaagttgtcttacgtgcaacattttcgcaaaaagttggcgctttatcaacactgtccctcctgttcagctactgacggtactgcggaactgactaaagtagtgcgtaacggcaaaagcaccgccggacatctgcgctagcggagtgtatactggcttactatgttggcactgatgagggtgtaagtgaagtgcttcatgtggcaggagaaaaaaggctgcatcggtgcgtcagcagaatatgtgatacaggatatattccgcttcctcgctcactgactcgctacgctcggtcgttcgactgtggcgagcggaaatggcttacgaacggggcggagatttcctggaagatgccaggaagatacttaacagggaagtgagagggtcgcggcaaagccgtttttccataggctccgcccccctgacaagcatcacgaaatctgacgctcaaatcagtggtggcgaaacctgacaggactataaagataccaggcgtttccccctggcggctccctcgtgcgctctcctgttcctgcctttcggtttgccggtgtcattcctctgttacggccgagtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggataaggctaaactgaaaggacaagttttggcgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcactttatagctagctcagcccttggtacaatgctagcgttttcattaaagaggagaaaggaagccatgagtaaaggtgaggaattatttactggtgttgttccgatcttagttgaactggacggcgatgttaacggtcataaattcagtgttcgtggtgaaggtgaaggtgatgcaaccaacggtaagctgaccctgaaattcatctgcactactggaaaattaccagtaccgtggcctactctggtgactaccctgacctatggtgttcagtgtttttctcgttaccctgaccacatgaagcaacatgatttcttcaaatctgcaatgccggaaggttatgtacaggagcgcaccatttctttcaaagacgatggcacgtataaaacccgtgcagaggttaaatttgaaggtgacactctggtgaatcgtattgaactgaaaggcattgatttcaaagaggacggcaatattttaggccacaaactggaatataacttcaactcccataacgtttacatcaccgcagacaaacaaaagaacggtatcaaagctaacttcaaaattcgccataacgttgaagacggtagcgtacagctggcggatcattaccaacagaacactccgattggagatgctcctgttttactgccggataaccactacctgtccacccagtctaaactgtcgaaggatccgaacgaaaagcgcgaccacatggtgttattagagttcgttaccgctagtggtatcacgcacggtatggatgaactctacaaataagtcagtttcacctgttttacgttaaaacccgcttcggcgggtttttacttttgggtttagccgaacgccccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaagaacctgtaaagtgataatgattatcatctacatatcacaacgtgcgtaaagggactataacaagacgcaaacggaggtaggctcactcctacttcggaaacttaaccgaagaactaggacggtattgtttgcgcttggaattggccttgaagtaagtcaggttttgacggaacgattagttacagggggggaacagtcgttggtcgccaccaagtcgatttttggcttacctcttatctcgtagttggtgagggttgggattcacgggacgagatccagcctaagtatattgtcacttctgattcgttcgatcacttactccccttacttatcctgcggctactgtttccgctggctcgtaagctctacgttcggcaatctacccgcgaggtcagacgtgacactcttaaactaaaaattggtagcttctttggctgaattgctggatcttattcgttcacccaataaaacggtacagcttcaagcaatatcctcagtaagttaatacccgttgtactattactttcacgaccgttcgacgttcccgctctatttattaagagctgtcacttcgagtctttagctcacttaggaattagctgagtttaggctcagccctcttgggttgcttgtactttcagagttattcgcacggctggttttgtcgagtggggaattgtggttgaccgaaagtccgctatccttcaacgccgaatcagctcttgccctttactatcttcaatctcttggaggctattacgggcgggggcaagagattagaactgcaagacacccgttgataatcgagtcgctcgatagattgtcgagagccggagagattagtacgttattcaaggcaatacgtgcagggttaatctgggcgcgttgtagtctacgctggcgtaagtccccaataacacgctcgtccggcgagtcacgatcctctaggcggtgttcaacgcgtacgccagctatttgggatacttagctacgttacacgtaagaatatcttagcggaggatcgccctgcttccgcttggacggataaacgggagagtgggcgcgtatagcgcaggcggtgtgaaggcttttaagtaattctagccctctttgaacggtatttcccaatttggagattaccggatagcgcgtttaaatgagtgtcagagaaacggaagccgaagtctttccattccggatgtttggaaatgctctgtttatagaagtcgatgaacttacggcagtcctctatattaaattcgaatttttcataccctttctgcgggctaccgtttttagtgtgcgtgctatgattgcggatgcgcagaatatcctcagacgggttgtagaatttgatgcttttcgcggaaaagaacactttcggtaacattttattcgcacccggcaggagcttgtacacgattttcttatagccttcacccttgttttctttgatcgctttatcgtcgaagatcttgttgttcttcttgttcattacgcccagatagtatttgtcgtctttgatgaacaggattgcggtgttgtccggctctttgttcttatcccagccgttcgccagcgtgctgttttcgaagttcagtttgaatttctcgtcagagtaaggcttctgcgtgatgtagttgcggattttattgtagagagggacgatgtttgccagttcgaagtaacattcttcgaacaccagatagaagtgttcatctttatccagaatgttcgccttgtcctcgctctggctgatgtggaagattttgagcttgtgtaataagttattcgtctgatctaataagtctttaattgctttcacatcgtcctccgcagatgcttgaagcagatctttcttaccctgattctggtacttgatagagatctgcgccagattgtctttgttttgagcaatttcgtcgaagatcatcgggattgccgcaaagttcgccagaatttcctcaaaacgacactgtttatcaatatcacgatgtttattaaattcctcaagtgccagtttgatagtttctaagctcaggtatttagctttttctgttttctttgcaatcagttcctgttccttcttggacgggttgtccagatttttcggcgcgatttgttgggtgatgtattccaaaactgccgtgccgatcacgctatagtcatcgaaaacttgttgactgagatcggtcagagatttgtcgtttttaaagtaaatcttagacagatctagtttctgcgctttgaggtcgtcaaagagcagggacagagtttctttaatagatttctcttccacggttttgaacgccgcaatctgctcataaaagctctgcatcgtggtgacaacgtcgctatcatcttccagtttatcaattacgaaggatttagattcggtgtccgataaaatctgtttaaacagaacggacattttatactttttcagggttttgtcgttgatttgttggctatacaggttaatgtattcgttgatgcccttacgcttggtgttttcgccgttaacaaatttgccaccaataatggtgttgaatttggtgatgccagattgattcaggtaattgttgaaattagcgatttcgaaaacctcgtccagtgagaaaacacgctggttaacttcggaggttttatagtcgatgtcgaaggtcagttcttccgccagatctttcttgatctgttcatagttaatagcttccggtgctttgtctttcagagattcatatttcgctttgttttccagaaacttcggcaggttgtcgtccacgatacgataaataatagaggtcggaatatcgttgctcgaatatacattcttacggttttcatgaaaacctttgaaatacgtcgtccagcctttgaaagacttgatgatttcggtattcgaatatgacctgatcaaagataaacgtttcaccgaagataagttctttttccactgtccgattaccatcaacttcaaatctagcggtgcgaacaagttcaacgatgaaattaacttattactgaaagagaaagctaatgacgtacacatcttatctattgatcgcggtgaacgtcatttagcatactatacactggtagatggtaaaggtaatattattaaacaggatactttcaatattatcggtaatgaccgtatgaaaaccaactatcacgataagctggcggcgatcgaaaaagatcgtgattctgcgcgtaaagattggaagaaaattaacaatatcaaagaaatgaaagaaggctatctgagccaagtggtgcacgagatcgcaaaactggtgattgaatataacgctatcgtggttttcgaagatctgaactttggttttaaacgtggtcgcttcaaagtagaaaaacaggtgtaccaaaaactggaaaaaatgctgattgaaaaactgaactatctggtttttaaagacaacgaatttgacaaaacgggtggcgtactccgtgcctatcagctgaccgctccgttcgaaacgttcaagaaaatgggtaaacaaacggggattatctattatgtgccagctggtttcacctccaagatttgtccagttacgggcttcgttaaccagctgtacccgaaatacgagagcgttagcaaatctcaagaatttttcagcaaattcgacaagatctgctataatctggataaaggctatttcgagttcagcttcgattacaaaaacttcggcgataaagcggctaaaggtaagtggactattgctagctttggtagccgtctgattaactttcgcaactccgacaaaaaccataattgggacacgcgtgaagtgtatccgaccaaagaactggaaaaattactgaaagactattccggacactcagaagggttataggaatagtcactactggggtaagcacttcggaaattatattattctcgcttcttattgcggtaacgtgatcctgaacgatacttattactttgtaatttacttaacgtcggagtccctgcaatcttctagtacccgcttcccgaatacaggagataactttttaacactcaagagttgcttcgtgcttagccagtcttggatttgattgctctaatccttcaacgtgtcaaagacagtgtatctggtcaagtaaagtctagagaaaggcgtagtcagttacggagttatcccaccttagtgttactccgatttaatttctgctttctttgatttctacccgactttcgccgtgacttcaatagagaggcaggctcttgctatttctttcaagggcttgtccaactacctaattaagataaagatacggcagttgacgcactgccgataatttctttacgtcagcgaaattaaatcgagcaccagtcgtagagtcgcggttgcctagcagtttatctcgcgtacgggccttcgctacttacacgatacctagtacgtggattcgggtagcaccagaagtctatagcatgtgcatacctttggtcgaaaaaaaaagcccgcactgtcaggtgcgggcttttttcagtgtttccttgccggattacgccccgccctgccactcatcgcagtattgttgtaattcattaagcattctgccgacatggaagccatcacaaacggcatgatgaacttggatcgccagtggcattaacaccttgtcgccttgcgtataatattttcccatagtgaaaacgggggcgaagaagttgtccatatttgctacgtttaaatcaaaactggtgaaactcacccagggattggcactgacgaaaaacatattttcgataaac

SEQ ID NO: 10 - protein, 1132 aa, Escherichia phage lambda (Bacteriophage lambda)
  MISC_FEATURE (814)..(821): box 1 in Figure 7
  MISC_FEATURE (958)..(966): box 2 in Figure 7
MGKGSSKGHTPREAKDNLKSTQLLSVIDAISEGPIEGPVDGLKSVLLNSTPVLDTEGNTNISGVTVVFRAGEQEQTPPEGFESSGSETVLGTEVKYDTPITRTITSANIDRLRFTFGVQALVETTSKGDRNPSEVRLLVQIQRNGGWVTEKDITIKGKTTSQYLASVVMGNLPPRPFNIRMRRMTPDSTTDQLQNKTLWSSYTEIIDVKQCYPNTALVGVQVDSEQFGSQQVSRNYHLRGRILQVPSNYNPQTRQYSGIWDGTFKPAYSNNMAWCLWDMLTHPRYGMGKRLGAADVDKWALYVIGQYCDQSVPDGFGGTEPRITCNAYLTTQRKAWDVLSDFCSAMRCMPVWNGQTLTFVQDRPSDKTWTYNRSNVVMPDDGAPFRYSFSALKDRHNAVEVNWIDPNNGWETATELVEDTQAIARYGRNVTKMDAFGCTSRGQAHRAGLWLIKTELLETQTVDFSVGAEGLRHVPGDVIEICDDDYAGISTGGRVLAVNSQTRTLTLDREITLPSSGTALISLVDGSGNPVSVEVQSVTDGVKVKVSRVPDGVAEYSVWELKLPTLRQRLFRCVSIRENDDGTYAITAVQHVPEKEAIVDNGAHFDGEQSGTVNGVTPPAVQHLTAEVTADSGEYQVLARWDTPKVVKGVSFLLRLTVTADDGSERLVSTARTTETTYRFTQLALGNYRLTVRAVNAWGQQGDPASVSFRIAAPAAPSRIELTPGYFQITATPHLAVYDPTVQFEFWFSEKQIADIRQVETSTRYLGTALYWIAASINIKPGHDYYFYIRSVNTVGKSAFVEAVGRASDDAEGYLDFFKGKITESHLGKELLEKVELTEDNASRLEEFSKEWKDASDKWNAMWAVKIEQTKDGKHYVAGIGLSMEDTEEGKLSQFLVAANRIAFIDPANGNETPMFVAQGNQIFMNDVFLKRLTAPTITSGGNPPAFSLTPDGKLTAKNADISGSVNANSGTLSNVTIAENCTINGTLRAEKIVGDIVKAASAAFPRQRESSVDWPSGTRTVTVTDDHPFDRQIVVLPLTFRGSKRTVSGRTTYSMCYLKVLMNGAVIYDGAANEAVQVFSRIVDMPAGRGNVILTFTLTSTRHSADIPPYTFASDVQVMVIKKQALGISVV

SEQ ID NO: 11 - protein, 1132 aa, Artificial Sequence
  gpJ 591
MGKGSSKGHTPREAKDNLKSTQLLSVIDAISEGPIEGPVDGLKSVLLNSTPVLDTEGNTNISGVTVVFRAGEQEQTPPEGFESSGSETVLGTEVKYDTPITRTITSANIDRLRFTFGVQALVETTSKGDRNPSEVRLLVQIQRNGGWVTEKDITIKGKTTSQYLASVVMGNLPPRPFNIRMRRMTPDSTTDQLQNKTLWSSYTEIIDVKQCYPNTALVGVQVDSEQFGSQQVSRNYHLRGRILQVPSNYNPQTRQYSGIWDGTFKPAYSNNMAWCLWDMLTHPRYGMGKRLGAADVDKWALYVIGQYCDQSVPDGFGGTEPRITCNAYLTTQRKAWDVLSDFCSAMRCMPVWNGQTLTFVQDRPSDKTWTYNRSNVVMPDDGAPFRYSFSALKDRHNAVEVNWIDPNNGWETATELVEDTQAIARYGRNVTKMDAFGCTSRGQAHRAGLWLIKTELLETQTVDFSVGAEGLRHVPGDVIEICDDDYAGISTGGRVLAVNSQTRTLTLDREITLPSSGTALISLVDGSGNPVSVEVQSVTDGVKVKVSRVPDGVAEYSVWELKLPTLRQRLFRCVSIRENDDGTYAITAVQHVPEKEAIVDNGAHFDGEQSGTVNGVTPPAVQHLTAEVTADSGEYQVLARWDTPKVVKGVSFLLRLTVTADDGSERLVSTARTTETTYRFTQLALGNYRLTVRAVNAWGQQGDPASVSFRIAAPAAPSRIELTPGYFQITATPHLAVYDPTVQFEFWFSEKQIADIRQVETSTRYLGTALYWIAASINIKPGHDYYFYIRSVNTVGKSAFVEAVGRASDDAEGYLDFFKGKITESHLGKELLEKVELTEDNASRLEEFSKEWKDASDKWNAMWAVKIEQTKDGKHYVAGIGLSMEDTEEGKLSQFLVAANRIAFIDPANGNETPMFVAQGNQIFMNDVFLKRLTAPTITSGGNPPAFSLTPDGKLTAKNADISGNVNANSGTLNNVTINENCQIKGKLSANQIEGDIVKTVSKSFPRTNSYASGTITVRISDDQKFDRQVMIPPVLFRGGKHENFNSNNQQSYWYSTCRLRVTRNGQEIFNQSTTDAQGVFSSVIDMPAGQGTLTLTFTVSSSGANNWTPTTSISDLLVVVMKKSTAGISIS

SEQ ID NO: 12 - protein, 1159 aa, Artificial Sequence
  gpJ Z2145
MGKGSSKGHTPREAKDNLKSTQLLSVIDAISEGPIEGPVDGLKSVLLNSTPVLDTEGNTNISGVTVVFRAGEQEQTPPEGFESSGSETVLGTEVKYDTPITRTITSANIDRLRFTFGVQALVETTSKGDRNPSEVRLLVQIQRNGGWVTEKDITIKGKTTSQYLASVVMGNLPPRPFNIRMRRMTPDSTTDQLQNKTLWSSYTEIIDVKQCYPNTALVGVQVDSEQFGSQQVSRNYHLRGRILQVPSNYNPQTRQYSGIWDGTFKPAYSNNMAWCLWDMLTHPRYGMGKRLGAADVDKWALYVIGQYCDQSVPDGFGGTEPRITCNAYLTTQRKAWDVLSDFCSAMRCMPVWNGQTLTFVQDRPSDKTWTYNRSNVVMPDDGAPFRYSFSALKDRHNAVEVNWIDPNNGWETATELVEDTQAIARYGRNVTKMDAFGCTSRGQAHRAGLWLIKTELLETQTVDFSVGAEGLRHVPGDVIEICDDDYAGISTGGRVLAVNSQTRTLTLDREITLPSSGTALISLVDGSGNPVSVEVQSVTDGVKVKVSRVPDGVAEYSVWELKLPTLRQRLFRCVSIRENDDGTYAITAVQHVPEKEAIVDNGAHFDGEQSGTVNGVTPPAVQHLTAEVTADSGEYQVLARWDTPKVVKGVSFLLRLTVTADDGSERLVSTARTTETTYRFTQLALGNYRLTVRAVNAWGQQGDPASVSFRIAAPAAPSRIELTPGYFQITATPHLAVYDPTVQFEFWFSEKQIADIRQVETSTRYLGTALYWIAASINIKPGHDYYFYIRSVNTVGKSAFVEAVGRASDDAEGYLDFFKGEIGKTHLAQELWTQIDNGQLAPDLAEIRTSITDVSNEITQTVNKKLEDQSAAIQQIQKVQVDTNNNLNSMWAVKLQQMQDGRLYIAGIGAGIENTSDGMQSQVLLAADRIAMINPANGNTKPMFVGQGDQIFMNEVFLKYLTAPTITSGGNPPAFSLTSDGKLTAKNADISGSVNANSGTLNNVTINENCRVLGKLSANQIEGDLVKTVGKAFPRDSRAPERWPSGTITVRVYDDQPFDRQIVIPAVAFSGAKHEREHTDIYSSCRLIVRKNGAEIYNRTALDNTLIYSGVIDMPAGHGHMTLEFSVSAWLVNNWYPTASISDLLVVVMKKATAGISIS

SEQ ID NO: 13 - protein, 1131 aa, Artificial Sequence
  1A2
MGKGSSKGHTPREAKDNLKSTQLLSVIDAISEGPIEGPVDGLKSVLLNSTPVLDTEGNTNISGVTVVFRAGEQEQTPPEGFESSGSETVLGTEVKYDTPITRTITSANIDRLRFTFGVQALVETTSKGDRNPSEVRLLVQIQRNGGWVTEKDITIKGKTTSQYLASVVMGNLPPRPFNIRMRRMTPDSTTDQLQNKTLWSSYTEIIDVKQCYPNTALVGVQVDSEQFGSQQVSRNYHLRGRILQVPSNYNPQTRQYSGIWDGTFKPAYSNNMAWCLWDMLTHPRYGMGKRLGAADVDKWALYVIGQYCDQSVPDGFGGTEPRITCNAYLTTQRKAWDVLSDFCSAMRCMPVWNGQTLTFVQDRPSDKTWTYNRSNVVMPDDGAPFRYSFSALKDRHNAVEVNWIDPNNGWETATELVEDTQAIARYGRNVTKMDAFGCTSRGQAHRAGLWLIKTELLETQTVDFSVGAEGLRHVPGDVIEICDDDYAGISTGGRVLAVNSQTRTLTLDREITLPSSGTALISLVDGSGNPVSVEVQSVTDGVKVKVSRVPDGVAEYSVWELKLPTLRQRLFRCVSIRENDDGTYAITAVQHVPEKEAIVDNGAHFDGEQSGTVNGVTPPAVQHLTAEVTADSGEYQVLARWDTPKVVKGVSFLLRLTVTADDGSERLVSTARTTETTYRFTQLALGNYRLTVRAVNAWGQQGDPASVSFRIAAPAAPSRIELTPGYFQITATPHLAVYDPTVQFEFWFSEKQIADIRQVETSTRYLGTALYWIAASINIKPGHDYYFYIRSVNTVGKSAFVEAVGRASDDAEGYLDFFKGKITESHLGKELLEKVELTEDNASRLEEFSKEWKDASDKWNAMWAVKIEQTKDGKHYVAGIGLSMEDTEEGKLSQFLVAANRIAFIDPANGNETPMFVAQGNQIFMNDVFLKRLTAPTITSGGNPPAFSLTPDGKLTAKNADISGNVNANSGTLNNVTINENCRVLGKLSANQIEGDLVKTVGKAFPRDSRAPERWPSGTITVRVYDDQPFDRQIVIPAVAFSGAKHEKEHTDIYSSCRLIVRKNGAEIYNRTALDNTLIYSGVIDMPAGHGHMTLEFSVSAWLVNNWYPTASISDLLVVVMKKATAGITIS

SEQ ID NO: 14 - protein, 774 aa, Escherichia phage lambda (Bacteriophage lambda)
MAVKISGVLKDGTGKPVQNCTIQLKARRNSTTVVVNTVGSENPDEAGRYSMDVEYGQYSVILQVDGFPPSHAGTITVYEDSQPGTLNDFLCAMTEDDARPEVLRRLELMVEEVARNASVVAQSTADAKKSAGDASASAAQVAALVTDATDSARAASTSAGQAASSAQEASSGAEAASAKATEAEKSAAAAESSKNAAATSAGAAKTSETNAAASQQSAATSASTAATKASEAATSARDAVASKEAAKSSETNASSSAGRAASSATAAENSARAAKTSETNARSSETAAERSASAAADAKTAAAGSASTASTKATEAAGSAVSASQSKSAAEAAAIRAKNSAKRAEDIASAVALEDADTTRKGIVQLSSATNSTSETLAATPKAVKVVMDETNRKAPLDSPALTGTPTAPTALRGTNNTQIANTAFVLAAIADVIDASPDALNTLNELAAALGNDPDFATTMTNALAGKQPKNATLTALAGLSTAKNKLPYFAENDAASLTELTQVGRDILAKNSVADVLEYLGAGENSAFPAGAPIPWPSDIVPSGYVLMQGQAFDKSAYPKLAVAYPSGVLPDMRGWTIKGKPASGRAVLSQEQDGIKSHTHSASASGTDLGTKTTSSFDYGTKTTGSFDYGTKSTNNTGAHAHSLSGSTGAAGAHAHTSGLRMNSSGWSQYGTATITGSLSTVKGTSTQGIAYLSKTDSQGSHSHSLSGTAVSAGAHAHTVGIGAHQHPVVIGAHAHSFSIGSHGHTITVNAAGNAENTVKNIAFNYIVRLA

SEQ ID NO: 15 - protein, 194 aa, Escherichia phage lambda (Bacteriophage lambda)
MAFRMSEQPRTIKIYNLLAGTNEFIGEGDAYIPPHTGLPANSTDIAPPDIPAGFVAVFNSDEASWHLVEDHRGKTVYDVASGDALFISELGPLPENFTWLSPGGEYQKWNGTAWVKDTEAEKLFRIREAEETKKSLMQVASEHIAPLQDAADLEIATKEETSLLEAWKKYRVLLNRVDTSTAPDIEWPAVPVME

SEQ ID NO: 16 - protein, 1194 aa, Artificial Sequence
  STF Lambda-WW11.2
MAVKISGVLKDGTGKPVQNCTIQLKARRNSTTVVVNTVGSENPDEAGRYSMDVEYGQYSVILQVDGFPPSHAGTITVYEDSQPGTLNDFLCAMTEDDARPEVLRRLELMVEEVARNASVVAQSTADAKKSAGDASASAAQVAALVTDATDSARAASTSAGQAASSAQEASSGAEAASAKATEAEKSAAAAESSKNAAATSAGAAKTSETNAAASQQSAATSASTAATKASEAATSARDAVASKEAAKSSETNASSSAGRAASSATAAENSARAAKTSETNARSSETAAERSASAAADAKTAAAGSASTASTKATEAAGSAVSASQSKSAAEAAAIRAKNSAKRAEDIASAVALEDADTTRKGIVQLSSATNSTSETLAATPKAVKVVMDETNRKAPLDSPALTGTPTAPTALRGTNNTQIANTAFVLAAIADVIDASPDALNTLNELAAALGNDPDFATTMTNALAGKQPKNATLTALAGLSTAKNKLPYFAENDAASLTELTQVGRDILAKNSVADVLEYLGAGENSAKQDGLSLIGQCNSIADLRNVEPVMDGQRILVKQHTAGTGKGGGTFQAKLSGVGLTDDNGVTVKTVGGAAWVRVGADRVNPYMYGALGSGNDDTLAVQACLNSGKATNITDVHHVSNVTMNKSSASIYGSGLHYTRLHQLPAAVGSLLTIADTCSLTVIDSLGLYGTGYKQGTAFTTGTRGIDVLTPTGLSNNYPDHTTSDPRRDLVITKVHIAGFDEYGLNVDSGNFSVTTESVLINHIKQVGARCATTDWTWTNIQINTCGKQCLILDGCGNGRIIGGKFIWANWLPYGASGQFPGITINNSQNMVINGIEVQDCGGNGIEFTDSYSISMNGLNTNRNGINSTAEFYNLVFNRSDVEINGFVGLNYKVNSGSPDNSSSGNLKFLSSDCNVTLNGKLETGYMGIEFIGDTRVIQPTSSYIKLNGLVNYAGSGIQTINEVPTFDGVSTTPVYVPISSVVGQTNGLRLSQANKDKVIYTRKVGPEGVTMAAVITPTIGGAEIFNLMAIGTGFSSASNSLYFQLEIAADGSQNVSLLLSGDGTTQILPGTLPAALKLQSGEPYHVALGAKAGYFWWSILNLNTGKRIRRSFRGAYLPKPFASIFGLVSSPVFFSGAGIGDSGCSGVGAKIYLGSFSDENDYVSSRYYSLVNPVDPTKMYSFRILDSTI

SEQ ID NO: 17 - protein, 757 aa, Artificial Sequence
  STF Lambda-75
MAVKISGVLKDGTGKPVQNCTIQLKARRNSTTVVVNTVGSENPDEAGRYSMDVEYGQYSVILQVDGFPPSHAGTITVYEDSQPGTLNDFLCAMTEDDARPEVLRRLELMVEEVARNASVVAQSTADAKKSAGDASASAAQVAALVTDATDSARAASTSAGQAASSAQEASSGAEAASAKATEAEKSAAAAESSKNAAATSAGAAKTSETNAAASQQSAATSASTAATKASEAATSARDAVASKEAAKSSETNASSSAGRAASSATAAENSARAAKTSETNARSSETAAERSASAAADAKTAAAGSASTASTKATEAAGSAVSASQSKSAAEAAAIRAKNSAKRAEDIASAVALEDADTTRKGIVQLSSATNSTSETLAATPKAVKVVMDETNRKAPLNSPALTGTPTTPTARQGTNNTQIASTAFVMAAIAALVDSSPDALNTLNELAAALGNDPNFATTMTNALAGKQPKDATLTALAGLATAADRFPYFTGNDVASLATLTKVGRDILAKSTVAAVIEYLGLRELGTSGEKIPLLSTANTWTNRQTLSGGLSGELSGNAATATKLKTARKIAGVGFDGSSDISISAKNVNAFALRQTGNTVNGDTSVGWNWDSGAYNALIGGASALILHFNINAGSCPAVQFRVNYKNGGISYRSARDGYGFELGWSDFYTTTRKPSAGDVGAYTQAECNSRFITGIRLGGLSSVQTWNGPGWSDRSGYVVTGSVNGNRDELIDTTQARPIQYCINGTWYNAGSI

SEQ ID NO: 18 - protein, 177 aa, Artificial Sequence
  Accessory protein of Lambda-STF75
MMHLKNIIAGNPKTKEQYQLTKQFNIKWLYSDDGKNWYEEQKNFQPDTLKMVYDHNGVIICIEKDVSAINPEGASVVELPDITANRRADISGKWMFKDGVVIKRTYTEEEQRQQAENEKQSLLQLVRDKTQLWDSQLRLGIISDENKQKLTEWMLFAQKVESTDTSSLPVTYPEQPE

SEQ ID NO: 19 - protein, 745 aa, Artificial Sequence
  STF Lambda-EB6
MAVKISGVLKDGTGKPVQNCTIQLKARRNSTTVVVNTVGSENPDEAGRYSMDVEYGQYSVILQVDGFPPSHAGTITVYEDSQPGTLNDFLCAMTEDDARPEVLRRLELMVEEVARNASVVAQSTADAKKSAGDASASAAQVAALVTDATDSARAASTSAGQAASSAQEASSGAEAASAKATEAEKSAAAAESSKNAAATSAGAAKTSETNAAASQQSAATSASTAATKASEAATSARDAVASKEAAKSSETNASSSAGRAASSATAAENSARAAKTSETNARSSETAAERSASAAADAKTAAAGSASTASTKATEAAGSAVSASQSKSAAEAAAIRAKNSAKRAEDIASAVALEDADTTRKGIVQLSSATNSTSETLAATPKAVKIAMDNANARLAKDRNGADIPNKPLFIQNLGLQETVNKAGNAVQKTGDTLSGGLTFENDSILAWIRNTDWAKIGFKNDADSDTDSYMWFETGDNGNEYFKWRSRQSTTTKDLMNLKWDALYVLVNAIVNGEVISKSANGLRIAYGNYGFFIRNDGSNTYFMLTNSGDNMGTYNGLRPLWINNATGAVSMGRGLNVSGETLSDRFAINSSNGMWIQMRDNNAIFGKNIVNTDSAQALLRQNHADRKFMIGGLGNKQFGIYMINNSRTANGTDGQAYMDNNGNWLCGAQVIPGNYGNFDSRYVRDVRLGTRVVQLMARGGRYEKAGHAITGLRIIGEVDGDDEAIFRPIQKYINGTWYNVAQV

SEQ ID NO: 20 - protein, 175 aa, Artificial Sequence
  Accessory protein of Lambda-EB6
MQHLKNIKSGNPKTKEQYQLTKNFDVIWLWSEDGKNWYEEVNNFQDDTIKIVYDENNIIVAITKDASTLNPEGFSVVEIPDITANRRADDSGKWMFKDGAVVKRIYTADEQQQQAESQKAALLSEAENVIQPLERAVRLNMATDEERARLESWERYSVLVSRVDTAKPEWPQKPE

SEQ ID NO: 21 - protein, 757 aa, Artificial Sequence
  STF Lambda-23
MAVKISGVLKDGTGKPVQNCTIQLKARRNSTTVVVNTVGSENPDEAGRYSMDVEYGQYSVILQVDGFPPSHAGTITVYEDSQPGTLNDFLCAMTEDDARPEVLRRLELMVEEVARNASVVAQSTADAKKSAGDASASAAQVAALVTDATDSARAASTSAGQAASSAQEASSGAEAASAKATEAEKSAAAAESSKNAAATSAGAAKTSETNAAASQQSAATSASTAATKASEAATSARDAVASKEAAKSSETNASSSAGRAASSATAAENSARAAKTSETNARSSETAAERSASAAADAKTAAAGSASTASTKATEAAGSAVSASQSKSAAEAAAIRAKNSAKRAEDIASAVALEDADTTRKGIVQLSSATNSTSETLAATPKAVKVVMDETNRKAPLNSPALTGTPTTPTARQGTNNTQIASTAFVMAAIAALVDSSPDALNTLNELAAALGNDPNFATTMTNALAGKQPKDATLTALAGLATAADRFPYFTGNDVASLATLTKVGRDILAKSTVAAVIEYLGLRELGTSGEKIPLLSTANTWTNRQTFSGGLSGGLSGNAATATKLKTARKIAGVGFDGSSDISISAKNVNAFALRQTGNTVNGDTSVGWNWDSGAYNALIGGASALILHFNINAGSCPAVQFRVNYKNGGISYRSARDGYGFELGWSDFYTTTRKPSAGDVGAYTRAECNSRFITGIRLGGLSSVQTWNGPGWSDRSGYVVTGSVNGNRDELIDTTQARPIQYCINGTWYNAGSI

SEQ ID NO: 22 - protein, 177 aa, Artificial Sequence
  Accessory protein of Lambda-STF23
MMHLKNITAGNPKTKEQYQLTKQFNIKWLYSDDGKNWYEEQKNFQPDTLKMVYDHNGVIICIEKDVSAINPEGASVVELPDITANRRADISGKWLFKDGVVIKRTYTEEEQRQQAENEKQSLLQLVRDKTQLWDSQLRLGIISDENKQKLTEWMLYAQKVESTDTSSLPVTFPEQPE

SEQ ID NO: 23 - protein, 853 aa, Escherichia phage lambda (Bacteriophage lambda)
  MISC_FEATURE (187)..(208): distal region box in Figure 9A
  MISC_FEATURE (301)..(414): proximal region box in Figure 9A
MAEPVGDLVVDLSLDAARFDEQMARVRRHFSGTESDAKKTAAVVEQSLSRQALAAQKAGISVGQYKAAMRMLPAQFTDVATQLAGGQSPWLILLQQGGQVKDSFGGMIPMFRGLAGAITLPMVGATSLAVATGALAYAWYQGNSTLSDFNKTLVLSGNQAGLTADRMLVLSRAGQAAGLTFNQTSESLSALVKAGVSGEAQIASISQSVARFSSASGVEVDKVAEAFGKLTTDPTSGLTAMARQFHNVSAEQIAYVAQLQRSGDEAGALQAANEAATKGFDDQTRRLKENMGTLETWADRTARAFKSMWDAVLDIGRPDTAQEMLIKAEAAYKKADDIWNLRKDDYFVNDEARARYWDDREKARLALEAARKKAEQQTQQDKNAQQQSDTEASRLKYTEEAQKAYERLQTPLEKYTARQEELNKALKDGKILQADYNTLMAAAKKDYEATLKKPKQSSVKVSAGDRQEDSAHAALLTLQAELRTLEKHAGANEKISQQRRDLWKAESQFAVLEEAAQRRQLSAQEKSLLAHKDETLEYKRQLAALGDKVTYQERLNALAQQADKFAQQQRAKRAAIDAKSRGLTDRQAEREATEQRLKEQYGDNPLALNNVMSEQKKTWAAEDQLRGNWMAGLKSGWSEWEESATDSMSQVKSAATQTFDGIAQNMAAMLTGSEQNWRSFTRSVLSMMTEILLKQAMVGIVGSIGSAIGGAVGGGASASGGTAIQAAAAKFHFATGGFTGTGGKYEPAGIVHRGEFVFTKEATSRIGVGNLYRLMRGYATGGYVGTPGSMADSRSQASGTFEQNNHVVINNDGTNGQIGPAALKAVYDMARKGARDEIQTQMRDGGLFSGGGR

SEQ ID NO: 24 - protein, 859 aa, Artificial Sequence
  gpH-IAI
MAEPVGDLVVDLSLDAARFDEQMARVRRHFSGTESDAKKTAAVVEQSLSRQALAAQKAGISVGQYKAAMRMLPAQFTDVATQLAGGQSPWLILLQQGGQVKDSFGGMIPMFRGLAGAITLPMVGATSLAVATGALAYAWYQGNSTLSDFNKTLVLSGNQAGLTADRMLVLSRAGQAAGLTFNQTSESLTALVNAGVRGGEQFEAISQSVARFSSASGVEVDKVAEAFGKLTTDPTSGLTAMARQFHNVTAEQIAYVAQLQRSGDEAGALQAANEAATKGFDDQTRRLKENMGTLETWADRTARAFKSMWDSVLDIGRPDTAQGMLEKAEKAFDEADKKWQWYQSRSHRRGKTSAFLANLRGAWEDRANAQLGLSAATLQADLEKAREMAAKDWAESEASRLKYTEEAQKAYERLQTPLEKYTARQEELNKALKDGKILQADYNTLMAAAKKDYEATLKKPKQSSVKVSAGDRQEDSAHAALLTLQAELRTLEKHAGANEKISQQRRDLWKAESQFAVLEEAAQRRQLSAQEKSLLAHKDETLEYKRQLAALGDKVTYQERLNALAQQADKFAQQQRAKRAAIDAKSRGLTDRQAEREATEQRLKEQYGDNPLALNNVMSEQKKTWAAEDQLRGNWMAGLKSGWSEWEESATDSMSQVKSAATQTFDGIAQNMAAMLTGSEQNWRSFTRSVLSMMTEILLKQAMVGIVGSIGSAIGGAVGGGASASGGTAIQAAAAKFHFATGGFTGTGGKYEPAGIVHRGEFVFTKEATSRIGVGNLYRLMRGYATGGYVGTPGSMADSRSQASGTFEQNNHVVINNDGTNGQIGPAALKAVYDMARKGARDEIQTQMRDGGLFSGGGR

SEQ ID NO: 25 - DNA, 3396 bp, Artificial Sequence
  nucleic acid encoding H591
atgggtaaaggaagcagtaaggggcataccccgcgcgaagcgaaggacaacctgaagtccacgcagttgctgagtgtgatcgatgccatcagcgaagggccgattgaaggtccggtggatggcttaaaaagcgtgctgctgaacagtacgccggtgctggacactgaggggaataccaacatatccggtgtcacggtggtgttccgggctggtgagcaggagcagactccgccggagggatttgaatcctccggctccgagacggtgctgggtacggaagtgaaatatgacacgccgatcacccgcaccattacgtctgcaaacatcgaccgtctgcgctttaccttcggtgtacaggcactggtggaaaccacctcaaagggtgacaggaatccgtcggaagtccgcctgctggttcagatacaacgtaacggtggctgggtgacggaaaaagacatcaccattaagggcaaaaccacctcgcagtatctggcctcggtggtgatgggtaacctgccgccgcgcccgtttaatatccggatgcgcaggatgacgccggacagcaccacagaccagctgcagaacaaaacgctctggtcgtcatacactgaaatcatcgatgtgaaacagtgctacccgaacacggcactggtcggcgtgcaggtggactcggagcagttcggcagccagcaggtgagccgtaattatcatctgcgcgggcgtattctgcaggtgccgtcgaactataacccgcagacgcggcaatacagcggtatctgggacggaacgtttaaaccggcatacagcaacaacatggcctggtgtctgtgggatatgctgacccatccgcgctacggcatggggaaacgtcttggtgcggcggatgtggataaatgggcgctgtatgtcatcggccagtactgcgaccagtcagtgccggacggctttggcggcacggagccgcgcatcacctgtaatgcgtacctgaccacacagcgtaaggcgtgggatgtgctcagcgatttctgctcggcgatgcgctgtatgccggtatggaacgggcagacgctgacgttcgtgcaggaccgaccgtcggataagacgtggacctataaccgcagtaatgtggtgatgccggatgatggcgcgccgttccgctacagcttcagcgccctgaaggaccgccataatgccgttgaggtgaactggattgacccgaacaacggctgggagacggcgacagagcttgttgaagatacgcaggccattgcccgttacggtcgtaatgttacgaagatggatgcctttggctgtaccagccgggggcaggcacaccgcgccgggctgtggctgattaaaacagaactgctggaaacgcagaccgtggatttcagcgtcggcgcagaagggcttcgccatgtaccgggcgatgttattgaaatctgcgatgatgactatgccggtatcagcaccggtggtcgtgtgctggcggtgaacagccagacccggacgctgacgctcgaccgtgaaatcacgctgccatcctccggtaccgcgctgataagcctggttgacggaagtggcaatccggtcagcgtggaggttcagtccgtcaccgacggcgtgaaggtaaaagtgagccgtgttcctgacggtgttgctgaatacagcgtatgggagctgaagctgccgacgctgcgccagcgactgttccgctgcgtgagtatccgtgagaacgacgacggcacgtatgccatcaccgccgtgcagcatgtgccggaaaaagaggccatcgtggataacggggcgcactttgacggcgaacagagtggcacggtgaatggtgtcacgccgccagcggtgcagcacctgaccgcagaagtcactgcagacagcggggaatatcaggtgctggcgcgatgggacacaccgaaggtggtgaagggcgtgagtttcctgctccgtctgaccgtaacagcggacgacggcagtgagcggctggtcagcacggcccggacgacggaaaccacataccgcttcacgcaactggcgctggggaactacaggctgacagtccgggcggtaaatgcgtgggggcagcagggcgatccggcgtcggtatcgttccggattgccgcaccggcagcaccgtcgaggattgagctgacgccgggctattttcagataaccgccacgccgcatcttgccgtttatgacccgacggtacagtttgagttctggttctcggaaaagcagattgcggatatcagacaggttgaaaccagcacgcgttatcttggtacggcgctgtactggatagccgccagtatcaatatcaaaccgggccatgattattacttttatatccgcagtgtgaacaccgttggcaaatcggcattcgtggaggccgtcggtcgggcgagcgatgatgcggaaggttacctggattttttcaaaggcaagataaccgaatcccatctcggcaaggagctgctggaaaaagtcgagctgacggaggataacgccagcagactggaggagttttcgaaagagtggaaggatgccagtgataagtggaatgccatgtgggctgtcaaaattgagcagaccaaagacggcaaacattatgtcgcgggtattggcctcagcatggaggacacggaggaaggcaaactgagccagtttctggttgccgccaatcgtatcgcatttattgacccggcaaacgggaatgaaacgccgatgtttgtggcgcagggcaaccagatattcatgaacgacgtgttcctgaagcgcctgacggcccccaccattaccagcggcggcaatcctccggccttttccctgacaccggacggaaagctgaccgctaaaaatgcggatatcagtggcaatgtgaatgcaaattcagggacgctcaacaatgtcacgattaatgaaaactgtcagattaaagggaaactgtcagccaatcagattgaaggcgatattgtcaaaacggtcagcaagtctttcccccgcacgaacagttatgccagtggcaccatcacggtaagaatcagtgatgatcagaaatttgaccggcaggtcatgataccgccagtgttattccgcggtggtaagcatgagaatttcaacagtaataaccaacagtcatactggtattcaacctgccggttaagagtgacccgcaatggtcaggagatttttaatcagtccacgacggatgctcagggcgtattttcctcagttatagatatgcctgccggacaggggacactgacactgacattcaccgtatcttcatcaggagcgaataactggacaccaacaaccagtatcagcgatctgctggttgtggtgatgaagaaatccacagcaggtatcagtatcagc

SEQ ID NO: 26 - DNA, 3477 bp, Artificial Sequence
  nucleic acid encoding Z2145
atgggtaaaggaagcagtaaggggcataccccgcgcgaagcgaaggacaacctgaagtccacgcagttgctgagtgtgatcgatgccatcagcgaagggccgattgaaggtccggtggatggcttaaaaagcgtgctgctgaacagtacgccggtgctggacactgaggggaataccaacatatccggtgtcacggtggtgttccgggctggtgagcaggagcagactccgccggagggatttgaatcctccggctccgagacggtgctgggtacggaagtgaaatatgacacgccgatcacccgcaccattacgtctgcaaacatcgaccgtctgcgctttaccttcggtgtacaggcactggtggaaaccacctcaaagggtgacaggaatccgtcggaagtccgcctgctggttcagatacaacgtaacggtggctgggtgacggaaaaagacatcaccattaagggcaaaaccacctcgcagtatctggcctcggtggtgatgggtaacctgccgccgcgcccgtttaatatccggatgcgcaggatgacgccggacagcaccacagaccagctgcagaacaaaacgctctggtcgtcatacactgaaatcatcgatgtgaaacagtgctacccgaacacggcactggtcggcgtgcaggtggactcggagcagttcggcagccagcaggtgagccgtaattatcatctgcgcgggcgtattctgcaggtgccgtcgaactataacccgcagacgcggcaatacagcggtatctgggacggaacgtttaaaccggcatacagcaacaacatggcctggtgtctgtgggatatgctgacccatccgcgctacggcatggggaaacgtcttggtgcggcggatgtggataaatgggcgctgtatgtcatcggccagtactgcgaccagtcagtgccggacggctttggcggcacggagccgcgcatcacctgtaatgcgtacctgaccacacagcgtaaggcgtgggatgtgctcagcgatttctgctcggcgatgcgctgtatgccggtatggaacgggcagacgctgacgttcgtgcaggaccgaccgtcggataagacgtggacctataaccgcagtaatgtggtgatgccggatgatggcgcgccgttccgctacagcttcagcgccctgaaggaccgccataatgccgttgaggtgaactggattgacccgaacaacggctgggagacggcgacagagcttgttgaagatacgcaggccattgcccgttacggtcgtaatgttacgaagatggatgcctttggctgtaccagccgggggcaggcacaccgcgccgggctgtggctgattaaaacagaactgctggaaacgcagaccgtggatttcagcgtcggcgcagaagggcttcgccatgtaccgggcgatgttattgaaatctgcgatgatgactatgccggtatcagcaccggtggtcgtgtgctggcggtgaacagccagacccggacgctgacgctcgaccgtgaaatcacgctgccatcctccggtaccgcgctgataagcctggttgacggaagtggcaatccggtcagcgtggaggttcagtccgtcaccgacggcgtgaaggtaaaagtgagccgtgttcctgacggtgttgctgaatacagcgtatgggagctgaagctgccgacgctgcgccagcgactgttccgctgcgtgagtatccgtgagaacgacgacggcacgtatgccatcaccgccgtgcagcatgtgccggaaaaagaggccatcgtggataacggggcgcactttgacggcgaacagagtggcacggtgaatggtgtcacgccgccagcggtgcagcacctgaccgcagaagtcactgcagacagcggggaatatcaggtgctggcgcgatgggacacaccgaaggtggtgaagggcgtgagtttcctgctccgtctgaccgtaacagcggacgacggcagtgagcggctggtcagcacggcccggacgacggaaaccacataccgcttcacgcaactggcgctggggaactacaggctgacagtccgggcggtaaatgcgtgggggcagcagggcgatccggcgtcggtatcgttccggattgccgcaccggcagcaccgtcgaggattgagctgacgccgggctattttcagataaccgccacgccgcatcttgccgtttatgacccgacggtacagtttgagttctggttctcggaaaagcagattgcggatatcagacaggttgaaaccagcacgcgttatcttggtacggcgctgtactggatagccgccagtatcaatatcaaaccgggccatgattattacttttatatccgcagtgtgaacaccgttggcaaatcggcattcgtggaggccgtcggtcgggcgagcgatgatgcggaaggttacctggattttttcaaaggcgagatagggaaaacccatctggctcaggagttgtggactcagattgataacggtcagcttgcgcctgacctggcggaaatcagaacgtccatcacggatgtcagtaatgaaatcacgcagaccgtcaataagaaactggaagaccagagtgcagcgatccagcagatacagaaggttcaggttgatacaaataataacctgaacagcatgtgggcagtgaagctgcagcagatgcaggacggacgcctttatattgcgggtatcggtgccggtattgagaacacctctgacggcatgcagagtcaggtgctgctggcggcagacaggattgcgatgattaatcctgcgaatggcaacacaaagccgatgtttgttggtcagggcgatcagatattcatgaatgaagtgttcctgaaatatctgacggctcccaccattaccagtggcggcaatcctccggcattttccctgacatcagacggaaagctgaccgctaaaaatgcggatatcagtggcagtgtgaatgcgaactccgggacgctcaacaacgtcacgattaacgagaactgtcgggttctgggaaaactgtccgcgaaccagattgaaggcgatctcgttaaaacagtgggcaaagctttcccccgggactcccgtgcaccggaacggtggccatcagggaccattaccgtcagggtttatgacgatcagccgtttgaccggcagattgttattccggcggtggcattcagcggcgctaaacatgagagagagcatactgatatttactcctcatgccgtctgatagtgcggaaaaacggtgctgaaatttataaccgtaccgcgctggataatacgctgatttacagtggcgttattgatatgcctgccggtcacggtcacatgacgctggagttttcggtgtcagcatggctggtgaataactggtatcccacagcaagtatcagcgatttgctggttgtggtgatgaagaaagccaccgcaggcatcagtatcagc

SEQ ID NO: 27 - DNA, 3393 bp, Artificial Sequence
  nucleic acid encoding 1A2
atgggtaaaggaagcagtaaggggcataccccgcgcgaagcgaaggacaacctgaagtccacgcagttgctgagtgtgatcgatgccatcagcgaagggccgattgaaggtccggtggatggcttaaaaagcgtgctgctgaacagtacgccggtgctggacactgaggggaataccaacatatccggtgtcacggtggtgttccgggctggtgagcaggagcagactccgccggagggatttgaatcctccggctccgagacggtgctgggtacggaagtgaaatatgacacgccgatcacccgcaccattacgtctgcaaacatcgaccgtctgcgctttaccttcggtgtacaggcactggtggaaaccacctcaaagggtgacaggaatccgtcggaagtccgcctgctggttcagatacaacgtaacggtggctgggtgacggaaaaagacatcaccattaagggcaaaaccacctcgcagtatctggcctcggtggtgatgggtaacctgccgccgcgcccgtttaatatccggatgcgcaggatgacgccggacagcaccacagaccagctgcagaacaaaacgctctggtcgtcatacactgaaatcatcgatgtgaaacagtgctacccgaacacggcactggtcggcgtgcaggtggactcggagcagttcggcagccagcaggtgagccgtaattatcatctgcgcgggcgtattctgcaggtgccgtcgaactataacccgcagacgcggcaatacagcggtatctgggacggaacgtttaaaccggcatacagcaacaacatggcctggtgtctgtgggatatgctgacccatccgcgctacggcatggggaaacgtcttggtgcggcggatgtggataaatgggcgctgtatgtcatcggccagtactgcgaccagtcagtgccggacggctttggcggcacggagccgcgcatcacctgtaatgcgtacctgaccacacagcgtaaggcgtgggatgtgctcagcgatttctgctcggcgatgcgctgtatgccggtatggaacgggcagacgctgacgttcgtgcaggaccgaccgtcggataagacgtggacctataaccgcagtaatgtggtgatgccggatgatggcgcgccgttccgctacagcttcagcgccctgaaggaccgccataatgccgttgaggtgaactggattgacccgaacaacggctgggagacggcgacagagcttgttgaagatacgcaggccattgcccgttacggtcgtaatgttacgaagatggatgcctttggctgtaccagccgggggcaggcacaccgcgccgggctgtggctgattaaaacagaactgctggaaacgcagaccgtggatttcagcgtcggcgcagaagggcttcgccatgtaccgggcgatgttattgaaatctgcgatgatgactatgccggtatcagcaccggtggtcgtgtgctggcggtgaacagccagacccggacgctgacgctcgaccgtgaaatcacgctgccatcctccggtaccgcgctgataagcctggttgacggaagtggcaatccggtcagcgtggaggttcagtccgtcaccgacggcgtgaaggtaaaagtgagccgtgttcctgacggtgttgctgaatacagcgtatgggagctgaagctgccgacgctgcgccagcgactgttccgctgcgtgagtatccgtgagaacgacgacggcacgtatgccatcaccgccgtgcagcatgtgccggaaaaagaggccatcgtggataacggggcgcactttgacggcgaacagagtggcacggtgaatggtgtcacgccgccagcggtgcagcacctgaccgcagaagtcactgcagacagcggggaatatcaggtgctggcgcgatgggacacaccgaaggtggtgaagggcgtgagtttcctgctccgtctgaccgtaacagcggacgacggcagtgagcggctggtcagcacggcccggacgacggaaaccacataccgcttcacgcaactggcgctggggaactacaggctgacagtccgggcggtaaatgcgtgggggcagcagggcgatccggcgtcggtatcgttccggattgccgcaccggcagcaccgtcgaggattgagctgacgccgggctattttcagataaccgccacgccgcatcttgccgtttatgacccgacggtacagtttgagttctggttctcggaaaagcagattgcggatatcagacaggttgaaaccagcacgcgttatcttggtacggcgctgtactggatagccgccagtatcaatatcaaaccgggccatgattattacttttatatccgcagtgtgaacaccgttggcaaatcggcattcgtggaggccgtcggtcgggcgagcgatgatgcggaaggttacctggattttttcaaaggcaagataaccgaatcccatctcggcaaggagctgctggaaaaagtcgagctgacggaggataacgccagcagactggaggagttttcgaaagagtggaaggatgccagtgataagtggaatgccatgtgggctgtcaaaattgagcagaccaaagacggcaaacattatgtcgcgggtattggcctcagcatggaggacacggaggaaggcaaactgagccagtttctggttgccgccaatcgtatcgcatttattgacccggcaaacgggaatgaaacgccgatgtttgtggcgcagggcaaccagatattcatgaacgacgtgttcctgaagcgcctgacggcccccaccattaccagcggcggcaatcctccggccttttccctgacaccggacggaaagctgaccgctaaaaatgcggatatcagcggtaacgtgaatgcgaactccgggacgctcaacaacgtcacgattaacgagaactgtcgggttctgggaaaattgtccgcgaaccagattgaaggcgatctcgttaaaacagtgggcaaagctttcccccgggactcccgtgcaccggagcggtggccatcaggaaccattaccgtcagggtttatgacgatcagccgtttgaccggcagattgttattccggcggtggcattcagcggcgctaaacatgagaaagagcatactgatatttactcctcatgccgtctgatagtgcggaaaaacggtgctgaaatttataaccgtaccgcgctggataatacgctgatttacagtggcgttattgatatgcctgccggtcacggtcacatgacactggagttttcggtgtcagcatggctggtaaataactggtatcccacagcaagtatcagcgatttgctggttgtggtgatgaagaaagccactgcaggcatcacgattagc

SEQ ID NO: 28 - DNA, 2325 bp, Escherichia phage lambda (Bacteriophage lambda)
atggcagtaaagatttcaggagtcctgaaagacggcacaggaaaaccggtacagaactgcaccattcagctgaaagccagacgtaacagcaccacggtggtggtgaacacggtgggctcagagaatccggatgaagccgggcgttacagcatggatgtggagtacggtcagtacagtgtcatcctgcaggttgacggttttccaccatcgcacgccgggaccatcaccgtgtatgaagattcacaaccggggacgctgaatgattttctctgtgccatgacggaggatgatgcccggccggaggtgctgcgtcgtcttgaactgatggtggaagaggtggcgcgtaacgcgtccgtggtggcacagagtacggcagacgcgaagaaatcagccggcgatgccagtgcatcagctgctcaggtcgcggcccttgtgactgatgcaactgactcagcacgcgccgccagcacgtccgccggacaggctgcatcgtcagctcaggaagcgtcctccggcgcagaagcggcatcagcaaaggccactgaagcggaaaaaagtgccgcagccgcagagtcctcaaaaaacgcggcggccaccagtgccggtgcggcgaaaacgtcagaaacgaatgctgcagcgtcacaacaatcagccgccacgtctgcctccaccgcggccacgaaagcgtcagaggccgccacttcagcacgagatgcggtggcctcaaaagaggcagcaaaatcatcagaaacgaacgcatcatcaagtgccggtcgtgcagcttcctcggcaacggcggcagaaaattctgccagggcggcaaaaacgtccgagacgaatgccaggtcatctgaaacagcagcggaacggagcgcctctgccgcggcagacgcaaaaacagcggcggcggggagtgcgtcaacggcatccacgaaggcgacagaggctgcgggaagtgcggtatcagcatcgcagagcaaaagtgcggcagaagcggcggcaatacgtgcaaaaaattcggcaaaacgtgcagaagatatagcttcagctgtcgcgcttgaggatgcggacacaacgagaaaggggatagtgcagctcagcagtgcaaccaacagcacgtctgaaacgcttgctgcaacgccaaaggcggttaaggtggtaatggatgagactaatcgtaaggcacctctggacagtccggcactgaccggaacgccaacagcaccaaccgcgctcaggggaacaaacaatacccagattgcgaacaccgcttttgtactggccgcgattgcagatgttatcgacgcgtcacctgacgcactgaatacgctgaatgaactggccgcagcgctcgggaatgatccagattttgctaccaccatgactaacgcgcttgcgggtaaacaaccgaagaatgcgacactgacggcgctggcagggctttccacggcgaaaaataaattaccgtattttgcggaaaatgatgccgccagcctgactgaactgactcaggttggcagggatattctggcaaaaaattccgttgcagatgttcttgaataccttggggccggtgagaattcggcctttccggcaggtgcgccgatcccgtggccatcagatatcgttccgtctggctacgtcctgatgcaggggcaggcgtttgacaaatcagcctacccaaaacttgctgtcgcgtatccatcgggtgtgcttcctgatatgcgaggctggacaatcaaggggaaacccgccagcggtcgtgctgtattgtctcaggaacaggatggaattaagtcgcacacccacagtgccagtgcatccggtacggatttggggacgaaaaccacatcgtcgtttgattacgggacgaaaacaacaggcagtttcgattacggcaccaaatcgacgaataacacgggggctcatgctcacagtctgagcggttcaacaggggccgcgggtgctcatgcccacacaagtggtttaaggatgaacagttctggctggagtcagtatggaacagcaaccattacaggaagtttatccacagttaaaggaaccagcacacagggtattgcttatttatcgaaaacggacagtcagggcagccacagtcactcattgtccggtacagccgtgagtgccggtgcacatgcgcatacagttggtattggtgcgcaccagcatccggttgttatcggtgctcatgcccattctttcagtattggttcacacggacacaccatcaccgttaacgctgcgggtaacgcggaaaacaccgtcaaaaacattgcatttaactatattgtgaggcttgcataa

SEQ ID NO: 29 - DNA, 585 bp, Escherichia phage lambda (Bacteriophage lambda)
atggcattcagaatgagtgaacaaccacggaccataaaaatttataatctgctggccggaactaatgaatttattggtgaaggtgacgcatatattccgcctcataccggtctgcctgcaaacagtaccgatattgcaccgccagatattccggctggctttgtggctgttttcaacagtgatgaggcatcgtggcatctcgttgaagaccatcggggtaaaaccgtctatgacgtggcttccggcgacgcgttatttatttctgaactcggtccgttaccggaaaattttacctggttatcgccgggaggggaatatcagaagtggaacggcacagcctgggtgaaggatacggaagcagaaaaactgttccggatccgggaggcggaagaaacaaaaaaaagcctgatgcaggtagccagtgagcatattgcgccgcttcaggatgctgcagatctggaaattgcaacgaaggaagaaacctcgttgctggaagcctggaagaagtatcgggtgttgctgaaccgtgttgatacatcaactgcacctgatattgagtggcctgctgtccctgttatggagtaa

SEQ ID NO: 30 - DNA, 3585 bp, Artificial Sequence
  nucleic acid encoding STF Lambda-WW11.2
atggcagtaaagatttcaggagtcctgaaagacggcacaggaaaaccggtacagaactgcaccattcagctgaaagccagacgtaacagcaccacggtggtggtgaacacggtgggctcagagaatccggatgaagccgggcgttacagcatggatgtggagtacggtcagtacagtgtcatcctgcaggttgacggttttccaccatcgcacgccgggaccatcaccgtgtatgaagattcacaaccggggacgctgaatgattttctctgtgccatgacggaggatgatgcccggccggaggtgctgcgtcgtcttgaactgatggtggaagaggtggcgcgtaacgcgtccgtggtggcacagagtacggcagacgcgaagaaatcagccggcgatgccagtgcatcagctgctcaggtcgcggcccttgtgactgatgcaactgactcagcacgcgccgccagcacgtccgccggacaggctgcatcgtcagctcaggaagcgtcctccggcgcagaagcggcatcagcaaaggccactgaagcggaaaaaagtgccgcagccgcagagtcctcaaaaaacgcggcggccaccagtgccggtgcggcgaaaacgtcagaaacgaatgctgcagcgtcacaacaatcagccgccacgtctgcctccaccgcggccacgaaagcgtcagaggccgccacttcagcacgagatgcggtggcctcaaaagaggcagcaaaatcatcagaaacgaacgcatcatcaagtgccggtcgtgcagcttcctcggcaacggcggcagaaaattctgccagggcggcaaaaacgtccgagacgaatgccaggtcatctgaaacagcagcggaacggagcgcctctgccgcggcagacgcaaaaacagcggcggcggggagtgcgtcaacggcatccacgaaggcgacagaggctgcgggaagtgcggtatcagcatcgcagagcaaaagtgcggcagaagcggcggcaatacgtgcaaaaaattcggcaaaacgtgcagaagatatagcttcagctgtcgcgcttgaggatgcggacacaacgagaaaggggatagtgcagctcagcagtgcaaccaacagcacgtctgaaacgcttgctgcaacgccaaaggcggttaaggtggtaatggatgagactaatcgtaaggcacctctggacagtccggcactgaccggaacgccaacagcaccaaccgcgctcaggggaacaaacaatacccagattgcgaacaccgcttttgtactggccgcgattgcagatgttatcgacgcgtcacctgacgcactgaatacgctgaatgaactggccgcagcgctcgggaatgatccagattttgctaccaccatgactaacgcgcttgcgggtaaacaaccgaagaatgcgacactgacggcgctggcagggctttccacggcgaaaaataaattaccgtattttgcggaaaatgatgccgccagcctgactgaactgactcaggttggcagggatattctggcaaaaaattccgttgcagatgttcttgaataccttggggccggtgagaattcggctaagcaggatggcctgagtctaattggtcagtgtaatagtattgctgatttaagaaacgtagaaccagttatggatggtcaacgcattcttgttaaacagcatactgctggaacaggtaaaggtggtggcacattccaggcaaagctttctggggtaggtttaacagatgataatggtgtaaccgttaagacggttgggggtgcagcatgggttcgtgttggagcagaccgagttaacccatacatgtatggtgcacttggttccggtaatgatgataccttagcagtacaggcatgtcttaatagtggtaaagctaccaatattacagatgttcaccatgtttctaacgttacaatgaataagagcagtgcctctatttatggttctggattgcactataccagactacatcaactacctgctgctgtaggttctttgcttactattgcagatacctgctcattgactgttattgatagtcttgggctgtatggcacgggttataagcagggtactgcttttaccacaggtactcgcggcattgatgtactaaccccaactggtttatccaataactacccagaccatactacttcagacccacgcagagatttggttattactaaggttcatattgcaggttttgatgagtatggccttaatgtggactctggtaactttagcgttactacagagtctgtattgattaaccacattaaacaggttggtgcacgttgtgccactactgactggacttggacaaacatccagattaatacctgcggtaaacaatgtcttattttggatggctgtggcaatggtcgtatcatcggaggtaagtttatctgggccaactggttaccatatggtgcatccggtcagtttccgggtattaccatcaataacagtcaaaacatggttatcaacggtattgaggtgcaggactgtggtggtaatggtattgagtttactgatagctactcaatcagcatgaatggattgaatactaaccgtaacggtattaactctacggcagaattctataacctggtgttcaatcgttctgatgtagaaatcaatggttttgttggtcttaactataaagttaatagtggctcaccagacaattctagctcaggcaaccttaagttcttgtcgagtgattgtaatgtaactcttaatggaaaacttgagacaggctatatgggtatagagttcattggcgatactcgtgtaattcagccaacgtcttcttacattaaacttaacggtctggttaactatgctggttctggtattcagactattaatgaagtacctacttttgatggagttagtactactcctgtatacgtaccaatttcttctgttgttgggcagaccaatggattacgcctgtctcaagctaacaaagacaaggttatttatactcgtaaggttgggccagaaggtgtaacaatggctgctgttattactccaacaattgggggggcagagatatttaacctcatggctattggtacaggttttagctctgcaagtaacagtctgtatttccagttagaaatagctgctgatggttctcagaatgtgtctttacttctgagtggggatggtactacgcagattctgcctggtactttacctgctgcacttaagttacaatccggcgaaccgtatcatgtagctttgggggctaaggctggatacttctggtggagtattttaaatctcaatactggtaagcgtatccgtcgttccttccgaggtgcttacttacctaaaccattcgcttctatctttggtttagtcagttctccagtattcttctctggggcaggtattggtgattcaggatgctctggtgttggtgccaagatttatcttggttccttctctgacgagaatgattatgtatcctctcgttattatagtttggttaacccagttgacccaactaagatgtatagcttccgtatattagacagtactatttaa

SEQ ID NO: 31 - DNA, 2274 bp, Artificial Sequence
  nucleic acid encoding STF Lambda-75
atggcagtaaagatttcaggagtcctgaaagacggcacaggaaaaccggtacagaactgcaccattcagctgaaagccagacgtaacagcaccacggtggtggtgaacacggtgggctcagagaatccggatgaagccgggcgttacagcatggatgtggagtacggtcagtacagtgtcatcctgcaggttgacggttttccaccatcgcacgccgggaccatcaccgtgtatgaagattcacaaccggggacgctgaatgattttctctgtgccatgacggaggatgatgcccggccggaggtgctgcgtcgtcttgaactgatggtggaagaggtggcgcgtaacgcgtccgtggtggcacagagtacggcagacgcgaagaaatcagccggcgatgccagtgcatcagctgctcaggtcgcggcccttgtgactgatgcaactgactcagcacgcgccgccagcacgtccgccggacaggctgcatcgtcagctcaggaagcgtcctccggcgcagaagcggcatcagcaaaggccactgaagcggaaaaaagtgccgcagccgcagagtcctcaaaaaacgcggcggccaccagtgccggtgcggcgaaaacgtcagaaacgaatgctgcagcgtcacaacaatcagccgccacgtctgcctccaccgcggccacgaaagcgtcagaggccgccacttcagcacgagatgcggtggcctcaaaagaggcagcaaaatcatcagaaacgaacgcatcatcaagtgccggtcgtgcagcttcctcggcaacggcggcagaaaattctgccagggcggcaaaaacgtccgagacgaatgccaggtcatctgaaacagcagcggaacggagcgcctctgccgcggcagacgcaaaaacagcggcggcggggagtgcgtcaacggcatccacgaaggcgacagaggctgcgggaagtgcggtatcagcatcgcagagcaaaagtgcggcagaagcggcggcaatacgtgcaaaaaattcggcaaaacgtgcagaagatatagcttcagctgtcgcgcttgaggatgcggacacaacgagaaaggggatagtgcagctcagcagtgcaaccaacagcacgtctgaaacgcttgctgcaacgccaaaggcggttaaggtggtaatggatgagactaatcgtaaagcgcccttaaacagtcctgcgctgaccggaacgccaacaacgccaactgcgcgacagggaacgaataatacccaaatcgcaagcacggctttcgttatggctgcgattgccgcccttgtagattcgtcacctgacgcactgaatacgctgaacgagttagcggcggcgctgggaaacgacccgaattttgcgaccaccatgactaacgcgcttgcgggtaagcaaccgaaagatgccaccctgacggcgctggccgggcttgctactgcggcagacaggtttccgtattttacggggaatgatgtcgccagcctggcaaccctgacaaaagtcgggcgggatattcttgcgaaatcgaccgttgctgccgttatcgaatacctcggtttacgagaactcggcacaagcggggagaaaataccgttactcagtacagcgaatacctggaccaatcgacaaacactcagcggtggcctttctggggaactgtccggcaatgccgctactgcaacaaagctgaaaacagcacgaaaaattgctggagttggttttgatggttctagcgatatttcaattagtgccaaaaatgtcaatgcatttgcactccgacaaacaggtaatactgttaatggtgatacatccgttggatggaattgggatagtggtgcatataacgccctgattggtggtgcatctgcattaattcttcactttaatataaatgctggtagctgtcctgccgtacaattccgtgtgaattataaaaatggtggcatttcctacaggtcggctcgtgatggttatgggtttgaattaggttggtcagatttctataccacgacacgaaaaccttcagcgggagatgttggtgcatatacgcaggcagaatgtaactcaaggtttattacaggtattcgccttggcggtctgtcatctgttcagacatggaatggtcccggctggtctgacaggtcaggttatgtcgttacgggttcagttaacggaaaccgtgatgaattaattgatacaacacaggcaaggccaattcagtattgcattaatgggacgtggtataacgcggggagtatttaa

SEQ ID NO: 32 - DNA, 534 bp, Artificial Sequence
  nucleic acid encoding lambda-STF75 accessory protein
atgatgcacttaaaaaacattattgctggcaaccctaaaacaaaagagcaataccagctaacaaagcaatttaacatcaaatggctttattcagatgatggaaaaaactggtatgaggaacaaaagaatttccagccagacactttgaaaatggtctatgaccataacggcgttattatttgtattgaaaaggatgtttcagcaattaatccggaaggcgcaagcgtcgttgaattacctgatattacagcaaatcgccgggctgatatttcggggaaatggatgttcaaagatggcgtggtgataaagcgaacttataccgaggaagagcagagacaacaagcggaaaatgaaaagcaaagcctgttgcaacttgtcagggataaaacccagctatgggactcacagctacggctgggcatcatttccgacgagaataagcagaaattaaccgagtggatgctctttgcgcagaaagtcgaatctacagacacctccagcctgccagtaacgtatcccgaacaaccagaatga

SEQ ID NO: 33 - DNA, 2238 bp, Artificial Sequence
  nucleic acid encoding STF  Lambda-EB6
atggcagtaaagatttcaggagtcctgaaagacggcacaggaaaaccggtacagaactgcaccattcagctgaaagccagacgtaacagcaccacggtggtggtgaacacggtgggctcagagaatccggatgaagccgggcgttacagcatggatgtggagtacggtcagtacagtgtcatcctgcaggttgacggttttccaccatcgcacgccgggaccatcaccgtgtatgaagattcacaaccggggacgctgaatgattttctctgtgccatgacggaggatgatgcccggccggaggtgctgcgtcgtcttgaactgatggtggaagaggtggcgcgtaacgcgtccgtggtggcacagagtacggcagacgcgaagaaatcagccggcgatgccagtgcatcagctgctcaggtcgcggcccttgtgactgatgcaactgactcagcacgcgccgccagcacgtccgccggacaggctgcatcgtcagctcaggaagcgtcctccggcgcagaagcggcatcagcaaaggccactgaagcggaaaaaagtgccgcagccgcagagtcctcaaaaaacgcggcggccaccagtgccggtgcggcgaaaacgtcagaaacgaatgctgcagcgtcacaacaatcagccgccacgtctgcctccaccgcggccacgaaagcgtcagaggccgccacttcagcacgagatgcggtggcctcaaaagaggcagcaaaatcatcagaaacgaacgcatcatcaagtgccggtcgtgcagcttcctcggcaacggcggcagaaaattctgccagggcggcaaaaacgtccgagacgaatgccaggtcatctgaaacagcagcggaacggagcgcctctgccgcggcagacgcaaaaacagcggcggcggggagtgcgtcaacggcatccacgaaggcgacagaggctgcgggaagtgcggtatcagcatcgcagagcaaaagtgcggcagaagcggcggcaatacgtgcaaaaaattcggcaaaacgtgcagaagatatagcttcagctgtcgcgcttgaggatgcggacacaacgagaaaggggatagtgcagctcagcagtgcaaccaacagcacgtctgaaacgcttgctgcaacgccaaaggcggttaagattgcgatggataatgccaatgcccgtctggcaaaagaccggaacggagcagatattcccaataagccgctgtttatccaaaacctcggtttacaggaaacggtaaacaaggctggtaacgccgttcaaaagacaggcgataccttgtccggcggacttacttttgaaaacgactcaatccttgcctggattcggaatactgactgggcaaagattggatttaaaaatgatgccgacagcgacactgattcatacatgtggtttgaaacaggcgacaacggcaatgaatatttcaaatggagaagccgccagagcaccacaacaaaagacctgatgaatcttaaatgggatgctttgtatgttcttgtcaatgccattgtaaatggcgaagtcatatcaaaatcagcaaacggcctacgtattgcttatggtaattacggattctttattcgtaatgatggttcaaatacatacttcatgttgacaaactccggtgacaacatggggacttataacggattaaggccattatggattaataacgctactggcgctgtttcgatggggcgtggtcttaatgtttcaggggagacactttcagaccgttttgctattaacagcagtaatggtatgtggattcagatgcgcgataacaacgctatctttgggaaaaatatagttaacactgatagcgctcaggcgttacttcgccagaatcacgccgaccgaaagttcatgataggtggactggggaacaagcaatttggcatctacatgattaataactcaaggacagccaatggcaccgatggtcaggcgtacatggataataacggtaactggctttgtggtgcgcaagttattcccggcaattatggcaattttgactcacgctatgtgagagatgtccgacttggcacacgtgttgttcaattgatggcgcgtggtggtcgttatgaaaaagccggacacgcaattaccggattaagaatcattggtgaagtagatggcgatgatgaagccatcttcaggccaatacaaaaatacatcaatggcacatggtataacgtcgcacaggtgtaa

SEQ ID NO: 34 - DNA, 528 bp, Artificial Sequence
  nucleic acid encoding Lambda-EB6 accessory protein
atgcagcatttaaaaaatattaagtctggaaatcctaaaacgaaagaacaatatcagctaacaaagaattttgatgttatctggttatggtccgaagacggtaaaaactggtatgaagaagtaaataactttcaggacgacaccataaagattgtatacgacgaaaataatattattgttgccataaccaaagatgcctcaacgcttaatcccgaaggctttagcgtcgttgagattccagatataacagccaatcgtcgtgccgatgattcagggaagtggatgtttaaggacggagctgtggttaaacggatttatacggcagacgagcaacaacaacaggccgaatcacaaaaggccgcgttactttccgaagcagaaaacgttattcagccactggaacgcgctgtcagactgaatatggcgacggatgaggaacgcgcacgactggagtcatgggaacgctacagtgttctggtcagccgtgtggatacggcaaagccagaatggccacaaaagcctgaataa

SEQ ID NO: 35 - DNA, 2274 bp, Artificial Sequence
  nucleic acid encoding STF Lambda-23
atggcagtaaagatttcaggagtcctgaaagacggcacaggaaaaccggtacagaactgcaccattcagctgaaagccagacgtaacagcaccacggtggtggtgaacacggtgggctcagagaatccggatgaagccgggcgttacagcatggatgtggagtacggtcagtacagtgtcatcctgcaggttgacggttttccaccatcgcacgccgggaccatcaccgtgtatgaagattcacaaccggggacgctgaatgattttctctgtgccatgacggaggatgatgcccggccggaggtgctgcgtcgtcttgaactgatggtggaagaggtggcgcgtaacgcgtccgtggtggcacagagtacggcagacgcgaagaaatcagccggcgatgccagtgcatcagctgctcaggtcgcggcccttgtgactgatgcaactgactcagcacgcgccgccagcacgtccgccggacaggctgcatcgtcagctcaggaagcgtcctccggcgcagaagcggcatcagcaaaggccactgaagcggaaaaaagtgccgcagccgcagagtcctcaaaaaacgcggcggccaccagtgccggtgcggcgaaaacgtcagaaacgaatgctgcagcgtcacaacaatcagccgccacgtctgcctccaccgcggccacgaaagcgtcagaggccgccacttcagcacgagatgcggtggcctcaaaagaggcagcaaaatcatcagaaacgaacgcatcatcaagtgccggtcgtgcagcttcctcggcaacggcggcagaaaattctgccagggcggcaaaaacgtccgagacgaatgccaggtcatctgaaacagcagcggaacggagcgcctctgccgcggcagacgcaaaaacagcggcggcggggagtgcgtcaacggcatccacgaaggcgacagaggctgcgggaagtgcggtatcagcatcgcagagcaaaagtgcggcagaagcggcggcaatacgtgcaaaaaattcggcaaaacgtgcagaagatatagcttcagctgtcgcgcttgaggatgcggacacaacgagaaaggggatagtgcagctcagcagtgcaaccaacagcacgtctgaaacgcttgctgcaacgccaaaggcggttaaggtggtaatggatgagactaatcgtaaagccccattaaacagcccggcgctgaccggaacgccaacaacaccaactgcgcgacagggaacgaataatacccaaatcgcaagcacggctttcgttatggctgcgattgccgcccttgtagattcgtcacctgatgcactgaacacgctgaacgagctggctgcggcgttgggcaacgacccgaattttgcgaccaccatgactaacgcgcttgcgggtaagcaaccgaaagatgccaccctgacggcgctggccgggcttgctactgcggcagacaggtttccgtattttacggggaatgatgtcgccagcctggcaaccctgacaaaagtcgggcgggatattcttgcgaaatcgaccgttgctgccgttatcgaatacctcggtttacgagaactcggcacaagcggggagaaaataccgttactcagtacagcgaatacctggaccaatcgacaaacattcagcggtggcctttctgggggactgtccggcaatgccgctactgcaacaaagctgaaaacagcacgaaaaattgctggagttggttttgatggttctagcgatatttcaattagtgccaaaaatgtcaatgcatttgcactccgacaaacaggtaatactgttaatggtgatacatccgttggatggaattgggatagtggtgcatataacgccctgattggtggtgcatctgcattaattcttcactttaatataaatgctggtagctgtcctgccgtacaattccgtgtgaattataaaaatggtggcatttcctacaggtcggctcgtgatggttatgggtttgaattaggttggtcagatttctataccacgacacgaaaaccttcagcgggagatgttggtgcatatacgcgggcagaatgtaactcaaggtttattacaggtattcgccttggcggtctgtcatctgttcagacatggaatggtcccggctggtctgacaggtcaggttatgtcgttacgggttcagttaacggaaaccgtgatgaattaattgatacaacacaggcaaggccaattcagtattgcattaatgggacgtggtataacgcggggagtatttaa

SEQ ID NO: 36 - DNA, 531 bp, Artificial Sequence
  nucleic acid encoding lambda-STF23 accessory protein
atgatgcacttaaaaaacattactgctggcaaccctaaaacaaaagagcaataccagctaacaaagcaatttaacatcaaatggctttattcagatgatggaaaaaactggtatgaggaacaaaagaatttccagccagacactttgaaaatggtctatgaccataacggcgttattatttgtattgaaaaggatgtttcagcaattaatccggaaggcgcaagcgtcgttgaattacctgatattacagcaaatcgccgggctgatatttcggggaaatggttgttcaaagatggcgtagtgataaagcgaacttataccgaggaagagcagaggcaacaagcggaaaatgaaaagcaaagcctgttgcaacttgtcagggataaaacccagctatgggactcacagctacggctgggcatcatttccgacgagaataaacaaaaattaaccgagtggatgctctatgcgcagaaagtcgaatctacagacacctccagcctgccagtaacgtttcccgaacaaccagaa

SEQ ID NO: 37 - protein, 6 aa, Artificial Sequence
  Insertion site SAGDAS
SAGDAS

SEQ ID NO: 38 - protein, 6 aa, Artificial Sequence
  Insertion site ADAKKS
ADAKKS

SEQ ID NO: 39 - protein, 6 aa, Artificial Sequence
  Insertion site MDETNR
MDETNR

SEQ ID NO: 40 - protein, 6 aa, Artificial Sequence
  Insertion site SASAAA
SASAAA

SEQ ID NO: 41 - protein, 6 aa, Artificial Sequence
  Insertion site GAGENS
GAGENS

SEQ ID NO: 42 - protein, 6 aa, Artificial Sequence
  Insertion point 1 Figure 7
YLDFFK

SEQ ID NO: 43 - protein, 6 aa, Artificial Sequence
  Insertion point 2 Figure 7
NADISG

SEQ ID NO: 44 - protein, 1186 aa, Artificial Sequence
  Chimeric STF-V10
MAVKISGVLKDGTGKPVQNCTIQLKARRNSTTVVVNTVGSENPDEAGRYSMDVEYGQYSVILQVDGFPPSHAGTITVYEDSQPGTLNDFLCAMTEDDARPEVLRRLELMVEEVARNASVVAQSTADAKKSAGDASASAAQVAALVTDATDSARAASTSAGQAASSAQEASSGAEAASAKATEAEKSAAAAESSKNAAATSAGAAKTSETNAAASQQSAATSASTAATKASEAATSARDAVASKEAAKSSETNASSSAGRAASSATAAENSARAAKTSETNARSSETAAERSASAAADAKTAAAGSASTASTKATEAAGSAVSASQSKSAAEAAAIRAKNSAKRAEDIASAVALEDADTTRKGIVQLSSATNSTSETLAATPKAVKVVMDETNRKAPLDSPALTGTPTAPTALRGTNNTQIANTAFVLAAIADVIDASPDALNTLNELAAALGNDPDFATTMTNALAGKQPKNATLTALAGLSTAKNKLPYFAENDAASLTELTQVGRDILAKNSVADVLEYLGAGENSAANDGFKFIGQCPDILTLRTIEPEKNGQRITLRQHTIGTGLGGGVFRAVLDGTGYTDDDGVVIKTAGGSVWLRVNADKVNPFMFGATGVADDTAALQKMLECGRAAELGTNVWKASNLELNNKSCSLSGSGLHVSRIEQISGATGALLTITQDCSLIYLSDCGLYGDGITAGTSGVTMETGNPGGAPSYPFNTAPDVRRDLYISNVHITGFDELGFDYPETNFSVSTHGLFIRNIKKTGAKIGTTDFTWTNLQIDTCGQECLVLDGAGNCRIIGAKLIWAGSENETPYSGLRISNSQNVNMTGVELQDCAYDGLYIKNSTVAISGLNTNRNSASSNLSYHNMVFENSIVTVDGYVCRNYAATSLYDLNSQAGNVRCIGSDSTVLINGIYESEVNSERLMGDNNLIQPYSGDLIINGLKNYYTYTGSVKNNIPTFDGVVTTATYVSAPSILGQGNMLKLTQSNKDKLLFSDKVSRHGCTIGLVLIPSFTGATTMTAFTLGSGYSPSGNSAVMQFIVNSSGVQTIAILLSGDGITQTLTSDLTTEQALASGGVYHFAMGFAPGRLWWSIIDINTGRRIRRAYRQPDLHAAFNSIFNSGTSSITAFSGPLAGDIACEGAGSHVYVGGFSSESDYAASRMYGLFTPVDLDKQYSFRTLNGNI

SEQ ID NO: 45 - protein, 1186 aa, Artificial Sequence
  Chimeric STF-V10f
MAVKISGVLKDGTGKPVQNCTIQLKARRNSTTVVVNTVGSENPDEAGRYSMDVEYGQYSVILQVDGFPPSHAGTITVYEDSQPGTLNDFLCAMTEDDARPEVLRRLELMVEEVARNASVVAQSTADAKKSAGDASASAAQVAALVTDATDSARAASTSAGQAASSAQEASSGAEAASAKATEAEKSAAAAESSKNAAATSAGAAKTSETNAAASQQSAATSASTAATKASEAATSARDAVASKEAAKSSETNASSSAGRAASSATAAENSARAAKTSETNARSSETAAERSASAAADAKTAAAGSASTASTKATEAAGSAVSASQSKSAAEAAAIRAKNSAKRAEDIASAVALEDADTTRKGIVQLSSATNSTSETLAATPKAVKVVMDETNRKAPLDSPALTGTPTAPTALRGTNNTQIANTAFVLAAIADVIDASPDALNTLNELAAALGNDPDFATTMTNALAGKQPKNATLTALAGLSTAKNKLPYFAENDAASLTELTQVGRDILAKNSVADVLEYLGAGENSAANDGFAFIGQCPDILTLRTIEPEKNGQRITLRQHTIGTGLGGGVFRAVLDGTGYTDDDGVVIKTAGGSVWLRVNADKVNPFMFGATGVADDTAALQKMLECGRAAELGTNVWKASNLELNNKSCSLSGSGLHVSRIEQISGATGALLTITQDCSLIYLSDCGLYGDGITAGTSGVTMETGNPGGAPSYPFNTAPDVRRDLYISNVHITGFDELGFDYPETNFSVSTHGLFIRNIKKTGAKIGTTDFTWTNLQIDTCGQECLVLDGAGNCRIIGAKLIWAGSENETPYSGLRISNSQNVNMTGVELQDCAYDGLYIKNSTVAISGLNTNRNSASSNLSYHNMVFENSIVTVDGYVCRNYAATSLYDLNSQAGNVRCIGSDSTVLINGIYESEVNSERLMGDNNLIQPYSGDLIINGLKNYYTYTGSVKNNIPTFDGVVTTATYVSAPSILGQGNMLKLTQSNKDKLLFSDKVSRHGCTIGLVLIPSFTGATTMTAFTLGSGYSPSGNSAVMQFIVNSSGVQTIAILLSGDGITQTLTSDLTTEQALASGGVYHFAMGFAPGRLWWSIIDINTGRRIRRAYRQPDLHAAFNSIFNSGTSSITAFSGPLAGDIACEGAGSHVYVGGFSSESDYAASRMYGLFTPVDLDKQYSFRTLNGNI

SEQ ID NO: 46 - protein, 1186 aa, Artificial Sequence
  Chimeric STF-V10a
MAVKISGVLKDGTGKPVQNCTIQLKARRNSTTVVVNTVGSENPDEAGRYSMDVEYGQYSVILQVDGFPPSHAGTITVYEDSQPGTLNDFLCAMTEDDARPEVLRRLELMVEEVARNASVVAQSTADAKKSAGDASASAAQVAALVTDATDSARAASTSAGQAASSAQEASSGAEAASAKATEAEKSAAAAESSKNAAATSAGAAKTSETNAAASQQSAATSASTAATKASEAATSARDAVASKEAAKSSETNASSSAGRAASSATAAENSARAAKTSETNARSSETAAERSASAAADAKTAAAGSASTASTKATEAAGSAVSASQSKSAAEAAAIRAKNSAKRAEDIASAVALEDADTTRKGIVQLSSATNSTSETLAATPKAVKVVMDETNRKAPLDSPALTGTPTAPTALRGTNNTQIANTAFVLAAIADVIDASPDALNTLNELAAALGNDPDFATTMTNALAGKQPKNATLTALAGLSTAKNKLPYFAENDAASLTELTQVGRDILAKNSVADVLEYLGAGENSAANDGAAHIGQCPDILTLRTIEPEKNGQRITLRQHTIGTGLGGGVFRAVLDGTGYTDDDGVVIKTAGGSVWLRVNADKVNPFMFGATGVADDTAALQKMLECGRAAELGTNVWKASNLELNNKSCSLSGSGLHVSRIEQISGATGALLTITQDCSLIYLSDCGLYGDGITAGTSGVTMETGNPGGAPSYPFNTAPDVRRDLYISNVHITGFDELGFDYPETNFSVSTHGLFIRNIKKTGAKIGTTDFTWTNLQIDTCGQECLVLDGAGNCRIIGAKLIWAGSENETPYSGLRISNSQNVNMTGVELQDCAYDGLYIKNSTVAISGLNTNRNSASSNLSYHNMVFENSIVTVDGYVCRNYAATSLYDLNSQAGNVRCIGSDSTVLINGIYESEVNSERLMGDNNLIQPYSGDLIINGLKNYYTYTGSVKNNIPTFDGVVTTATYVSAPSILGQGNMLKLTQSNKDKLLFSDKVSRHGCTIGLVLIPSFTGATTMTAFTLGSGYSPSGNSAVMQFIVNSSGVQTIAILLSGDGITQTLTSDLTTEQALASGGVYHFAMGFAPGRLWWSIIDINTGRRIRRAYRQPDLHAAFNSIFNSGTSSITAFSGPLAGDIACEGAGSHVYVGGFSSESDYAASRMYGLFTPVDLDKQYSFRTLNGNI

SEQ ID NO: 47 - DNA, 11615 bp, Artificial Sequence
  Example of payload sequence
gtttgcaataagggacaagttacgagtgtagacacgcagaattatccagcctttagtctttaggaaggcaaagctattgtacgcggtagccgtcgtagcaatttaccaactgtagaattattggacacacgtaacaagggcttacagttgaagtttaataaggtcacacgcaaaaccgctaaggaataatcgcaccgttagcgaaagaatatttcagagcggttagtaaaggttgagtaaagtgagattccaaagtgagcctttataaaaagtaaagagctataataaaaccgtcgatcggaaaacaatcgcctgaaatctcaagcacgttgccctttctaacgtcgctaaggtttcgtaaacccgtttgattaggaagaagaataagtaacccgattaggtttgagatcgcgggttatcggtttggattaaaagtggataccagcggagtcaacgccgacgcaaacgtacagtgatccaatcctgttccacggtcaagcacaatcagctagcaagatcttggaatagagtcgttgcaccgctttgatttacatgctctccattgcacaacattccggaaggactggcttctctgccatgatcggataatgaaaaacatcagtatgccctgtcatttttctttgggtgtcctcaaataattgccctcacgttatcgtatgtgacgcgctcatctatgctcgaagtattccttgttctcccatcttttaatagaaagtctttaatgaacgtgtcgttacgcagtgtatgaactcttgttttatagggcagactttggcgtggcctaagtgtgttcgataagaaggcaaggacaactagctgacgcgctgtaatacggatattatggcacggttgatacaaacgctgatatcctgatttgctaatgtgcccaacactttagttgagtgccacgttccgactacaagttgcttcaagaggggaatttggatttggcaatagccccccgtttctacctcaagaggcgacgagtattaaccgcgccagctttcggcacaagggccaaagaagattccaatttcttattcccgaataacctccgaatccctgcgggaaaatcaccgaccgaatagcctagaagcaagggggaacagataggtataattagcttaagagagtaccagccgtgacaacaccgtagtaaccacaaacttacgctggggcttctttggcggatttttacagatactaacaaggtgatttgaagtaccttagttgaggatttaaacgcgctatccggtagtctacaaattgggaaataccgttcaaagagggctagaattacttaaaagccttcacaccgcctgcgctatacgcgcccactctcccgtttatccgtccaagcggaagcagggcgaacttccgctaagatattcttacgtgtaacgtagctaagtatcccaaatagctggcgtacgcgttgaacaccgcctagaggatcgggagtcgccggacgagcgtgttattggggacttacgccagcgtagactacaacgcgcccagattaaccctgcacgtattgccttgaataacgtactaatctctccggctctcgacaatctatcgagcgactcgattatcaacgggtgtcttgcagttctaatctcttgcccccgcccgtaatagcctccaagtgattcaagatagtaaagggcaagagcttattcggcgttgaaggatagcggactttcggtcaaccacaattccccactcgacaaaaccagccgtgcgaagaactctgaaagtacaagcaacccaagagggctgagcctaaactcagctaattcctaagtgagctaaagactcgaagtgacagctattaataaatagagcgggaacgtcgaacggtcgtgaaagtaatagtacaacgggtattaacttactgaggatattgcttgaagctgtaccgttttattgggtgaacgaataagatccagcaattcagccaaagaagctaccaatttttagtttaagagtgtcacgtctgacctcgcgggtggatagccgaacgtagagcttacgagccagcggaaacagtagccgcaggataagtaaggggagtaagtgatcgaacgaatcagaagtgacaatatacttaggctggatctcgtcccgtgaatcccaaccctcaccaactacgagataagaggtaagccagaaatcggcatggtggcgaccaacgactgttccccccctgtaactaatcgttccgtcaaaacctgacttacttcaaggccaattccaagcgcaaacaataccgtcctagttcttcggttaagtttccgaagtaggagtgagcctacctccgtttgcgtcttgttaccactgacccagctatttactttgtattgcctgcaatcgaatttctgaactctcagatagtggggataacgggaaagttcctatatttgcgaactaacttagccgtccacctcgaagctacctactcacacccaccccgcgcggggtaaataaggcactaatcccagcttagagcttgcgtagcacttagccacaagttaattaacagttgtctggtagtttggcggtattagcgagatcctagaagcaaggcagagttagttctaacctaaagccacaaataagacaggttgccaaagcccgccggaaattaaatcttgctcagttcggtaacggagtttccctcccgcgtacttaattcccaataagaaacgcgcccaagtcctatcaggcaaaattcagccccttcccgtgttagaacgagggtaaaaatacaagccgattgaacaagggttgggggcttcaaatcgtcgtttaccccactttacaacggagggtaagtagttcaccctatagtacgaagcagaactatttcgaggggcgtgcaataatcgaatcttctgcggttgacttaacacgctagggacgtgccctcgattcagtcgcaggtactcctactcagactgcctcacacccagctagtcactgagcgataaaattgacccgccctctaaggtagcgagtacgtcccaaagggctccggacagggctatataggagagtttgatctcgccccgacaactgcaaccctcaactcccttagataatattgttagccgaagttgcacgacccgccgtccacggactgctcttagggtgtggctccttaatctgacaacgtgcaacccctatcgagggcgattgtttctgcgaaaggtgttgtcctaatagtcgcgacatttggcccttgtaggtgtgaaaccacttagcttcgcgccgtagtcctaaaggcccacctattgactttgtttcgggtagcactaggaatcttaacaatttgaatttggacgtggaacgcgtacaccttgatcttcgaataattctagggatttggaagtcctctacgttgacacacctacaatgctccaagtaaatatacgaataacgcgggcctcgcggagccgttccgaatcgtcacgtgttcgtttactgttaattggtggcaaataagcaatatcgtagtccgtcaggcccagccctgttatccacggcgttatttgtcaaattgcgtagaactggattgactgcctgacaatacctaattatcggtacgaagtccccgaatctgtccggctatttcactaatactttccaaacgccccgtatccaagaagaacgaatttatccacgctcccgtctttgggacgaataccgctacaagtggacagaggatcggtacgggcctctaataaatccaacactctacgccctcttcaagagctagaagaacagggtgcagttggaaagggaattatttcgtaaggcgagccaataccgtaattaattcggaagagttaacacgattggaagtaggaatagtttctaaccacggttactaatcctaataacggaacgctgtctgatagattagtgtcagcgctcactaccaaagaaaaataaaaagacgctgaaaagcgtctttttatttttcggtccagtgtaactcaggcaaaagcacgtaatattcgtactcaccaaacgaaactcatccggcgcatcgcgcttcttcctccgtaagcgtcacccccattacttaaagagtgcatgtgcatattttgttatcaataaaaaaggccgcgatttgcggccttattgttcgtcttgccggattagatagctaccggtgctttaatacccggatgcggatcatagccttcgatttcgaagtcctcaaaacgataatcgaagatgctttccggtttgcgtttgataatcagtttcgggagcgggcgtggctcacggcttaattgtaaatgcgtctgatccatgtgatttgagtacaggtgagtatccccaccagtccaaacaaagtcaccaacttccagatcacactgctgtgccatcatatgaactaataaggcgtaggaggcaatgttaaacggtaagcccagaaacacgtcgcaagaacgctggtacagttggcacgataacttaccatccgcaacatagaattgaaagaaggcatgacacggtgctaaagccattttgtctaattcccccacgttccatgcggacacgataatccggcgagagtccggatcatttttcagttggttaagaacggtagtgatctgatcaatatgccgaccatccggcgtaggccatgcacgccattgcttaccatacactggccctaagtcaccgttttcatctgcccactcatcccagatggtaacgttattctcgtgcaggtacgcaatgttcgtatcgccttgcagaaaccataataactcgtgaataatagaacggaggtggcaacgcttggtagtgaccagcgggaaaccgtcttgcaggttgaaacgcatctgatgaccaaagatagacagcgtaccagtgccagtacgatcattcttctgagtgccttcgtccagcactttttgcatcagttccagatactgtttcattttagcttccttagcttgcgaaatctcgataactcaaaaaatagtagtgatcttatttcattatggtgaaagttgtcttacgtgcaacattttcgcaaaaagttggcgctttatcaacactgtccgaatgacaaatggttacaattattgaacacccttcggggtgtttttttgtttctggtttcccgaggccgaacttttgttgcaatggctgtctaccctgtctacctgagtaaagaaaaatacatttaattcagtatattaacttgggtagacagcctttttttactgtctaccttctgtctaccctctctacctgattttacctgaatcagacagggaggtagacacggggtagacagtggataaaagcactctaccccactgaaagcagtgccattactggcatggttgccagtaaggttgataaggtagacaaggggagggacaactcaaaactttttaaacgagggggtaaaacgcagatcaaaacgatctcaagaagatcatcttattaatcagataaaatatttctagatttcagtgcaatttatctcttcaaatgtagcaccggcgcgccgtgaccaattattgaaggccgctaacgcggcctttttttgtttctggtttcccgaatagagcgacttctccccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaagaacctgtaaagtgataatgattatcatctacatatcacaacgtgcgtaaagggtaagtatgaaggtcgtgtactccatcgctaccaaattccagaaaacagacgctttcgagcgtcttttttcgttttggtcacgacgtacggtggaagattcgttaccaattgacagctagctcagtcctaggtatatacatacatgcttgtttgtttgtaaactactgttttcattaaagaggagaaaggaagccatgtccatctatcaggagtttgttaacaagtattccctgtctaaaaccctgcgttttgaactgatcccgcagggcaaaactttggaaaacattaaagcgcgtggcctgattctggatgacgaaaaacgtgcaaaggattacaagaaagctaaacagatcatcgacaaatatcaccagttctttatcgaagaaattctgtcctcggtgtgcatcagtgaggatctgttacagaattattctgatgtatactttaaacttaaaaagtccgatgacgataatctgcaaaaagatttcaagtcagccaaagataccatcaagaaacagatctcagaatatattaaagatagcgaaaagttcaaaaacctgtttaaccaaaacctcattgatgctaagaaaggccaagaatctgacctgatcttatggctgaaacagagcaaagataacggcattgaactgttcaaagctaatagcgacatcaccgatattgatgaagcgctcgaaatcatcaagtctttcaaaggctggacgacgtatttcaaaggttttcatgaaaaccgtaagaatgtatattcgagcaacgatattccgacctctattatttatcgtatcgtggacgacaacctgccgaagtttctggaaaacaaagcgaaatatgaatctctgaaagacaaagcaccggaagctattaactatgaacagatcaagaaagatctggcggaagaactgaccttcgacatcgactataaaacctccgaagttaaccagcgtgttttctcactggacgaggttttcgaaatcgctaatttcaacaattacctgaatcaatctggcatcaccaaattcaacaccattattggtggcaaatttgttaacggcgaaaacaccaagcgtaagggcatcaacgaatacattaacctctatagccaacaaatcaacgacaaaaccctgaaaaagtataaaatgtccgttctgtttaaacagattttatcggacaccgaatctaaatccttcgtaattgataaactggaagatgatagcgacgttgtcaccacgatgcagagcttttatgagcagattgcggcgttcaaaaccgtcgaagagaaatctattaaagaaactctgtccctgctctttgacgacctcaaagcgcagaaactagatctgtctaagatttactttaaaaacgacaaatctctgaccgatctcagtcaacaagttttcgatgactatagcgtgatcggcacggcagttttggaatacatcacccaacaaatcgcgccgaaaaatctggacaacccgtccaagaaggaacaggaactgattgcaaagaaaacagaaaaagctaaatacctgagcttagaaactatcaaactggcacttgaggaatttaataaacatcgtgatattgataaacagtgtcgttttgaggaaattctggcgaactttgcggcaatcccgatgatcttcgacgaaattgctcaaaacaaagacaatctggcgcagatctctatcaagtaccagaatcagggtaagaaagatctgcttcaagcatctgcggaggacgatgtcaaagcaattaaagacttattagatcagacgaataacttattacacaagctcaaaatcttccacatcagccagagcgaggacaaggcgaacattctggataaagatgaacacttctatctggtgttcgaagaatgttacttcgaactggcaaacatcgtacctctctacaataaaatccgcaactacatcacgcagaagccttacagtgacgagaaattcaaactgaacttcgaaaacagcacgctggcgaacggctgggataagaacaaagagccggacaacaccgcaatcctgttcatcaaagacgacaaatactatctgggcgtaatgaacaagaagaacaacaagatcttcgacgataaagcgatcaaagaaaacaagggtgaaggctataagaaaatcgtgtacaagctcctgccgggtgcgaacaaaatgttaccgaaagtgttcttttccgcgaaaagcatcaaattctacaacccgtctgaggatattctgcgcatccgcaatcatagcacgcacactaaaaacggtagcccgcagaaagggtatgaaaaattcgaatttaatatagaggactgccgtaaattcatcgacttctataaacagagcatttccaaacatccggaatggaaagacttcggcttccgtttctctgacactcagcgctataatagcatcgacgagttctaccgcgaagtggagaatcagggctataaactgaccttcgagaacattagtgagtcgtacatcgactccgttgtgaatcagggtaaactgtacctgtttcagatctataataaagactttagcgcgtacagcaaaggccgcccgaatctgcacaccctttactggaaagcattatttgacgaacgtaacctgcaagatgtggtgtataaactgaacggtgaggcggaacttttctaccgtaaacagagtatcccgaagaaaatcacgcatccggcaaaagaagctattgccaacaaaaacaaagacaacccgaagaaagaaagtgtattcgaatatgacctgatcaaagataaacgtttcaccgaagataagttctttttccactgtccgattaccatcaacttcaaatctagcggtgcgaacaagttcaacgatgaaattaacttattactgaaagagaaagctaatgacgtacacatcttatctattgatcgcggtgaacgtcatttagcatactatacactggtagacggtaaaggtaatattattaaacaggatactttcaatattatcggtaatgaccgtatgaaaaccaactatcacgataagctggcggcgatcgaaaaagatcgtgattctgcgcgtaaagattggaagaaaattaacaatatcaaagaaatgaaagaaggctatctgagccaagtggtgcacgagatcgcaaaactggtgattgaatataacgctatcgtggttttcgaagatctgaactttggttttaaacgtggtcgcttcaaagtagaaaaacaggtgtaccaaaaactggaaaaaatgctgattgaaaaactgaactatctggtttttaaagacaacgaatttgacaaaacgggtggcgtactccgtgcctatcagcttaccgctccgttcgaaacgtttaagaaaatgggtaaacaaacggggattatctattatgtgccagccggtttcacctccaagatttgtccagttacgggcttcgttaaccagctttacccgaaatacgagagcgttagcaaatctcaagaatttttcagcaaattcgacaagatctgctataatctggataaaggctatttcgagttcagctttgattacaaaaacttcggcgataaagcggctaaaggtaagtggactattgctagctttggtagccgtctgattaactttcgcaactccgacaaaaaccataattgggacacgcgtgaagtgtatccgaccaaagaactggaaaaattactgaaagactattccatcgaatatggtcatggggagtgcattaaagcggcgatttgcggtgaatccgataagaaatttttcgccaaactgaccagcgtgcttaacaccattctccaaatgcgtaattctaaaacgggtacggagcttgactacctgatttctccggtagccgacgttaacggcaacttcttcgattctcgtcaagcaccgaaaaatatgccacaagacgcggatgccaacggtgcataccatatcggccttaaaggcttaatgttattaggccgtatcaagaataatcaggagggcaagaaattaaatctggttatcaaaaacgaagaatacttcgagttcgttcagaatcgtaacaattaatgtatgcttaagcagatcggtaataaagacgaacaataagacgctgaaaagcgtcttttttcgttttggtcctgttccggcgcgatagtgtgaacatgctatagacttctggtgctacccgactgacaattaatcatccggctcgtataatgctagcaatttctactgttgtagatcattccggaacgttccagcgctgcaatttctactgttgtagatctgatttttcacatgttacctttcaatttctactgttgtagatccgaaaacgtaaagcttcagctgtaatttctactgttgtagatatcatatctggcgttaatggagtttcgtgacgaacaataagtcctccctaacggggggcaatttttattgataacaaaagtaacttcgagcttgtctacctcctagctcgtaaattgcacgctgatagtctcccaattgcgaaggaccaaaacgaaaaaacaccctttcgggtgtcttttctggaatttggtacgcagtactaggtatcgtgtaagtagcgaaggcccgtacgcgagataaactgctaggcaaccgcgactctacgactggtgctcgatttaatttcgctgacgtaaagaaattatcggcagtgcgtcaactgccgtatctttatcttaattaggtagttggacaagcccttgaaagaaatagcaagagcctgcctctctattgaagtcacggcgaaagtcgggtagaaatcaaagaaagcagaaattaaatcggagtaatactaagttgggataactccgtaactgactacgcctttctctagactttacttgaccagatacactgtctttgacacgttgaaggattagagcaatcaaatccaagactggctaagcacgaagcaactcttgagtgttaaaaagttacttcctgtattcgggacgagggtactagaagattgcagggactccgacgttaagtaaattacaaagtaataagtatcgttcaggatcacgttaccgcaataagaagcgagaataatataatttccgaagtgcttaccccagtagtgactattcctataacccttctgagtgtccggaggcggaaatttgccacgaaagagaaagtatttccccgacaataataaaggggcgctcctcagcttttccacttggttgggtaagctaggcaactctgaaaggagtttcggcgaagtgaagccgacacctttgaattgttttaggggcgttattcgagggcaatcggagctaacttcaagactacttctttgttgaatactaaatagtgcaaaggtcgtgtttcctcaaggatactccgctaacaatataggattccaatcagattcagcactggcggtacgggtgttgcggtgaggcgttcgggtttacggctcgaagctagcacggtaggaagcctgacaatcaccaagcaaaagggccgtcgaaggcccacaagatacgaaagctctcgaagccttatccttgaccgatccacctatttaggcagttacgcacaaaagctacccaataatccgtgacaggcacaatatcacggaacaaaaccgaaaactctcgtacacggttaggttttcgctaggaagaataaacctctatcttgattataagaaggctccccaagcacccccaaaaccgaaatagcg

SEQ ID NO: 48 - protein, 1196 aa, Artificial Sequence
  Chimeric STF-V10h
MAVKISGVLKDGTGKPVQNCTIQLKARRNSTTVVVNTVGSENPDEAGRYSMDVEYGQYSVILQVDGFPPSHAGTITVYEDSQPGTLNDFLCAMTEDDARPEVLRRLELMVEEVARNASVVAQSTADAKKSAGDASASAAQVAALVTDATDSARAASTSAGQAASSAQEASSGAEAASAKATEAEKSAAAAESSKNAAATSAGAAKTSETNAAASQQSAATSASTAATKASEAATSARDAVASKEAAKSSETNASSSAGRAASSATAAENSARAAKTSETNARSSETAAERSASAAADAKTAAAGSASTASTKATEAAGSAVSASQSKSAAEAAAIRAKNSAKRAEDIASAVALEDADTTRKGIVQLSSATNSTSETLAATPKAVKVVMDETNRKAPLDSPALTGTPTAPTALRGTNNTQIANTAFVLAAIADVIDASPDALNTLNELAAALGNDPDFATTMTNALAGKQPKNATLTALAGLSTAKNKLPYFAENDAASLTELTQVGRDILAKNSVADVLEYLGAGENSGSATDVMIQLAANDGFKFIGQCPDILTLRTIEPEKNGQRITLRQHTIGTGLGGGVFRAVLDGTGYTDDDGVVIKTAGGSVWLRVNADKVNPFMFGATGVADDTAALQKMLECGRAAELGTNVWKASNLELNNKSCSLSGSGLHVSRIEQISGATGALLTITQDCSLIYLSDCGLYGDGITAGTSGVTMETGNPGGAPSYPFNTAPDVRRDLYISNVHITGFDELGFDYPETNFSVSTHGLFIRNIKKTGAKIGTTDFTWTNLQIDTCGQECLVLDGAGNCRIIGAKLIWAGSENETPYSGLRISNSQNVNMTGVELQDCAYDGLYIKNSTVAISGLNTNRNSASSNLSYHNMVFENSIVTVDGYVCRNYAATSLYDLNSQAGNVRCIGSDSTVLINGIYESEVNSERLMGDNNLIQPYSGDLIINGLKNYYTYTGSVKNNIPTFDGVVTTATYVSAPSILGQGNMLKLTQSNKDKLLFSDKVSRHGCTIGLVLIPSFTGATTMTAFTLGSGYSPSGNSAVMQFIVNSSGVQTIAILLSGDGITQTLTSDLTTEQALASGGVYHFAMGFAPGRLWWSIIDINTGRRIRRAYRQPDLHAAFNSIFNSGTSSITAFSGPLAGDIACEGAGSHVYVGGFSSESDYAASRMYGLFTPVDLDKQYSFRTLNGNI

SEQ ID NO: 49 - protein, 1132 aa, Artificial Sequence
  A8
MGKGSSKGHTPREAKDNLKSTQLLSVIDAISEGPIEGPVDGLKSVLLNSTPVLDTEGNTNISGVTVVFRAGEQEQTPPEGFESSGSETVLGTEVKYDTPITRTITSANIDRLRFTFGVQALVETTSKGDRNPSEVRLLVQIQRNGGWVTEKDITIKGKTTSQYLASVVMGNLPPRPFNIRMRRMTPDSTTDQLQNKTLWSSYTEIIDVKQCYPNTALVGVQVDSEQFGSQQVSRNYHLRGRILQVPSNYNPQTRQYSGIWDGTFKPAYSNNMAWCLWDMLTHPRYGMGKRLGAADVDKWALYVIGQYCDQSVPDGFGGTEPRITCNAYLTTQRKAWDVLSDFCSAMRCMPVWNGQTLTFVQDRPSDKTWTYNRSNVVMPDDGAPFRYSFSALKDRHNAVEVNWIDPNNGWETATELVEDTQAIARYGRNVTKMDAFGCTSRGQAHRAGLWLIKTELLETQTVDFSVGAEGLRHVPGDVIEICDDDYAGISTGGRVLAVNSQTRTLTLDREITLPSSGTALISLVDGSGNPVSVEVQSVTDGVKVKVSRVPDGVAEYSVWELKLPTLRQRLFRCVSIRENDDGTYAITAVQHVPEKEAIVDNGAHFDGEQSGTVNGVTPPAVQHLTAEVTADSGEYQVLARWDTPKVVKGVSFLLRLTVTADDGSERLVSTARTTETTYRFTQLALGNYRLTVRAVNAWGQQGDPASVSFRIAAPAAPSRIELTPGYFQITATPHLAVYDPTVQFEFWFSEKQIADIRQVETSTRYLGTALYWIAASINIKPGHDYYFYIRSVNTVGKSAFVEAVGRASDDAEGYLDFFKGKITESHLGKELLEKVELTEDNASRLEEFSKEWKDASDKWNAMWAVKIEQTKDGKHYVAGIGLSMEDTEEGKLSQFLVAANRIAFIDPANGNETPMFVAQGNQIFMNDVFLKRLTAPTITSGGNPPAFSLTPDGKLTAKNADISGSVNANSGTLNNVTINENCQIKGKLSANQIEGDIVKTVSKSFPRTNSYASGTITVRISDDQKFDRQVMIPPVLFRGGKHENFNSNNQQSYWYSTCRLRVTRNGQEIFNQSTTDAQGVFSSVIDMPAGQGTLTLTFTVSSSGANNWTPTTSISDLLVVVMKKSTAGISIS

SEQ ID NO: 50 - protein, 812 aa, Artificial Sequence
  chimeric STF lambda-P2
MAVKISGVLKDGTGKPVQNCTIQLKARRNSTTVVVNTVGSENPDEAGRYSMDVEYGQYSVILQVDGFPPSHAGTITVYEDSQPGTLNDFLCAMTEDDARPEVLRRLELMVEEVARNASVVAQSTADAKKSAGDASASAAQVAALVTDATDSARAASTSAGQAASSAQEASSGAEAASAKATEAEKSAAAAESSKNAAATSAGAAKTSETNAAASQQSAATSASTAATKASEAATSARDAVASKEAAKSSETNASSSAGRAASSATAAENSARAAKTSETNARSSETAAERSASAAADAKTAAAGSASTASTKATEAAGSAVSASQSKSAAEAAAIRAKNSAKRAEDIASAVALEDADTTRKGIVQLSSATNSTSETLAATPKAVKVVMDETNRKAPLNSPALTGTPTTPTARQGTNNTQIANTAFVMAAIAALVDSSPDALNTLNELAAALGNDPNFATTMTNALAGKQPKDATLTALAGLATAADRFPYFTGNDVASLATLTKVGRDILAKSTVAAVIEYLGLQETVNRAGNAVQKNGDTLSGGLTFENDSILAWIRNTDWAKIGFKNDADGDTDSYMWFETGDNGNEYFKWRSRQSTTTKDLMTLKWDALNILVNAVINGCFGVGTTNALGGSSIVLGDNDTGFKQNGDGILDVYANSQRVFRFQNGVAIAFKNIQAGDSKKFSLSSSNTSTKNITFNLWGASTRPVVAELGDEAGWHFYSQRNTDNSVIFAVNGQMQPSNWGNFDSRYVKDVRLGTRVVQLMARGGRYEKAGHTITGLRIIGEVDGDDEAIFRPIQKYINGTWYNVAQV

SEQ ID NO: 51 - DNA, 3558 bp, Artificial Sequence
  nucleic acid encoding chimeric STF-V10
atggcagtaaagatttcaggagtcctgaaagacggcacaggaaaaccggtacagaactgcaccattcagctgaaagccagacgtaacagcaccacggtggtggtgaacacggtgggctcagagaatccggatgaagccgggcgttacagcatggatgtggagtacggtcagtacagtgtcatcctgcaggttgacggttttccaccatcgcacgccgggaccatcaccgtgtatgaagattcacaaccggggacgctgaatgattttctctgtgccatgacggaggatgatgcccggccggaggtgctgcgtcgtcttgaactgatggtggaagaggtggcgcgtaacgcgtccgtggtggcacagagtacggcagacgcgaagaaatcagccggcgatgccagtgcatcagctgctcaggtcgcggcccttgtgactgatgcaactgactcagcacgcgccgccagcacgtccgccggacaggctgcatcgtcagctcaggaagcgtcctccggcgcagaagcggcatcagcaaaggccactgaagcggaaaaaagtgccgcagccgcagagtcctcaaaaaacgcggcggccaccagtgccggtgcggcgaaaacgtcagaaacgaatgctgcagcgtcacaacaatcagccgccacgtctgcctccaccgcggccacgaaagcgtcagaggccgccacttcagcacgagatgcggtggcctcaaaagaggcagcaaaatcatcagaaacgaacgcatcatcaagtgccggtcgtgcagcttcctcggcaacggcggcagaaaattctgccagggcggcaaaaacgtccgagacgaatgccaggtcatctgaaacagcagcggaacggagcgcctctgccgcggcagacgcaaaaacagcggcggcggggagtgcgtcaacggcatccacgaaggcgacagaggctgcgggaagtgcggtatcagcatcgcagagcaaaagtgcggcagaagcggcggcaatacgtgcaaaaaattcggcaaaacgtgcagaagatatagcttcagctgtcgcgcttgaggatgcggacacaacgagaaaggggatagtgcagctcagcagtgcaaccaacagcacgtctgaaacgcttgctgcaacgccaaaggcggttaaggtggtaatggatgagactaatcgtaaggcacctctggacagtccggcactgaccggaacgccaacagcaccaaccgcgctcaggggaacaaacaatacccagattgcgaacaccgcttttgtactggccgcgattgcagatgttatcgacgcgtcacctgacgcactgaatacgctgaatgaactggccgcagcgctcgggaatgatccagattttgctaccaccatgactaacgcgcttgcgggtaaacaaccgaagaatgcgacactgacggcgctggcagggctttccacggcgaaaaataaattaccgtattttgcggaaaatgatgccgccagcctgactgaactgactcaggttggcagggatattctggcaaaaaattccgttgcagatgttcttgaataccttggggccggtgagaattcggcggcaaatgatggcttcaaattcatcggtcagtgcccagacatcttgaccctgcgtactatcgagccggaaaaaaacggtcagcgtatcaccttacgtcaacatacgattggcactggcttaggcggtggcgttttccgtgcagttctggacggcactggctataccgatgacgacggtgtggtgatcaaaaccgctgggggcagcgtttggctgcgtgtcaacgctgacaaagttaacccgttcatgttcggtgcaaccggagtagcggacgacaccgccgccctgcaaaaaatgctggaatgcggtcgtgcggcggaactggggactaacgtatggaaagcaagcaatctggaactgaacaacaaatcttgctctctgtccggcagtggcctgcacgtttctcgtattgaacagatttccggtgcaaccggagcattgttaaccatcacccaagactgttcgctgatttacctgtccgattgtggcctgtacggcgatggcatcaccgcaggcacgagcggtgttactatggaaacgggtaatccgggtggcgctccgtcttaccctttcaataccgctccggacgttcgtcgtgacctgtacatctctaacgtgcacatcacgggcttcgacgagctgggttttgattatccggaaaccaatttctctgtttcgacgcatggcctcttcatccgtaacatcaaaaaaacgggtgcaaagattggtactacggacttcacttggactaacctgcaaattgatacttgcggtcaggaatgtctggtgctggacggtgcgggtaactgccgtattattggtgcaaaactgatttgggcaggtagcgaaaacgaaacgccatactctggcctgcgtattagcaactctcaaaatgtaaatatgactggcgtagagttacaagactgcgcgtatgatggtttatacatcaagaactctacggttgcaatttcaggcttaaacaccaatcgcaatagcgcatcctctaatctgtcctaccataacatggtattcgaaaattctattgtaactgttgatggttatgtgtgtcgtaactacgcggcgacttcgctgtacgacctgaacagccaagcaggcaacgtccgttgcatcggtagcgacagcaccgttttaatcaacggcatctacgaaagcgaagtcaatagcgagcgcctgatgggtgataacaacctgatccagccgtatagtggtgatctgatcattaacggcctgaaaaattactacacctatactggtagcgtaaaaaacaacattccgaccttcgacggcgttgttactacggcaacctatgtgagcgcaccgtctattctgggtcagggcaatatgctcaaactgacccagtctaataaagacaaactgttatttagcgataaagttagccgtcatggctgtaccatcggcttagttctgattccgtcctttacgggcgcgaccactatgacggcgttcacgctgggtagcggttactctccatccggtaactccgccgtgatgcagttcattgttaacagttccggtgtacaaaccattgcgattttattatccggcgacggtattacccaaaccctgaccagcgatctgaccacggaacaagcactggcgagcggtggcgtgtatcattttgcaatgggttttgcgccgggtcgtttatggtggagcattatcgatattaacacgggcaggcgtattcgtcgcgcctaccgtcagccggatctgcacgcggcgttcaactctatcttcaactccggcacgtcgtctattaccgcatttagcgggccactggcgggcgacattgcttgcgaaggtgcaggtagccatgtatacgttggcggtttttcgtcggaatctgattacgcggctagccgtatgtatggcctgttcactccggtcgatctggacaagcagtatagcttccgtaccctgaacggtaacatt

SEQ ID NO: 52 - DNA, 3558 bp, Artificial Sequence
  nucleic acid encoding STF-V10f
atggcagtaaagatttcaggagtcctgaaagacggcacaggaaaaccggtacagaactgcaccattcagctgaaagccagacgtaacagcaccacggtggtggtgaacacggtgggctcagagaatccggatgaagccgggcgttacagcatggatgtggagtacggtcagtacagtgtcatcctgcaggttgacggttttccaccatcgcacgccgggaccatcaccgtgtatgaagattcacaaccggggacgctgaatgattttctctgtgccatgacggaggatgatgcccggccggaggtgctgcgtcgtcttgaactgatggtggaagaggtggcgcgtaacgcgtccgtggtggcacagagtacggcagacgcgaagaaatcagccggcgatgccagtgcatcagctgctcaggtcgcggcccttgtgactgatgcaactgactcagcacgcgccgccagcacgtccgccggacaggctgcatcgtcagctcaggaagcgtcctccggcgcagaagcggcatcagcaaaggccactgaagcggaaaaaagtgccgcagccgcagagtcctcaaaaaacgcggcggccaccagtgccggtgcggcgaaaacgtcagaaacgaatgctgcagcgtcacaacaatcagccgccacgtctgcctccaccgcggccacgaaagcgtcagaggccgccacttcagcacgagatgcggtggcctcaaaagaggcagcaaaatcatcagaaacgaacgcatcatcaagtgccggtcgtgcagcttcctcggcaacggcggcagaaaattctgccagggcggcaaaaacgtccgagacgaatgccaggtcatctgaaacagcagcggaacggagcgcctctgccgcggcagacgcaaaaacagcggcggcggggagtgcgtcaacggcatccacgaaggcgacagaggctgcgggaagtgcggtatcagcatcgcagagcaaaagtgcggcagaagcggcggcaatacgtgcaaaaaattcggcaaaacgtgcagaagatatagcttcagctgtcgcgcttgaggatgcggacacaacgagaaaggggatagtgcagctcagcagtgcaaccaacagcacgtctgaaacgcttgctgcaacgccaaaggcggttaaggtggtaatggatgagactaatcgtaaggcacctctggacagtccggcactgaccggaacgccaacagcaccaaccgcgctcaggggaacaaacaatacccagattgcgaacaccgcttttgtactggccgcgattgcagatgttatcgacgcgtcacctgacgcactgaatacgctgaatgaactggccgcagcgctcgggaatgatccagattttgctaccaccatgactaacgcgcttgcgggtaaacaaccgaagaatgcgacactgacggcgctggcagggctttccacggcgaaaaataaattaccgtattttgcggaaaatgatgccgccagcctgactgaactgactcaggttggcagggatattctggcaaaaaattccgttgcagatgttcttgaataccttggggccggtgagaattcggcggcaaatgatggcttcgcattcatcggtcagtgcccagacatcttgaccctgcgtactatcgagccggaaaaaaacggtcagcgtatcaccttacgtcaacatacgattggcactggcttaggcggtggcgttttccgtgcagttctggacggcactggctataccgatgacgacggtgtggtgatcaaaaccgctgggggcagcgtttggctgcgtgtcaacgctgacaaagttaacccgttcatgttcggtgcaaccggagtagcggacgacaccgccgccctgcaaaaaatgctggaatgcggtcgtgcggcggaactggggactaacgtatggaaagcaagcaatctggaactgaacaacaaatcttgctctctgtccggcagtggcctgcacgtttctcgtattgaacagatttccggtgcaaccggagcattgttaaccatcacccaagactgttcgctgatttacctgtccgattgtggcctgtacggcgatggcatcaccgcaggcacgagcggtgttactatggaaacgggtaatccgggtggcgctccgtcttaccctttcaataccgctccggacgttcgtcgtgacctgtacatctctaacgtgcacatcacgggcttcgacgagctgggttttgattatccggaaaccaatttctctgtttcgacgcatggcctcttcatccgtaacatcaaaaaaacgggtgcaaagattggtactacggacttcacttggactaacctgcaaattgatacttgcggtcaggaatgtctggtgctggacggtgcgggtaactgccgtattattggtgcaaaactgatttgggcaggtagcgaaaacgaaacgccatactctggcctgcgtattagcaactctcaaaatgtaaatatgactggcgtagagttacaagactgcgcgtatgatggtttatacatcaagaactctacggttgcaatttcaggcttaaacaccaatcgcaatagcgcatcctctaatctgtcctaccataacatggtattcgaaaattctattgtaactgttgatggttatgtgtgtcgtaactacgcggcgacttcgctgtacgacctgaacagccaagcaggcaacgtccgttgcatcggtagcgacagcaccgttttaatcaacggcatctacgaaagcgaagtcaatagcgagcgcctgatgggtgataacaacctgatccagccgtatagtggtgatctgatcattaacggcctgaaaaattactacacctatactggtagcgtaaaaaacaacattccgaccttcgacggcgttgttactacggcaacctatgtgagcgcaccgtctattctgggtcagggcaatatgctcaaactgacccagtctaataaagacaaactgttatttagcgataaagttagccgtcatggctgtaccatcggcttagttctgattccgtcctttacgggcgcgaccactatgacggcgttcacgctgggtagcggttactctccatccggtaactccgccgtgatgcagttcattgttaacagttccggtgtacaaaccattgcgattttattatccggcgacggtattacccaaaccctgaccagcgatctgaccacggaacaagcactggcgagcggtggcgtgtatcattttgcaatgggttttgcgccgggtcgtttatggtggagcattatcgatattaacacgggcaggcgtattcgtcgcgcctaccgtcagccggatctgcacgcggcgttcaactctatcttcaactccggcacgtcgtctattaccgcatttagcgggccactggcgggcgacattgcttgcgaaggtgcaggtagccatgtatacgttggcggtttttcgtcggaatctgattacgcggctagccgtatgtatggcctgttcactccggtcgatctggacaagcagtatagcttccgtaccctgaacggtaacatt

SEQ ID NO: 53 - DNA, 3558 bp, Artificial Sequence
  nucleic acid encoding STF-V10a
atggcagtaaagatttcaggagtcctgaaagacggcacaggaaaaccggtacagaactgcaccattcagctgaaagccagacgtaacagcaccacggtggtggtgaacacggtgggctcagagaatccggatgaagccgggcgttacagcatggatgtggagtacggtcagtacagtgtcatcctgcaggttgacggttttccaccatcgcacgccgggaccatcaccgtgtatgaagattcacaaccggggacgctgaatgattttctctgtgccatgacggaggatgatgcccggccggaggtgctgcgtcgtcttgaactgatggtggaagaggtggcgcgtaacgcgtccgtggtggcacagagtacggcagacgcgaagaaatcagccggcgatgccagtgcatcagctgctcaggtcgcggcccttgtgactgatgcaactgactcagcacgcgccgccagcacgtccgccggacaggctgcatcgtcagctcaggaagcgtcctccggcgcagaagcggcatcagcaaaggccactgaagcggaaaaaagtgccgcagccgcagagtcctcaaaaaacgcggcggccaccagtgccggtgcggcgaaaacgtcagaaacgaatgctgcagcgtcacaacaatcagccgccacgtctgcctccaccgcggccacgaaagcgtcagaggccgccacttcagcacgagatgcggtggcctcaaaagaggcagcaaaatcatcagaaacgaacgcatcatcaagtgccggtcgtgcagcttcctcggcaacggcggcagaaaattctgccagggcggcaaaaacgtccgagacgaatgccaggtcatctgaaacagcagcggaacggagcgcctctgccgcggcagacgcaaaaacagcggcggcggggagtgcgtcaacggcatccacgaaggcgacagaggctgcgggaagtgcggtatcagcatcgcagagcaaaagtgcggcagaagcggcggcaatacgtgcaaaaaattcggcaaaacgtgcagaagatatagcttcagctgtcgcgcttgaggatgcggacacaacgagaaaggggatagtgcagctcagcagtgcaaccaacagcacgtctgaaacgcttgctgcaacgccaaaggcggttaaggtggtaatggatgagactaatcgtaaggcacctctggacagtccggcactgaccggaacgccaacagcaccaaccgcgctcaggggaacaaacaatacccagattgcgaacaccgcttttgtactggccgcgattgcagatgttatcgacgcgtcacctgacgcactgaatacgctgaatgaactggccgcagcgctcgggaatgatccagattttgctaccaccatgactaacgcgcttgcgggtaaacaaccgaagaatgcgacactgacggcgctggcagggctttccacggcgaaaaataaattaccgtattttgcggaaaatgatgccgccagcctgactgaactgactcaggttggcagggatattctggcaaaaaattccgttgcagatgttcttgaataccttggggccggtgagaattcggcggcaaatgatggcgcggcacacatcggtcagtgcccagacatcttgaccctgcgtactatcgagccggaaaaaaacggtcagcgtatcaccttacgtcaacatacgattggcactggcttaggcggtggcgttttccgtgcagttctggacggcactggctataccgatgacgacggtgtggtgatcaaaaccgctgggggcagcgtttggctgcgtgtcaacgctgacaaagttaacccgttcatgttcggtgcaaccggagtagcggacgacaccgccgccctgcaaaaaatgctggaatgcggtcgtgcggcggaactggggactaacgtatggaaagcaagcaatctggaactgaacaacaaatcttgctctctgtccggcagtggcctgcacgtttctcgtattgaacagatttccggtgcaaccggagcattgttaaccatcacccaagactgttcgctgatttacctgtccgattgtggcctgtacggcgatggcatcaccgcaggcacgagcggtgttactatggaaacgggtaatccgggtggcgctccgtcttaccctttcaataccgctccggacgttcgtcgtgacctgtacatctctaacgtgcacatcacgggcttcgacgagctgggttttgattatccggaaaccaatttctctgtttcgacgcatggcctcttcatccgtaacatcaaaaaaacgggtgcaaagattggtactacggacttcacttggactaacctgcaaattgatacttgcggtcaggaatgtctggtgctggacggtgcgggtaactgccgtattattggtgcaaaactgatttgggcaggtagcgaaaacgaaacgccatactctggcctgcgtattagcaactctcaaaatgtaaatatgactggcgtagagttacaagactgcgcgtatgatggtttatacatcaagaactctacggttgcaatttcaggcttaaacaccaatcgcaatagcgcatcctctaatctgtcctaccataacatggtattcgaaaattctattgtaactgttgatggttatgtgtgtcgtaactacgcggcgacttcgctgtacgacctgaacagccaagcaggcaacgtccgttgcatcggtagcgacagcaccgttttaatcaacggcatctacgaaagcgaagtcaatagcgagcgcctgatgggtgataacaacctgatccagccgtatagtggtgatctgatcattaacggcctgaaaaattactacacctatactggtagcgtaaaaaacaacattccgaccttcgacggcgttgttactacggcaacctatgtgagcgcaccgtctattctgggtcagggcaatatgctcaaactgacccagtctaataaagacaaactgttatttagcgataaagttagccgtcatggctgtaccatcggcttagttctgattccgtcctttacgggcgcgaccactatgacggcgttcacgctgggtagcggttactctccatccggtaactccgccgtgatgcagttcattgttaacagttccggtgtacaaaccattgcgattttattatccggcgacggtattacccaaaccctgaccagcgatctgaccacggaacaagcactggcgagcggtggcgtgtatcattttgcaatgggttttgcgccgggtcgtttatggtggagcattatcgatattaacacgggcaggcgtattcgtcgcgcctaccgtcagccggatctgcacgcggcgttcaactctatcttcaactccggcacgtcgtctattaccgcatttagcgggccactggcgggcgacattgcttgcgaaggtgcaggtagccatgtatacgttggcggtttttcgtcggaatctgattacgcggctagccgtatgtatggcctgttcactccggtcgatctggacaagcagtatagcttccgtaccctgaacggtaacatt

SEQ ID NO: 54 - DNA, 3588 bp, Artificial Sequence
  nucleic acid encoding STF-V10h
atggcagtaaagatttcaggagtcctgaaagacggcacaggaaaaccggtacagaactgcaccattcagctgaaagccagacgtaacagcaccacggtggtggtgaacacggtgggctcagagaatccggatgaagccgggcgttacagcatggatgtggagtacggtcagtacagtgtcatcctgcaggttgacggttttccaccatcgcacgccgggaccatcaccgtgtatgaagattcacaaccggggacgctgaatgattttctctgtgccatgacggaggatgatgcccggccggaggtgctgcgtcgtcttgaactgatggtggaagaggtggcgcgtaacgcgtccgtggtggcacagagtacggcagacgcgaagaaatcagccggcgatgccagtgcatcagctgctcaggtcgcggcccttgtgactgatgcaactgactcagcacgcgccgccagcacgtccgccggacaggctgcatcgtcagctcaggaagcgtcctccggcgcagaagcggcatcagcaaaggccactgaagcggaaaaaagtgccgcagccgcagagtcctcaaaaaacgcggcggccaccagtgccggtgcggcgaaaacgtcagaaacgaatgctgcagcgtcacaacaatcagccgccacgtctgcctccaccgcggccacgaaagcgtcagaggccgccacttcagcacgagatgcggtggcctcaaaagaggcagcaaaatcatcagaaacgaacgcatcatcaagtgccggtcgtgcagcttcctcggcaacggcggcagaaaattctgccagggcggcaaaaacgtccgagacgaatgccaggtcatctgaaacagcagcggaacggagcgcctctgccgcggcagacgcaaaaacagcggcggcggggagtgcgtcaacggcatccacgaaggcgacagaggctgcgggaagtgcggtatcagcatcgcagagcaaaagtgcggcagaagcggcggcaatacgtgcaaaaaattcggcaaaacgtgcagaagatatagcttcagctgtcgcgcttgaggatgcggacacaacgagaaaggggatagtgcagctcagcagtgcaaccaacagcacgtctgaaacgcttgctgcaacgccaaaggcggttaaggtggtaatggatgagactaatcgtaaggcacctctggacagtccggcactgaccggaacgccaacagcaccaaccgcgctcaggggaacaaacaatacccagattgcgaacaccgcttttgtactggccgcgattgcagatgttatcgacgcgtcacctgacgcactgaatacgctgaatgaactggccgcagcgctcgggaatgatccagattttgctaccaccatgactaacgcgcttgcgggtaaacaaccgaagaatgcgacactgacggcgctggcagggctttccacggcgaaaaataaattaccgtattttgcggaaaatgatgccgccagcctgactgaactgactcaggttggcagggatattctggcaaaaaattccgttgcagatgttcttgaataccttggggccggtgagaattcggggagcgctacagacgttatgattcagctggcggcaaatgatggcttcaaattcatcggtcagtgcccagacatcttgaccctgcgtactatcgagccggaaaaaaacggtcagcgtatcaccttacgtcaacatacgattggcactggcttaggcggtggcgttttccgtgcagttctggacggcactggctataccgatgacgacggtgtggtgatcaaaaccgctgggggcagcgtttggctgcgtgtcaacgctgacaaagttaacccgttcatgttcggtgcaaccggagtagcggacgacaccgccgccctgcaaaaaatgctggaatgcggtcgtgcggcggaactggggactaacgtatggaaagcaagcaatctggaactgaacaacaaatcttgctctctgtccggcagtggcctgcacgtttctcgtattgaacagatttccggtgcaaccggagcattgttaaccatcacccaagactgttcgctgatttacctgtccgattgtggcctgtacggcgatggcatcaccgcaggcacgagcggtgttactatggaaacgggtaatccgggtggcgctccgtcttaccctttcaataccgctccggacgttcgtcgtgacctgtacatctctaacgtgcacatcacgggcttcgacgagctgggttttgattatccggaaaccaatttctctgtttcgacgcatggcctcttcatccgtaacatcaaaaaaacgggtgcaaagattggtactacggacttcacttggactaacctgcaaattgatacttgcggtcaggaatgtctggtgctggacggtgcgggtaactgccgtattattggtgcaaaactgatttgggcaggtagcgaaaacgaaacgccatactctggcctgcgtattagcaactctcaaaatgtaaatatgactggcgtagagttacaagactgcgcgtatgatggtttatacatcaagaactctacggttgcaatttcaggcttaaacaccaatcgcaatagcgcatcctctaatctgtcctaccataacatggtattcgaaaattctattgtaactgttgatggttatgtgtgtcgtaactacgcggcgacttcgctgtacgacctgaacagccaagcaggcaacgtccgttgcatcggtagcgacagcaccgttttaatcaacggcatctacgaaagcgaagtcaatagcgagcgcctgatgggtgataacaacctgatccagccgtatagtggtgatctgatcattaacggcctgaaaaattactacacctatactggtagcgtaaaaaacaacattccgaccttcgacggcgttgttactacggcaacctatgtgagcgcaccgtctattctgggtcagggcaatatgctcaaactgacccagtctaataaagacaaactgttatttagcgataaagttagccgtcatggctgtaccatcggcttagttctgattccgtcctttacgggcgcgaccactatgacggcgttcacgctgggtagcggttactctccatccggtaactccgccgtgatgcagttcattgttaacagttccggtgtacaaaccattgcgattttattatccggcgacggtattacccaaaccctgaccagcgatctgaccacggaacaagcactggcgagcggtggcgtgtatcattttgcaatgggttttgcgccgggtcgtttatggtggagcattatcgatattaacacgggcaggcgtattcgtcgcgcctaccgtcagccggatctgcacgcggcgttcaactctatcttcaactccggcacgtcgtctattaccgcatttagcgggccactggcgggcgacattgcttgcgaaggtgcaggtagccatgtatacgttggcggtttttcgtcggaatctgattacgcggctagccgtatgtatggcctgttcactccggtcgatctggacaagcagtatagcttccgtaccctgaacggtaacatt

SEQ ID NO: 55 - DNA, 2032 bp, Artificial Sequence
  nucleic acid encoding A8
atgggtaaaggaagcagtaaggggcataccccgcgcgaagcgaaggacaacctgaagtccacgcagttgctgagtgtgatcgatgccatcagcgaagggccgattgaaggtccggtggatggcttaaaaagcgtgctgctgaacagtacgccggtgctggacactgaggggaataccaacatatccggtgtcacggtggtgttccgggctggtgagcaggagcagactccgccggagggatttgaatcctccggctccgagacggtgctgggtacggaagtgaaatatgacacgccgatcacccgcaccattacgtctgcaaacatcgaccgtctgcgctttaccttcggtgtacaggcactggtggaaaccacctcaaagggtgacaggaatccgtcggaagtccgcctgctggttcagatacaacgtaacggtggctgggtgacggaaaaagacatcaccattaagggcaaaaccacctcgcagtatctggcctcggtggtgatgggtaacctgccgccgcgcccgtttaatatccggatgcgcaggatgacgccggacagcaccacagaccagctgcagaacaaaacgctctggtcgtcatacactgaaatcatcgatgtgaaacagtgctacccgaacacggcactggtcggcgtgcaggtggactcggagcagttcggcagccagcaggtgagccgtaattatcatctgcgcgggcgtattctgcaggtgccgtcgaactataacccgcagacgcggcaatacagcggtatctgggacggaacgtttaaaccggcatacagcaacaacatggcctggtgtctgtgggatatgctgacccatccgcgctacggcatggggaaacgtcttggtgcggcggatgtggataaatgggcgctgtatgtcatcggccagtactgcgaccagtcagtgccggacggctttggcggcacggagccgcgcatcacctgtaatgcgtacctgaccacacagcgtaaggcgtgggatgtgctcagcgatttctgctcggcgatgcgctgtatgccggtatggaacgggcagacgctgacgttcgtgcaggaccgaccgtcggataagacgtggacctataaccgcagtaatgtggtgatgccggatgatggcgcgccgttccgctacagcttcagcgccctgaaggaccgccataatgccgttgaggtgaactggattgacccgaacaacggctgggagacggcgacagagcttgttgaagatacgcaggccattgcccgttacggtcgtaatgttacgaagatggatgcctttggctgtaccagccgggggcaggcacaccgcgccgggctgtggctgattaaaacagaactgctggaaacgcagaccgtggatttcagcgtcggcgcagaagggcttcgccatgtaccgggcgatgttattgaaatctgcgatgatgactatgccggtatcagcaccggtggtcgtgtgctggcggtgaacagccagacccggacgctgacgctcgaccgtgaaatcacgctgccatcctccggtaccgcgctgataagcctggttgacggaagtggcaatccggtcagcgtggaggttcagtccgtcaccgacggcgtgaaggtaaaagtgagccgtgttcctgacggtgttgctgaatacagcgtatgggagctgaagctgccgacgctgcgccagcgactgttccgctgcgtgagtatccgtgagaacgacgacggcacgtatgccatcaccgccgtgcagcatgtgccggaaaaagaggccatcgtggataacggggcgcactttgacggcgaacagagtggcacggtgaatggtgtcacgccgccagcggtgcagcacctgaccgcagaagtcactgcagacagcggggaatatcaggtgctggcgcgatgggacacaccgaaggtggtgaagggcgtgagtttcctgctccgtctgaccgtaacagcggacgacggcagtgagcggctggtcagcacggcccggacgacggaaaccacat

SEQ ID NO: 56 - DNA, 2045 bp, Artificial Sequence
  nucleic acid encoding STF lambda-P2
atggcagtaaagatttcaggagtcctgaaagacggcacaggaaaaccggtacagaactgcaccattcagctgaaagccagacgtaacagcaccacggtggtggtgaacacggtgggctcagagaatccggatgaagccgggcgttacagcatggatgtggagtacggtcagtacagtgtcatcctgcaggttgacggttttccaccatcgcacgccgggaccatcaccgtgtatgaagattcacaaccggggacgctgaatgattttctctgtgccatgacggaggatgatgcccggccggaggtgctgcgtcgtcttgaactgatggtggaagaggtggcgcgtaacgcgtccgtggtggcacagagtacggcagacgcgaagaaatcagccggcgatgccagtgcatcagctgctcaggtcgcggcccttgtgactgatgcaactgactcagcacgcgccgccagcacgtccgccggacaggctgcatcgtcagctcaggaagcgtcctccggcgcagaagcggcatcagcaaaggccactgaagcggaaaaaagtgccgcagccgcagagtcctcaaaaaacgcggcggccaccagtgccggtgcggcgaaaacgtcagaaacgaatgctgcagcgtcacaacaatcagccgccacgtctgcctccaccgcggccacgaaagcgtcagaggccgccacttcagcacgagatgcggtggcctcaaaagaggcagcaaaatcatcagaaacgaacgcatcatcaagtgccggtcgtgcagcttcctcggcaacggcggcagaaaattctgccagggcggcaaaaacgtccgagacgaatgccaggtcatctgaaacagcagcggaacggagcgcctctgccgcggcagacgcaaaaacagcggcggcggggagtgcgtcaacggcatccacgaaggcgacagaggctgcgggaagtgcggtatcagcatcgcagagcaaaagtgcggcagaagcggcggcaatacgtgcaaaaaattcggcaaaacgtgcagaagatatagcttcagctgtcgcgcttgaggatgcggacacaacgagaaaggggatagtgcagctcagcagtgcaaccaacagcacgtctgaaacgcttgctgcaacgccaaaggcggttaaggtggtaatggatgagactaatcgtaaagcgccattaaacagccctgcactgaccggaacgccaacgacgccaactgcgcgacagggaacgaataatactcagatcgcaaacacggctttcgttatggccgcgattgccgcccttgtagactcgtcgcctgacgcactgaatacgctgaacgagctggcggcggcgctgggcaatgacccgaattttgctaccaccatgactaatgcgcttgcgggtaagcaaccgaaagatgctaccctgacggcgctggcggggcttgctactgcggcagacaggtttccgtattttacggggaatgatgttgccagcctggcgaccctgacaaaagtcgggcgggatattctggctaaatcgaccgttgccgccgttatcgaatatctcggtttacaggaaacggtaaaccgagccgggaacgccgtgcaaaaaaatggcgataccttgtccggtggacttacttttgaaaacgactcaatccttgcctggattcgaaatactgactgggcgaagattggatttaaaaatgatgccgatggtgacactgattcatacatgtggtttgaaacgggggataacggcaatgaatatttcaaatggagaagccgccagagtaccacaacaaaagacctgatgacgttgaaatgggatgcactaaatattcttgttaatgccgtcattaatggctgttttggagttggtacgacgaatgcactaggtggtagctctattgttcttggtgataatgataccggatttaaacagaatggagacggtattcttgatgtttatgctaacagtcagcgtgtattccgttttcagaatggagtggctattgcttttaaaaatattcaggcaggtgatag

SEQ ID NO: 57 - protein, 175 aa, Artificial Sequence
  P2 accessory protein 1
MQHLKNIKSGNPKTKEQYQLTKNFDVIWLWSEDGKNWYEEVKNFQPDTIKIVYDENNIIVAITRDASTLNPEGFSVVEVPDITSNRRADDSGKWMFKDGAVVKRIYTADEQQQQAESQKAALLSEAENVIQPLERAVRLNMATDEERARLESWERYSVLVSRVDPANPEWPEMPQ

SEQ ID NO: 58 - DNA, 525 bp, Artificial Sequence
  nucleic acid encoding P2 accessory protein 1
atgcagcatttaaagaacattaagtcaggtaatccaaaaacaaaagagcaatatcagctaacaaagaattttgatgttatctggttatggtccgaagacggaaaaaactggtatgaggaagtgaagaactttcagccagacacaataaagattgtttacgatgaaaataatattattgtcgctatcaccagagatgcttcaacgcttaatcctgaaggttttagcgttgttgaggttcctgatattacctccaaccgacgtgctgacgactcaggtaaatggatgtttaaggatggtgctgtggttaaacggatttatacggcagatgaacagcaacaacaggcagaatcacaaaaggccgcgttactttccgaagcggaaaacgttattcagccactggaacgcgctgtcaggctgaatatggcgacggatgaggaacgtgcacgactggagtcatgggaacgttacagcgttctggtcagccgtgtggatcctgcaaatcctgaatggccggaaatgccgcaa

SEQ ID NO: 59 - protein, 1158 aa, Artificial Sequence
  Sequence of Z2145 gpJ of Figure 7
  MISC_FEATURE (9)..(9): Xaa may be any amino acid
  MISC_FEATURE (495)..(495): Xaa may be any amino acid
  MISC_FEATURE (813)..(820): box 1
  MISC_FEATURE (985)..(993): box 2
MGKGGGKGXTPVEAKDNLKSTQMMSVIDAIGEGPIEGPVKGLQSILVNKTPLTDTDGNPVIHGVTAVWRAGEQEQTPPEGFESSGAETALGVEVTKAKPVTRTITSANIDRLRVTFGVQSLLETTSKGDRNPSSVRLLIQLQRNGNWVTEKDVTINGKTTSQFLASVILDNLPPRPFNIRMVRETADSTSDQLQNKTLWSSYTEIIDVKQCYPNTAIVGLQVDAEQFGGQQMTVNYHIRGRIIQVPSNYDPEKRTYSGIWDGSLKPAYSNNPAWCLWDMLTHPRYGMGKRLGAADVDKWALYAIAQYCDQMVPDGFGGTEPRMTFNAYLSQQRKAWDVLSDFCSAMRCMPVWNGQTLTFVQDSPSDVVWPYTNSDVVVDDNGVGFRYSFSALKDRHTAVEVNYTDPQNGWQTSTELVEDPEAILRYGRNLLKMDAFGCTSRGQAHRAGLWVIKTGLLETQTVDFTLGSQGLRHTPGDIIEICDNDYAGTMTGGRXLSIDAASRTLTLDREVTLPETGAATVNLINGSGKPVSVAITAHPAPDRIQVSTLPDGVETYGVWGLSLPSLRRRLFRCVSVRENTDGTFAITAVQHVPEKEAIVDNGARFEPQSGTLNSVIPPAVQHLTVEVSAADGQYLAQAKWDTPKVVKGVSFMLRLTVAADDGSERLVSTARTTETTYRFTQLAPGNYRLTVRAVNAWGQQGDPASVSFRIAAPAAPSQIELTPGYFQITAVPRLAVYDPTVQFEFWFSETRITDIRQVETTARYLGTGLYWIAASINIKPGHDYYFYIRSVNTVGKSAFVEAVGQPSDDASGYLDFFKGEIGKTHLAQELWTQIDNGQLAPDLAEIRTSITDVSNEITQTVNKKLEDQSAAIQQIQKVQVDTNNNLNSMWAVKLQQMQDGRLYIAGIGAGIENTSDGMQSQVLLAADRIAMINPANGNTKPMFVGQGDQIFMNEVFLKYLTAPTITSGGNPPAFSLTSDGKLTAKNADISGSVNANSGTLNNVTINENCRVLGKLSANQIEGDLVKTVGKAFPRDSRAPERWPSGTITVRVYDDQPFDRQIVIPAVAFSGAKHEREHTDIYSSCRLIVRKNGAEIYNRTALDNTLIYSGVIDMPAGHGHMTLEFSVSAWLVNNWYPTASISDLLVVVMKKATAGISIS

SEQ ID NO: 60 - protein, 1131 aa, Artificial Sequence
  Sequence of 1A2 gpJ of Figure 7
  MISC_FEATURE (814)..(821): box 1
  MISC_FEATURE (958)..(966): box 2
MGKGSSKGHTPREAKDNLKSTQLLSVIDAISEGPVEGPVDGLKSVLLNSTPVLDSEGNTNISGVTVVFRAGEQEQTPPEGFESSGSETVLGTEVKYDTPITRTITSANIDRLRFTFGVQALVETTSKGDRNPSEVRLLVQIQRNGGWVTEKDITIKGKTTSQYLASVVVGNLPPRPFNIRMRRMTPDSTTDQLQNKTLWSSYTEIIDVKQCYPNTALVGVQVDSEQFGSQQVSRNYHLRGRILQVPSNYNPQTRQYSGIWDGTFKPAYSNNMAWCLWDMLTHPRYGMGKRLGAADVDKWALYVIGQYCDQSVPDGFGGTEPRITCNAYLTTQRKAWDVLSDFCSAMRCMPVWNGQTLTFVQDRPSDKVWTYNRSNVVMPDDGAPFRYSFSALKDRHNAVEVNWIDPNNGWETAAELVEDTQAIARYGRNVTKMDAFGCTSRGQAHRAGLWLIKTELLETQTVDFSVGAEGLRHVPGDVIEICDDDYAGISIGGRVLAVNSQTRTLTLDREITLPFSGTTLISLVDGSGNPVSVEVQSVTDGVKVKVSRVPDGVAEYSVWGLKLPTLRQRLFRCVSIRENDDGTYAITAVQHVPEKEAIVDNGAHFDGDQSGTVNGVTPPAVQHLTAEVTADSGEYQVLARWDTPKVVKGVSFLLRLTVTADDGSERLVSTARTTETTYRFTQLALGNYRLTVRAVNAWGQQGDPASVSFRIAAPAAPSRIELTPGYFQITATPHLAVYDPTVQFEFWFSEKRIADIRQVETTARYLGTGLYWIAASINIKPGHDYYFYIRSVNTVGKSAFVEAVGRASDDAEGYLDFFKGKITESHLGKELLEKVDLTEDNASRLDQFSKEWKDANDKWNAMWGVKIEQTEDGKHYVAGLGLSMEDTEEGKLSQFLVAANRIAFIDPSNGNTRPMFVGQGDQIFMNDVFLKRLTAPTITSGGNPPAFSLTPDGRLTAKNADISGNVNANSGTLNNVTINENCRVLGKLSANQIEGDLVKTVGKAFPRDSRAPERWPSGTITVRVYDDQPFDRQIVIPAVAFSGAKHEKEHTDIYSSCRLIVRKNGAEIYNRTALDNTLIYSGVIDMPAGHGHMTLEFSVSAWLVNNWYPTASISDLLVVVMKKATAGITIS

SEQ ID NO: 61 - protein, 1132 aa, Artificial Sequence
  Sequence of 591 gpJ of Figure 7
  MISC_FEATURE (814)..(821): box 1
  MISC_FEATURE (958)..(966): box 2
MGKGSSKGHTPREAKDNLKSTQLLSVIDAISEGPVEGPVDGLKSVLLNSTPVLDSEGNTNISGVTVVFRAGEQEQTPPEGFESSGSETVLGTEVKYDTPITRTITSANIDRLRFTFGVQALVETTSKGDRNPSEVRLLVQIQRNGGWVTEKDITIKGKTTSQYLASVVVGSLPPRPFNIRMRRMTPDSTTDQLQNKTLWSSYTEIIDVKQCYPNTALVGVQVDSEQFGSQQVSRNYHLRGRILQVPSNYNPQTRQYSGIWDGTFKPAYSNNPAWCLWDMLTHPRYGMGKRLGAADVDKWALYVIGQYCDQSVPDGFGGTEPRITCNAYLTTQRKAWDVLSDFCSAMRCMPVWNGQTLTFVQDRPSDKVWTYNRSNVVMPDDGAPFRYSFSALKDRHNAVEVNWIDPNNGWETATELVEDTQAIARYGRNVTKMDAFGCTSRGQAHRAGLWLIKTELLETQTVDFSVGAEGLRHVPGDVIEICDDDYAGISTGGRVLAVNSQTRTLTLDREITLPSSGTTLISLVDGSGNPVSVEVQSVTDGLKVKVNRVPDGVAEYSVWGLKLPTLRQRLFRCVSIRENDDGTYAITAVQHVPEKEAIVDNGAHFDGDQSSTVNGVTPPAVQHLTAEVTADSGEYQVLARWDTPKVVKGVSFLLRLTVTADDGSERLVSTARTTETTYRFTQLALGNYRLTVRAVNAWGQQGDPASVSFRIAAPAAPSRIELTPGYFQITATPHLAVYDPTVQFEFWFSEKRITDIRQVETTARYLGTALYWIAASINIKPGHDYYFYIRSVNTVGKSAFVEAVGRASDDAEGYLDFFKGKITESHLGKELLEKVELTEDNASRLEEFSNEWKDASDKWNAMWAVKIEQTKDGKHYVAGIGLSMEDTEEGKLSQFLVAANRIAFIDPANGNETPMFVAQGNQIFMNDVFLKRLTAPTITSGGNPPAFSLTPDGKLTAKNADISGNVNANSGTLNNVTINENCQIKGKLSANQIEGDIVKTVSKSFPRTNSYASGTITVRISDDQKFDRQVMIPPVLFRGGKHENFNSNNQQSYWYSTCRLRVTRNGQEIFNQSTTDAQGVFSSVIDMPAGQGTLTLTFTVSSSGANNWTPTTSISDLLVVVMKKSTAGISIS

SEQ ID NO: 62 - protein, 859 aa, Artificial Sequence
  Sequence of E6BTD4-ECOLX gpH  of Figure 9A
  MISC_FEATURE (187)..(208): distal region box
  MISC_FEATURE (301)..(420): proximal region box
MAEPVGDLVVDLSLDAARFDEQMARVRRHFSGTESDAKKTAAVVEQSLSRQALAAQKAGISVGQYKAAMRMLPAQFTDVATQLAGGQSPWLILLQQGGQVKDSFGGMIPMFRGLAGAITLPMVGATSLAVATGALAYAWYQGNSTLSDFNKTLVLSGNQAGLTADRMLVLSRAGQAAGLTFNQTSESLTALVNAGVRGGEQFEAISQSVARFSSASGVEVDKVAEAFGKLTTDPTSGLTAMARQFHNVTAEQIAYVAQLQRSGDEAGALQAANEAATKGFDDQTRRLKENMGTLETWADRTARAFKSMWDSVLDIGRPDTAQGMLEKAEKAFDEADKKWQWYQSRSHRRGKTSAFLANLRGAWEDRANAQLGLSAATLQADLEKAREMAAKDWAESEASRLKYTEEAQKAYERLQTPLEKYTARQEELNKALKDGKILQADYNTLMAAAKKDYEATLKKPKQSGVKVSAGDRQEDSAHAALLTLQAELRTLEKHAGANEKISQQRRDLWKAESQFAVLEEAAQRRQLSAQEKSLLAHKDETLEYKRQLAALGDKVTYQERLNALAQQADKFAQQQRAKRAAIDAKSRGLTDRQAEREATEQRLKEQYGDNPLALNNVMSEQKKTWAAEDQLRGSWMAGLKSGWSEWEESATDSMSQVKSAATQTFDGIAQNMAAMLTGSEQNWRSFTRSVLSMMTEILLKQAMVGIVGSIGSAIGGAVGGGASASGGTAIQAAAAKFHFATGGFTGTGGKYEPAGIVHRGEFVFTKEATSRIGVGNLYRLMRGYATGGYVGTPGSMADSRSQASGTFEQNNHVVINNDGTNGQIGPAALKAVYDMARKGARDEIQTQMRDGGLFSGGGR

SEQ ID NO: 63 - protein, 686 aa, Artificial Sequence
  Lambda-STF29
MAVKISGVLKDGTGKPVQNCTIQLKARRNSTTVVVNTVGSENPDEAGRYSMDVEYGQYSVILQVDGFPPSHAGTITVYEDSQPGTLNDFLCAMTEDDARPEVLRRLELMVEEVARNASVVAQSTADAKKSETAAASSKNAAKTSETNAANSAQAAAASQTASANSATAAKKSETSAKNSETATKASEKNAKSSQTAAKTSETNAKDSEANAKVSETAAANSAKASAASQTAAKASEDAAREYANQTAEPYRYVLQPLPDVWIPFNDSLDMITGYSPGYKKVKIGDNVVQVASDKQVNFSRASTATYINKSGELKTAEINEPRFEKEGLLIEGQRTNYMLNSATPASWGKSANMNVAEVGTDSFGFTYGKFVCNESLIGQSTTLNMAVVSTSGAVDVSGDNKCVTTSCRFKTDLELLLRIRFEAFDGSASSNLGYAIVNTRSLLVEITGVAADRLTARVNKDEATGWIFVEATIQASKETYITSAIQYAPKKGGVVESGDYIYLATPQVEDGSCVSSFIISGTTAATRASDMVTVPIKNNLYNLPFTVLCEVHKNWYKTPNAAPRVFDTGGHQTGAAIILGFGSSADGPDGFPYCDIGGSNRRVNENASLKKMVMGMRVKSDQSTCAVSNGRISSETKTTWEYIRSTATIRIGGQTTAGLRHLFGHVRNFRLWHKELTDAQLGEVVE

SEQ ID NO: 64 - protein, 824 aa, Artificial Sequence
  Lambda-STF118
MAVKISGVLKDGTGKPVQNCTIQLKARRNSTTVVVNTVGSENPDEAGRYSMDVEYGQYSVILQVDGFPPSHAGTITVYEDSQPGTLNDFLCAMTEDDARPEVLRRLELMVEEVARNASVVAQSTADAKKSAGDASASAAQVAALVTDATDSARAASTSAGQAASSAQEASSGAEAASAKATEAEKSAAAAESSKNAAATSAGAAKTSETNAAASQQSAATSASTAATKASEAATSARDAVASKEAAKSSETNASSSAGRAASSATAAENSARAAKTSETNARSSETAAERSASAAADAKTAAAGSASTASTKATEAAGSAVSASQSKSAAEAAAIRAKNSAKRAEDIASAVALEDADTTRKGIVQLSSATNSTSETLAATPKAVKVVMDETNRKAPLNSPALTGTPTTPTARQGTNNTQIANTAFVMAAIAALVDSSPDALNTLNELAAALGNDPNFATTMTNALAGKQPKDATLAALAGLATAADRFPYFTGNDVASLATLTKVGRDILAKSTVSAVIEYLGLQETVNRAGNAVQKNGDTLSGGLTFENDSILAWIRNTDWAKIGFKNDADGDTDSYMWFETGDNGNEYFKWRSRQSTTTKDLMNLKWDALYVLVKALFSSEVKISTVNALRIFNSSFGAIFRRSEENLYIIPTRENEGENGDIGPLRPFGINLRTGVVSVGNGARIDGGLALGTNNALGGNSIVLGDNDTGFKQNGDGNLDVYANNVHVMRFVSGSIQSNKTINITGRVNPSDYGNFDSRYVRDIRLGTRVVQTMQKGVMYEKAGHVITGLGIVGEVDGDDPAVFRPIQKYINGTWYNVAQV

SEQ ID NO: 65 - protein, 92 aa, Artificial Sequence
  Lambda-STF29 accessory protein
VRDFTLRFSDKADFRAFLRKLNWEEDEELQNAILVDEIGFTFSESGVSADGEPEYTRDEGYFVNIRLLDDGFDESVFREWVVTPERPLREWF

SEQ ID NO: 66 - protein, 175 aa, Artificial Sequence
  Lambda-STF118 accessory protein
MQHLKNITAGNPKTVAQYQLTKNFDVIWLWSEEGKNWYEEVSNFQEDTIKIVYDENNIIVGITRDASTLNPEGFSVVEVPDITSNRRADDSGKWMFKDGAVIKRIYTADEQEQQAESQKAALLSEAESVILPLERAVRLNMATDEERSRLEAWERYSVLVSRVDPANPEWPEMPQ